(19) United States
(12) Patent Application Publication
(10) Pub. No.: US 2013/0131465 A1
(43) Pub. Date: May 23, 2013

(54) BIOMEASUREMENT DEVICE, BIOMEASUREMENT METHOD, CONTROL PROGRAM FOR A BIOMEASUREMENT DEVICE, AND RECORDING MEDIUM WITH SAID CONTROL PROGRAM RECORDED THEREON

(71) Applicants: Yoshiro Yamamoto, Osaka-shi (JP); Norihiro Matsuoka, Osaka-shi (JP); Shinichiro Azuma, Osaka-shi (JP); Tomohisa Kawata, Osaka-shi (JP)

(72) Inventors: Yoshiro Yamamoto, Osaka-shi (JP); Norihiro Matsuoka, Osaka-shi (JP); Shinichiro Azuma, Osaka-shi (JP); Tomohisa Kawata, Osaka-shi (JP)

(73) Assignee: Sharp Kabushiki Kaisha, Osaka-shi, Osaka (JP)

(21) Appl. No.: 13/811,429

(22) PCT Filed: Jul. 14, 2011
(86) PCT No.: PCT/JP2011/066054

§ 371 Date: Jan. 22, 2013

(30) Foreign Application Priority Data
Jul. 26, 2010 (JP) 2010-167054
Jul. 26, 2010 (JP) 2010-167055
Jul. 26, 2010 (JP) 2010-167078
Jul. 26, 2010 (JP) 2010-167079
Jun. 29, 2011 (JP) 2011-144822

(51) Int. Cl. G06F 19/00; A61B 5/00

(52) U.S. Cl. CPC A61B 5/7271
USPC 600/301; 600/300

(57) ABSTRACT

An analysis device (1) of the present invention includes: an index calculating section (23) for, with use of one or more parameters including a biometric parameter obtained on the basis of biometric signal information, deriving measurement result information indicative of a state of a living body; and a measurement method storage section (31) for storing, in correspondence with each other, (i) a measurement item measurable by the analysis device and (ii) parameter specifying information specifying a parameter for use in measurement, the index calculating section (23) deriving the measurement result information for the measurement item with use of the parameter specified by the parameter specifying information corresponding to the measurement item.

### TECHNICAL FIELD

The present invention relates to a biometric device for measuring a state of a living body.

### BACKGROUND ART

There has been widely used a technique of sensing a living body with use of a sensor and measuring a state of the living body on the basis of signal information obtained from the sensor.

Patent Literature 1, for example, discloses a biometric information measuring device including (i) a sensor (sensor attachment head) to be attached to a body of a user and (ii) a main body for measuring, on the basis of signal information obtained from the sensor, a plurality of parameters (biometric information) of the user. This biometric information measuring device, for example, (i) detects an attachment site of the attached sensor so as to select a parameter measurable at the detected attachment site and (ii) adjusts, in correspondence with the attachment site, an amplification degree of a signal of the signal information outputted from the sensor. With this arrangement, Patent Literature 1 provides a biometric information measuring device that is not limited in terms of application or attachment site of a sensor and that can thus be widely used.

Patent Literature 2 discloses a wireless biometric information detecting system that uses a plurality of wireless biometric information sensor modules so as to detect and collect a continuous parameter (biometric information) regardless of time or place. This wireless biometric information detecting system compares (i) a parameter collected by a sensor module with (ii) a parameter collected by another sensor module, and thus evaluates and determines presence or absence of abnormality in a body.

Further, cough symptoms, as a specific example, have conventionally been diagnosed on the basis of self-reported information by a patient, and have not been evaluated objectively as a result.

In view of the above problem, there has been proposed, as disclosed in Patent Literature 3, a detecting device that evaluates a cough with high accuracy by (i) detecting a sound from a throat of a subject with use of a microphone and (ii) analyzing a frequency band included in the detected sound. Further, Patent Literature 4 discloses a cough detecting device that detects (i) a voice of a subject with use of a microphone and (ii) a body motion of the subject with use of an accelerometer so as to detect a cough on the basis of the voice and the body motion.

Alternatively, there have been known, as specific examples, a pulse oximetry method and a flow sensor method each as a simple examination method for sleep apnea syndrome. The pulse oximetry method checks for apnea by measuring a blood oxygen saturation (SpO₂) or a pulse. Patent Literatures 5 and 6 each disclose an example of such a method.

In addition, as disclosed in Patent Literature 7, it has been a common practice to increase measurement accuracy by measuring, besides a blood oxygen saturation, a breath sound, a snoring sound, a body motion or a posture. There has also been a simple examination method that uses a flow sensor for measuring an airflow through a mouth or a nose.

The technique disclosed in Patent Literature 5 displays a change in an index of apnea together with a change in other related physiological indexes (for example, an exercise amount, obesity information, and a blood pressure) so as to motivate a subject to do therapy to relieve a symptom of apnea syndrome.

### CITATION LIST

Patent Literature 1
Japanese Patent Application Publication, Tokukai, No. 2003-102692 A (Publication Date: Apr. 8, 2003)

Patent Literature 2
Japanese Patent Application Publication, Tokukai, No. 2005-160983 A (Publication Date: Jun. 23, 2005)

Patent Literature 3
Japanese Patent Application Publication, Tokukai, No. 2009-233103 A (Publication Date: Oct. 15, 2009)

Patent Literature 4
PCT International Publication No. 2007/040022, Pamphlet (Publication Date: Apr. 12, 2007)

Patent Literature 5
Japanese Patent Application Publication, Tokukai, No. 2008-5964 A (Publication Date: Jan. 17, 2008)

Patent Literature 6
Japanese Patent Application Publication, Tokukai, No. 2008-110108 A (Publication Date: May 15, 2008)

Patent Literature 7
Japanese Patent Application Publication, Tokukai, No. 2009-240610 A (Publication Date: Oct. 22, 2009)

### SUMMARY OF INVENTION

### Technical Problem

Conventional techniques (particularly Patent Literatures 1 and 2), however, merely (i) select, in correspondence with an attachment site, not to use a parameter that could not be measured or (ii) correct obtained signal information in correspondence with an attachment site. Thus, conventional techniques, in a case of carrying out a process of analyzing or recognizing a parameter obtained, carry out such a process with necessary information missing. Conventional techniques, as a result, problematically (i) fail to carry out a measurement for a particular measurement item (measurement purpose) and consequently (ii) output a measurement result having low accuracy. An inaccurate measurement result will in turn lead to a problem of a final determination being erroneous or determination accuracy being low.

The present invention has been accomplished in view of the above problem. It is an object of the present invention to provide (i) a biometric device, (ii) a biometric method, (iii) a program for controlling a biometric device, and (iv) a recording medium on which the control program is stored, each of which measures a state of a living body by a suitable method in correspondence with a measurement purpose so as to derive a measurement result having higher accuracy.

### Solution to Problem

In order to solve the above problem, a biometric device of the present invention is a biometric device for measuring a state of a living body with use of biometric signal information obtained from the living body, the biometric device including: measurement result deriving means for deriving, with use of one or more parameters including at least a biometric parameter obtained on a basis of the biometric signal information, measurement result information indicative of the state of the living body; and a measurement method storage section in which (i) a measurement item measurable by the biometric device and (ii) parameter specifying information specifying a parameter for use in measurement of the measurement item are stored in correspondence with each other, the measurement result deriving means deriving the measurement result information for the measurement item with use of the parameter specified by the parameter specifying information corresponding to the measurement item.

According to the above arrangement the biometric device stores, in the measurement method storage section, a measurement item and parameter specifying information in correspondence with each other. A measurement item refers to a purpose (that is, what state of a living body the biometric device is to measure) of measurement that can be carried out by the biometric device. In other words, a measurement item refers to a kind of measurement. Parameter specifying information refers to information that specifies a parameter to be used by the measurement result deriving means in deriving measurement result information in order to carry out a measurement for a measurement item.

The measurement result deriving means, in a case where the biometric device carries out a measurement for a measurement item, derives measurement result information indicative of a state of a living body with use of a parameter specified by parameter specifying information corresponding to the above measurement item.

The measurement result deriving means may use either a single parameter or a plurality of parameters in order to derive measurement result information. The one or more parameters to be used, however, include at least a biometric parameter obtained on the basis of biometric signal information obtained from the living body.

With the above arrangement, (i) the measurement result deriving means derives measurement result information with use of one or more parameters corresponding to a measurement item, and (ii) such one or more parameters always include a biometric parameter of the living body. Thus, the biometric device measures, in correspondence with a measurement purpose, a state of a living body with use of a parameter suited for the purpose, and can consequently derive a measurement result having higher accuracy.

In order to solve the above problem, a biometric method of the present invention is a biometric method for use by a biometric device for measuring a state of a living body with use of biometric signal information obtained from the living body, (i) a measurement item measurable by the biometric device and (ii) parameter specifying information specifying one or more parameters for use in measurement of the measurement item being stored in the biometric device in correspondence with each other, the parameter specifying information specifying at least one biometric parameter obtained on a basis of the biometric signal information, the biometric method including the steps of: (a) identifying the one or more parameters specified by the parameter specifying information corresponding to the measurement item; and (b) deriving, with use of the one or more parameters identified in the step (a), measurement result information indicative of the state of the living body, the state relating to the measurement item.

The biometric device may be in the form of a computer. In this case, the present invention encompasses in its scope (i) a program for controlling a biometric device, the program causing a computer to function as each of the means so as to provide the biometric device in the form of a computer and (ii) a computer-readable recording medium on which the above control program is stored.

### Advantageous Effects of Invention

In order to solve the above problem, a biometric device of the present invention is a biometric device for measuring a state of a living body with use of biometric signal information obtained from the living body, the biometric device including: measurement result deriving means for deriving, with use of one or more parameters including at least a biometric parameter obtained on a basis of the biometric signal information, measurement result information indicative of the state of the living body; and a measurement method storage section in which (i) a measurement item measurable by the biometric device and (ii) parameter specifying information specifying a parameter for use in measurement of the measurement item are stored in correspondence with each other, the measurement result deriving means deriving the measurement result information for the measurement item with use of the parameter specified by the parameter specifying information corresponding to the measurement item.

In order to solve the above problem, a biometric method of the present invention is a biometric method for use by a biometric device for measuring a state of a living body with use of biometric signal information obtained from the living body, (i) a measurement item measurable by the biometric device and (ii) parameter specifying information specifying one or more parameters for use in measurement of the measurement item being stored in the biometric device in correspondence with each other, the parameter specifying information specifying at least one biometric parameter obtained on a basis of the biometric signal information, the biometric method including the steps of: (a) identifying the one or more parameters specified by the parameter specifying information corresponding to the measurement item; and (b) deriving, with use of the one or more parameters identified in the step (a), measurement result information indicative of the state of the living body, the state relating to the measurement item.

The present invention can, as a result, measure a state of a living body by a suitable method in correspondence with a measurement purpose so as to derive a measurement result having higher accuracy.

### BRIEF DESCRIPTION OF DRAWINGS

FIG. 1 is a block diagram illustrating an essential configuration of an analysis device (biometric device) of an embodiment of the present invention.

FIG. 2 is a diagram schematically illustrating a configuration of a biometric system of an embodiment of the present invention.

FIG. 3A is a table illustrating a data structure of information stored in a measurement method storage section of the analysis device.

FIG. 3B is a table illustrating a data structure of information stored in a measurement method storage section of the analysis device.

FIG. 4 is a diagram illustrating how data flows between main members of the analysis device from (i) a time point at which the analysis device receives an instruction to start a biometric process to (ii) a time point at which the analysis device outputs a measurement result of the process.

FIG. 5 (a) through (d) are tables showing a specific example of an apnea degree calculation rule, and (e) is a table showing a specific example of assessment criterion information for an apnea degree.

FIG. 6 (a) through (d) are tables showing a specific example of a sleep depth calculation rule, and (e) is a table showing a specific example of assessment criterion information for a sleep depth.

FIG. 7 (a) through (d) are tables showing a specific example of an asthma severity calculation rule, and (e) is a table showing a specific example of assessment criterion information for an asthma severity.

FIG. 8 (a) through (d) are tables showing a specific example of a heart activity calculation rule, and (e) is a table showing a specific example of assessment criterion information for a heart activity.

FIG. 9 (a) through (d) are tables showing a specific example of a digestive organ activity calculation rule, and (e) is a table showing a specific example of assessment criterion information for a digestive organ activity.

FIG. 10 (a) through (d) are tables showing a specific example of a circulatory organ activity calculation rule, and (e) is a table showing a specific example of assessment criterion information for a circulatory organ activity.

FIG. 11 (a) through (d) are tables showing a specific example of a cough severity calculation rule, and (e) is a table showing a specific example of assessment criterion information for a cough severity.

FIG. 12 is a diagram illustrating an example display of a measurement result produced by the analysis device through a biometric process for measurement item “1: APNEA DEGREE MEASUREMENT”.

FIG. 13 is a diagram illustrating an example display of a measurement result produced by the analysis device through a biometric process for measurement item “2: SLEEP STATE MEASUREMENT”.

FIG. 14 is a diagram illustrating an example display of a measurement result produced by the analysis device through a biometric process for measurement item “3: ASTHMA MEASUREMENT”.

FIG. 15 is a diagram illustrating an example display of a measurement result produced by the analysis device through a biometric process for measurement item “4: HEART MONITORING”.

FIG. 16 is a diagram illustrating an example display of a measurement result produced by the analysis device through a biometric process for measurement item “5: DIGESTIVE ORGAN MONITORING”.

FIG. 17 is a diagram illustrating an example display of a measurement result produced by the analysis device through a biometric process for measurement item “6: CIRCULATORY ORGAN MONITORING”.

FIG. 18 is a diagram illustrating an example display of a measurement result produced by the analysis device through a biometric process for measurement item “7: COUGH MONITORING”.

FIG. 19 is a flowchart illustrating a flow of a biometric process carried out by the analysis device.

FIG. 20 is a diagram illustrating an example display, as a measurement result, of a long-term tendency of a state of a subject.

FIG. 21 is a block diagram illustrating an essential configuration of an analysis device (biometric device) of another embodiment of the present invention.

FIG. 22 is a table illustrating a data structure of information stored in a parameter attribute storage section of the analysis device.

FIG. 23 is a diagram illustrating an example display screen displayed in a display section to indicate a measurement result produced by the analysis device through a biometric process.

FIG. 24 is a diagram illustrating an example design screen for use by a user to design a calculation formula.

FIG. 25 is a table illustrating a data structure of information stored in a measurement method storage section of an analysis device (biometric device) of still another embodiment of the present invention.

FIG. 26 is a block diagram illustrating an essential configuration of an analysis device of an embodiment of the present invention.

FIG. 27 is a diagram schematically illustrating a configuration of a biometric system of an embodiment of the present invention.

FIG. 28 is a block diagram illustrating an essential configuration of an acoustic sensor.

FIG. 29 is a cross-sectional view illustrating a configuration of an acoustic sensor (an acoustic sensor or a sound sensor).

FIG. 30 is a diagram illustrating an example of an attribute information input screen displayed in a display section.

FIG. 31 is a diagram illustrating a specific example of a correspondence table that is stored in a measurement method storage section and that indicates a correspondence relationship between attribute information and algorithms.

FIG. 32 is a table showing specific examples of algorithms, stored in a measurement method storage section, for respective information processings.

FIG. 33 is a diagram illustrating an example of a measurement result information output screen displayed in a display section.

FIG. 34 is a flowchart illustrating a flow of a biometric process carried out by an analysis device of an embodiment of the present invention.

FIG. 35 (a) and (b) are each a diagram illustrating a waveform of sound data gathered by an acoustic sensor in a case where a heart sound is normal but an attachment state is poor.

FIG. 36 (a) is a diagram illustrating a frequency spectrum of sound data obtained through a fast Fourier transform (FFT) process for the sound data illustrated in (a) of FIG. 35, and (b) is a diagram illustrating a frequency spectrum of sound data obtained through a FFT process for the sound data illustrated in (b) of FIG. 35.

FIG. 37 (a) and (b) are each a diagram illustrating either (i) a waveform of sound data gathered by an acoustic sensor in a case where a heart sound is normal and an attachment state is good (improved) or (ii) a waveform of sound data stored in a sound source storage section 232 and serving as a sample of a normal heart sound.

FIG. 38 (a) is a diagram illustrating a frequency spectrum of sound data obtained through a FFT process for the sound data illustrated in (a) of FIG. 37, and (b) is a diagram illustrating a frequency spectrum of sound data obtained through a FFT process for the sound data illustrated in (b) of FIG. 37.

FIG. 39 (a) and (b) are each a diagram illustrating a waveform of sound data gathered by an acoustic sensor in a case where a heart sound is abnormal.

FIG. 40 (a) is a diagram illustrating a frequency spectrum of sound data obtained through a FFT process for the sound data illustrated in (a) of FIG. 39, and (b) is a diagram illustrating a frequency spectrum of sound data obtained through a FFT process for the sound data illustrated in (b) of FIG. 39.

FIG. 41 is a block diagram illustrating an essential configuration of an analysis device of another embodiment of the present invention.

FIG. 42 is a diagram illustrating a specific example of a correspondence table that is stored in an attachment position information storage section and that indicates a correspondence relationship between “MEASUREMENT SITE/MEASUREMENT ITEM” and “ATTACHMENT POSITION”.

FIG. 43 is a diagram illustrating an example of an attachment position input screen displayed in a display section of another embodiment of the present invention.

FIG. 44 is a diagram illustrating an example of an attachment position input screen displayed in a display section of another embodiment of the present invention.

FIG. 45 is a flowchart illustrating a flow of a biometric process carried out by an analysis device of another embodiment of the present invention.

FIG. 46 is a block diagram illustrating an essential configuration of an analysis device of still another embodiment of the present invention.

FIG. 47 is a table illustrating a data structure of a sound source database stored in a sound source storage section of an analysis device of still another embodiment of the present invention.

FIG. 48 is a flowchart illustrating a flow of a biometric process carried out by an analysis device of still another embodiment of the present invention.

FIG. 49 is a diagram illustrating an example of how a plurality of acoustic sensors of a biometric system of an embodiment of the present invention are attached.

FIG. 50 is a block diagram illustrating an essential configuration of an acoustic sensor of another embodiment of the present invention.

FIG. 51 is a table showing a specific example of attribute information for a plurality of acoustic sensors which attribute information is stored in an attribute information storage section of an analysis device of another embodiment of the present invention.

FIG. 52 is a diagram illustrating another example of how a plurality of acoustic sensors of a biometric system of an embodiment of the present invention are attached.

FIG. 53 is a table showing a specific example of carrier intensity information collected by an attachment position estimating section of an analysis device of still another embodiment of the present invention.

FIG. 54 is a table showing a specific example of attribute information that is stored in an attribute information storage section of an analysis device of still another embodiment of the present invention and that includes information on an approximate attachment position estimated by an attachment position estimating section.

FIG. 55 is a diagram schematically illustrating a configuration of a symptom detecting device of an embodiment of the present invention.

FIG. 56 is a flowchart illustrating an example flow of a process carried out by the symptom detecting device.

FIG. 57 is a table listing experimental results of an Example of the present invention.

FIG. 58 is a table listing experimental results of another Example of the present invention.

FIG. 59 shows the experimental results of FIG. 58 in graph form.

FIG. 60 is a diagram schematically illustrating a configuration of a measuring device of an embodiment of the present invention.

FIG. 61 (a) is a diagram illustrating a maximum value setting method, and (b) is a diagram illustrating an example of how an assessment sound changes as an amplitude value approaches its maximum.

FIG. 62 is a flowchart illustrating an example flow of a process carried out by the measuring device.

FIG. 63 is a diagram schematically illustrating a configuration of a measuring device of another embodiment of the present invention.

FIG. 64 is a flowchart illustrating an example flow of a process carried out by the measuring device.

### DESCRIPTION OF EMBODIMENTS

### Embodiment 1

### Embodiment 1-1

The following description will discuss an embodiment of the present invention with reference to drawings.

A biometric device of the present invention obtains biometric signal information from, for example, a sensor for sensing a state of a living body, and measures various states and symptoms of the living body with use of parameters obtained from the biometric signal information. In the present embodiment, the biometric device (i) senses, with use of a biometric sensor, a state of a human (hereinafter referred to as “subject”) as an example of a living body serving as an examinee of the biometric device, and (ii) measures a state and a symptom of the subject. However, the biometric device of the present invention is not limited to this. It is possible to measure a state of an animal (such as a dog) other than humans by dealing with the animal as an examinee (living body) and obtaining biometric signal information of the animal.

The present embodiment will discuss, as an example, a case where the biometric device of the present invention is in the form of an information processing device (such as a personal computer) which is provided separately from various sensors for obtaining the biometric signal information. Therefore, in the present embodiment, the biometric signal information obtained by the various sensors is supplied to a biometric device via appropriate wireless or wired communication means. However, the biometric device of the present invention is not limited to the above configuration, and may be contained in each of the various sensors themselves.

[Biometric System]

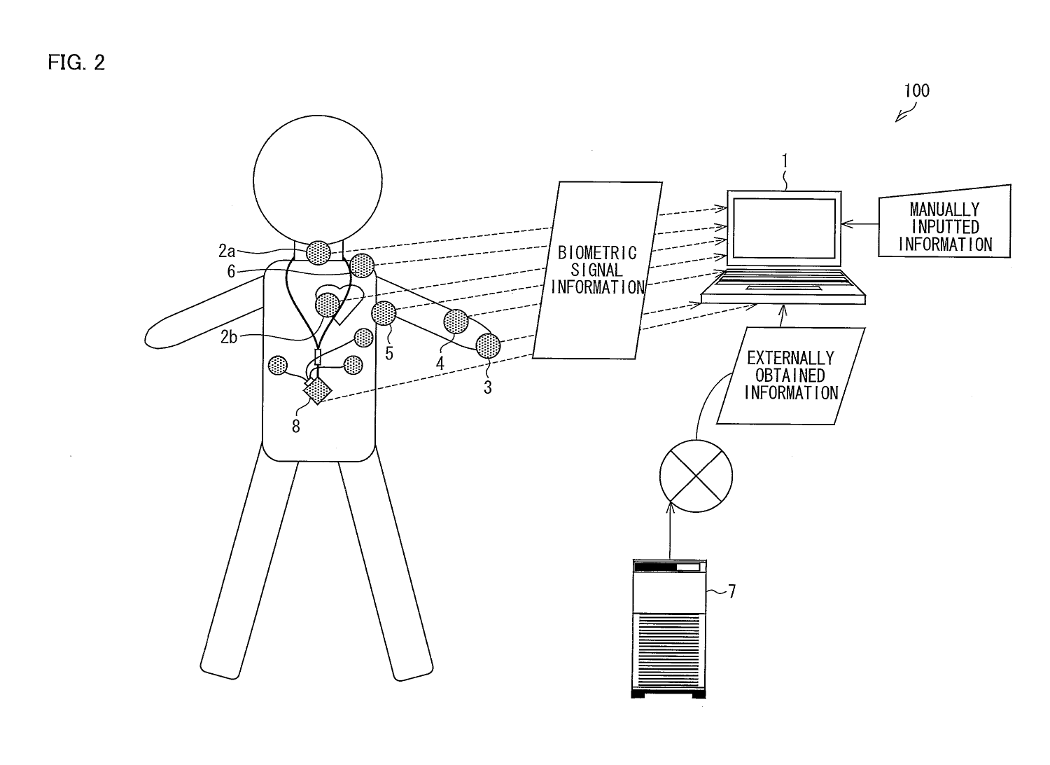

FIG. 2 is a diagram schematically illustrating a configuration of the biometric system 100 of the embodiment of the present invention. The biometric system 100 of the present invention may include at least one biometric sensor (2 to 6 and 8) and an analysis device (biometric device) 1. Further, as illustrated in FIG. 2, the biometric system 100 may include an information providing device 7 for providing various types of information regarding measurement of the subject.

A biometric sensor is a sensor for sensing a state of a subject and supplying detected biometric signal information to an analysis device 1. It is necessary to provide at least one biometric sensor, and as illustrated in FIG. 2, a plurality of biometric sensors may be provided. The example illustrated in FIG. 2 includes, as the plurality of biometric sensors, an acoustic sensor 2 (acoustic sensors 2a, 2b) for detecting sounds emitted from a subject, a pulse oximeter 3 for measuring percutaneous arterial blood oxygen saturation (SpO₂) of a subject, a pulse wave sensor 4 for detecting a pulse wave of a subject, a clinical thermometer 5 for measuring a body temperature of a subject, and an acceleration sensor 6 for detecting motion of a body (body motion) of a subject. Further, an electrocardiograph 8 for detecting an electrical activity of a heart of a subject may be provided as a biometric sensor. Various sensors transmit, to the analysis device 1, biometric signal information (such as a sound, SpO₂, pulse wave, body temperature, acceleration, and electrocardiogram) detected by the various sensors.

For example, the acoustic sensors 2a, 2b are contact microphones attached to a body of a subject to detect a sound emitted from the subject. A tackiness agent layer is provided on a surface of an acoustic sensor 2. Because of the tackiness agent layer, the acoustic sensor 2 can be attached to a body surface of the subject. A position for attaching the acoustic sensor 2 can be anywhere as long as the acoustic sensor 2 can pick up effectively an objective sound, and, for example, the acoustic sensor 2a for detecting a breath sound and a cough sound of a subject is attached near an airway, and an acoustic sensor 2b for detecting a heart sound, a heart rate, etc. of the subject is attached to a left portion of a chest region (as seen from the subject).

The acoustic sensor 2a transmits sound data of the breath sound detected to the analysis device 1 as biometric signal information. The acoustic sensor 2b transmits sound data of the heart sound detected to the analysis device 1 as the biometric signal information.

The pulse oximeter 3 includes an LED that emits red light and an LED that emits infrared light, and oxygen saturation in arterial blood is measured on the basis of light quantity of transmitted light that is generated such that light emitted from the LEDs has transmitted through a fingertip of the subject. Further, a pulse rate may be measured. The pulse oximeter 3 transmits, to the analysis device 1, measurement data, serving as the biometric signal information, in which measured SpO₂ and measuring time correspond to each other.

The electrocardiograph 8 detects an electrical activity of a heart. In the present embodiment, the electrocardiograph 8 is, similarly to other biometric sensors, used not for measuring a rest state (electrocardiogram) of a subject for a short time, but for continuously measuring a state of the subject in daily living. Accordingly, a Holter electrocardiograph is preferably employed as the electrocardiograph 8. The Holter electrocardiograph can continuously measure electrocardiogram of a subject in daily living for a long time (one day (24 hours) or longer). The electrocardiograph 8 includes electrodes to be attached to a body of a subject and a measuring instrument main body. The measuring instrument main body controls each electrode, analyzes an electric signal obtained from the each electrode, and creates an electrocardiogram. Further, in the present embodiment, the measuring instrument main body has a function of communicating with the analysis device 1, and transmits, to the analysis device 1, data of the created electrocardiogram serving as the biometric signal information. It should be noted that the electrocardiograph 8 is preferably compact and lightweight, and shaped to be excellent in portability so as not to interfere with a subject's daily living. The analysis device 1 can analyze an electrocardiogram supplied from the electrocardiograph 8, and extract a parameter showing an activity state of a heart such as a heart rate and a QRS width.

The analysis device 1 measures a state of a subject on the basis of biometric signal information obtained from the biometric sensor. The analysis device 1 extracts one or a plurality (various types) of information regarding a subject. Then, the subject is subjected to a biometric process with use of the one or plurality of information serving as a parameter(s). Thus, a measurement result can be obtained.

The analysis device 1 of the present invention can select or cancel, in accordance with a purpose of measurement (i.e., which state of a subject is to be measured; a measurement item), which parameter is used or is not used for the biometric process. This makes it possible to carry out an accurate assessment that meets a purpose of measurement.

Further, in order to improve accuracy of a measurement result of a biometric process, the analysis device 1 can extract a parameter for use from (i) externally obtained information obtained from devices (information providing device 7, etc.) other than a biometric sensor and (ii) manually inputted information directly inputted to the analysis device 1.

It should be noted here that a parameter obtained from the biometric signal information of the biometric sensor is referred to as “biometric parameter”, and that a parameter obtained from the externally obtained information or the manually inputted information is referred to as “external parameter”. These terms are used when two parameters need to be distinguished in terms of their properties.

The biometric parameter reflects a physiological state of a subject. Specific examples of the biometric parameter encompass “sound volume” and “frequency” obtained from sound data (biometric signal information) detected by the acoustic sensor 2. Further, in a case where a waveform is to be patterned, “presence or absence”, “length”, “the number”, etc. of the waveforms may be extracted as biometric parameters by analyzing a pattern of the waveform. Further, for example, “the number of heart rates”, “PP interval”, “RR interval”, “PQ time”, “QRS width”, “P wave height”, “P wave width”, “S wave height”, “S wave width”, “T wave height”, and “T wave width” may be extracted as biometric parameters from an electrocardiogram (biometric signal information) detected by the electrocardiograph 8. The biometric parameters are, however, not limited to the above.

The biometric parameter reflects a physiological state of a subject as described above, whereas the external parameter reflects an environmental condition outside the body. Specific examples of the external parameter encompass (i) specification information (for example, version information and what kind of information the biometric sensor functions to detect) of the biometric sensor, (ii) set position information (chest region, abdominal region, back, vicinity of airway, etc.) of the biometric sensor, (iii) subject (examinee) information (age, sex, hours of sleeping, previous mealtime, amount of exercise, history of disease, etc. of a subject) regarding the subject, and (iv) a measurement environment (ambient temperature, atmospheric pressure, humidity, etc.) in which the subject is present. The external parameter is, however, not limited to these.

The analysis device 1 derives a measurement result by appropriately combining the external parameter with the biometric parameter. This makes it possible to carry out accurate assessment that meets a purpose of measurement. The following description will discuss an arrangement of the analysis device 1 in more detail.

[Arrangement of Analysis Device 1]

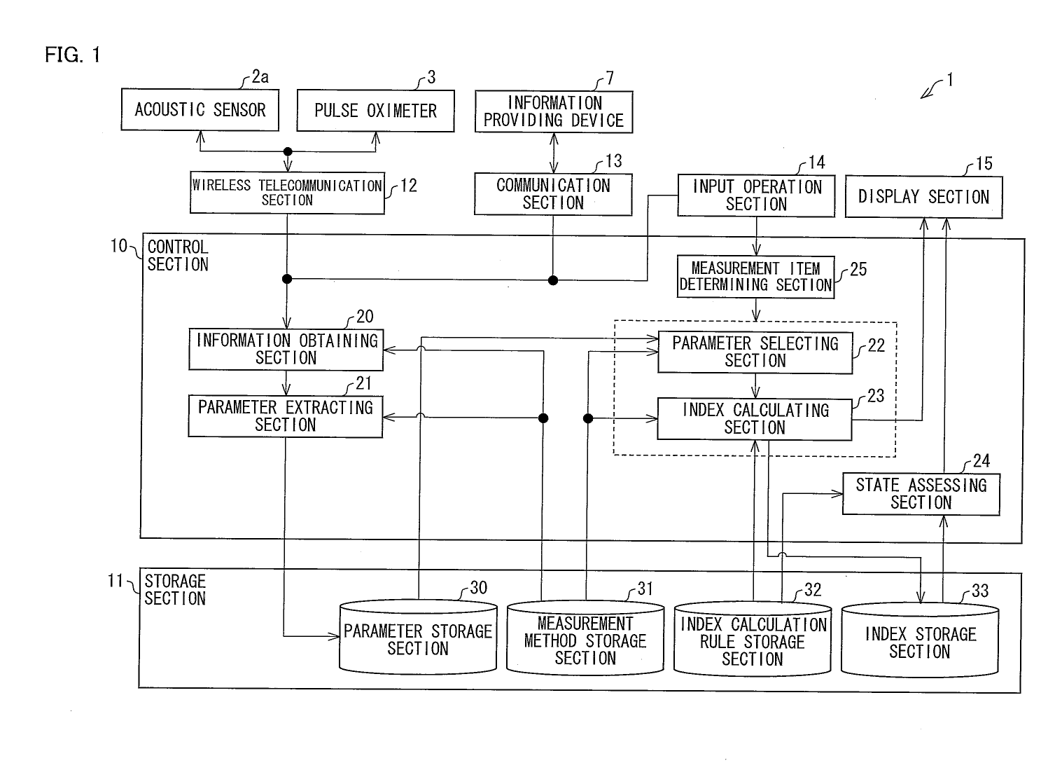

FIG. 1 is a block diagram illustrating an essential configuration of the analysis device 1 of an embodiment of the present invention.

As illustrated in FIG. 1, the analysis device 1 of the present embodiment includes a control section 10, a storage section 11, a wireless telecommunication section 12, a communication section 13, an input operation section 14, and a display section 15.

The wireless telecommunication section 12 wirelessly telecommunicates with various biometric sensors in the biometric system 100. It is assumed that a near field wireless telecommunications means such as Bluetooth® communication or WiFi communication, etc. are employed as wireless telecommunications means, and the wireless telecommunications means performs a near field wireless telecommunications directly with various biometric sensors. Alternatively, a LAN may be constructed so that the wireless telecommunication section 12 carries out wireless telecommunications with various biometric sensors via the LAN.

It should be noted that, in a case where the analysis device 1 communicates with biometric sensors with use of wired telecommunications, the analysis device 1 does not need to include the wireless telecommunication section 12. It is, however, preferable that communication between the analysis device 1 and each of the biometric sensors be carried out wirelessly. By using wireless telecommunications, attaching a biometric sensor to a subject is easier, and a restriction on a subject's activity is reduced under a measurement environment. This makes it possible to reduce a stress and burden on a subject.

The communication section 13 communicates with an external device (information providing device 7 or the like) via a wide area network. For example, the communication section 13 transmits/receives information to/from information providing device 7 via the Internet or the like. In particular, the analysis device 1 receives, from the information providing device 7 via the communication section 13, externally obtained information to be used to extract an external parameter for use in a biometric process. Examples of the externally obtained information obtained by the communication section 13 are assumed to encompass (i) information on weather, an ambient temperature, an atmospheric pressure, and humidity on a particular date, and (ii) specification information of biometric sensor(s) to be used. By referring to, for example, the specification information, the analysis device 1 can determine which parameter(s) of the biometric sensors should be used depending on a measurement item, or can learn compatibility and incompatibility of a plurality of biometric sensors when the plurality of biometric sensors are simultaneously used.

The input operation section 14 is used in order that a user (including a subject him/herself or an operator that carries out measurement) inputs an instruction signal to the analysis device 1. The input operation section 14 is constituted by an appropriate input device such as a keyboard having a plurality of buttons (arrow keys, enter key, character entry keys, etc.), a mouse, a touch panel, a touch sensor, a stylus, or a combination of a voice input section and a voice recognition section. In the present embodiment, a user directly inputs, to the analysis device 1, with use of the input operation section 14, information (manually inputted information) necessary to carry out measurement suitable for a purpose (measurement item) of measurement to be started. For example, parameters of a subject, such as age, sex, average hours of sleeping, hours of sleeping on a measurement date, previous mealtime, content of the meal, and amount of exercise, are inputted to the analysis device 1.

The display section 15 displays (i) a measurement result of a biometric process carried out by the analysis device 1 and (ii) as a GUI (graphical user interface) screen, an operation screen that a user uses to operate the analysis device 1. For example, a user displays (i) an input screen which is used to input the parameters, (ii) an operation screen through which the user designates a measurement item and instructs the start of measurement, and (iii) a result display screen for displaying the measurement result of a biometric process that has been carried out. The display section 15 is constituted by, for example, a display device such as an LCD (liquid crystal display).

The control section 10 carries out integrated control of sections that the analysis device 1 includes, and includes, as functional blocks, an information obtaining section 20, a parameter extracting section 21, a parameter selecting section 22, an index calculating section 23, a state assessing section 24, and a measurement item determining section 25. Each of these functional blocks can be provided in such a manner that a CPU (central processing unit) reads out, to a RAM (random access memory) (not shown) or the like, a program stored in a memory device (storage section 11) constituted by a ROM (read only memory), etc., and executes the program.

The storage section 11 stores various data read out when (i) a control program and (ii) an OS program both executed by the control section 10, (iii) an application program executed by the control section 10 in order to carry out various functions that the analysis device 1 has, and (vi) various data read out when the application program is executed. In particular, various programs and data to be read out when a biometric process is carried out by the analysis device 1 are stored in the storage section 11. Specifically, the storage section 11 includes a parameter storage section 30, a measurement method storage section 31, an index calculation rule storage section 32, and an index storage section 33.

It should be noted that the analysis device 1 includes a temporary storage section (not shown). The temporary storage section is a so-called working memory for temporarily storing, in the course of various kinds of processing carried out by the analysis device 1, data for use in calculation, a calculation result, etc., and is constituted by a RAM, etc.

The information obtaining section 20 of the control section 10 obtains various kinds of information necessary for a biometric process. Specifically, the information obtaining section 20 obtains biometric signal information from a biometric sensor via the wireless telecommunication section 12. Further, the information obtaining section 20 obtains externally obtained information from the information providing device 7 via the communication section 13. Further, the information obtaining section 20 obtains, via the input operation section 14, manually inputted information that has been inputted to the analysis device 1. For example, the information obtaining section 20 obtains, from the acoustic sensor 2a, sound data of a breath sound of a subject as biometric signal information.

In a case where a measurement item at the time when the analysis device 1 carries out a biometric process has been determined, the information obtaining section 20 may communicate with each of the biometric sensors, and check whether or not each of the biometric sensors necessary for the measurement of the measurement item is in a communicable state (active state).

The parameter extracting section 21 extracts, from various types of information obtained by the information obtaining section 20, a parameter for use in a biometric process. The parameter extracting section 21 extracts (i) a biometric parameter from biometric signal information obtained from the biometric sensor and (ii) an external parameter from either externally obtained information which is obtained from outside or manually inputted information inputted to the analysis device 1.

In the present embodiment, the parameter extracting section 21 is configured to extract a default parameter from predetermined biometric signal information. The parameter extracting section 21 is configured to extract, for example, “sound volume” and “frequency” from sound data. However, in a case where another parameter is needed because of a measurement item, the parameter extracting section 21 obtains, with reference to the measurement method storage section 31, such another parameter in accordance with an extraction method stored in the measurement method storage section 31. The expression “another parameter” refers to, for example, a maximum value among values of frequency detected during a period of n minutes, and is a parameter that is extracted through a more complicated analysis procedure. The parameter extracting section 21 stores, in the parameter storage section 30, each extracted parameter in correspondence with the obtained biometric signal information or the biometric sensor.

The measurement item determining section 25 determines a purpose of measurement of a biometric process that the analysis device 1 is to carry out, i.e., determines a measurement item. There are some methods for determining a measurement item. The simplest method is arranged such that the analysis device 1 presents measurable measurement items to a user via the display section 15, and causes the user to select a measurement items via the input operation section 14. The measurement item determining section 25 transmits, to sections of the analysis device 1, information on the measurement item specified by the user.

The parameter selecting section 22 selects a parameter necessary to carry out a biometric process for the measurement item specified by the user. The parameter selecting section 22 refers to parameter specifying information stored in the measurement method storage section 31, and selects a parameter that matches the measurement item specified.

Operations of the parameter selecting section 22 will be described later on the basis of a data structure of the measurement method storage section 31.

The index calculating section 23 calculates, with use of the parameter selected by the parameter selecting section 22, an index corresponding to the specified measurement item. The index calculating section 23 reads out an index calculation rule that (i) is stored in the index calculation rule storage section 32 and (ii) corresponds to the specified measurement item, and calculates an index of the specified measurement item in accordance with the index calculation rule.

For example, in a case where the specified measurement item is “apnea degree measurement”, the index calculating section 23 calculates the index “apnea degree” in accordance with an “apnea degree calculation rule” stored in the index calculation rule storage section 32. A data structure of the index calculation rule will be described later.

The index calculating section 23 causes the index calculated to be stored in the index storage section 33. It should be noted that, in a case where indexes are regularly calculated in a regular measurement, such indexes may each be stored in correspondence with a measurement date and information about a subject (examinee information).

The state assessing section 24 assesses a state of a subject on the basis of the index calculated by the index calculating section 23. Assessment criterion information is stored in the index calculation rule storage section 32, and the state assessing section 24 assesses, in accordance with the assessment criterion information, a state of a subject on the basis of an index calculated. For example, the state assessing section 24 assesses a state of the subject regarding the measurement item in three levels (specifically “NORMAL”, “CAUTION”, or “ABNORMAL”).

A measurement result supplied from the index calculating section 23 and the state assessing section 24 (that is, an index and a result of determination of a state of the subject) is supplied to the display section 15. This makes it possible to easily present a measurement result to the user.

The parameter storage section 30 stores parameters extracted by the parameter extracting section 21. The extracted parameters are managed in each type of the parameters so that the analysis device 1 can recognize the extracted parameters. The expression “type of the parameters” refers to, for example, “sound volume” and “frequency”. Further, in a case where a plurality of subjects are subjected to measurement with use of a plurality of biometric sensors, it is desirable to manage the parameters for each subject ID or for each biometric sensor ID.

The measurement method storage section 31 stores parameter specifying information in which a type of a parameter for use in a biometric process is specified for each measurement item.

In a case where attachment positions of the biometric sensors are, depending on measurement items, different from each other even if biometric sensors of an identical kind are used, the measurement method storage section 31 may store attachment position designation information for each measurement item and for each type of biometric sensor. Therefore, sections of the analysis device 1 can (i) detect an error (such as a case where a biometric sensor is not attached to an appropriate position, or a case where the sections cannot be communicated with a biometric sensor attached to an appropriate position) when a measurement item is specified, and can thus (ii) correct the error appropriately.

The measurement method storage section 31 may further store an eventually calculated index in correspondence with a measurement item. The index calculating section 23 can therefore recognize which index should be calculated when the measurement item is specified. In a case where, for example, the measurement item “apnea degree measurement” is specified, the index calculating section 23 recognizes that it is to calculate the index “apnea degree” corresponding to the “apnea degree measurement”.

Detailed description of a data structure of data stored in the measurement method storage section 31 will be described later with reference to drawings.

The index calculation rule storage section 32 is a section in which an index calculation rule to be used to calculate an index is stored for each measurement item. The index calculation rule shows an algorithm for all steps that end with a step of calculating an index with use of a selected parameter. In a case where, for example, the measurement item “apnea degree measurement” is specified, the index calculating section 23 can (i) read out the “apnea degree calculation rule” from the index calculation rule storage section 32, and (ii) calculate the index “apnea degree” in accordance with the algorithm indicated by the “apnea degree calculation rule”. Further, assessment criterion information for assessing a state of a subject on the basis of an index calculated is stored in the index calculation rule storage section 32 in correspondence with a measurement item. In a case where, for example, the index “apnea degree” has been calculated, the state assessing section 24 (i) refers to assessment criterion information for an apnea degree, and (ii) assesses, in accordance with the assessment criterion, a state of the subject regarding the measurement item “apnea degree measurement”.

Detailed description of a data structure of data stored in the index calculation rule storage section 32 will be described later with reference to drawings.

The index storage section 33 stores an index calculated by the index calculating section 23. It is preferable that indexes be regularly calculated and also that a calculated index be stored in correspondence with a measurement date and time and subject information. This makes it possible to observe a change of the same index of the same person over time, so that it is possible to assess a state (specifically, normal or abnormal) of the subject more accurately.

[As to Measurement Method Storage Section 31]

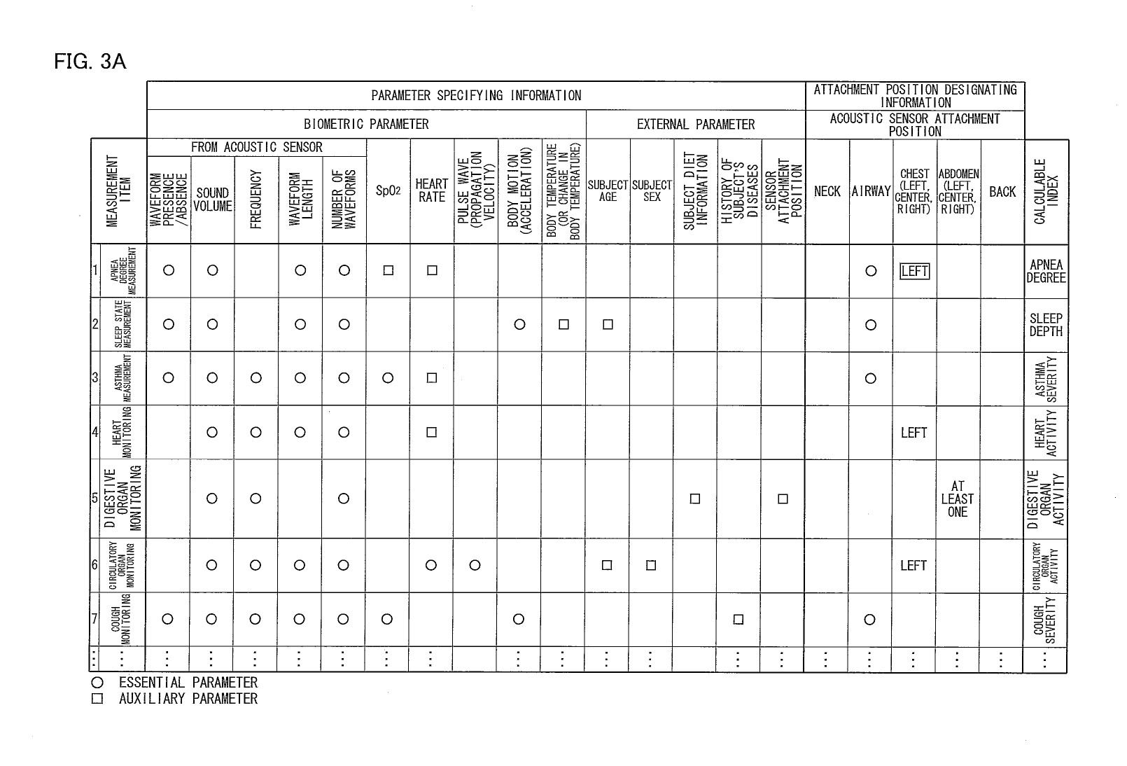

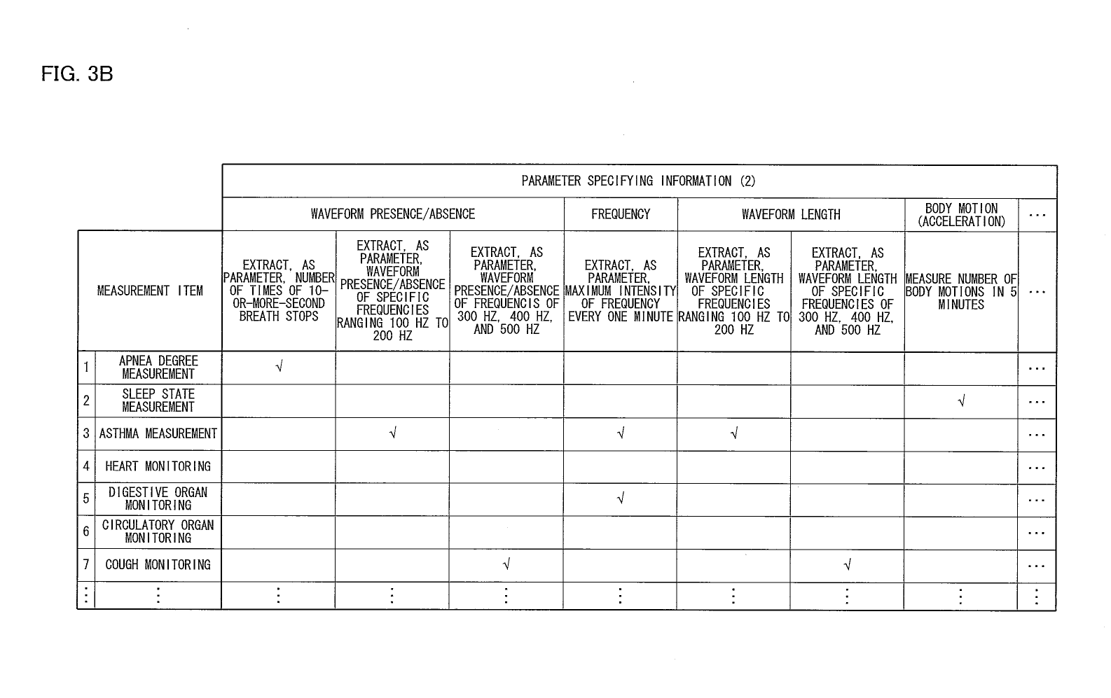

FIGS. 3A and 3B are tables each illustrating a data structure of information stored in the measurement method storage section 31. Specifically, FIG. 3A is a specific example showing a correspondence, with measurement items, of (i) parameter specifying information about versatile parameters, (ii) attachment position designating information, and (iii) corresponding indexes. FIG. 3B is a specific example showing a correspondence between parameter specifying information about special parameters and measurement items.

For each measurement item, a necessary parameter (hereinafter referred to as “essential parameter”) and a supplementary parameter (“supplementary parameter”) to improve accuracy correspond to each other as the parameter specifying information (see FIGS. 3A and 3B). In the examples shown in FIGS. 3A and 3B, a circle represents an essential parameter, and a square represents a supplementary parameter.

With the above arrangement, in a case where the measurement item determining section 25 has determined a measurement item, sections of the control section 10 that carry out a biometric process (particularly, the parameter selecting section 22) can learn, on the basis of the measurement item determined, a parameter necessary for the biometric process to be started.

In order to carry out, for example, a biometric process for the measurement item “1: APNEA DEGREE MEASUREMENT”, the sections can recognize that parameters indicative of presence or absence of a waveform, a sound volume, a waveform length, and the number of waveforms are essential, whereas parameters indicative of SpO₂ and a heart rate are arbitrarily used.

Further, in the present embodiment, a biometric sensor (particularly, the acoustic sensor 2) can be attached to various positions of a body of a subject, so that it is desirable that an optimal attachment position be determined in order to carry out an accurate measurement suitable for a measurement item. In view of the circumstances, as illustrated in FIG. 3A, the attachment position designating information is stored in correspondence with each measurement item.

For example, an acoustic sensor is necessarily attached to an airway in the example shown in FIG. 3A, so that each section of the control section 10 can recognize that it is to obtain essential parameters (indicative of presence or absence of waveform, a sound volume, a waveform length, and the number of waveforms) for a breath sound that can be collected from the vicinity of the airway.

Further, as illustrated in FIG. 3A, necessary parameters are stored as divided into biometric parameters and external parameters. This allows the information obtaining section 20 to recognize whether to obtain necessary information from (i) the biometric sensor or (ii) the information providing device 7 or an input by a user.

It should be noted that the present embodiment assumes that, as an example, biometric sensors to be used are determined in advance (FIG. 2), and that a correspondence between (i) those biometric sensors and (ii) parameters that can be extracted can be recognized in advance as described below.

It is possible to extract parameters indicative of presence or absence of waveform, a sound volume, a frequency, a waveform length, and the number of waveforms from biometric signal information of the acoustic sensor 2a (its attachment position may be any position, and is specified by the attachment position designating information). In a case where the acoustic sensor 2a is attached to a left portion of the chest, it is possible to extract a parameter indicative of a heart rate in addition to the above parameters.

It is possible to extract a parameter indicative of a heart rate from biometric signal information of the acoustic sensor 2b (its attachment position is fixed to the left portion of the chest).

It is possible to extract a parameter indicative of SpO₂ from biometric signal information of the pulse oximeter 3 (its attachment position is fixed to a fingertip). A parameter indicative of a pulse rate may be extracted in addition.

It is possible to extract parameters indicative of (i) a propagation velocity of a pulse wave and (ii) the number of pulse rates from biometric signal information of the pulse wave sensor 4 (its attachment position may be any position, and is specified by the attachment position designating information).

It is possible to extract parameters indicative of a body temperature and a change in body temperature from biometric signal information of the clinical thermometer 5 (its attachment position may be any position, and is specified by the attachment position designating information).

It is possible to extract a parameter indicative of body motion from biometric signal information of the acceleration sensor 6 (its attachment position may be any position, and is specified by the attachment position designating information).

As described above, in a case where attachment positions vary depending on a parameter intended to be extracted, optimal attachment positions for respective sensors other than the acoustic sensor 2a may be set in advance with use of the attachment position designating information. That is, the attachment position designating information is not limited to the example shown in FIG. 3A.

According to the above structure, in a case where a measurement item has been determined, the information obtaining section 20 of the analysis device 1 can recognize a parameter necessary for the measurement, and recognize from which biometric sensor a biometric information signal should be obtained. Further, the information obtaining section 20 can recognize a right attachment position of a biometric sensor, and present the right attachment position to a user.

However, a configuration of the analysis device 1 of the present invention is not limited to the above structure. In a use case where it is unnecessary to know a correspondence between a biometric sensor and a parameter, e.g., to recognize from which biometric sensor a parameter is to be obtained, only a correspondence between a measurement item and a parameter, i.e., which parameter is to be used for a measurement item, may be defined in the measurement method storage section 31 while the correspondence between the biometric sensor and the parameter is not stored. This makes it possible to simplify the configuration of the analysis device 1, and to reduce a processing load of the analysis device 1.

As illustrated in FIG. 3A, kinds of indexes which can be calculated for each measurement item may be stored in the measurement method storage section 31 in correspondence with the measurement item. The index calculating section 23 can therefore recognize which index should be calculated in a case where a measurement item has been determined.

As illustrated in FIG. 3B, in the present embodiment, a parameter for use in a particular measurement item may be (i) associated with a special parameter that is defined in detail in terms of how to extract the parameter and (ii) stored for each measurement item.

For example, the parameter “Presence or Absence of Waveform” is used for a biometric process regarding a measurement item “3: ASTHMA MEASUREMENT”. However, the presence or absence of waveform needs to be extracted as a parameter while the waveform is limited to a particular frequency of 100 Hz to 200 Hz.

As described above, for the parameter “Presence or Absence of Waveform”, which can be generally used for a large number of measurement items, a special parameter which limits a frequency, i.e., “Presence or Absence of Waveform Having a Particular Frequency of 100 Hz to 200 Hz”, is associated with the measurement item “3: ASTHMA MEASUREMENT”.

According to the above arrangement, the parameter selecting section 22 can decide that it is necessary to use a special parameter “Presence or Absence of Waveform Having a Particular Frequency of 100 Hz to 200 Hz” in a case where the measurement item “3: ASTHMA MEASUREMENT” is measured. If the parameter is not stored in the measurement method storage section 31, the parameter selecting section 22 requests the parameter extracting section 21 to extract the parameter “Presence or Absence of Waveform Having a Particular Frequency of 100 Hz to 200 Hz”.

The parameter extracting section 21 may be arranged to simultaneously extract all parameters (shown in FIGS. 3A and 3B) that are assumed to be needed. Alternatively, the parameter extracting section 21 may be arranged to extract both versatile parameters and special parameters in response to the request from the parameter selecting section 22.

As described above, however, it is preferable that the parameter extracting section 21 be arranged to extract a very versatile parameter(s) (shown in FIG. 3A) by default, and to extract a special parameter(s) (shown in FIG. 3B) if necessary in response to the request from the parameter selecting section 22.

According to the above arrangement, the parameter selecting section 22 may be ready to immediately obtain, from the parameter storage section 30, a versatile parameter whose extraction process is unlikely to result in vain. Meanwhile, since a special parameter is used only for a particular measurement item, the special parameter is extracted if necessary. Therefore, no extraction process for a special parameter results in vain.

The above arrangement makes it possible to reduce a processing load of the analysis device 1 and to improve a processing efficiency.

[Data Flow]

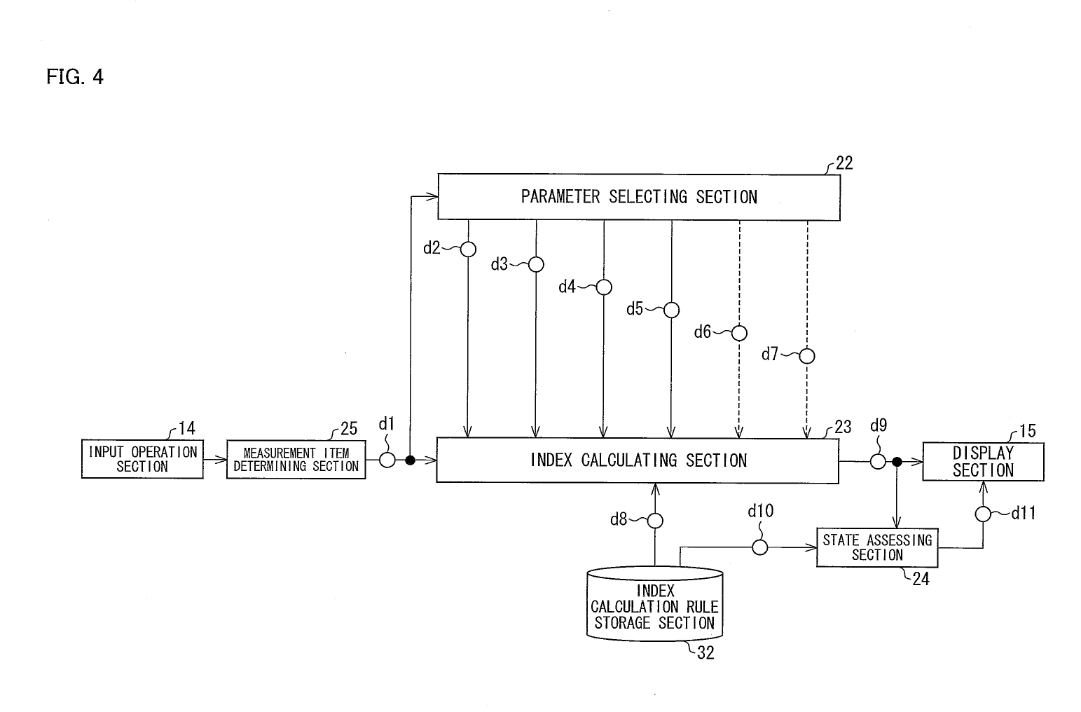

FIG. 4 is a diagram illustrating how data flows between main members of the analysis device 1 from (i) a time point at which the analysis device 1 receives an instruction to start a biometric process to (ii) a time point at which the analysis device outputs a measurement result of the process.

The following description will discuss a specific example in which the measurement item “1: APNEA DEGREE MEASUREMENT” has been selected.

The measurement item determining section 25 accepts, via the input operation section 14, not only an instruction to start a biometric process, but also information on a measurement item that a user has selected, and determines the measurement item as the “1: APNEA DEGREE MEASUREMENT”. The measurement item determining section 25 transmits the determined measurement item d1 to the parameter selecting section 22, the index calculating section 23, and the state assessing section 24.

The parameter selecting section 22 specifies necessary parameters by referring to the measurement method storage section 31 (FIGS. 3A and 3B) on the basis of the measurement item d1 transmitted, and obtains, from the parameter storage section 30, the parameters specified, i.e., presence or absence of waveform (breath) d2, (breath) sound volume d3, waveform (breath) length d4, the number of waveforms (breaths) d5, SpO₂ d6, and a heart rate d7. Then, the parameter selecting section 22 supplies the parameters to the index calculating section 23. In the present embodiment, the presence or absence of waveform (of breath) d2 indicates the number of times a subject stops breathing for 10 or more seconds (see FIG. 3B).

Among these, the SpO₂ d6 and the heart rate d7 are arbitrary and supplementary parameters, so that the SpO₂ d6 and the heart rate d7 may not be supplied to the index calculating section 23 if the parameter storage section 30 does not store the SpO₂ d6 and the heart rate d7.

The index calculating section 23 reads out, from the index calculation rule storage section 32, an index calculation rule on the basis of the measurement item d1 transmitted. In this example, the index calculating section 23 reads out an apnea degree calculation rule d8. The apnea degree calculation rule d8 shows an algorithm for calculating an apnea degree with use of the above parameters d2 through d7. The index calculating section 23 calculates an apnea degree d9 with use of the parameters d2 through d7 in accordance with the apnea degree calculation rule d8.

The state assessing section 24 reads out assessment criterion information for the calculated index from the index calculation rule storage section 32. In this example, the state assessing section 24 reads out assessment criterion information d10 on the apnea degree d9 calculated. The assessment criterion information d10 is information indicative of a determination criterion for assessing a state of a subject regarding apnea on the basis of the apnea degree d9. The state assessing section 24 assesses, (i) in accordance with the assessment criterion information d10 and (ii) on the basis of the apnea degree d9, whether the state or symptom of the subject regarding apnea is normal, caution, or abnormal, and outputs a state assessment result d11.

A measurement result indicative of the apnea degree d9 and the state assessment result d11 are supplied to and displayed on the display section 15. A user can therefore check a measurement result for a specified measurement item at the display section 15.

It should be noted that in a case where, for example, the analysis device 1 is contained in the biometric sensor and does not include the display section 15, it is impossible to output complicated information such as the apnea degree d9. In such a case, the analysis device 1 may include a light emitting section so as to notify a user of the state assessment result d11 by emitting light of green, yellow, red, or the like in accordance with a state assessment result. The light emitting section may alternatively be arranged to emit light in patterns such as starting emitting light, stopping emitting light, and blinking as appropriate in accordance with a state assessment result. Further, the analysis device 1 may alternatively include sound outputting section so as to notify a user of the state assessment result d11 with use of a sound or sound effect in accordance with a state assessment result.

The following description will discuss in detail a specific example of a data structure of the index calculation rule storage section 32 in which the apnea degree calculation rule d8 and the assessment criterion information d10 are stored.

[As to Index Calculation Rule Storage Section 32]

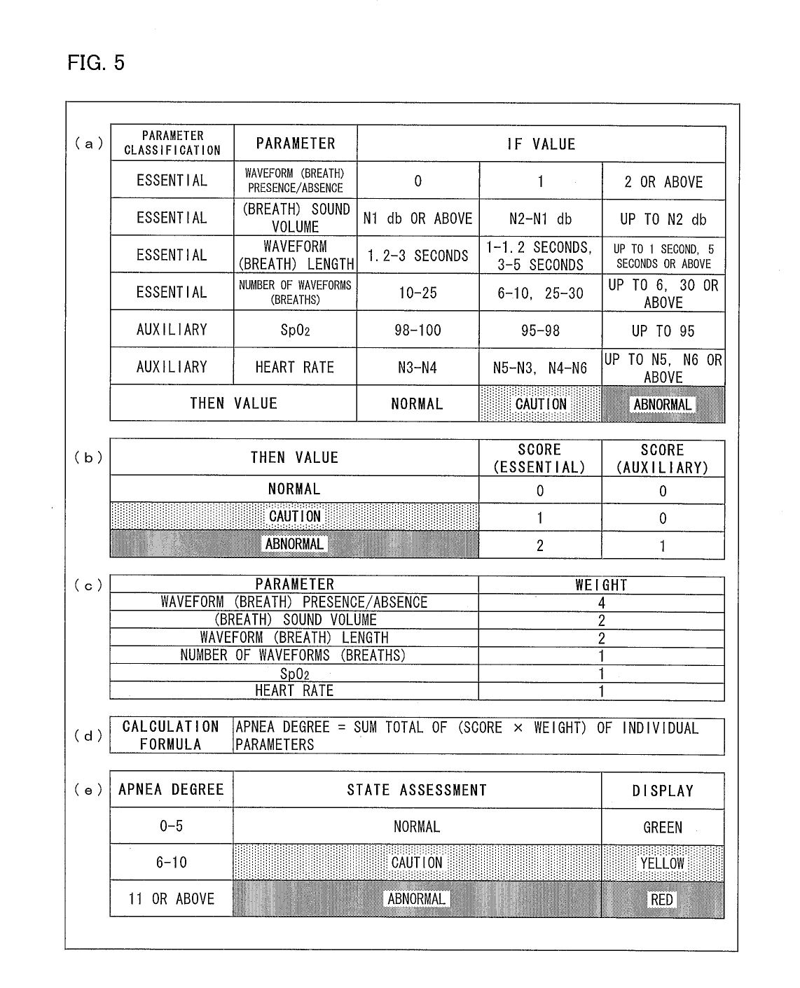

FIGS. 5 through 11 are tables each showing a data structure of an index calculation rule and assessment criterion information stored in the index calculation rule storage section 32. FIGS. 5 through 11 show specific examples of index calculation rules and assessment criterion information corresponding to respective seven measurement items shown in FIGS. 3A and 3B.

(a) through (d) of FIG. 5 are tables showing a specific example of an apnea degree calculation rule, and (e) of FIG. 5 is a table showing a specific example of assessment criterion information for an apnea degree.

Sleep apnea syndrome is a symptom in which a person falls into a state of apnea or hypopnea a predetermined or more times while he/she is sleeping. A criterion of a state of apnea can be when a person stops breathing by an airflow through a mouth or nose for 10 seconds or more, and a criterion of a state of hypopnea can be when the amount of ventilation is reduced to 50% or less for 10 seconds or more.

In order to detect such a state of apnea or hypopnea as above, it is possible to analyze (i) a sleeping stage with use of brainwaves, electro-oculogram, chin muscle electromyography, (ii) a breath pattern with use of an airflow through a mouth or nose and a motion of a chest/abdominal region, and (iii) a percutaneous arterial blood oxygen saturation (SpO₂) with use of a pulse oximeter.

In view of the above circumstances, the present embodiment uses, as parameters for assessment of an apnea degree, (i) the presence or absence of breath (the number of times a subject stops breathing for 10 or more seconds), (ii) a sound volume of a breath sound, (iii) the length of a breath (combination of a length of time of exhalation and a length of time of inhalation), (iv) the number of breaths per unit time period, and (v) a parameter indicative of SpO₂. In the present embodiment, as the “apnea degree” is higher, the possibility of sleep apnea syndrome is higher. It should be noted that the above example parameters for use in assessment of the apnea degree are merely examples, so that the present invention is not limited to the above examples. For example, a parameter indicative of a pulse rate can be used in addition to the above parameters.

As illustrated in (a) of FIG. 5, the apnea degree calculation rule contains a correspondence for evaluating each parameter obtained from the parameter selecting section 22 in three levels (which determine whether each parameter has a normal value, a caution value, or an abnormal value). The correspondence is tabulated in an example of (a) of FIG. 5. However, (a) of FIG. 5 is merely an example. Accordingly, (a) of FIG. 5 is not intended to limit the present invention.

Three thresholds (IF values) are stored for each parameter in correspondence with the parameter, and the three IF values are respectively associated with evaluation results (THEN values) in three levels of “NORMAL”, “CAUTION”, or “ABNORMAL”. That is, a THEN value of the parameter is determined depending on which of the three IF values the value of the parameter falls into.

In a case where, for example, a parameter outputted from the parameter selecting section 22 and indicative of the presence or absence of waveform (breath) d2 indicating the number of times a subject stops breathing for 10 or more seconds shows 0 (zero) times, the index calculating section 23 evaluates that the presence or absence of waveform (breath) d2 is “NORMAL” (IF d2=0, THEN d2=NORMAL). Similarly, the index calculating section 23 evaluates all the supplied parameters d2 through d7 by the three levels.

It should be noted that thresholds stored as IF values of the table are not limited to the example shown in (a) of FIG. 5, and may be set as appropriate on the basis of medical grounds or experiences.

As illustrated in (b) of FIG. 5, the apnea degree calculation rule contains score information for giving a score according to the evaluation to a parameter which has been evaluated by the three levels. In the example shown in (b) of FIG. 5, the score information is tabulated. However, (b) of FIG. 5 is merely an example, so that (b) of FIG. 5 is not intended to limit the present invention.

In accordance with the score information shown in (b) of FIG. 5, the index calculating section 23 gives scores to essential parameters as follows: 0 (zero) to a parameter evaluated as “NORMAL”; 1 to a parameter evaluated as “CAUTION”; and 2 to a parameter evaluated as “ABNORMAL”. That is, in the present embodiment, as to essential parameters, a total sum of scores is increased as the number of items evaluated as “ABNORMAL” regarding apnea is increased. As to an auxiliary parameter, parameters evaluated as “NORMAL” and “CAUTION” are each given a score of 0, and a parameter evaluated as “ABNORMAL” is given a score of 1.

In a case where a parameter indicative of the presence or absence of waveform (breath) d2 is evaluated as, for example, “NORMAL”, the parameter is given a score of “0” because the parameter indicative of the presence or absence of waveform (breath) d2 is essential.

As illustrated in (c) of FIG. 5, the apnea degree calculation rule may contain weighting information for giving a weight to a score calculated for each parameter. In the example shown in (c) of FIG. 5, weighting information is expressed in a table. However, (c) of FIG. 5 is merely an example. Accordingly, (c) of FIG. 5 is not intended to limit the present invention. Weighting information is stored in correspondence with each parameter. A large value of the weighting indicates that the parameter is information of greater importance and has much influence on calculation of the index.

In a case where the apnea degree is calculated in the example shown in (c) of FIG. 5, the presence or absence of waveform (breath) d2 indicative of the number of times a subject stops breathing for 10 or more seconds is important information that should be considered most carefully. Accordingly, a weighting thereof is set to “4”. On the contrary, less important parameters such as the number of waveforms (breaths), SpO₂, and the heart rate do not need to be given weightings, that is, the weightings thereof may each be set to 1″.

The parameter indicative of the presence or absence of waveform (breath) d2, which has been given the above score of “0”, is given a weighting of “4”, so that the final score of the parameter is “0×4=0”. The index calculating section 23 similarly performs the calculation “score×weighting value=final score” for each of the parameters d2 through d7.

As illustrated in (d) of FIG. 5, the apnea degree calculation rule has a mathematical formula to be used for calculating the index “apnea degree” on the basis of the score of each parameter. The mathematical formula of (d) of FIG. 5 is merely an example, so that the mathematical formula is not intended to limit to the present invention.

The index calculating section 23 calculates an apnea degree of each of the parameters d2 through d7 in accordance with the mathematical formula shown in (d) of FIG. 5 to thereby obtain a final score of each of the parameters d2 through d7.

Further, as illustrated in (e) of FIG. 5, assessment criterion information for assessing a state of a subject regarding the index “apnea degree” is stored in the index calculation rule storage section 32. In the example illustrated in (e) of FIG. 5, the assessment criterion information is expressed in a table. However, (e) of FIG. 5 is merely an example. Accordingly, (e) of FIG. 5 is not intended to limit the present invention.

In the table of the assessment criterion information as illustrated in (e) of FIG. 5, a state assessment result to be assessed corresponds to a value of the apnea degree calculated. The state assessing section 24 assesses a state regarding apnea of a subject in accordance with the assessment criterion information shown in (e) of FIG. 5. In a case where a result of the calculation of the apnea degree is, for example, “3”, the state assessing section 24 determines that the state of the apnea of the subject is “NORMAL”.

It should be noted that the table of the assessment criterion information may correspond to information defining a method for displaying the state assessment result. In the example shown in (e) of FIG. 5, for example, the state assessment result “NORMAL” corresponds to the display “GREEN”. This means that the state assessment result is displayed by characters in green or is notified by a green lamp. Since the state assessment result is supplied as color-coded as described above, a user can understand the state assessment result more intuitively.

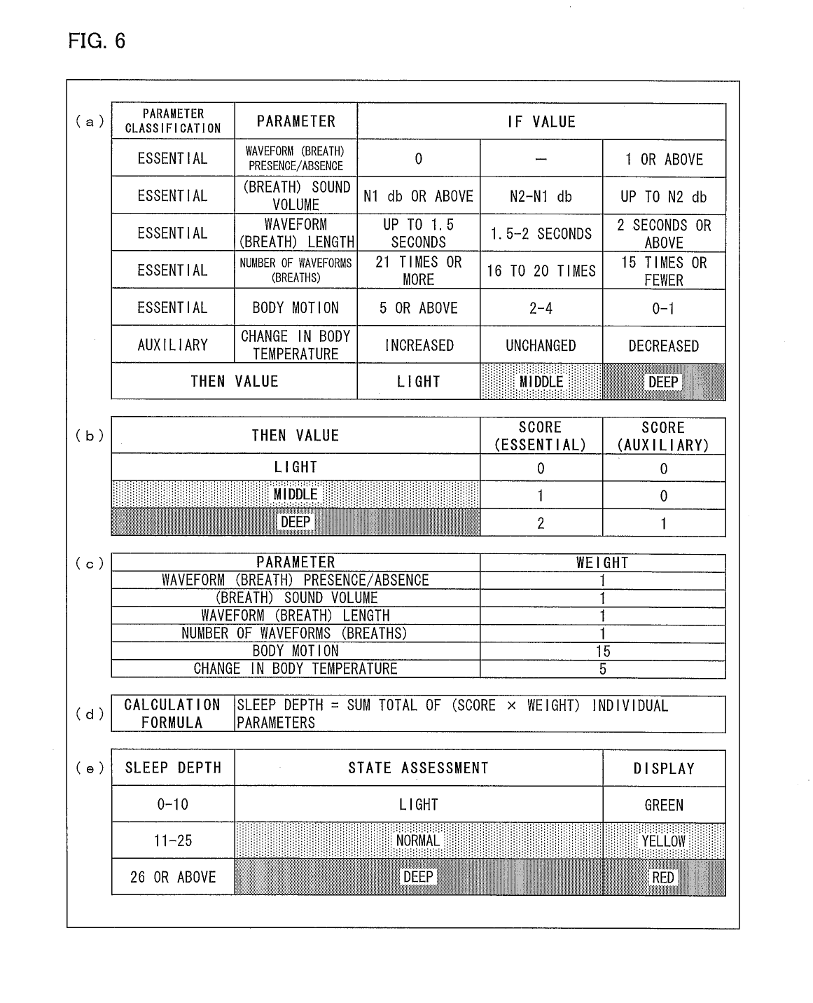

(a) through (d) of FIG. 6 are tables showing a specific example of a sleep depth calculation rule, and (e) of FIG. 6 is a table showing a specific example of assessment criterion information for a sleep depth. A larger value of “sleep depth” in the present embodiment indicates that a subject sleeps more deeply. A calculation procedure and a state determination procedure of the sleep depth based on various types of information shown in (a) through (e) of FIG. 6 are different from those of (a) through (e) of FIG. 6 in a parameter and a threshold to be used, and are similar to those of (a) through (e) of FIG. 6 in regard to the rest of the points. Accordingly, the description thereof will not be repeated. Note, however, that in a case where the sleep depth is assessed, lightness or deepness of sleep is assessed, instead of the presence or absence of abnormality.

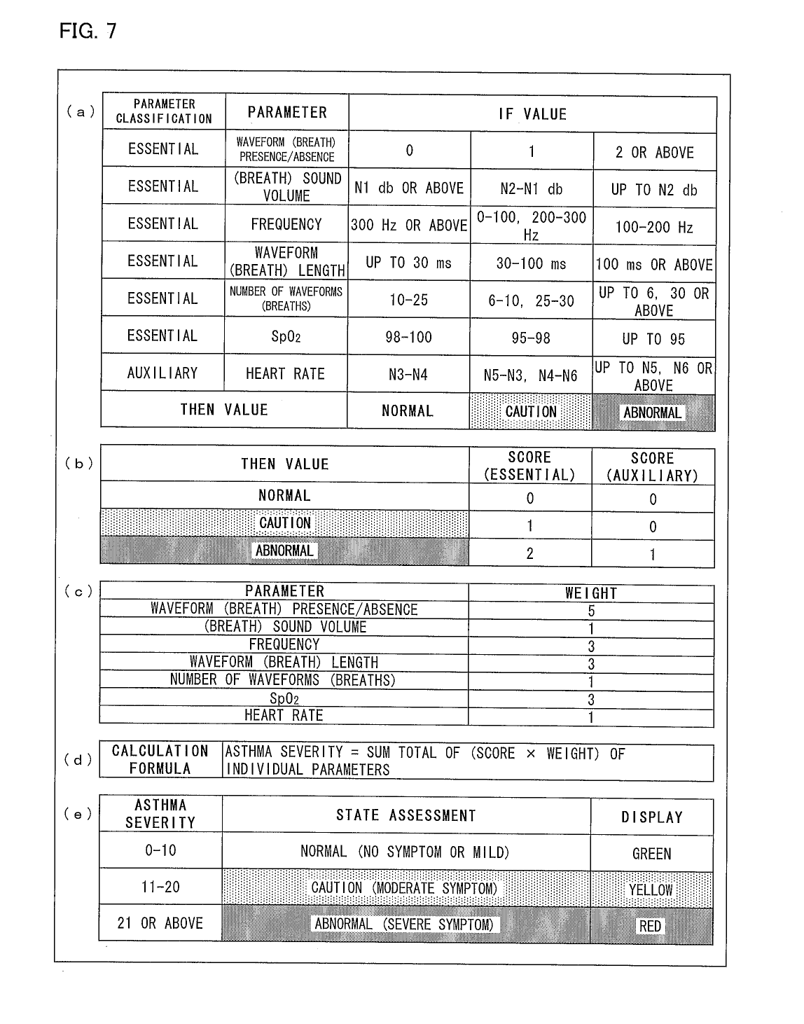

(a) through (d) of FIG. 7 are tables showing a specific example of an asthma severity calculation rule, and (e) of FIG. 7 is a table showing a specific example of assessment criterion information for an asthma severity. A larger value of “asthma severity” in the present embodiment indicates that a symptom of asthma is heavier. A calculation procedure and a state determination procedure of the asthma severity based on various types of information shown in (a) through (e) of FIG. 7 are different from those of (a) through (e) of FIG. 5 in a parameter and a threshold to be used, and are similar to those of (a) through (e) of FIG. 5 in regard to the rest of the points. Accordingly, the description thereof will not be repeated.

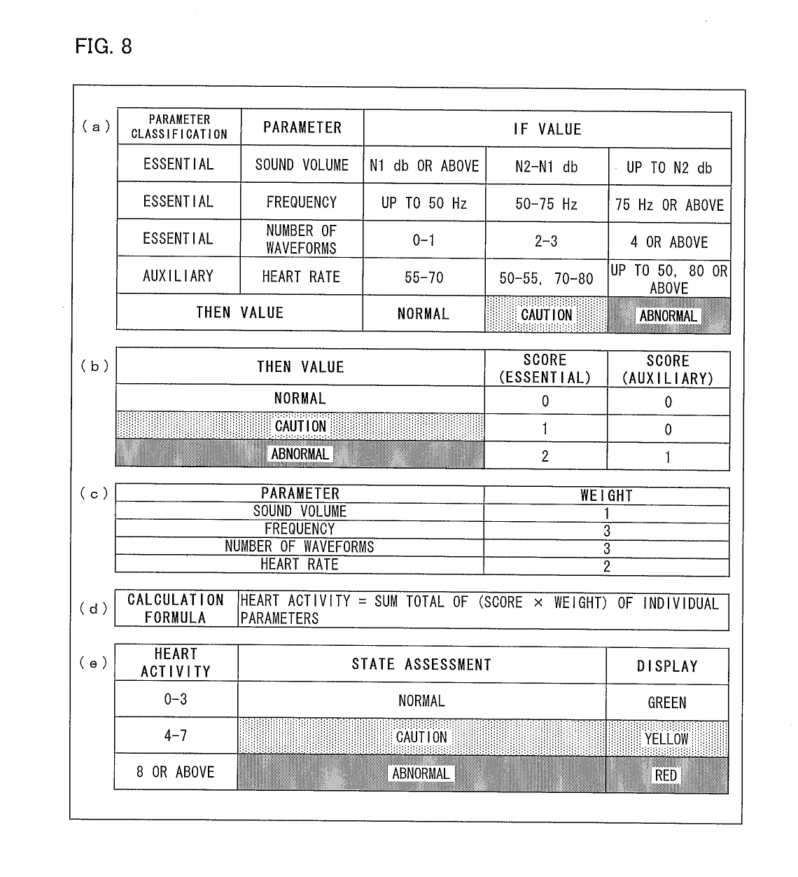

(a) through (d) of FIG. 8 are tables showing a specific example of a heart activity calculation rule, and (e) of FIG. 8 is a table showing a specific example of assessment criterion information for a heart activity. A larger value of “heart activity” in the present embodiment indicates that an activity of a heart is less stable, that is, the activity of the heart is abnormal. A calculation procedure and a state determination procedure of the heart activity based on various types of information shown in (a) through (e) of FIG. 8 are different from those of (a) through (e) of FIG. 5 in a parameter and a threshold to be used, and are similar to those of (a) through (e) of FIG. 5 in regard to the rest of the points. Accordingly, the description thereof will not be repeated.

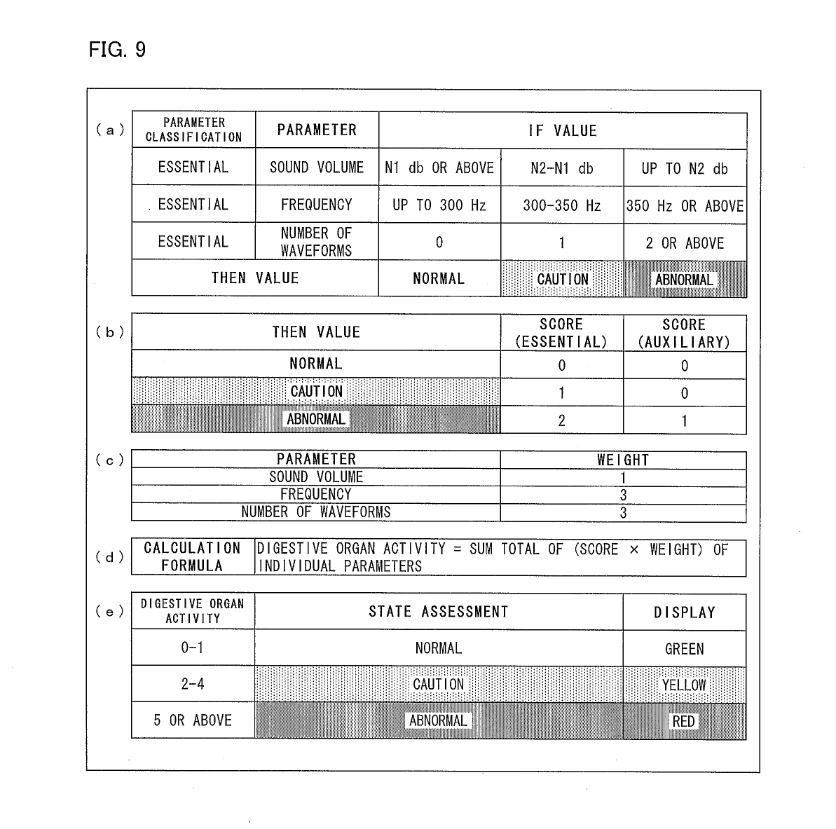

(a) through (d) of FIG. 9 are tables showing a specific example of a digestive organ activity calculation rule, and (e) of FIG. 9 is a table showing a specific example of assessment criterion information for a digestive organ activity. A larger value of “digestive organ activity” in the present embodiment indicates that an activity of a digestive organ is less stable, that is, the activity of the digestive organ is abnormal. A calculation procedure and a state determination procedure of the digestive organ activity based on various types of information shown in (a) through (e) of FIG. 9 are different from those of (a) through (e) of FIG. 5 in a parameter and a threshold to be used, and are similar to those of (a) through (e) of FIG. 5 in regard to the rest of the points. Accordingly, the description thereof will not be repeated.

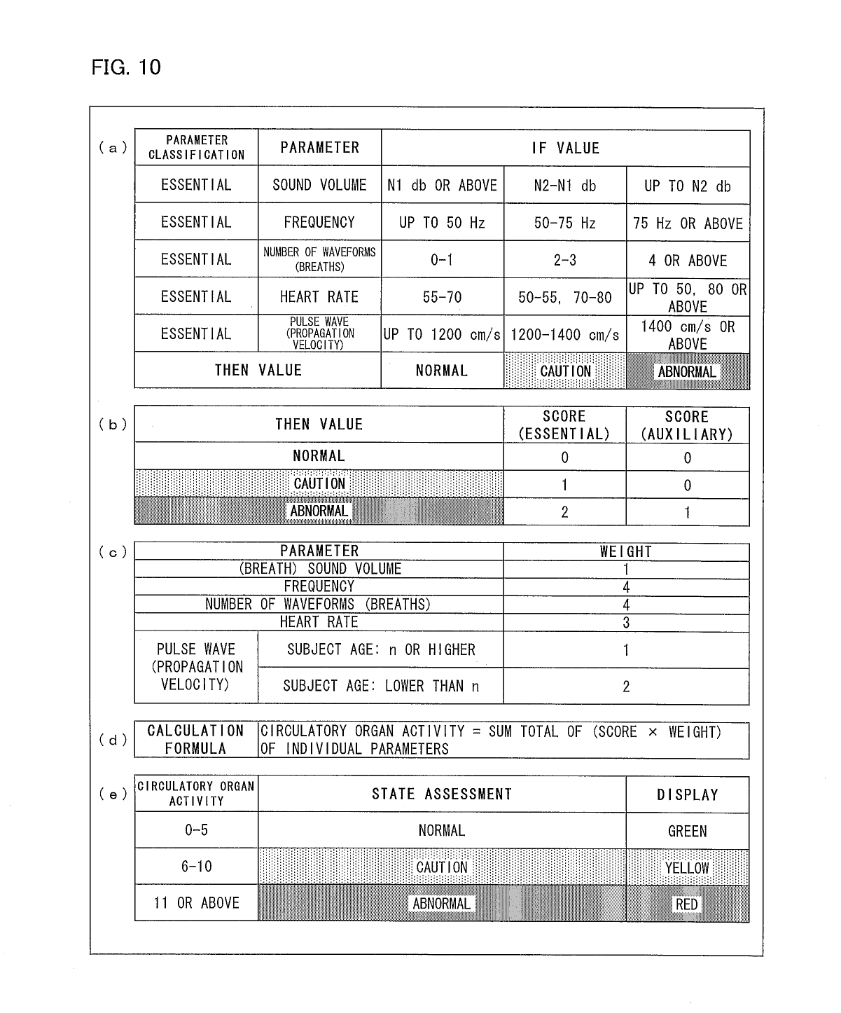

(a) through (d) of FIG. 10 are tables showing a specific example of a circulatory organ activity calculation rule, and (e) of FIG. 10 is a table showing a specific example of assessment criterion information for a circulatory organ activity. A larger value of “circulatory organ activity” in the present embodiment indicates that an activity of a circulatory organ is less stable, that is, the activity of the circulatory organ is abnormal. A calculation procedure and a state determination procedure of the circulatory organ activity based on various types of information shown in (a) through (e) of FIG. 10 are different from those of (a) through (e) of FIG. 5 in a parameter and a threshold to be used, and are similar to those of (a) through (e) of FIG. 5 in regard to the rest of the points. Accordingly, the description thereof will not be repeated.

It should be noted that in a case where the circulatory organ activity is to be calculated in the present embodiment, a subject's age may be used as an auxiliary external parameter. A state of health of circulatory organs (particularly, blood vessel) is largely affected by a subject's age. Accordingly, in the case where the circulatory organ activity is calculated in consideration of the subject's age, the state of the subject can be assessed so as to be suited for the subject's age.

For example, the IF values (threshold) of the essential parameter “PULSE WAVE (PROPAGATION VELOCITY)” shown in (a) of FIG. 10 may be changeable in accordance with a subject's age. More specifically, assume that, for example, the normal IF value “less than 1200 cm/s”, the caution IF value “1200 cm/s or more but less than 1400 cm/s”, and the abnormal IF value “1400 cm/s or more” shown in (a) of FIG. 10 are IF values of “subject's age=less than 30 years old”. In this case, “100” is added to each of the IF values shown in (a) of FIG. 10 when the subject's age is 30 years old or more but less than 40 years old, and “200” is added to each of the IF values shown in (a) of FIG. 10 when the subject's age is 40 years old or more but less than 50 years old”. Subsequently, it is considered that a threshold is corrected in accordance with the subject's age (by further adding “200” to the IF value as the subject's age increases by 10 years). That is, in a case where the subject is 51 years old, the normal IF value becomes “less than 1600 cm/s”.

Alternatively, as shown in, for example, (c) of FIG. 10, it is possible to calculate the circulatory organ activity more accurately by changing a weighting value of the parameter indicative of the pulse wave (propagation velocity) in accordance with the subject's age.

In the present embodiment, another index “arteriosclerosis degree” may be calculated with use of the parameter which is also used to calculate the circulatory organ activity. A mathematical formula for the arteriosclerosis degree may be additionally stored in the index calculation rule storage section 32 as an arteriosclerosis degree calculation rule.

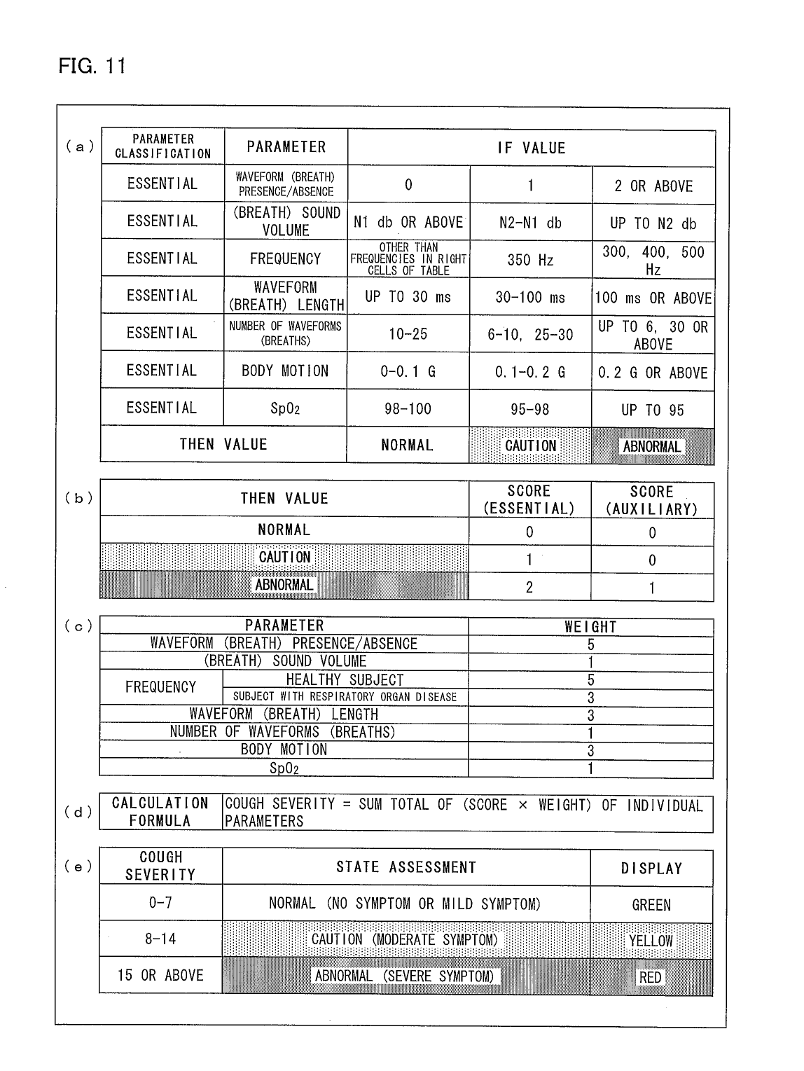

(a) through (d) of FIG. 11 are tables showing a specific example of a cough severity calculation rule, and (e) is a table showing a specific example of assessment criterion information for a cough severity. A larger value of “cough severity” in the present embodiment indicates that a symptom of cough is more serious, that is, it is highly possible that the symptom of cough is abnormal. A calculation procedure and a state determination procedure of the symptom of cough based on various types of information shown in (a) through (e) of FIG. 11 are different from those of (a) through (e) of FIG. 5 in a parameter and a threshold to be used, and are similar to those of (a) through (e) of FIG. 5 in regard to the rest of the points. Accordingly, the description thereof will not be repeated.

It should be noted that in the present embodiment, a history of a subject's diseases may be used as an auxiliary external parameter in order to calculate the cough severity. A patient of the respiratory disease often emits a characteristic cough (cough having a particular frequency), so that an influence of a cough caused by the original respiratory disease should be subtracted from the cough severity. Thus, it is possible to calculate the cough severity more accurately by changing, as shown in, for example, (c) of FIG. 11, a weighting value of a parameter indicative of a frequency depending on whether or not the subject is a patient of the respiratory disease.

As described above, the index calculating section 23 processes, in accordance the index calculation rule for the measurement item, a parameter which has been selected in accordance with a measurement item, and obtains an index by calculation. This makes it possible to carry out a biometric process that is suitable for the measurement item and has high accuracy.

[Measurement Result Display Example]

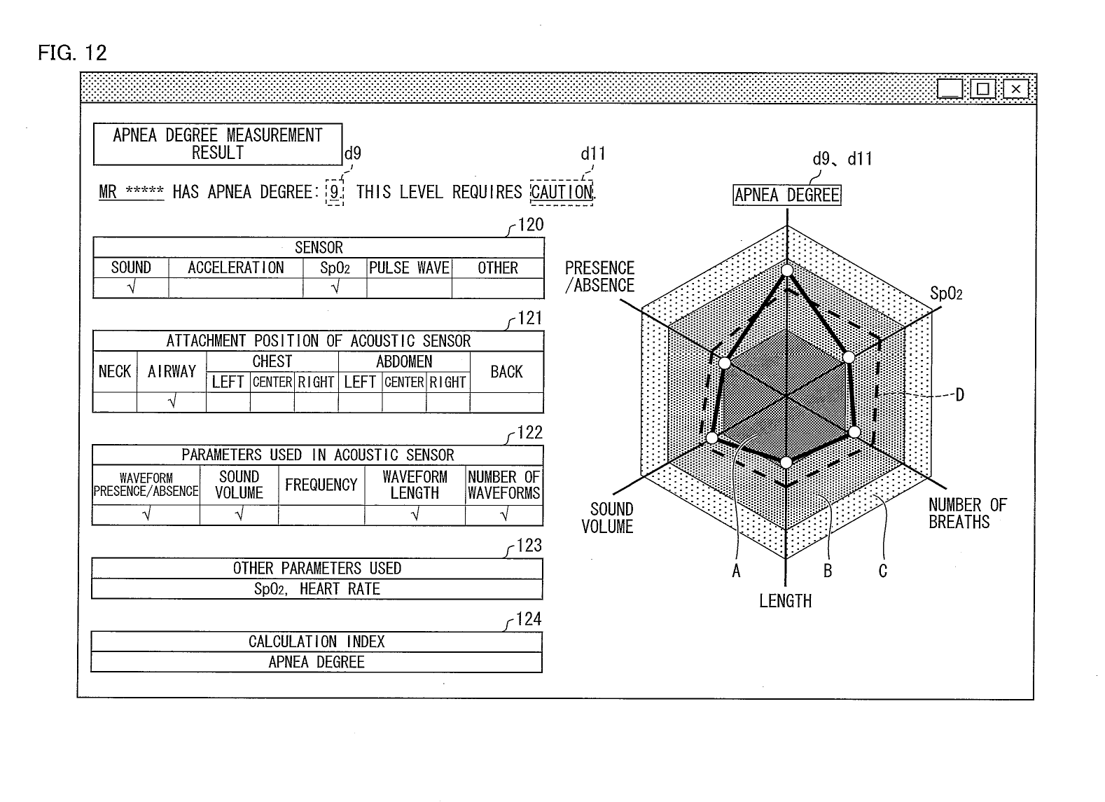

FIGS. 12 through 18 are diagrams each illustrating an example display screen in a case where a measurement result that the analysis device 1 obtains by carrying out a biometric process is displayed to the display section 15.

FIG. 12 is a diagram illustrating an example display of a measurement result produced by the analysis device 1 through a biometric process for measurement item “1: APNEA DEGREE MEASUREMENT”.

As illustrated in FIG. 12, at least the index calculated by the index calculating section 23 (herein referred to as “apnea degree d9”) and the state assessment result d11 as assessed by the state assessing section 24 are displayed as a measurement result. It is preferable that the apnea degree d9 and the state assessment result d11 be displayed in such a form as to be easily understood for a user. The apnea degree d9 and the state assessment result d11 may be displayed with use of sentences, or may be displayed with use of various graphs. For example, the measurement result may be displayed with use of sentences and a radar chart as illustrated in FIG. 12.

In the radar chart shown in FIG. 12, values are plotted on respective axes. The radar chart of FIG. 12 is such that (i) the calculated index is plotted on an axis extending upwardly from the center in a longitudinal direction, (ii) parameters which have been used for calculating the index are plotted on axes extending to other directions, (iii) 0 (zero) is set to the center, and (iv) ends of the axes are set to maximum values which can be potentially obtained from the calculation. In this case, regions of “NORMAL”, “CAUTION”, and “ABNORMAL” may be plotted in the radar chart in advance so that a user can easily understand evaluation of each value in the three levels.

As a value of the calculated index is smaller, that is, as the calculated index is closer to the center of the radar chart, the calculated index indicates “NORMAL”. Accordingly, the region A, which is the closest to the center, means “NORMAL”, the intermediate region B indicates “CAUTION”, and the outer region C indicates “ABNORMAL”.

However, depending on a parameter to be used, a value may be “NORMAL” in the intermediate region, and the value, if too small or too large, may be “CAUTION” or “ABNORMAL”. For such a parameter, the region A, which is the closest to the center, and the outer region C each indicate “ABNORMAL”, and the intermediate region B indicates “NORMAL”. Further, a vicinity of a boundary between the region A and the region B, and a vicinity of a boundary between the region A and the region C indicates “CAUTION”.

As a matter of course, boundary positions of the regions are changed by assessment criterion information on an index or IF values of respective parameters. Accordingly, lengths of respective axes from the center to the boundary positions may be different from each other. Further, all the axes on which the index and the parameters are plotted do not need to be placed on a same plane, and a plurality of radar charts can be created and displayed in a case where a display region is large.

Further, a nationwide mean value, an ideal value, a previous measurement value of the same subject, etc. may be plotted and displayed as with a broken line D so that those values can be compared with a measurement result (solid line) of this time.

Further, the information obtaining section 20, the parameter selecting section 22, and the index calculating section 23 may supply, to the display section 15, various types of information obtained by referring to the measurement method storage section 31. For example, the information obtaining section 20 in the example shown in FIG. 12 displays (i) information 120 indicative of a type of a biometric sensor which has been used (communicated) for measurement of the measurement item “apnea degree measurement” and (ii) information 121 indicative of an attachment position of the biometric sensor in a case where the attachment position has been specified by attachment position designating information. The parameter selecting section 22 displays, as information on the measurement item “apnea degree measurement”, (i) information 122 on a parameter selected as an essential parameter and (ii) information 123 on a parameter selected as an auxiliary parameter. The index calculating section 23 displays information 124 on an index corresponding to the measurement item “apnea degree measurement”.

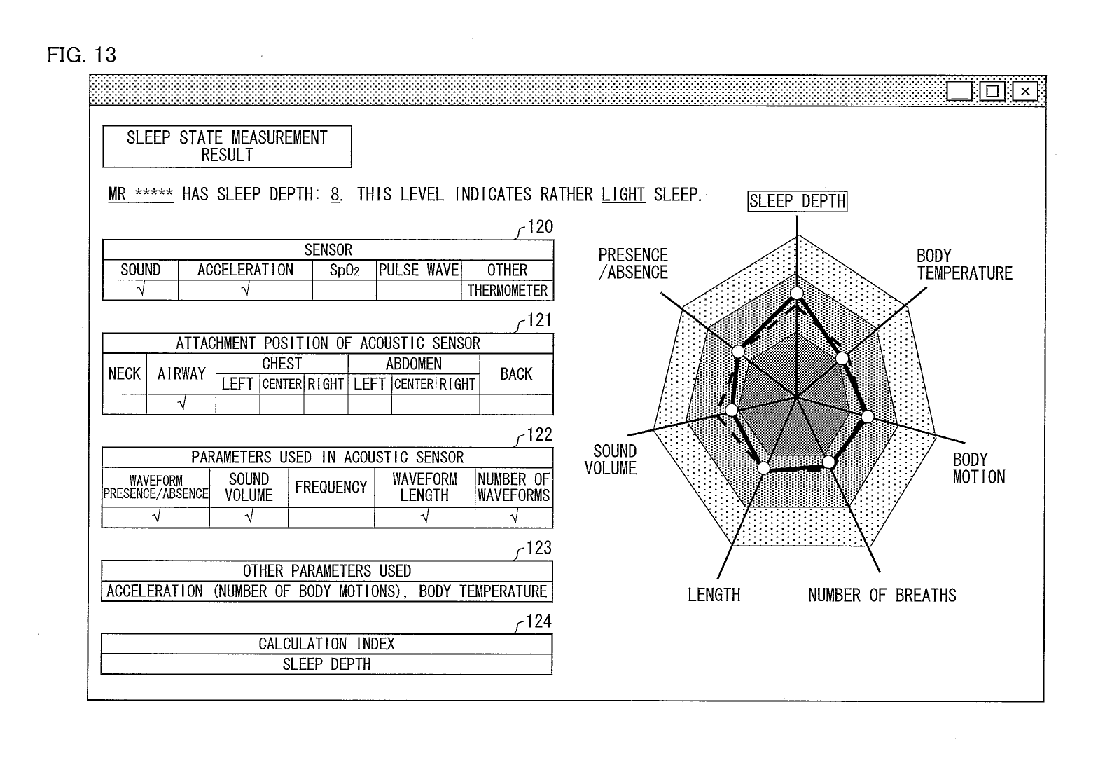

FIG. 13 is a diagram illustrating an example display of a measurement result produced by the analysis device 1 through a biometric process for measurement item “2: SLEEP STATE MEASUREMENT”.

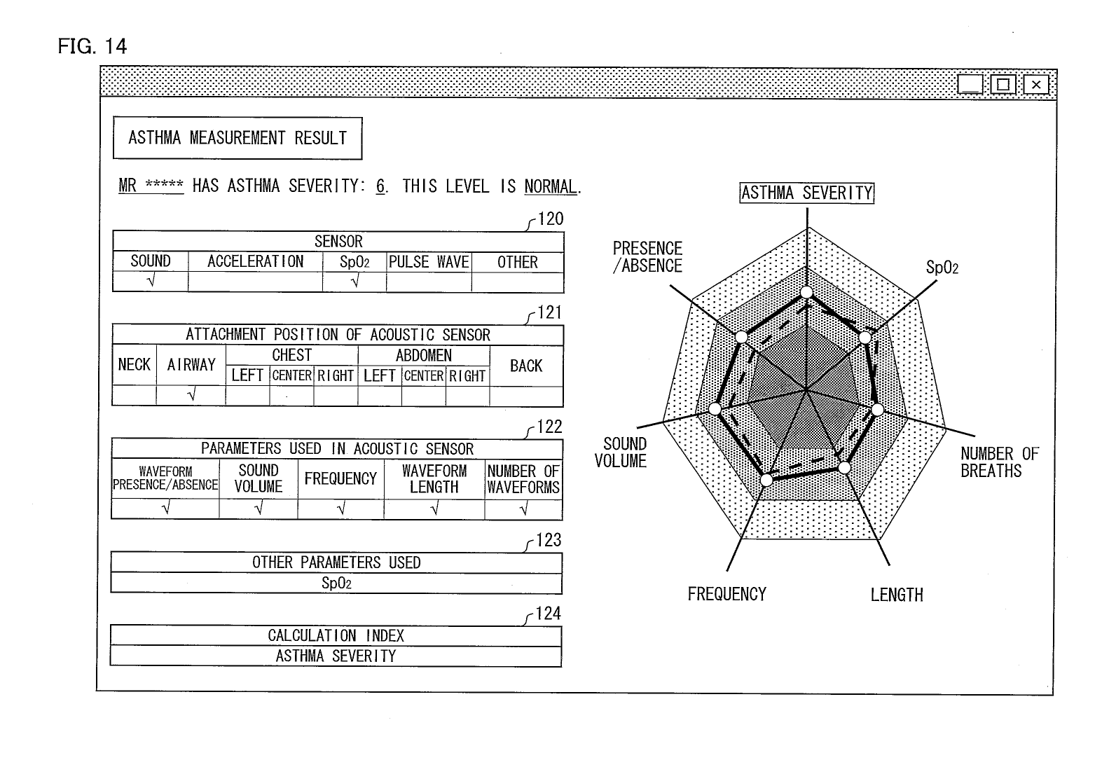

FIG. 14 is a diagram illustrating an example display of a measurement result produced by the analysis device 1 through a biometric process for measurement item “3: ASTHMA MEASUREMENT”.

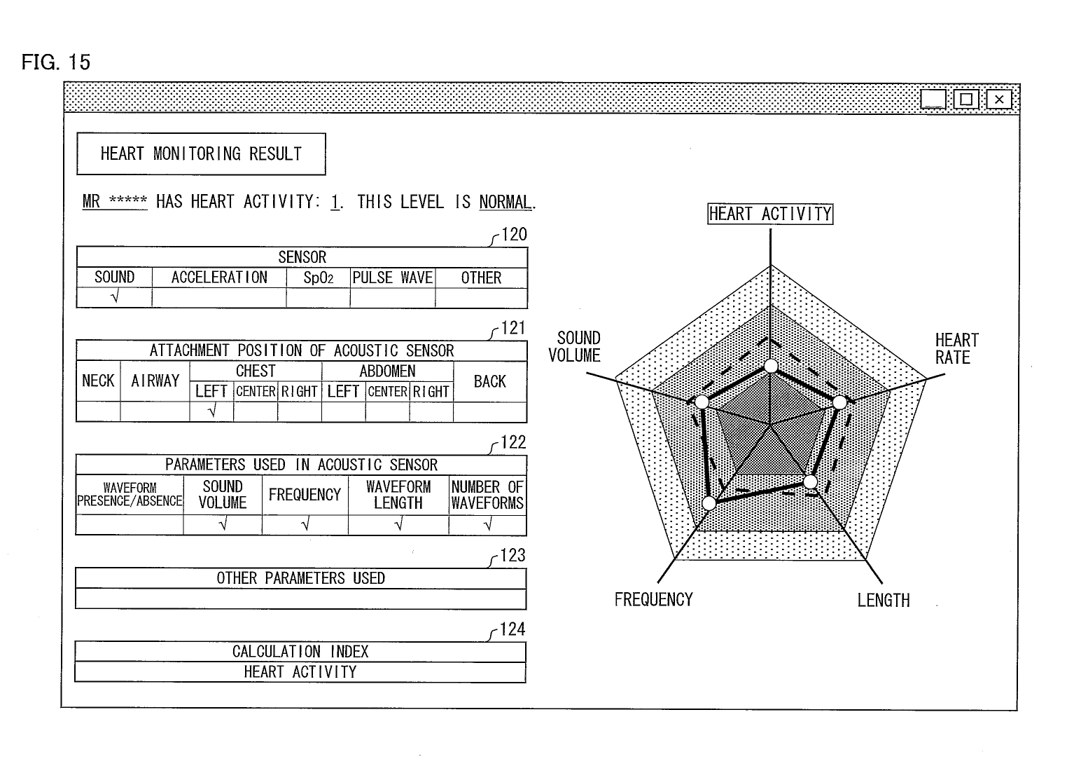

FIG. 15 is a diagram illustrating an example display of a measurement result produced by the analysis device 1 through a biometric process for measurement item “4: HEART MONITORING”.

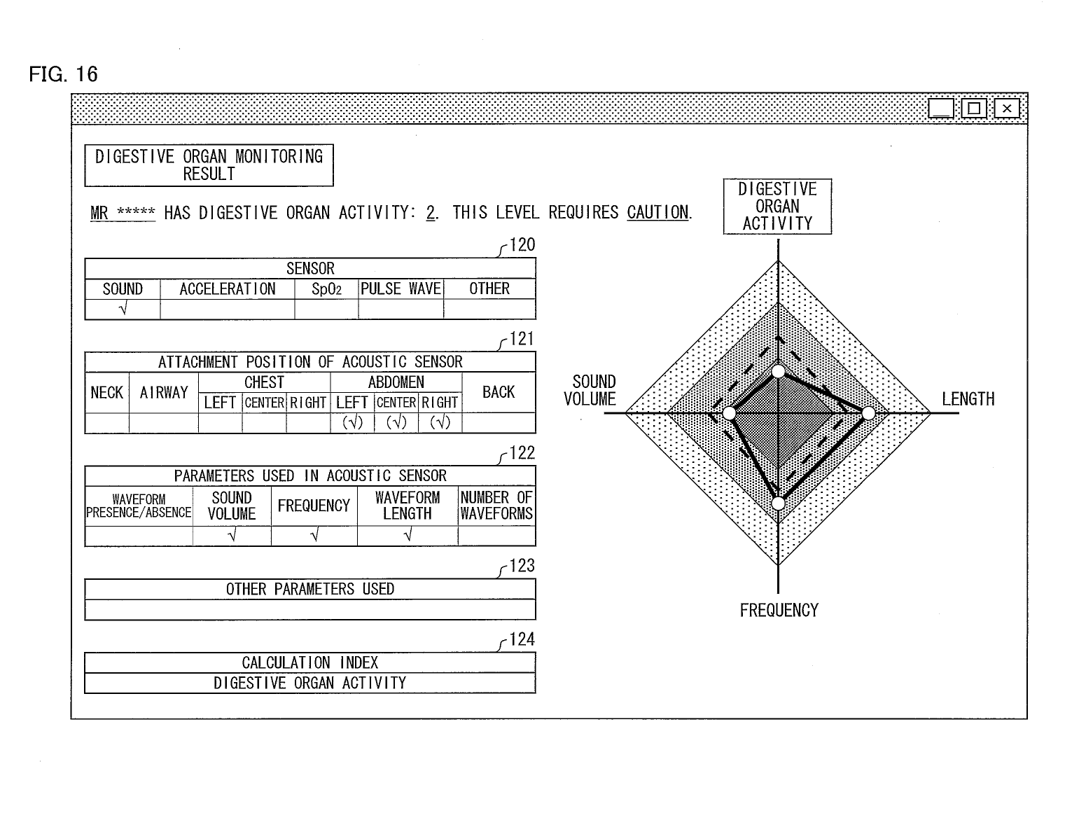

FIG. 16 is a diagram illustrating an example display of a measurement result produced by the analysis device 1 through a biometric process for measurement item “5: DIGESTIVE ORGAN MONITORING”.

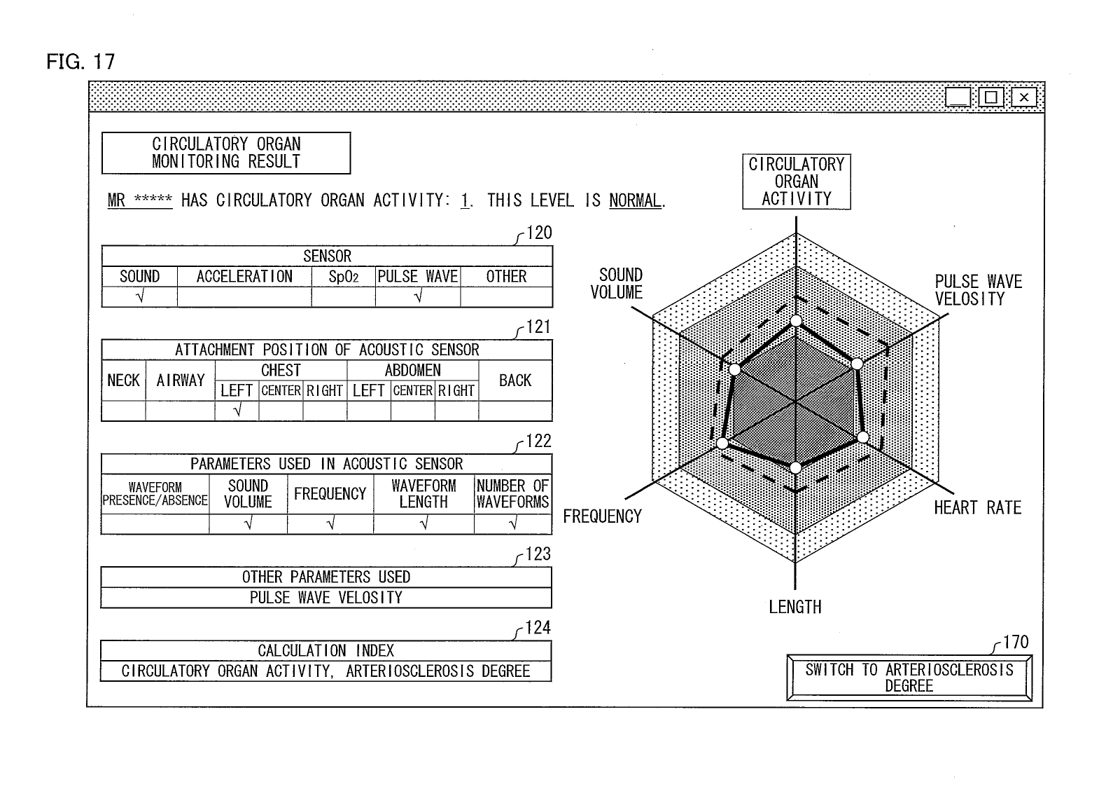

FIG. 17 is a diagram illustrating an example display of a measurement result produced by the analysis device 1 through a biometric process for measurement item “6: CIRCULATORY ORGAN MONITORING”. In the present embodiment, the index calculating section 23 of the analysis device 1 can calculate the index “arteriosclerosis degree” with use of the parameter identical to that for use in the measurement item “6: CIRCULATORY ORGAN MONITORING”. Therefore, a user may change the radar chart to a radar chart of the index “arteriosclerosis degree” by operating a switching button 170 that is displayed on the display section 15.

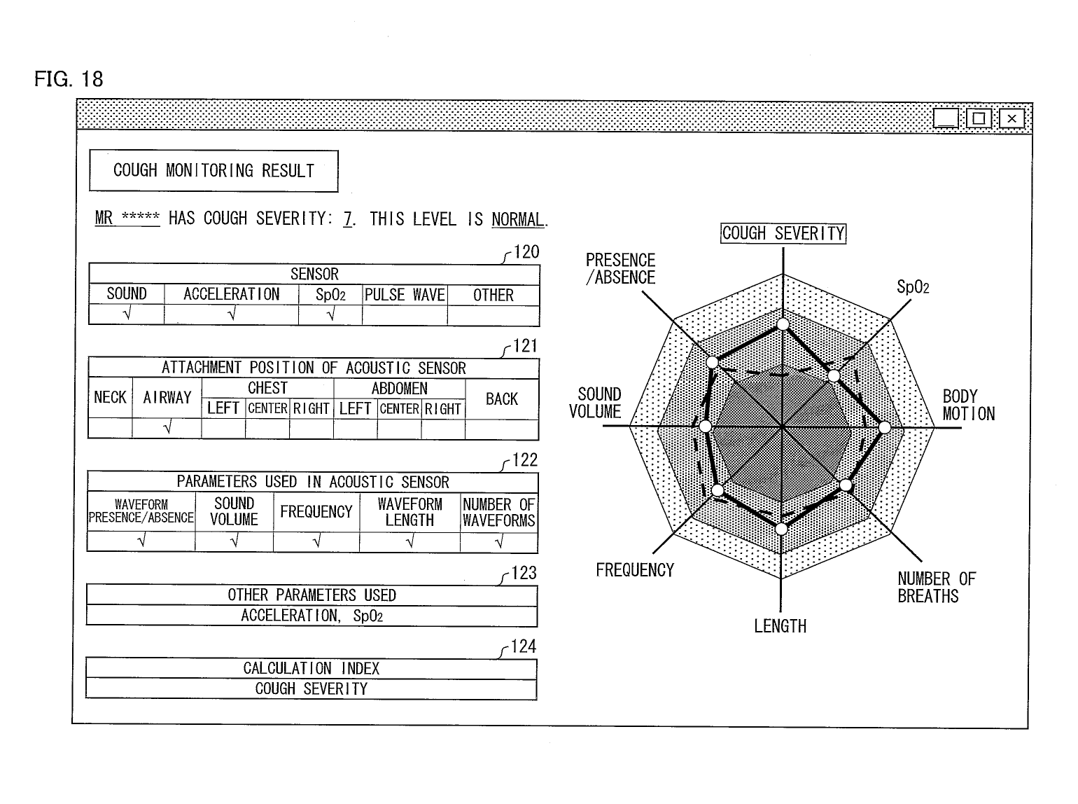

FIG. 18 is a diagram illustrating an example display of a measurement result produced by the analysis device 1 through a biometric process for measurement item “7: COUGH MONITORING”.

According to the above arrangement, a user can easily learn a measurement result regarding a selected measurement item by checking information displayed on the display section 15.

The following description will discuss a series of steps regarding a biometric process carried out by the analysis device 1, specifically, from (i) a step in which a user starts to carry out measurement to (ii) a step in which a measurement result is displayed as described above.

[Biometric Process Flow]

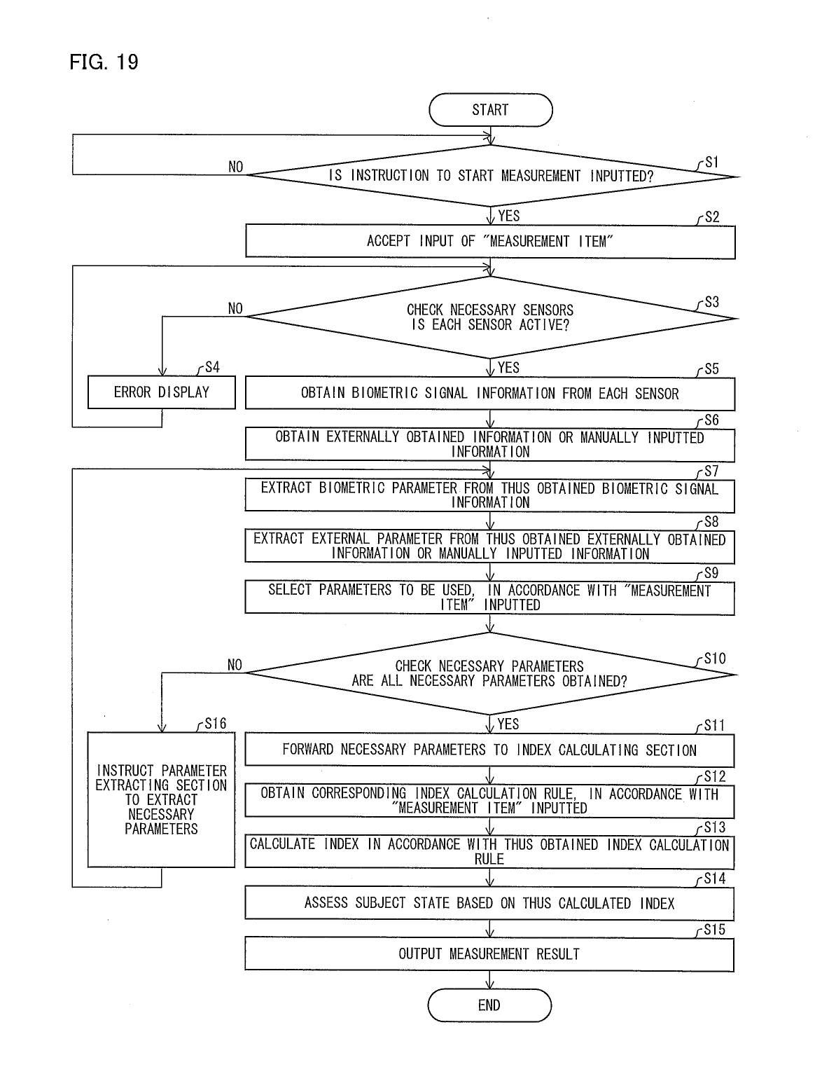

FIG. 19 is a flowchart illustrating a flow of a biometric process carried out by the analysis device 1.

In a case where the analysis device 1 has received, via the input operation section 14, an instruction to start carrying out measurement with respect to a subject (YES in S1), the measurement item determining section 25 accepts an input of a measurement item (S2). For example, in a case where a user has selected the measurement item “apnea degree measurement”, the measurement item determining section 25 determines that the measurement item of the biometric process to be started is “1: APNEA DEGREE MEASUREMENT”.

Next, the information obtaining section 20 refers to the measurement method storage section 31 so as to check whether or not biometric sensors, all of which are necessary to carry out measurement of the measurement item determined, are in an active state (S3). In the example described above, it is possible to understand the following (i) and (ii) on the basis of the parameter specifying information and the attachment position designating information shown in FIG. 3A: (i) to carry out the biometric process whose measurement item is “1: APNEA DEGREE MEASUREMENT”, presence/absence of a waveform in the vicinity of an airway, a sound volume in the vicinity of the airway, a length of the waveform, and the number of waveforms are essential biometric parameters; and (ii) to carry out the biometric process whose measurement item is “1: APNEA DEGREE MEASUREMENT”, an SpO₂ and a heart rate are auxiliary parameters. In view of this, the information obtaining section 20 checks, among the acoustic sensor 2a attached in the vicinity of the airway, the acoustic sensor 2b attached to a left portion of the chest, and the pulse oximeter 3, whether or not at least the acoustic sensor 2a is in the active state.

Here, in a case where such an essential biometric sensor is in an inactive state (NO in S3), the information obtaining section 20 preferably notifies the user via the display section 15 that the biometric sensor is in the inactive state and cannot carry out measurement (S4). In addition, in this case, it is more preferable that the information obtaining section 20 notify, in a manner easily understood by the user (for example, with use of a drawing), the user of (i) what kind of biometric sensor is necessary and (ii) which position is an appropriate attachment position (the position in the vicinity of the airway or the position of the left portion of the chest).

In a case where it is confirmed that the biometric sensor(s) which is necessary for the measurement is in the active state (YES in S3), the information obtaining section 20 obtains biometric signal information from the biometric sensor(s) (S5). In the example described above, the information obtaining section 20 obtains (i) at least sound data in the vicinity of the airway from the acoustic sensor 2a, and (ii) if necessary, sound data of a heart sound from the acoustic sensor 2b and measurement data of an SpO₂ from the pulse oximeter 3.

Further, the information obtaining section 20 can obtain, if necessary, (i) externally obtained information (weather, an ambient temperature, humidity, atmospheric pressure, etc., on a date on which the measurement is carried out) from the information providing device 7 and (ii) manually inputted information (an ID of the subject, a name of the subject, an age of the subject, a sex of the subject, etc.), which is inputted via the input operation section 14 (S6).

Next, the parameter extracting section 21 extracts a biometric parameter from the biometric signal information obtained (S7). The parameter extracting section 21 can extract, by referring to the measurement method storage section 31, (i) only parameters used for the measurement item “1: APNEA DEGREE MEASUREMENT” selected, or (ii) all extractable parameters among the parameters shown in FIG. 3A. Further, in a case where the information obtaining section 20 has obtained the externally obtained information and the manually inputted information described above, the parameter extracting section 21 extracts external parameters from the externally obtained information and the manually inputted information (S8). The parameter extracting section 21 causes the parameter storage section 30 to store the parameters extracted.

Then, the parameter selecting section 22 refers to the measurement method storage section 31 (see FIGS. 3A and 3B), so as to select, from among the parameters stored in the parameter storage section 30, parameters to be used for the measurement item determined (S9). In the example described above, the parameter selecting section 22 selects the following parameters, each of which is associated with the measurement item “1: APNEA DEGREE MEASUREMENT”: presence/absence of a waveform (airway); a sound volume; a length of the waveform; the number of waveforms; an SpO₂; and a heart rate. In a case where the parameter selecting section 22 has obtained from the parameter storage section 30 all the parameters necessary for the measurement (YES in S10), the parameter selecting section 22 supplies such parameters to the index calculating section 23 (S11).

Next, the index calculating section 23 reads out, from the index calculation rule storage section 32, an index calculation rule corresponding to the measurement item selected (S12), and then calculates an index of the measurement item in accordance with the index calculation rule (S13). In the example described above, the index calculating section 23 reads out an “apnea degree calculation rule” (see, for example, (a) through (d) of FIG. 5) corresponding to the measurement item “1: APNEA DEGREE MEASUREMENT”, and calculates an apnea degree with use of the parameters supplied from the parameter selecting section 22. The apnea degree thus calculated is stored in the index storage section 33 together with information on the date on which the measurement is carried out, the ID of the subject, etc.

Further, the state assessing section 24 assesses a state of the subject on the basis of the index calculated (S14). The state assessing section 24 carries out assessment in accordance with assessment criterion information corresponding to the measurement item selected. In the example described above, the state assessing section 24 assesses, in accordance with assessment criterion information (see, for example, (e) of FIG. 5) corresponding to the measurement item “1: APNEA DEGREE MEASUREMENT”, whether the apnea degree of the subject is normal, needs caution, or is abnormal.

The index calculating section 23 supplies the calculated index to the display section 15, and the state assessing section 24 supplies a result of the assessment to the display section 15. The display section 15 displays a measurement result so as to present the measurement result to the user (S15). The measurement result is a result obtained through a series of steps of the biometric process (shown in FIG. 19) carried out by the analysis device 1, and includes at least (i) the calculated index and (ii) a result of the assessment as to the state of the subject. In addition, the measurement result can include accessory information such as information on the parameters used and information on what index is calculated. Each of FIGS. 12 through 18 shows an example of how to display the measurement result.

Meanwhile, in a case where any of the parameters necessary for the measurement is not stored in the parameter storage section 30 in S10 (NO in S10), the parameter selecting section 22 preferably instructs the parameter extracting section 21, on the basis of the parameter specifying information stored in the measurement method storage section 31, to extract such a parameter (S16). For example, according to the parameter specifying information shown in FIG. 3B, the “APNEA DEGREE MEASUREMENT” requires, for the parameter of “PRESENCE/ABSENCE OF WAVEFORM”, a parameter of “number of times a subject stops breathing for 10 or more seconds”. Accordingly, the parameter selecting section 22 instructs the parameter extracting section 21 to extract such a parameter. In accordance with the instruction received from the parameter selecting section 22, the parameter extracting section 21 (i) extracts the parameter, (ii) causes the parameter storage section 30 to store the parameter, and (iii) make a response to the parameter selecting section 22. This method allows the analysis device 1 to have such an arrangement that a parameter used for various measurement items is extracted as a default, while a specific parameter related to a specific measurement item is extracted if necessary. This arrangement makes it possible to (i) reduce a processing load of the biometric process and (ii) improve processing efficiency.

The example described above deals with a case in which obtaining of the biometric signal information and extraction of the parameters are carried out on receipt of an instruction to start the biometric process. Note, however, that it is possible that (i) the steps before the extraction of the parameters, that is, the steps S3 through S8, can be carried out in advance (regularly, if necessary) irrespective of the instruction to start the biometric process, and (ii) all the parameters necessary for the measurement are stored in the parameter storage section 30 all the time.

[Variation: Assessment Based on Long-Term Index Transition]

In the above explanation, the biometric system 100 has the arrangement in which the analysis device 1 calculates a single index by carrying out a single biometric process, and assesses the state of the subject on the basis of the single index thus calculated. Note, however, that the arrangement of the analysis device 1 of the present invention is not limited to this.

For example, the analysis device 1 can (i) carry out measurement for a single measurement item a plurality of times at different times and dates (that is, a biometric parameter is obtained repeatedly), and (ii) calculate an index a plurality of times. Then, the analysis device 1 can assess the state of the subject by (i) obtaining a statistic of the indexes obtained through calculation carried out a plurality of times, or (ii) finding, for example, a rate of change of the index over time. This arrangement makes it possible to learn not only a temporary state of the subject by carrying out measurement singly but also a long-term tendency of the state of the subject. This makes it possible to carry out measurement which (i) is suitable for the measurement item and (ii) has high accuracy.

In order to measure a long-term tendency, the analysis device 1 of the present invention has such an arrangement that the measurement method storage section 31 stores, for each of the measurement items, repeated measurement instruction information designating timing for repeated calculation of a corresponding index so that the measurement item and the repeated measurement instruction information are associated with each other.

In a case where the user selects a certain measurement item and inputs an instruction to start the biometric process, each of the sections of the control section 10 illustrated in FIG. 1 (i) refers to the measurement method storage section 31, (ii) reads out repeated measurement instruction information associated with the certain measurement item determined by the measurement item determining section 25, and (iii) recognize timing at which the measurement for the certain measurement item is to be carried out. The repeated measurement instruction information designates, for example, (i) time intervals at which the measurement is regularly carried out, and/or (ii) a time period during which the measurement is regularly carried out. The repeated measurement instruction may be, for example, to “calculate an index once a day for one (1) month”. Alternatively, the repeated measurement instruction information can more specifically set a time period during which the measurement is carried out.

Then, each of the sections of the control section 10 regularly carries out the above-described biometric process in accordance with the repeated measurement instruction information. In the example described above, for example, the index calculating section 23 (i) calculates the index once every 24 hours, (ii) causes the index to be associated with the ID of the subject and the date on which the measurement is carried out, and (iii) causes the index storage section 33 to keep storing the index for 31 days.

In a case where the indexes obtained during the time period designated by the repeated measurement instruction information are accumulated in the index storage section 33, the state assessing section 24 assesses, on the basis of the indexes thus accumulated, the state of the subject which state is measured in terms of the measurement item selected. In the example described above, the index storage section 33 maintains the indexes obtained over a month. The state assessing section 24 assesses the state of the subject on the basis of these indexes. It is possible that the index calculation rule storage section 32 stores, for each of the measurement items, (i) information on how to use the indexes in the assessment, and/or (ii) information on assessment criterion.

The process carried out by the state assessing section 24 is, for example, (i) a process of analyzing a transition of the index by plotting a value of the index on a two-dimensional graph in which an ordinate indicates the value of the index and an abscissa indicates a time, or (ii) a process of calculating statistics of the indexes, such as a mean value, a maximum value, a minimum value, and/or dispersion of values. The state assessing section 24, for example, compares (i) a result of the analysis obtained as such with (ii) a reference value so as to assess, in terms of the measurement item selected, the state of the subject (for example, whether the state of the subject is normal, needs caution, or is abnormal).

Further, the state assessing section 24 can, by comparing (i) the previous indexes accumulated in accordance with the repeated measurement instruction information with (ii) the index obtained by carrying out the biometric process singly after the previous indexes were obtained, assess the latest state of the subject at a time that the biometric process is carried out. This comparison of the previous indexes and the latest index with each other makes it possible to assess the latest state of the subject more precisely.

In this case, for example, an analysis method is stored for each of the measurement items in the measurement method storage section 31, which analysis method indicates, for example, (i) which time period in the past is selected to obtain the target previous indexes for the comparison, or (ii) how to compare the newest index with the previous indexes.

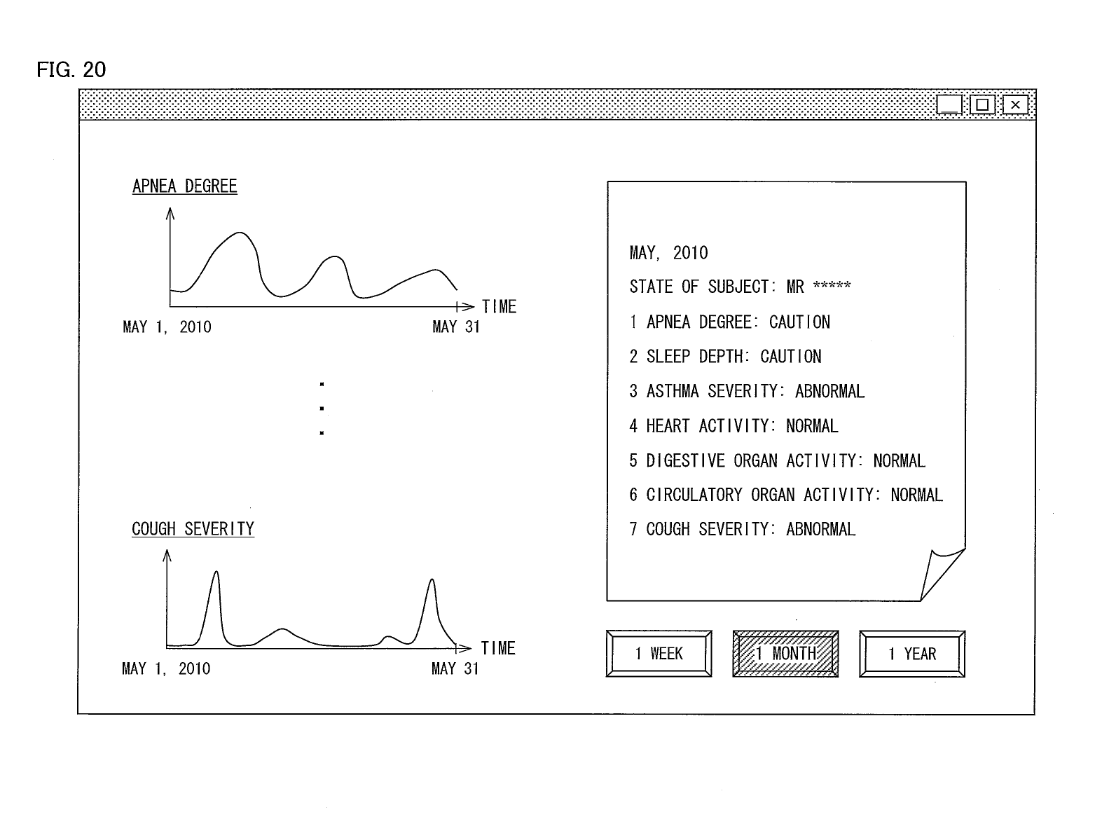

FIG. 20 is a diagram illustrating an example display, as a measurement result, of a long-term tendency of a state of a subject.

As illustrated in FIG. 20, the display section 15 can display, for each of the measurement items, the two dimensional graph created by the state assessing section 24. With this arrangement, the user can easily understand how the index of the subject has changed over a month. Further, it is possible to display, on the basis of a statistic of the indexes obtained over a month, a comprehensive result of one-month assessment of the state of the subject. This arrangement allows the user to easily understand the long-term tendency of the state of the subject.

Note that the two dimensional graph illustrated in FIG. 20 is merely an example, and the present invention is not limited to this. For example, it is possible to have an arrangement in which a range of the abscissa (time) to be displayed is changed, if necessary. For example, by changing a time period for the measurement from “one (1) month” to “one (1) year”, it is possible to display, on the basis of the indexes of the subject, collected over a year, a comprehensive result of one-year assessment of the state of the subject. As illustrated in FIG. 20, by having such a setting that an option button for a time period for the measurement is displayed so that the user can select a time period for the measurement, it is possible for the user to change the time period for the measurement with a simple operation.

[Variation: Determination of Measurement Item]

In the above explanation, the measurement item determining section 25 of the analysis device 1 determines, as a target measurement item of the biometric process to be carried out, the measurement item selected by the user via the input operation section 14. Note, however, that the arrangement of the analysis device 1 of the present invention is not limited to this.

For example, the analysis device 1 may have (i) an arrangement in which the measurement item determining section 25 determines a measurement item in accordance with which one(s) of biometric sensors is in the active state, or (ii) an arrangement in which several candidates are selected and the user selects one of the candidates.

What kind of biometric sensor is necessary is determined for each of the measurement items. The measurement item determining section 25 checks, via the information obtaining section 20, which one(s) of the biometric sensors is in the active state, and identifies a measurement item(s) with which measurement can be carried out with use of the biometric signal information received from such biometric sensor(s). Here, in a case where a single measurement item is identified, the measurement item determining section 25 determines the identified measurement item as the measurement item of the biometric process to be carried out. On the other hand, in a case where a plurality of measurement items remain as the candidates, the measurement item determining section 25 causes the display section 15 to display only these measurement items as options so that the user selects one of the measurement items thus displayed.

### Embodiment 1-2

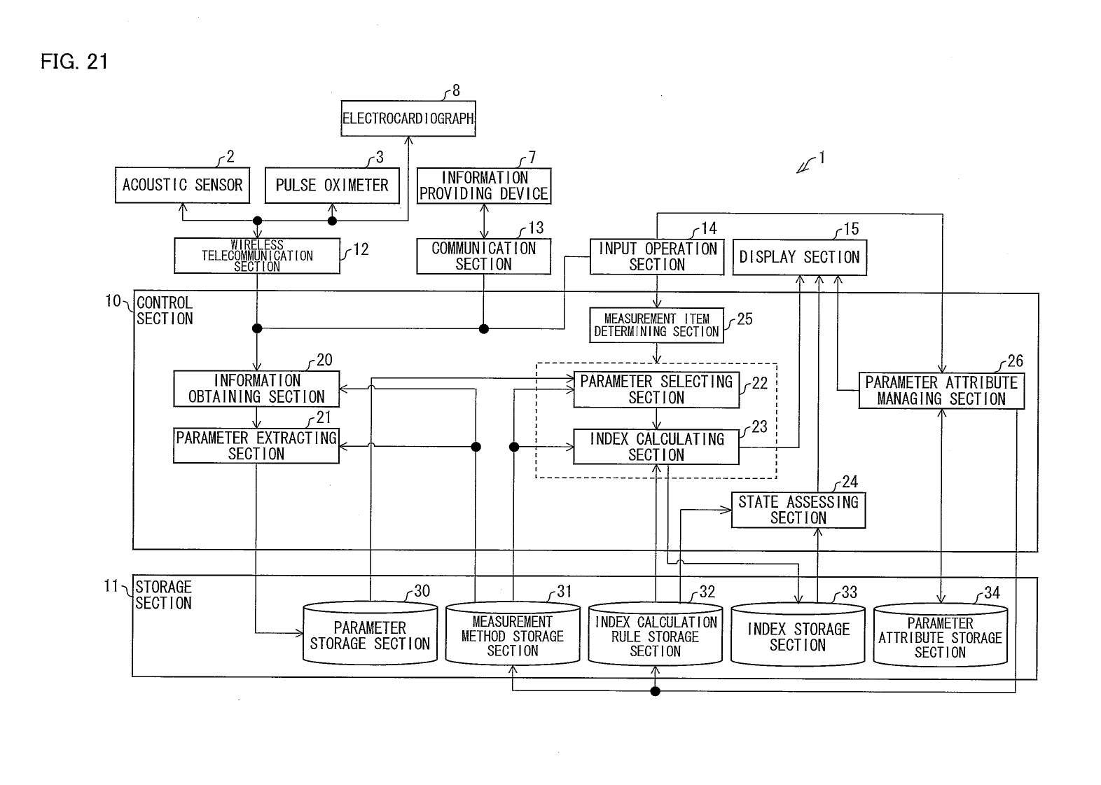

Another embodiment of the present invention is described below with reference to FIGS. 21 through 24. For convenience of explanation, members that have functions identical to those of members illustrated in the drawings of the aforementioned embodiment are given identical reference numerals, and an explanation of content which is identical to content explained in the aforementioned embodiment is omitted here.

In the aforementioned embodiment, a biometric device (analysis device 1) of the present invention merely notifies the user, by the use of information 122 on parameters and information 123 on parameters, whether the user has selected parameters for calculating an index corresponding to a target measurement item (see FIGS. 12 through 18).

However, actually, inside the analysis device 1, the parameters are different from each other in magnitude of an influence on calculation of the index. For example, in a case where the index “APNEA DEGREE” corresponding to a measurement item “apnea degree measurement” is to be calculated, the parameter “PRESENCE/ABSENCE OF WAVEFORM” has the largest influence on calculation of “APNEA DEGREE”, and the parameters “HEART RATE” and “SpO₂” each have a small influence on the calculation of “APNEA DEGREE” as compared with the other parameters (see the apnea degree calculation rule shown in (b) and (c) of FIG. 5). This is because (i) the score of a parameter changes depending on whether the parameter is “ESSENTIAL” or “AUXILIARY” and (ii) parameters are different from each other in value of weighting.

As described above, what parameter has the greatest importance in index calculation varies. For this reason, it is preferable that in a case where a measurement result is presented to a user, not only (i) whether the user has selected parameters but also (ii) magnitude (importance) of an influence caused to calculation of the index by each of the parameters used in the measurement be clearly presented to the user.

In the present embodiment, the analysis device 1 manages, for each of indexes, magnitude of an influence which is caused by each of the parameters used in calculation. The analysis device 1 carries out the management in such a manner that the magnitude is represented by “PRIORITY” for example, and is managed as “PARAMETER ATTRIBUTE”. The analysis device notifies the user of “PARAMETER ATTRIBUTE” of each of the parameters together with the measurement result. With this arrangement, the biometric device (analysis device 1) of the present embodiment can (i) provide a user with a measurement result having a large amount of information and therefore (ii) improve the user's convenience.

[Arrangement of Analysis Device 1]

FIG. 21 is a block diagram illustrating an essential configuration of the analysis device 1 of the present embodiment.

The analysis device 1 of the present embodiment is different from the analysis device 1 illustrated in FIG. 1 in the following points. First, a storage section 11 of the analysis device 1 of the present embodiment further includes a parameter attribute storage section 34 for storing a parameter attribute of each of the parameters. Secondly, a control section 10 of the analysis device 1 of the present embodiment further includes a parameter attribute managing section 26 as a functional block. The parameter attribute managing section 26 manages the parameter attributes stored in the parameter attribute storage section 34.

Note that the analysis device 1 may communicate with an electrocardiograph 8 wirelessly so as to obtain an electrocardiogram of a subject from the electrocardiograph 8.

[As to Parameter Attribute Storage Section 34]

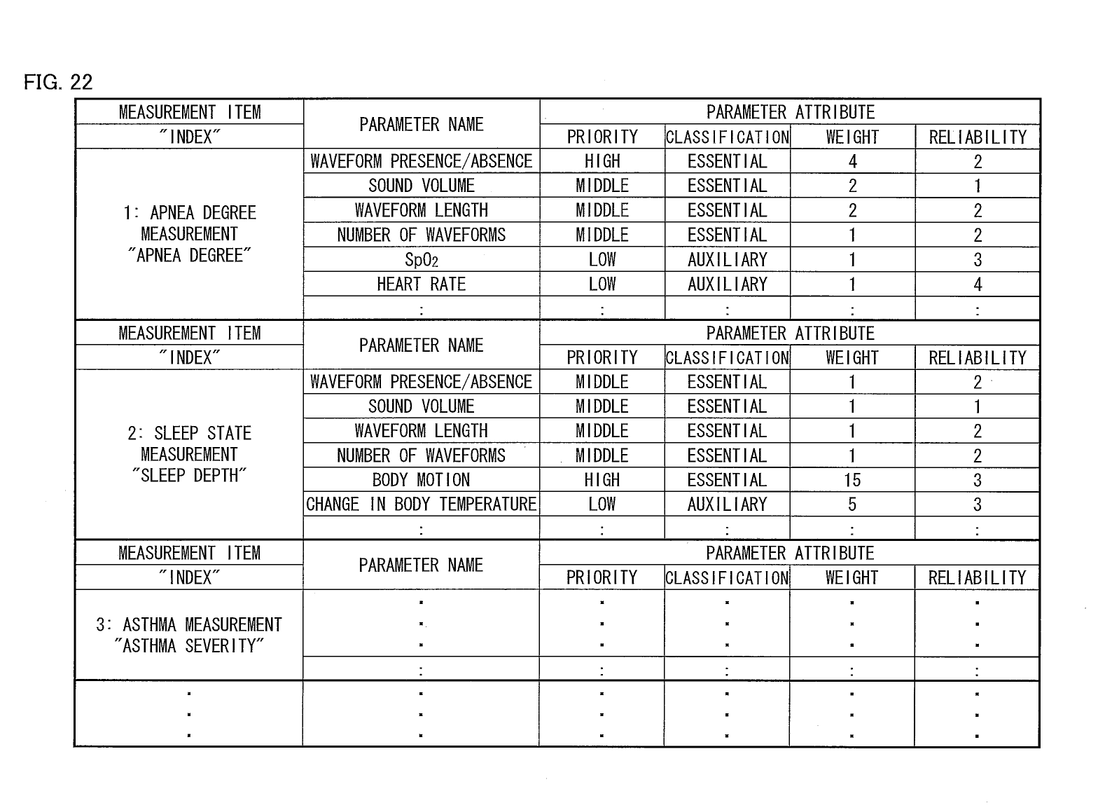

FIG. 22 is a table illustrating a data structure of information stored in the parameter attribute storage section 34.

In the present embodiment, the parameter attribute managing section 26 of the analysis device 1 (i) manages, as “PARAMETER ATTRIBUTE”, magnitude of an influence on index calculation, and (ii) causes the parameter attribute storage section 34 to store, for each of indexes, a parameter attribute of each of the parameters.

In the present embodiment, the parameter attribute is constituted by several elements. As illustrated in FIG. 22, the parameter attribute includes, for example, elements such as “PRIORITY”, “CLASSIFICATION” and “WEIGHTING”. Further, the parameter attribute can also include other elements such as “RELIABILITY”. Note that the data structure illustrated in FIG. 22 is merely an example, and the data structure of the parameter attribute of the present invention is not limited to this. That is, magnitude (parameter attribute) of an influence on index calculation can be represented by an element other than the elements described above.

The element “CLASSIFICATION” is such information that in a case where the parameters are classified into “ESSENTIAL” parameters and “AUXILIARY” parameters, the information indicates which one of an “ESSENTIAL” parameter and an “AUXILIARY” parameter a corresponding parameter belongs to. For example, in the example illustrated in FIG. 22, in a case where the index “APNEA DEGREE” is to be calculated for the measurement item “1: APNEA DEGREE MEASUREMENT”, the classification of the parameter “PRESENCE/ABSENCE OF WAVEFORM” is “ESSENTIAL”. This shows that the parameter “PRESENCE/ABSENCE OF WAVEFORM” is essential for calculation of the index “APNEA DEGREE”. The parameter attribute managing section 26 recognizes that (i) the parameter whose element “CLASSIFICATION” is “ESSENTIAL” has a large influence on the calculation of the index and (ii) a parameter whose element “CLASSIFICATION” is “AUXILIARY” has a small influence on the calculation of the index.

The element “WEIGHTING” is a value constituting an index calculation rule, as shown in (c) of each of FIGS. 5 through 11. Specifically, the value of “WEIGHTING” is a multiplier of a score in a calculation formula of an index, which score is obtained for each of the parameters. That is, the parameter attribute managing section 26 recognizes that the larger the value of “WEIGHTING” of the parameter is, the larger the influence of the parameter on the calculation of the index is.

The element “RELIABILITY” is information indicative of how reliable a value of the parameter is. The larger the value of “RELIABILITY” is, the more accurate the value of the parameter is likely to be. Accordingly, it is preferable to cause the parameter having a high value of “RELIABILITY” to have a large influence on the calculation of the index. With this arrangement, it is likely to heighten accuracy of the calculation of the index. In the present embodiment, the value of “RELIABILITY” has been determined and fixed in advance. The value of “RELIABILITY” can be determined in accordance with, for example, accuracy of biometric sensors. For example, (i) since the parameters “PRESENCE/ABSENCE OF WAVEFORM” and “SOUND VOLUME” are obtained with use of an acoustic sensor 2 that is likely to be influenced by noise due to an attachment environment and a life environment, these parameters are set to have low values of “RELIABILITY”, and (ii) since the parameter “SpO₂” is obtained with use of a pulse oximeter 3 that is unlikely to be influenced by an ambient environment, the parameter is set to have a high value of “RELIABILITY”. It is also possible that since the parameter “HEART RATE” is obtained on the basis of biometric signal information obtained from two biometric sensors, namely, the acoustic sensor 2 and the electrocardiograph 8, i.e., the value of the parameter “HEART RATE” is accurate as compared with the other parameters, the parameter is set to have a high value of “RELIABILITY”.

The element “PRIORITY” is a value which directly indicates magnitude of an influence of a parameter on index calculation. As a matter of course, it is considered that the higher a value of “PRIORITY” of the parameter is, the larger the influence of the parameter on the calculation of the index is. The parameter attribute managing section 26 recognizes the element “PRIORITY” in this manner. As described above, magnitude of an influence of a parameter on index calculation is directly indicated by the element “PRIORITY”, and is presented to the user. This makes it possible for the user to understand importance of each of the parameters intuitively.

In the present embodiment, the parameter attribute managing section 26 expresses the element “PRIORITY” in three levels, namely, “HIGH”, “MIDDLE”, and “LOW”. The level “PRIORITY: HIGH” indicates that among all the parameters used in calculation of the index, a corresponding parameter has the largest influence (most important) on the calculation of the index. The level “PRIORITY: LOW” indicates that among all the parameters used in the calculation of the index, a corresponding parameter has the smallest influence (not important) on the calculation of the index.

Further, in the present embodiment, the parameter attribute managing section 26 can determine the element “PRIORITY” by carrying out comprehensive evaluation on the basis of other elements. The following description specifically deals with the index “APNEA DEGREE” with reference to FIG. 22. Among parameters used in calculation of the index “APNEA DEGREE”, a parameter whose element “CLASSIFICATION” is “ESSENTIAL” and whose element “WEIGHTING” has the highest value is considered as being the most important parameter. As to the index “APNEA DEGREE”, such a parameter is the parameter “PRESENCE/ABSENCE OF WAVEFORM”. Accordingly, the parameter attribute managing section 26 sets the parameter “PRESENCE/ABSENCE OF WAVEFORM” of the index “APNEA DEGREE” to “PRIORITY: HIGH”. On the other hand, a parameter whose element “CLASSIFICATION” is “AUXILIARY” and whose element “WEIGHTING” has the smallest value is considered as being the least important parameter. Accordingly, the parameter attribute managing section 26 sets (i) the parameter “SpO₂” of the index “APNEA DEGREE” and (ii) the parameter “HEART RATE” of the index “APNEA DEGREE” both to “PRIORITY: LOW”. The parameter attribute managing section 26 sets the other parameters to “PRIORITY: MIDDLE”. In a case where “PRIORITY: HIGH” and “PRIORITY: LOW” are uniquely set, the parameter attribute managing section 26 can set only one parameter to “PRIORITY: HIGH” or “PRIORITY: LOW” by further taking the element “RELIABILITY” into consideration. For example, in the example described above, since the parameter “SpO₂” is lower than the parameter “HEART RATE” in “RELIABILITY”, the parameter attribute managing section 26 can set only the parameter “SpO₂” of the index “APNEA DEGREE” to “PRIORITY: LOW”.

Note that the element “PRIORITY” is not limited to the three-level evaluation described above, and can be expressed in another form, as long as the form allows the user to understand “PRIORITY” intuitively. For example, the parameter attribute managing section 26 can add, as “PRIORITY”, rank orders “FIRST PLACE”, “SECOND PLACE”, . . . to the most important parameter, the second most important parameter, . . . , respectively.

As described above, the parameter attribute stored in the parameter attribute storage section 34 is managed by the parameter attribute managing section 26 so that consistency between the parameter attribute, parameter specifying information (see FIGS. 3A and 3B) stored in a measurement method storage section 31, and an index calculation rule (see FIGS. 5 through 11) stored in an index calculation rule storage section 32 is maintained all the time. That is, in a case where the element “CLASSIFICATION” or the element “WEIGHTING” of any of the parameters, stored in the parameter attribute storage section 34, has been changed, the parameter attribute managing section 26 updates (i) the parameter specifying information, stored in the measurement method storage section 31, and (ii) the index calculation rule, stored in the index calculation rule storage section 32, so that the consistency between the parameter attribute stored in the parameter attribute storage section 34, the parameter specifying information, and the index calculation rule is maintained.

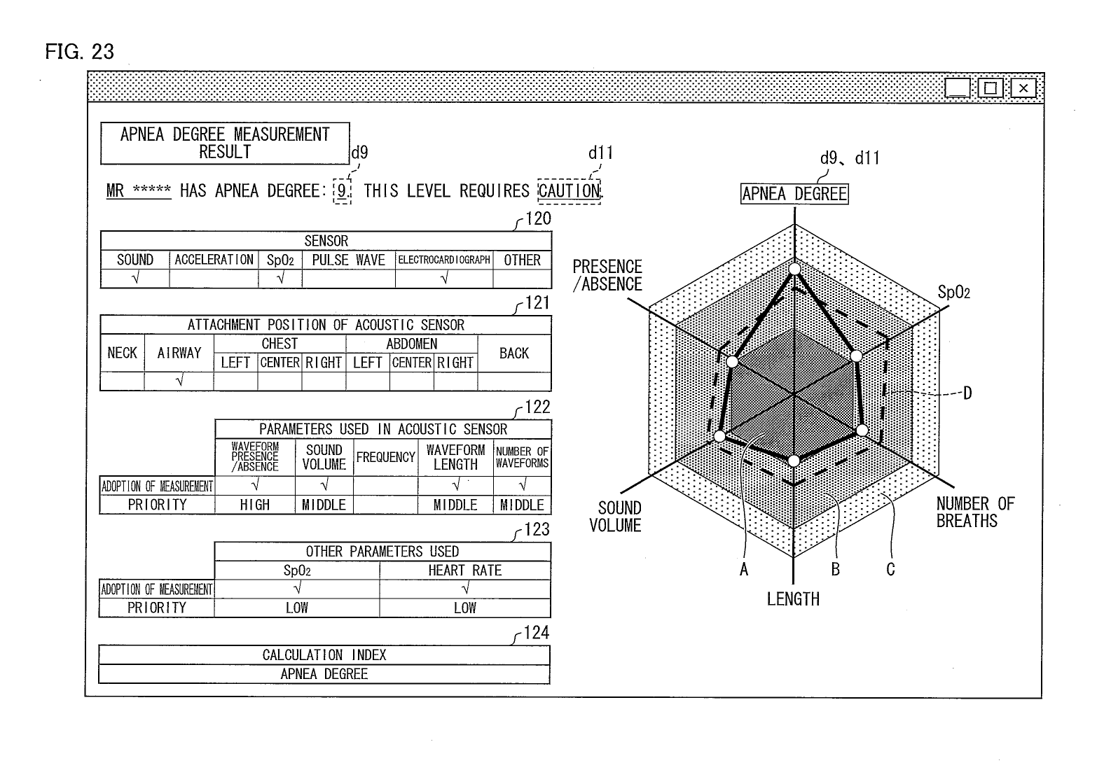

FIG. 23 is a diagram illustrating an example of how a measurement result is displayed on a display screen of a display section 15, which measurement result is obtained in such a manner that the analysis device 1 of the present embodiment carries out the biometric process. Specifically, FIG. 23 shows, as an example, how the measurement result is displayed, which measurement result is obtained in such a manner that the analysis device 1 carries out the biometric process for the measurement item “1: APNEA DEGREE MEASUREMENT”.

As compared with the measurement result illustrated in FIG. 12, the measurement result illustrated in FIG. 23 is further provided with the following information. That is, information 122 and information 123 on the parameters used in calculation of the index “APNEA DEGREE” include not only (i) whether the user has selected parameters but also (ii) information on magnitude (importance) of an influence of each of used parameters on the calculation of the index. In the example illustrated in FIG. 23, the importance described above is directly expressed as the element “PRIORITY” of each of the parameters, as an example.

In a case where the analysis device 1 displays the measurement result for the measurement item “1: APNEA DEGREE MEASUREMENT”, the parameter attribute managing section 26 (i) reads out, from the parameter attribute storage section 34, the parameter attribute (here, the element “PRIORITY”) of each of the parameters used in the calculation of the index “APNEA DEGREE”, and (ii) supplies the parameter attribute to a display control section (not shown). On the basis of (i) a result of calculation, supplied from an index calculating section 23, (ii) a result of assessment, supplied from the state assessing section 24, and (iii) the parameter attribute thus supplied, the display control section generates a measurement result screen illustrated in FIG. 23, and causes the display section 15 to display the measurement result screen.

According to the analysis device 1 of the present invention, the parameter attribute managing section 26 manages, as a parameter attribute such as “PRIORITY”, magnitude of an influence caused by each of parameters used in calculation. Then, in a case where the index has been calculated, a result of the calculation and “PRIORITY” of each of the parameters used are displayed together.

With the above arrangement, the user can (i) obtain a measurement result for the measurement item “1: APNEA DEGREE MEASUREMENT” as a value of “APNEA DEGREE” which is an easily understandable index, and (ii) by checking the element “PRIORITY” and/or the like, easily understand what parameter has been regarded as being an important parameter during a process in which the index has been calculated. As a result, the biometric device 1 (analysis device 1) of the present embodiment can (i) provide a user with a measurement result having a larger amount of information, and therefore (ii) improve the user's convenience.

[Variation: Designing Calculation Formula]

In each of the aforementioned embodiments, the parameters set for each of the indexes, and the parameter attribute of each of the parameters have been set and stored in the parameter attribute storage section 34 in advance.

However, the present invention is not limited to this, and may be arranged such that the parameters and the parameter attributes are either set and stored in the parameter attribute storage section 34 by the user arbitrarily, or stored in the parameter attribute storage section 34 once, and are then changed by the user arbitrarily.

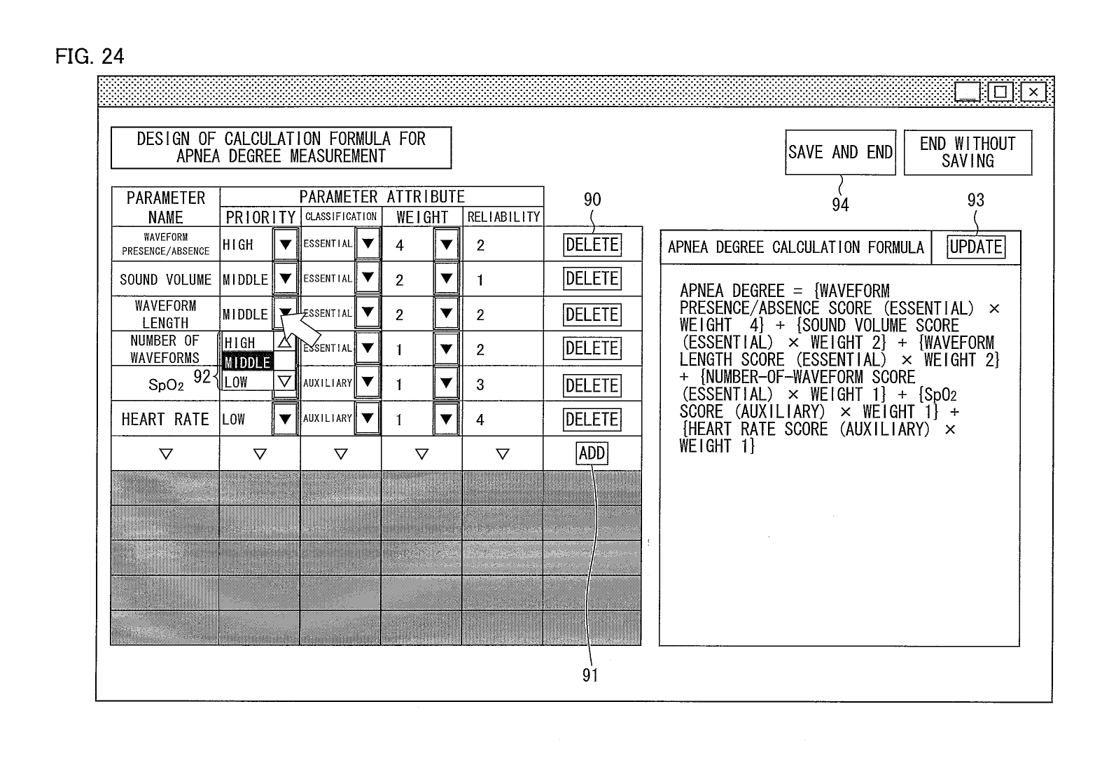

FIG. 24 is a diagram illustrating an example design screen for use by a user to design a calculation formula. FIG. 24 illustrates, as an example, a screen for designing a calculation formula which is used to calculate the index “APNEA DEGREE” for the measurement item “1: APNEA DEGREE MEASUREMENT”.

The user operates the display screen displayed on the display section 15 with use of an input operation section 14. The user can design the calculation formula by (i) selecting or canceling the parameter used to calculate the index, or (ii) changing the parameter attribute of the parameter. FIG. 24 is a specific example of the design screen, and is not indented to limit the arrangement of the analysis device 1 of the present invention.

The following description explains, with reference to FIG. 24, how to operate the design screen. Selection and cancellation of the parameter used in calculation of the index is carried out via (i) a deletion button 90 for deleting a row and (ii) an addition button 91 for adding a row in a table showing a list of the parameters. In a case where the user has selected the addition button 91 (by, for example, clicking the button 91 with use of a mouse), a list of parameters which can be used in the calculation of the index is displayed. With this list, the user can add a new parameter easily. As to a parameter which is not used in the calculation, it is possible to remove, from the list of the parameters to be used, such a parameter by selecting the deletion button 90 of a row of such a parameter.

Further, the user can edit the parameter attribute of each of the parameters to be used in the calculation. For example, it is possible to provide a drop-down form to a cell for an element which can be edited by the user. In this case, for example, in a case where the user has selected the cell of the target element that the user wants to edit, it is possible to display a list box 92. In the list box 92, values which can be set for the element are displayed as a list. The user can select a desired value from the list box 92 to set the element to the desired value. For example, in a case where the user has selected a value of “HIGH” from the list box 92, the element “PRIORITY” of the parameter “LENGTH OF WAVEFORM” is changed from “MIDDLE” to “HIGH”.

Note that it is not necessary that all the elements be editable. Since the element “RELIABILITY” depends on a characteristic of the biometric sensor for deriving a corresponding parameter, it is possible to have an arrangement in which the element “RELIABILITY” cannot be edited. Further, it is possible to have an arrangement in which the element “RELIABILITY” is not displayed on the design screen. Alternatively, it is possible to have an arrangement in which (i) elements which can be edited by the user are only the element “CLASSIFICATION” and “WEIGHTING” and (ii) the element “PRIORITY” is calculated automatically by the parameter attribute managing section 26 on the basis of “CLASSIFICATION” and “WEIGHTING” (or further on the basis of “RELIABILITY”). By contrast, it is also possible to have an arrangement in which (i) the element which can be edited by the user is only the element “PRIORITY” and (ii) the parameter attribute managing section 26 adjusts “CLASSIFICATION” and “WEIGHTING” on the basis of “PRIORITY”.

It is possible to present, as illustrated in FIG. 24, a calculation formula which is newly specified on the basis of an edited parameter(s) and an edited parameter attribute(s). Specifically, in a case where the user has selected an update button 93, the parameter attribute managing section 26 creates a new calculation formula on the basis of the edited parameter(s) and the edited parameter attribute(s), and causes the new calculation formula to be displayed in a predetermined region. If the user knows about the measurement item to a certain degree, the user can easily carry out setting of a parameter and a parameter attribute more appropriately by checking the calculation formula displayed.

In a case where a button 94 showing “SAVE AND END” is selected, the parameter attribute managing section 26 stores the parameter and the parameter attribute, both newly set by the user, in the parameter attribute storage section 34 so as to update content stored in the parameter attribute storage section 34. Further, the parameter attribute managing section 26 updates (i) the parameter specifying information stored in the measurement method storage section 31 and (ii) the index calculation rule stored in the index calculation rule storage section 32, so that the parameter specifying information, the index calculation rule, and such updated content, stored in the parameter attribute storage section 34, are consistent with one another.

### Embodiment 1-3

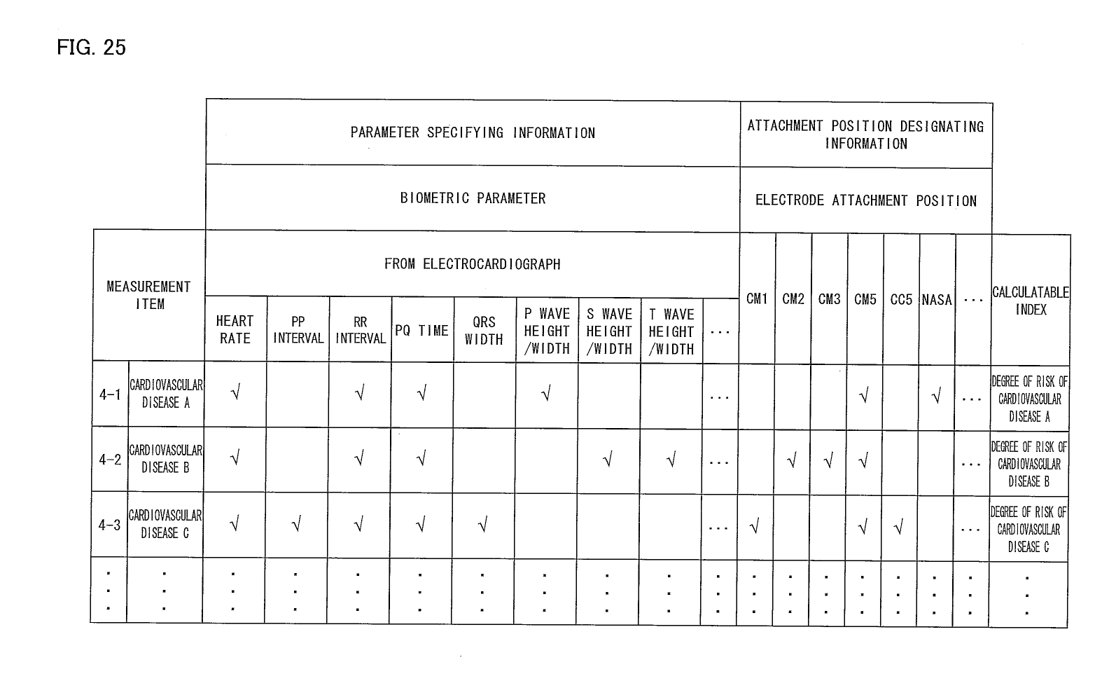

Another embodiment of the present invention is described below with reference to FIG. 25. For convenience of explanation, members that have functions identical to those of members illustrated in the drawings of the aforementioned embodiments are given identical reference numerals, and an explanation of content which is identical to that of the aforementioned embodiments is omitted here.

Each of the aforementioned embodiments explains such an example that a biometric device (analysis device 1) of the present invention employs biometric sensors (2 through 6 and 8), and specifically, the following seven measurement items can be measured: “1: APNEA DEGREE MEASUREMENT”; “2: SLEEP STATE MEASUREMENT”; “3: ASTHMA MEASUREMENT”; “4: HEART MONITORING”; “5: DIGESTIVE ORGAN MONITORING”; “6: CIRCULATORY ORGAN MONITORING”; and “7: COUGH MONITORING”. In each of the aforementioned embodiments, the analysis device 1 calculates in particular the index “HEART ACTIVITY” for the measurement item “4: HEART MONITORING”, and provides a measurement result employing three-level evaluation.

An analysis device 1 of the present embodiment has such an arrangement that further detailed measurement can be carried out for the measurement item “4: HEART MONITORING” on the basis of an electrocardiogram obtained from an electrocardiograph 8. Specifically, the analysis device 1 has an arrangement in which electrical activity of a heart of a subject is monitored and analyzed so as to measure a degree of risk of each of various cardiovascular diseases.

FIG. 25 is a table illustrating a data structure of information stored in a measurement method storage section 31. As illustrated in FIG. 25, the measurement method storage section 31 stores, for each of measurement items which can be measured by the analysis device 1, (i) parameter specifying information, (ii) attachment position designating information, and (iii) a corresponding calculable index so that the parameter specifying information, the attachment position designating information, and the corresponding calculable index are associated with each other.

The parameter specifying information identifies parameters which are necessary to calculate an index, in the same manner as parameter specifying information illustrated in FIG. 3A or 3B. For example, in a case where an index calculating section 23 of the analysis device 1 calculates the index “DEGREE OF RISK OF CARDIOVASCULAR DISEASE A” for the measurement item “4-1: CARDIOVASCULAR DISEASE A”, parameters which should be referred to by the index calculating section 23 are “HEART RATE”, “RR INTERVALS”, “PQ TIME”, and “P WAVE HEIGHT/WIDTH”. These biometric parameters related to a heart can be obtained from an electrocardiogram supplied from the electrocardiograph 8.

That is, in a case where a measurement item determining section 25 has determined that a target measurement item is the measurement item “4-1: CARDIOVASCULAR DISEASE A”, the parameter selecting section 22 selects the following parameters, as parameters to be used, from a parameter storage section 30 on the basis of the parameter specifying information: “HEART RATE”, “RR INTERVALS”, “PQ TIME”, and “P WAVE HEIGHT/WIDTH”.

There might be a case where an attachment position of each of electrodes of the electrocardiograph 8 differs depending on a measurement item (a target cardiovascular disease to be diagnosed). For such a case, it is possible to store the attachment position designating information for each of the measurement items. In the present embodiment, the attachment position designating information designates an attachment position pattern of each of the electrodes of the electrocardiograph 8, that is, a type of induction. With this arrangement, the analysis device 1 can (i) find a difference or a similarity in type of induction (electrode attachment position pattern), (ii) manage the electrocardiogram so that the type of induction and the electrocardiogram are associated with each other, and (iii) analyze the electrocardiogram. It is thus possible for the analysis device 1 to carry out assessment as to a risk of a target disease with higher accuracy.

In the present embodiment, an index calculation rule (not shown) is stored, for each of the indexes “DEGREE OF RISK OF CARDIOVASCULAR DISEASE A”, “DEGREE OF RISK OF CARDIOVASCULAR DISEASE B”, . . . , corresponding to the measurement items, respectively, is stored in an index calculation rule storage section 32.

The index calculating section 23 reads out, from the index calculation rule storage section 32, an index calculation rule for calculating a target risk, and calculates the index (degree of risk of cardiovascular disease) with use of the biometric parameters obtained from the electrocardiogram selected by the parameter selecting section 22.

A state assessing section 24 evaluates a degree of risk of the cardiovascular disease of the subject on the basis of the index calculated, and outputs a measurement result obtained to a display section 15. In the present embodiment, a parameter attribute managing section 26 can also cause the display section 15 to display, for each of the parameters used, a priority together with the measurement result.

### Embodiment 2

The present invention further relates to a biometric device for measuring a state of a living body, particularly, to a biometric device for collecting and evaluating a biometric sound.

[Background Technique]

Patent Literature 1 discloses a biometric information measuring device including (i) a sensor attachment head (sensor) to be attached to a body of a user and (ii) a main body for measuring, on the basis of signal information (biometric signal information/biometric sound signal information) obtained from the sensor, a plurality of parameters (biometric information) of the user. This biometric information measuring device, for example, (i) detects an attachment site of the attached sensor so as to select a parameter measurable at the detected attachment site and (ii) adjusts, in correspondence with the attachment site, an amplification degree of a signal of the biometric signal information outputted from the sensor. With this arrangement, Patent Literature 1 provides a biometric information measuring device that is not limited in terms of application or attachment site of a sensor and that can thus be widely used.

According to the biometric information measuring device of Patent Literature 1, it is possible to attach sensors to a plurality of positions of a body of a living body. For example, the sensors can be attached to wrists of the living body and a head of the living body, and can hang from a neck of the living body. Here, according to the technique of Patent Literature 1, a plurality of kinds of sensor are prepared and attached to the living body so as to sense various biological information such as a pulse wave, a pulse beat, GSR (Galvanic Skin Response), a skin temperature, a blood sugar level, and an acceleration.

As described above, according to the biometric information measuring device of Patent Literature 1, devices (a plurality of kinds of sensor), with which various biometric information can be measured, can be attached to various positions of a body so that measurement of biometric information can be carried out in accordance with not only a specific site but also such various positions of the body, to which the plurality of kinds of sensor are attached.

[Technical Problem]

However, with the conventional arrangement described above, various kinds of sensor are used, and there might be a case in which measurement cannot be carried out depending on an attachment site, and a parameter (biometric information) cannot be obtained as a result. Accordingly, in a case where a sensor is attached to an inappropriate position, there is a risk that a process might be carried out with insufficient information. This causes a problem that a measurement result having low accuracy is outputted. An inaccurate measurement result will in turn lead to a problem of a final determination being erroneous or determination accuracy being low.

The present invention has been accomplished in view of the above problem. It is an object of the present invention to provide a biometric device which has an improvement in accuracy of measurement by (i) collecting parameters with use of not a plurality of kinds of sensor but a single or a plurality of sensors of a single kind, and therefore (ii) preventing such a situation that only insufficient information can be obtained depending on limitation of an attachment site. Further, it is another object of the present invention to provide a biometric device which (i) has an improvement in accuracy of measurement by employing different processing methods for obtained parameters depending on attribute information of used sensors, and (ii) has an effect identical with such an effect that various measurement items can be measured with use of a plurality of kinds of sensor.

### Embodiment 2-1

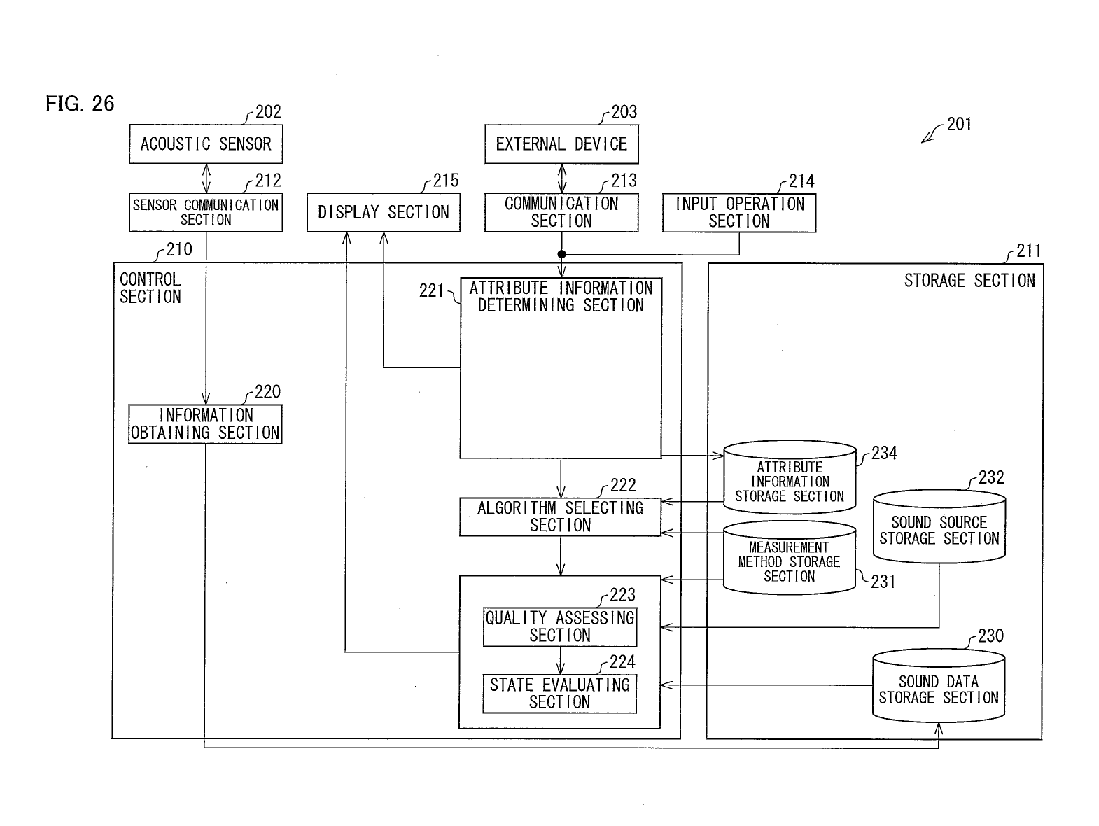

An embodiment of the present invention is described below with reference to FIGS. 26 through 40.

A biometric device of the present invention (i) obtains biometric signal information from, for example, a sensor for sensing a state of a living body, and (ii) measures various states and various symptoms of a subject with use of a parameter obtained from the biometric signal information.

In the present embodiment, (i) a living body is a human (hereinafter referred to as “subject”) as an example and (ii) a single acoustic sensor for obtaining a sound of the subject is used as a biometric sensor for sensing a state of the subject. The following description deals with a case in which the biometric device of the present invention is provided as a small information processing device which (i) is provided separately from the acoustic sensor and (ii) is excellent in transportability and portability. Accordingly, in the present embodiment, the biometric signal information obtained with use of a sensor is supplied to the biometric device via appropriate wireless or wired communication means. Note, however, that the biometric device of the present invention is not limited to this, and can be formed of an installed-type information processing device such as a personal computer. Further, the biometric device of the present invention is not limited to the above arrangement, and can be contained in the sensor itself.

Further, the biometric device of the present invention can deal with, as a living body, an animal (such as a dog) other than a human. In this case, the biometric device obtains a biometric sound of the animal so as to measure a state of the animal.

[Biometric System]

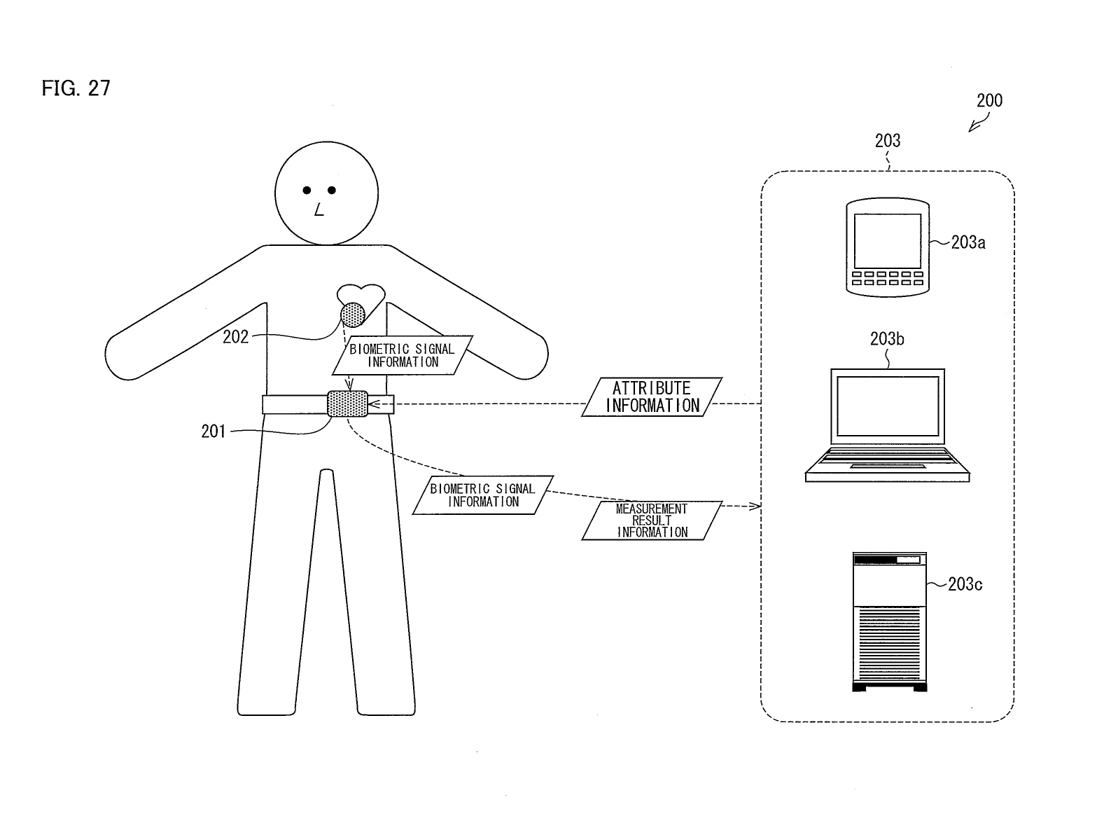

FIG. 27 is a diagram schematically illustrating a configuration of a biometric system 200 of an embodiment of the present invention. The biometric system 200 of the present invention includes at least (i) a single acoustic sensor (biometric sound sensor) 202 and (ii) an analysis device (biometric device) 201. Further, as illustrated in FIG. 27, the biometric system 200 can include an external device 203 for processing various kinds of information related to measurement of the subject.

The acoustic sensor 202 is a contact-type microphone which is to be attached to a body of the subject to detect a sound emitted from the subject. A tackiness agent layer is provided on a surface of the acoustic sensor 202. The acoustic sensor 202 is attached to a body surface of the subject via the tackiness agent layer. A position to which the acoustic sensor 202 is attached is not limited, as long as the acoustic sensor 202 can effectively pick up a target sound at the position. For example, in order to detect a breath sound of the subject or a cough sound of the subject, the acoustic sensor 202 is attached to a position of an airway or a position of a chest. In order to, for example, detect a heart sound or a heart rate, the acoustic sensor 202 is attached to a position of a left portion of the chest (as viewed from a subject side). In order to detect an abdominal sound of the subject, the acoustic sensor is attached to a position of an abdomen.

The acoustic sensor 202 detects a biometric sound emitted from the subject, and transmits, as biometric signal information, sound data of the biometric sound thus detected to an analysis device 201. For example, in the example illustrated in FIG. 27, the acoustic sensor 202 attached to a left portion of the chest detects a heart sound, and transmits, as the biometric signal information, sound data of the heart sound thus detected to the analysis device 201. Among the biometric signal information, particularly, the sound data outputted from the acoustic sensor 202 is herein referred to as “biometric sound signal information”.

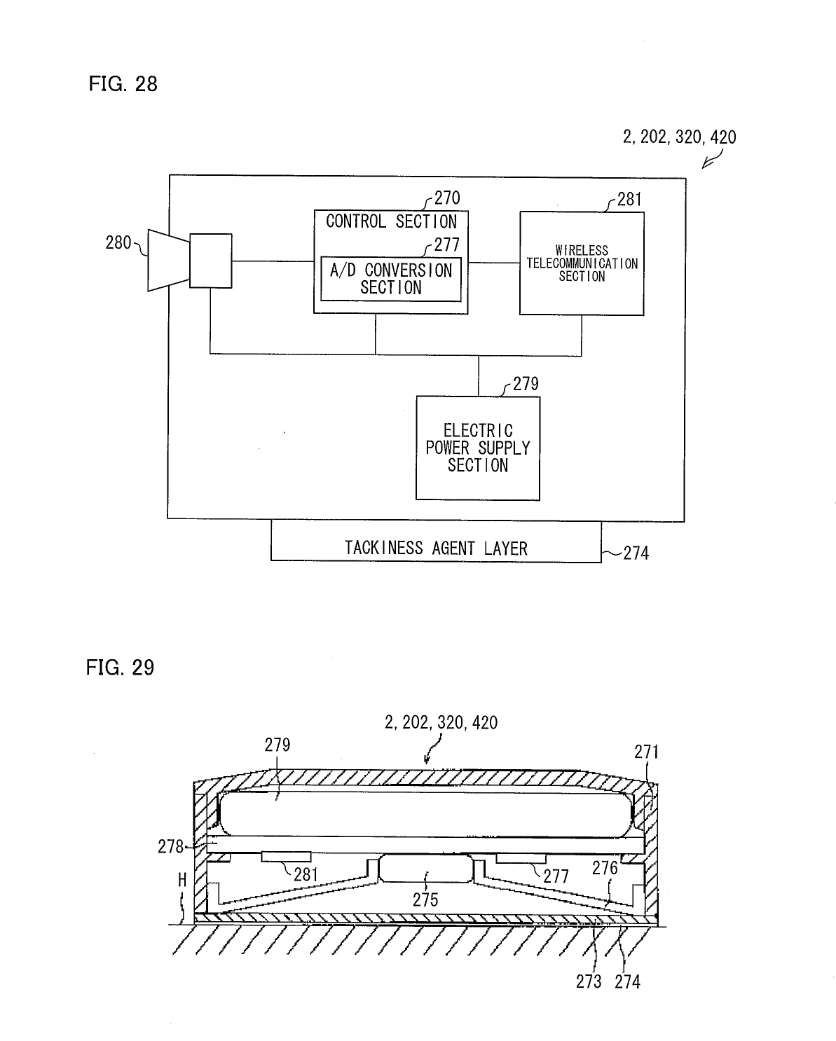

FIG. 28 is a block diagram illustrating an essential configuration of the acoustic sensor 202. As illustrated in FIG. 28, the acoustic sensor 202 includes a control section 270, an electric power supply section 279, a microphone section 280, a wireless telecommunication section 281, and a tackiness agent layer 274.

The electric power supply section 279 supplies electric power to respective circuits of the control section 270, the microphone section 280, and the wireless telecommunication section 281, and is constituted by a general storage battery. Alternatively, the electric power supply section 279 can be constituted by a connection section for connecting to an AC adapter or the like by cable. Further, in a case where the biometric system is supplied with electric power wirelessly, the electric power supply section 279 is constituted by a capacitor or the like for temporarily storing the electric power supplied.

The microphone section 280 collects a biometric sound emitted from the subject.

The tackiness agent layer 274 is an attachment mechanism which prevents the acoustic sensor 202 from being detached or away from the body surface of the subject due, for example, to gravity or friction of clothes or the like. The tackiness agent layer 274 is provided on an outer surface of the acoustic sensor 202. The tackiness agent layer 274 is formed of, for example, a sucker or absorbing gel, and provides a function of causing the acoustic sensor 202 to remain on the body surface.

The wireless telecommunication section 281 carries out wireless telecommunications with another device (the analysis device 201, the external device 203, or another biometric sensor). As wireless telecommunications means, short-distance wireless telecommunications means such as Bluetooth® communications and WiFi communications can be used so that the wireless telecommunication section 281 carries out short-distance wireless telecommunications with another device. Alternatively, a LAN may be set up so that the wireless telecommunication section 281 carries out wireless telecommunications with another device via the LAN thus set up.

In particular, the wireless telecommunication section 281 transmits the biometric sound signal information collected by the acoustic sensor 202 to the analysis device 201, and receives control data transmitted from the analysis device 201. The control data is information for use by the analysis device 201 to remotely operate the acoustic sensor 202 to (i) start or finish measurement or (ii) set a measurement condition.

Note that the acoustic sensor 202 and the analysis device 201 can be connected to each other via a cable. In this case, the acoustic sensor 202 includes, in place of the wireless telecommunication section 281, a communication section which carries out cable connections via a cable. The communication section transmits and receives various kinds of information to and from the analysis device 201 or the like via the cable.

The control section 270 controls each of sections of the acoustic sensor 202, and is formed of, for example, a microcomputer for a sensor. The control section 270 contains an analog/digital (A/D) conversion section 277 which is formed of an A/D converter or the like. The A/D conversion section 277 digitalizes the biometric sound collected by the microphone section 280, and outputs the biometric sound thus digitalized. Digitalized sound data is transmitted, as biometric sound signal information, to the analysis device 201 via the wireless telecommunication section 281.

FIG. 29 is a cross-sectional view illustrating an example configuration of the acoustic sensor 202. As illustrated in FIG. 29, the acoustic sensor 202 is a sound-collecting unit based on a so-called condenser microphone, and includes (i) a housing section 271 having such a cylindrical shape that one of end surfaces has an opening and (ii) a diaphragm 273 which is in closed contact with the housing section 271 so that the diaphragm 273 closes up the opening of the housing section 271. The acoustic sensor 202 further includes (i) a substrate 278 on which a first conversion section 275 and the A/D conversion section 277 (which serves as a second conversion section) are provided and (ii) the electric power supply section 279 serving as a battery for supplying electric power to the first conversion section 275 and the A/D conversion section 277.

As illustrated in FIG. 29, the microphone section 280 described above is formed of the diaphragm 273, the first conversion section 275, and an air chamber wall 276.

The tackiness agent layer 274 is provided on a surface of the diaphragm 273 so that the acoustic sensor 202 can be attached to a body surface (H) of the subject via the tackiness agent layer 274. A position to which the acoustic sensor 202 is attached is determined as appropriate so as to collect a sound (for example, a heart sound, a breath sound, or an abdominal sound) at a target measurement site effectively.

In a case where the subject emits a biometric sound, the diaphragm 273 vibrates slightly in accordance with a wavelength of the biometric sound. This slight vibration of the diaphragm 273 is transmitted to the first conversion section 275 via the air chamber wall 276 having a circular cone shape whose upper surface and bottom surface each have an opening.

The vibration transmitted via the air chamber wall 276 is converted into an electric signal by the first conversion section 275, and is then converted into a digital signal by the A/D conversion section 277. After that, the digital signal is transmitted to the analysis device 201 as the biometric sound signal information.

On the basis of the biometric sound signal information obtained from the acoustic sensor 202, the analysis device 201 measures a state of the subject. The analysis device 201 can obtain a measurement result by causing the biometric sound signal information obtained to be subjected to a biometric process. Specifically, the biometric process is constituted by a single or a plurality of information processings. The analysis device 201 carries out the single or plurality of information processings with respect to the obtained biometric sound signal information so as to derive measurement result information indicative of a state of the subject. The above single or plurality of information processings are, for example, (i) a quality assessing process which analyzes the biometric sound signal information (that is, the sound data) and which assesses quality (high quality or low quality) of the biometric sound signal information as quality of the sound data for use in the measurement and (ii) a state assessment processing which extracts various kinds of information (parameters) related to the subject from the biometric sound signal information and which evaluates the state of the subject on the basis of the parameters. Note, however, that the information processings, which are carried out by the analysis device 201 with respect to the biometric sound signal information to derive the measurement result information, are not limited to the processings described above. It is possible to further carry out various information processings (a third information processing, a fourth information processing . . . ). For example, the analysis device 201 can have a function of carrying out a noise removal processing as an information processing which noise removal processing removes, from the biometric sound signal information, a component such as a noise that is unnecessary for the analysis.

The analysis device 201 of the present invention stores a plurality of different algorithms for a single information processing. The plurality of different algorithms are prepared for respective pieces of attribute information of the acoustic sensor 202. The attribute information of the acoustic sensor 202 may be, but not limited to, for example (i) information (its attribute information name is “ATTACHMENT POSITION”) on which part of a body of a subject the acoustic sensor 202 is attached to, (ii) information (“MEASUREMENT SITE”) on what sound of the body of the subject is requested to be measured with use of the acoustic sensor 202, that is, a rough purpose of the measurement, and (3) information (“MEASUREMENT ITEM”) on what state (specific symptom) of the subject is requested to be measured with use of the acoustic sensor 202, that is, details of the purpose of the measurement.

Accordingly, even if only a single kind of sensor, namely, the acoustic sensor, is used, the analysis device 201 can carry out a single information processing with use of a plurality of different algorithms in accordance with the attribute information (the attachment position, the measurement site, and the measurement item) of the acoustic sensor 202. That is, even with use of only a single kind of sensor (the acoustic sensor), it is possible to (i) carry out various biometric processes in accordance with the attachment position of the acoustic sensor 202 or with the purpose of the measurement, and (ii) derive the measurement result information which is suitable for the purpose of the measurement. That is, the analysis device 201 can select an appropriate algorithm in accordance with the attribute information. As a result, with the analysis device 201, it is possible to improve accuracy of assessment of the state of the subject.

Details of how to determine the attribute information of the acoustic sensor 202 in the analysis device 201 will be described later. For example, it is possible to have an arrangement in which attribute information is designated by the user via the external device 203, and the attribute information thus designated is transmitted to the analysis device 201.

In the present embodiment, the analysis device 201 carries out the information processing “STATE ASSESSMENT PROCESSING” with use of various parameters related to the subject. For example, in order to improve accuracy of the measurement result, the analysis device 201 can extract parameters from (i) externally obtained information obtained from a device (for example, the external device 203) other than the acoustic sensor 202, and (ii) manually-inputted information directly inputted into the analysis device 201, and uses the parameters extracted.

Here, a parameter obtained from biometric (sound) signal information obtained from various biometric sensors (such as the acoustic sensor 202) is referred to as “biometric (sound) parameter”, and a parameter obtained from the externally obtained information or the manually-inputted information is referred to as “external parameter”. These terms are used in a case where it is necessary to distinguish such parameters in terms of their characteristics.

The biometric parameter reflects a physiological state of a subject. Specific examples of the biometric parameter encompass “sound volume” and “frequency distribution” obtained from sound data (biometric sound signal information) detected by the acoustic sensor 202. Further, in a case where a waveform is to be patterned, “intervals of the waveform”, “a cycle of the waveform”, “presence or absence”, “length”, “the number”, etc. of the waveforms may be extracted as biometric parameters by analyzing a pattern of the waveform.

The biometric parameter reflects a physiological state of a subject as described above, whereas the external parameter reflects an environmental condition outside the body. Specific examples of the external parameter encompass (i) specification information (for example, version information and what kind of information the biometric sensor functions to detect) of the biometric sensor, (ii) attachment position (chest region, abdominal region, back, vicinity of airway, etc.) of the biometric sensor, (iii) subject information (age, sex, hours of sleeping, previous mealtime, amount of exercise, history of disease, etc.) regarding the subject, and (iv) a measurement environment (ambient temperature, atmospheric pressure, humidity, etc.) in which the subject is present. The external parameter is, however, not limited to these.

The analysis device 201 derives the measurement result information with use of an appropriate combination of the biometric parameters and the external parameters. This makes it possible to carry out assessment which is suitable for the purpose of the measurement and which has higher accuracy.

As described above, the analysis device 201 carries out, with respect to the biometric sound signal information, at least one information processing. Then, the analysis device 201 (i) causes a display section of the analysis device 201 to display the measurement result information obtained and (ii) transmits the measurement result information obtained to the external device 203. The analysis device 201 can have an arrangement in which not only the measurement result information but also the biometric sound signal information (the sound data itself), obtained from the acoustic sensor 202 before the information processing is carried out, is transferred to the external device 203.

The external device 203 communicates with the analysis device 201 so as to transmit and receive, to and from the analysis device 201, various kinds of information for the biometric process carried out by the analysis device 201. Further, the external device 203 processes such information. In the biometric system 200 of the present embodiment, the external device 203 can be any device as long as the external device 203 can communicate with the analysis device 201. For example, the external device 203 can be formed of a portable terminal device 203a such as a mobile telephone and a PDA (personal digital assistant), a laptop personal computer 203b, or a data accumulation device 203c.

Next, the following description deals in greater detail with the arrangement of the analysis device 201 described above.

[Arrangement of Analysis Device 201]

FIG. 26 is a block diagram illustrating an essential configuration of the analysis device 201 of an embodiment of the present invention.

As illustrated in FIG. 26, the analysis device 201 of the present embodiment includes a control section 210, a storage section 211, a sensor communication section 212, an input operation section 214, and a display section 215. Further, the analysis device 201 has an electric power supply section (not shown) for supplying electric power to each of the sections described above. Note that the analysis device 201 can also include a communication section 213.

The sensor communication section 212 communicates with various biometric sensors (such as the acoustic sensor 202) in the biometric system 200. For example, in the present embodiment, the sensor communication section 212 is formed of wireless telecommunications means. As the wireless telecommunications means, short-distance wireless telecommunications means such as Bluetooth® communications and WiFi communications is used so that the sensor communication section 212 and the acoustic sensor 202 communicate with each other by the short-distance wireless telecommunications. Alternatively, a LAN can be set up so that the sensor communication section 212 and the acoustic sensor 202 communicate with each other via the LAN.

Note that the sensor communication section 212 of the analysis device 201 can communicate with the acoustic sensor 202 by cable communications means. However, it is preferable that the acoustic sensor 202 and the analysis device 201 communicate with each other wirelessly. By employing wireless telecommunications, it is possible to attach the acoustic sensor 202 to the subject easily. This reduces limitation in movement of the subject under the measurement environment, and as a result, reduces a stress or a load on the subject.

The communication section 213 communicates with various external devices such as the external device 203. For example, in the present embodiment, the communication section 213 communicates with an external device over a wide-area communication network. The communication section 213 transmits and receives information to and from an external device over a LAN or the Internet. For example, the analysis device 201 can receive, from an external information providing device via the communication section 213, externally obtained information for extracting external parameters used in the biometric process. Here, examples of the externally obtained information obtained by the communication section 213 encompass weather, ambient temperature, atmospheric pressure, and humidity on a certain date, and specification information of each of biometric sensors to be used. By, for example, referring to the specification information, the analysis device 201 can (i) determine which biometric sensor should be selected in accordance with a measurement item so as to use a parameter obtained from the biometric sensor selected, or (ii) in a case where the plurality of biometric sensors are simultaneously used, obtain information on a condition for a combination of a plurality of biometric sensors or information on what combination of the plurality of biometric sensors should not be used. Alternatively, the communication section 213 can receive, from the external device 203, (i) an instruction to start measurement, inputted into the external device 203 by the user, or (ii) selection of attribute information, inputted into the external device 203 by the user. Note that the communication section 213 can include either wireless telecommunications means or cable communications means. Which of the wireless telecommunications means and the cable communications means is employed is determined as appropriate in accordance with an embodiment of the biometric system 200.

The input operation section 214 is used by the user (the subject himself/herself or an operator who carries out measurement) to input an instruction signal into the analysis device 201. In a case where the analysis device 201 is in the form of a small information processing device as illustrated in FIG. 27, the input operation section 214 is constituted by an appropriate input device such as several buttons (arrow keys, enter key, character entry keys, etc.), a touch panel, a touch sensor, or a combination of a voice input section and a voice recognition section. Alternatively, in a case where the analysis device 201 is in the form of an installed-type information processing device, the input operation section 214 can include another input device (other than the input device described above) such as a keyboard constituted by a plurality of buttons (arrow keys, enter key, character entry keys, etc.), and a mouse. In the present embodiment, the user uses the input operation section 214 so as to (i) input an instruction to start measurement or an instruction to finish the measurement, or (ii) select attribute information such as an attachment position of the acoustic sensor 202, a measurement site of the acoustic sensor 202, and a measurement item of the acoustic sensor 202. Further, the user can input, with use of the input operation section 214, directly into the analysis device 201, information (manually-inputted information) which is necessary for the measurement. For example, parameters of a subject, such as age, sex, average hours of sleeping, hours of sleeping on a measurement date, previous mealtime, content of the meal, and amount of exercise, are inputted to the analysis device 201.

The display section 215 displays (i) a measurement result of a biometric process carried out by the analysis device 201 and (ii) as a GUI (graphical user interface) screen, an operation screen that a user uses to operate the analysis device 201. For example, the display section 215 displays (i) an input screen which is used by a user to input the parameters, (ii) an operation screen through which the user designates a measurement item and instructs the start of measurement, and (iii) a result display screen for displaying the measurement result of a biometric process that has been carried out. The display section 215 is constituted by, for example, a display device such as an LCD (liquid crystal display).

In the present embodiment, the analysis device 201 is in the form of a portable small information processing device. For this reason, there is a risk that the input operation section 214 and the display section 215, both included in the analysis device 201, might not be able to deal, as an interface section, with an amount of information which should be inputted and outputted sufficiently. In this case, it is preferable that the input operation section 214 and the display section 215 be in the form of an interface section included in a laptop personal computer 203b or in another installed-type information processing device.

According to the above arrangement, the display section 215 of the laptop personal computer 203b displays the operation screen described above, and the input operation section 214 (such as a keyboard and a mouse) of the laptop personal computer 203b accepts an instruction from the user. With this arrangement, the user can (i) easily input an instruction to start the measurement or an instruction to finish the measurement, or (ii) select attribute information such as an attachment position of the acoustic sensor 202, a measurement site of the acoustic sensor 202, and a measurement item of the acoustic sensor 202. This improves operability. The instruction or the attribute information inputted via the laptop personal computer 203b is transmitted to the communication section 213 of the analysis device 201 over the LAN. Further, the display section 215 of the laptop personal computer 203b can display a result display screen showing a measurement result which result display screen is larger than that of the display section 215 of the analysis device 201. This makes it possible to present a larger amount of information on the measurement result to the user in an easily understood manner. The measurement result information derived by the analysis device 201 is transmitted from the communication section 213 of the analysis device 201 to the laptop personal computer 203b over the LAN.

The control section 210 carries out integrated control of sections of the analysis device 201, and includes, as functional blocks, (i) an information obtaining section 220, (ii) an attribute information determining section 221, (iii) an algorithm selecting section 222, and (iv), as an information processing section, both a quality assessing section 223 and a state evaluating section 224. Each of these functional blocks can be provided in such a manner that a CPU (central processing unit) reads out, to a RAM (random access memory) (not shown) or the like, a program stored in a memory device (storage section 211) constituted by a ROM (read only memory), an NVRAM (non-volatile random access memory) or the like, and executes the program.

The storage section 211 stores various data read out when (i) a control program and (ii) an OS program both executed by the control section 210, (iii) an application program executed by the control section 210 in order to carry out various functions that the analysis device 201 has, and (vi) various data read out when the application program is executed. In particular, various programs and data to be read out when a biometric process is carried out by the analysis device 201 are stored in the storage section 211. Specifically, the storage section 211 includes a sound data storage section 230, a measurement method storage section 231, a sound source storage section 232, and an attribute information storage section 234.

It should be noted that the analysis device 201 includes a temporary storage section (not shown). The temporary storage section is a so-called working memory for temporarily storing, in the course of various kinds of processing carried out by the analysis device 201, data for use in calculation, a calculation result, etc., and is constituted by a RAM, etc.

The information obtaining section 220 of the control section 210 obtains various kinds of information which is necessary for a biometric process. Specifically, the information obtaining section 220 obtains biometric sound signal information (sound data) from the acoustic sensor 202 via the sensor communication section 212. The information obtaining section 220 causes the sound data storage section 230 to store the sound data obtained. When causing the sound data storage section 230 to store the sound data, the information obtaining section 220 can also cause the sound data storage section 230 to store (i) information on a date on which the sound data is created or (ii) subject information, together with the sound data. Note that it is preferable that the information obtaining section 220 cause the sound data storage section 230 to (i) store not all the sound data obtained and (ii) temporarily input the sound data into a RAM or the like (not shown) which can be referred to by the control section 210. According to the above arrangement, it is possible to carry out a real time process with respect to the sound data obtained. This makes it possible to (i) reduce a processing load in a case where not all the sound data but a part of the sound data is necessary and (ii) save a memory capacity of the sound data storage section 230.

The attribute information determining section 221 determines attribute information of the acoustic sensor 202 for use in the biometric process which is to be carried out by the analysis device 201. As an example, the attribute information determining section 221 determines (i) an attachment position of the acoustic sensor 202 and (ii) a rough purpose (measurement site) of measurement to be carried out by the acoustic sensor 202. In a case where details of the purpose of the measurement can be determined, it is possible to determine measurement items together with the details of the purpose of the measurement. How to determine the attribute information can be selected from a several methods.

In the present embodiment, the display section 215 of the external device 203 displays an input screen for the attribute information so as to let the user select attribute information via the input operation section 214 of the external device 203. The attribute information determining section 221 (i) receives, via the communication section 213, the attribute information selected by the user, and (ii) determines, on the basis of content of the attribute information, an attachment position and a measurement site (and measurement items) each of which is designated by the user.

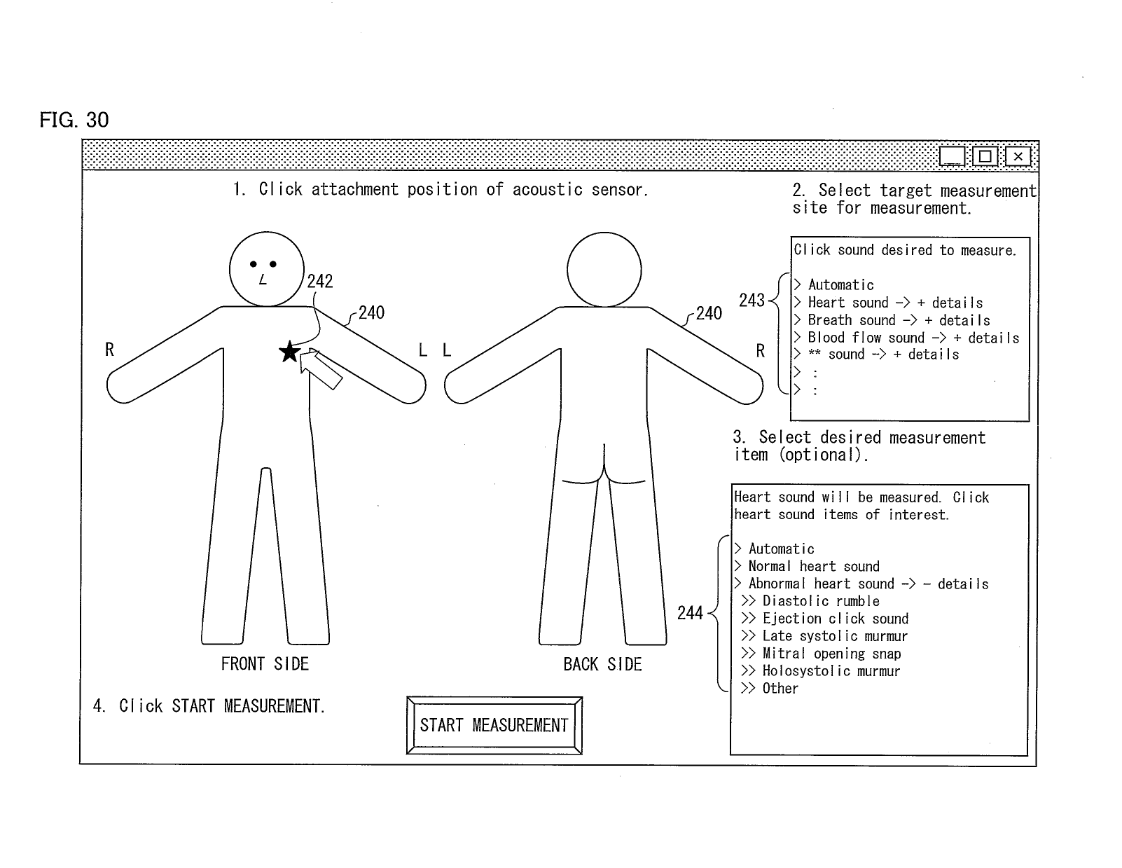

FIG. 30 is a diagram illustrating an example of the input screen of the attribute information displayed in the display section 215. As illustrated in FIG. 30, the attribute information determining section 221 causes the display section 215 to display a human body figure 240, and accepts selection of an attachment position. The user operates, for example, the input operation section (mouse) 14 to click a desired attachment position on the human body figure 240. It is thus possible to designate the attachment position of the acoustic sensor 202. In the example illustrated in FIG. 30, a black star sign 242 is displayed at the attachment position designated. In a case where the attachment position is designated as described above, the attribute information determining section 221 determines, as the attribute information “ATATCHEMENT POSITION”, the attachment position corresponding to the position (for example, “FRONT SIDE-CHEST-UPPER LEFT”) designated by the black star sign 242. The attribute information determining section 221 can display (i) outline stars for all possible attachment positions as candidates, or can display (ii) a list on which attachment positions are provided in a text format.

The attribute information determining section 221 causes the display section 215 to display candidates 243 for the measurement site, and accepts selection of the measurement site. The user operates the input operation section 214 to click a target measurement site. With this operation, it is possible to designate the measurement site of the acoustic sensor 202. Similarly, candidates 244 for a measurement item are displayed on the display section 215. The user clicks a desired measurement item so as to designate the measurement item of the acoustic sensor 202. The attribute information determining section 221 determines options selected by the user as the attribute information “MEASUREMENT SITE” and “MEASUREMENT ITEM”. As illustrated in FIG. 30, a purpose of the measurement can be such that the measurement site is roughly selected (for example, “HEART SOUND”, “BREATH SOUND”, “BLOOD FLOW SOUND” or the like is selected), or can be such that a specific name of a disease (the measurement item) is selected more specifically.

The attribute information determining section 221 transmits the attribute information determined as described above to the algorithm selecting section 222. Further, in a case where the attribute information thus determined is stored in a nonvolatile manner, the attribute information determining section 221 causes the attribute information storage section 234 to store the attribute information determined.

In accordance with the attribute information determined by the attribute information determining section 221, the algorithm selecting section 222 selects, from among a plurality of algorithms, an algorithm which should be carried out by each of various information processing sections of the analysis device 201. The measurement method storage section 231 stores a plurality of algorithms for each of the various information processes so that the plurality of algorithms and pieces of the attribute information are associated with each other. The algorithm selecting section 222 refers to the measurement method storage section 231, and selects, on the basis of the attribute information determined, an algorithm which should be carried out by each of the information processing sections.

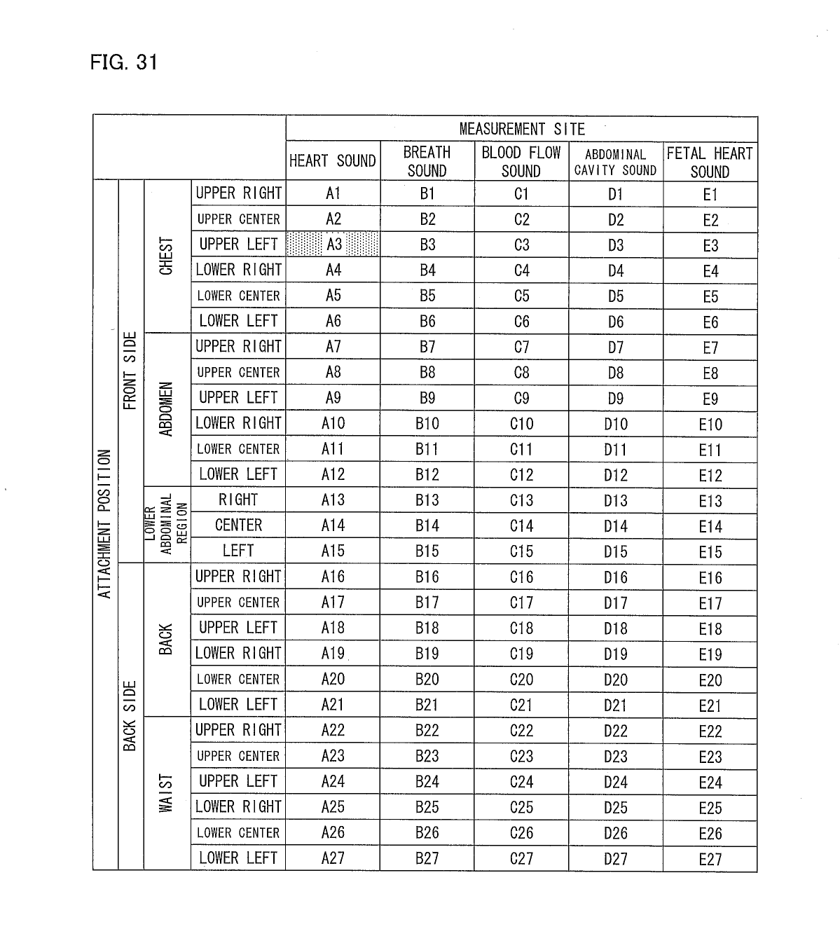

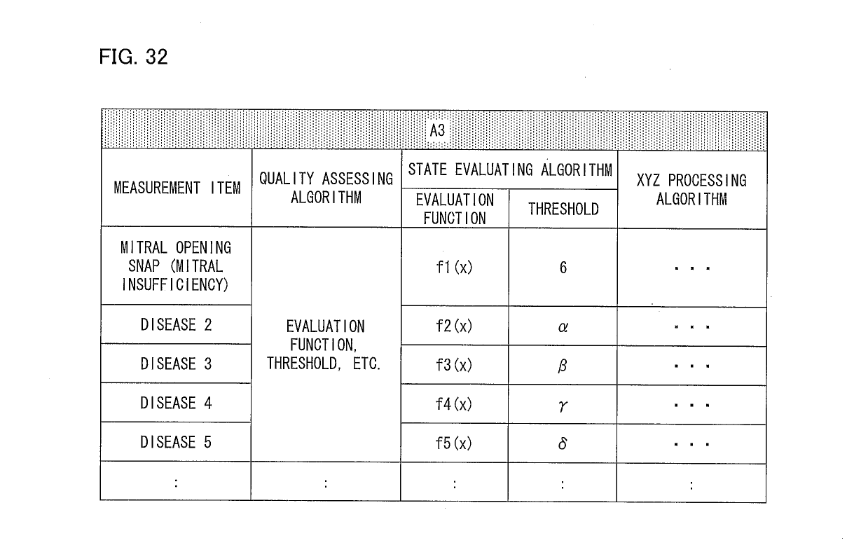

FIG. 31 is a diagram illustrating a specific example of a correspondence table which shows how the pieces of the attribute information stored in the measurement method storage section 231 and the plurality of algorithms correspond to each other. FIG. 32 is a table showing a specific example of an algorithm of each of the information processings, which algorithm is stored in the measurement method storage section 231.

As shown in FIG. 31, the analysis device 201 retains, in the measurement method storage section 231, information on how the pieces of the attribute information and the plurality of algorithms correspond to each other. In the example illustrated in FIG. 31, the information on such a correspondence relationship is retained as a correspondence table in a table format. Note, however, that a data structure of the information is not limited to this, as long as the correspondence relationship is retained.

In the correspondence table illustrated in FIG. 31, a set of algorithms are provided so that the set of algorithms are associated with each of attachment positions and each of measurement sites. In the example illustrated in FIG. 31, the number of variations of the attachment position is 27, and the number of variations of the measurement site is 5, as an example. Accordingly, 135 (27×5) algorithms are prepared in advance.

The algorithm selecting section 222 selects an algorithm on the basis of (i) the attachment position and the measurement site, transmitted from the attribute information determining section 221. For example, in a case where “FRONT SIDE-CHEST-UPPER LEFT” is selected as “ATATCHEMENT POSITION” and “HEART SOUND” is selected as “MEASUREMENT SITE”, the algorithm selecting section 222 refers to the correspondence table illustrated in FIG. 31, and selects the algorithm “A3”.

FIG. 32 illustrates a specific example of the algorithm A3 selected. In the present embodiment, the analysis device 201 includes the quality assessing section 223 and the state evaluating section 224 each as the information processing section. For this reason, the algorithm A3 includes at least (i) a quality assessing algorithm A3 for a quality assessing process to be carried out by the quality assessing section 223 and (ii) a state evaluating algorithm A3 for a state evaluating process to be carried out by the state evaluating section 224. Here, in a case where the analysis device 201 includes the third information processing section and the fourth information processing section, the algorithm A3 includes (i) an algorithm A3 for an information processing to be carried out by the third information processing section and (ii) an algorithm A3 for an information processing to be carried out by the fourth information processing section.

In the example illustrated in FIG. 32, since a single quality assessing algorithm A3 is provided for each of the attachment positions and each of the measurement sites, the same quality assessing algorithm A3 is shared irrespective of the measurement items. In this case, the algorithm selecting section 222 selects the quality assessing algorithm A3, and instructs the quality assessing section 223 to carry out the quality assessing process in accordance with the algorithm thus selected.

In the example illustrated in FIG. 32, a plurality of state evaluating algorithms A3 are prepared for the respective measurement items. For this reason, the algorithm selecting section 222 selects a corresponding algorithm on the basis of a determined measurement item. For example, in a case where the user selects “MITRAL OPENING SNAP (disease name: mitral incompetence)” as “MEASUREMENT ITEM”, the algorithm selecting section 222 selects, from among the state evaluating algorithms A3 shown in FIG. 32, an algorithm which includes an evaluation function “f1(x)” and a threshold “6”. The algorithm selecting section 222 instructs the state evaluating section 224 to carry out the state evaluating process in accordance with the algorithm selected. It is possible that in a case where no measurement item is determined by the attribute information determining section 221, the algorithm selecting section 222 instructs the state evaluating section 224 to carry out all the state evaluating algorithms A3.

As illustrated in FIG. 32, the algorithm (for example, the algorithm A3) uniquely determined with reference to the correspondence table includes a pair of algorithms, namely, the quality assessing algorithm and the state evaluating algorithm, and the quality assessing algorithm is selected before the state evaluating algorithm is selected. The state evaluating algorithm is prepared for each of the measurement items. With this arrangement, as to, for example, the algorithms A3 for evaluating the heart sound, different state evaluating algorithms are prepared for respective characteristics (the measurement items or target diseases) of a heart murmur of the heart sound. Accordingly, the quality assessing section 223 can carry out detailed evaluation for each of various diseases on the basis of the sound data obtained from a single kind of acoustic sensor 202.

The quality assessing section 223 carries out a quality assessing process. The quality assessing process is one of information processes included in the biometric process carried out by the analysis device 201. Specifically, the quality assessing process is such that the biometric sound signal information (i.e., the sound data) obtained from the acoustic sensor 202 is analyzed so as to assess whether quality of the biometric sound signal information is good or bad as the sound data for use in the measurement. The quality assessing section 223 processes the sound data in accordance with the quality assessing algorithm selected by the algorithm selecting section 222. Then, the quality assessing section 223 determines whether or not the sound data collected has sufficient quality to achieve the predetermined purpose of the measurement. For example, in a case where the measurement site “HEART SOUND” is selected but a sound volume of the heart sound in the sound data is insufficient, the quality assessing section 223 determines that the quality of the sound data is insufficient. The quality assessing section 223 can output a result of the assessment of the quality of the sound data to the display section 215. This makes it possible for the user to adjust the attachment position of the acoustic sensor 202 attached to the subject, and improve the attachment state. Alternatively, the information obtaining section 220 can obtain the sound data from the acoustic sensor 202 again in accordance with an instruction received from the quality assessing section 223. The quality assessing section 223 transmits, to an information processing section (for example, the state evaluating section 224) for carrying out the following process, only the sound data for which the quality assessing section 223 has determined that the quality is high. With this arrangement, it is possible to prevent such a case in which the processing is carried out with insufficient sound data.

The state evaluating section 224 carries out the state evaluating process. The state evaluating process is one of information processes included in the biometric process carried out by the analysis device 201. Specifically, the state evaluating process is such that various kinds of information (parameters) related to the subject is extracted from the biometric sound signal information, and a state of the subject is evaluated on the basis of the parameters thus extracted. The state evaluating section 224 processes the sound data in accordance with the state evaluating algorithm selected by the algorithm selecting section 222. Then, the state evaluating section 224 derives measurement result information in accordance with a selected measurement item. For example, in a case where the measurement item “MITRAL OPENING SNAP (disease name: mitral incompetence)” is selected, the state evaluating section 224 (i) extracts various parameters from the sound data, (ii) multiplies each of the various parameters by an evaluation function “f1(x)”, and then (iii) compares a value obtained with a threshold “6”. After that, on the basis of a result of such comparison, the state evaluating section 224 evaluates presence or absence of abnormality in terms of the mitral incompetence. Further, the state evaluating algorithm can include, irrespective of the presence or absence of abnormality, calculation for finding a heart rate from the sound data. The state evaluating section 224 outputs, as the measurement result information, to the display section 215, (i) a result of the evaluation about the presence or absence of abnormality, (ii) the heart rate, and (iii) other derived information.

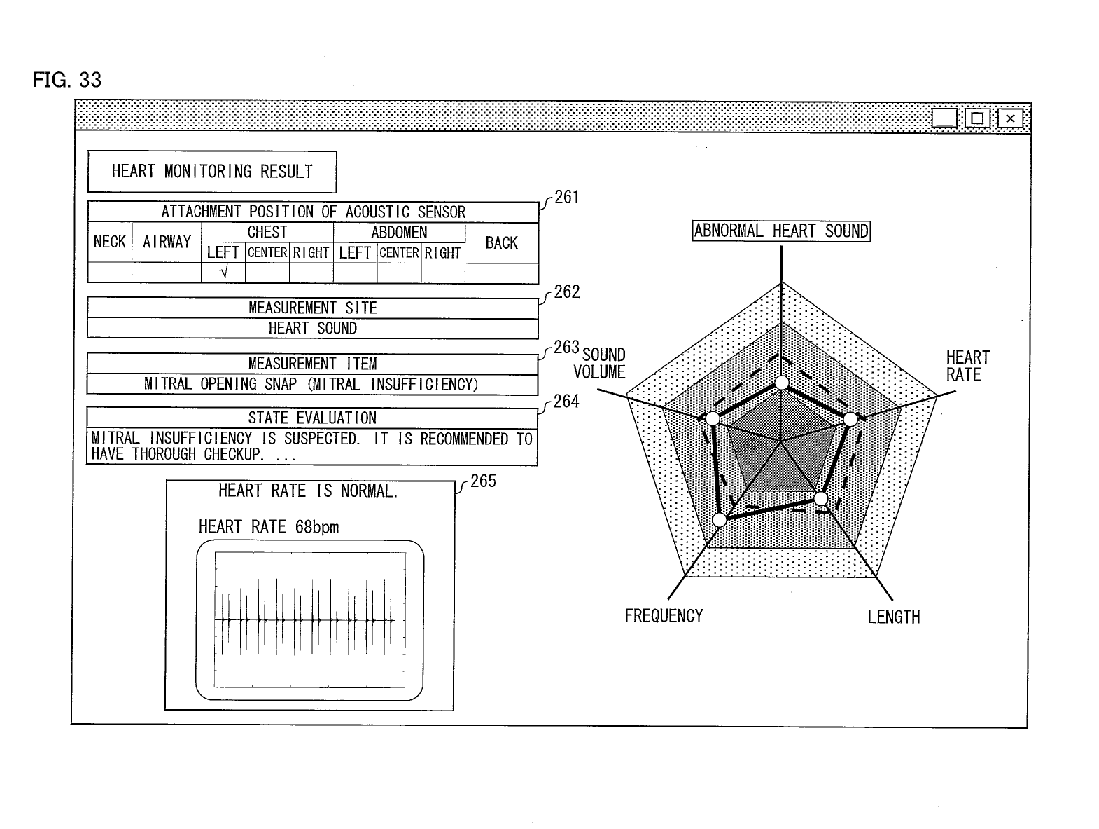

FIG. 33 is a view showing an example of an output screen of the measurement result information, displayed on the display section 215. As shown in FIG. 33, the state evaluating section 224 outputs a state evaluation result 264 of the subject in terms of the measurement item selected. In the example shown in FIG. 33, the state evaluation result 264 includes evaluation regarding whether the state of the subject is normal or abnormal (or caution, wait-and-see process required, etc.), in terms of the selected measurement item “MITRAL OPENING SNAP (disease name: mitral incompetence)”. Further, the state evaluating section 224 can cause selected attribute information (an attachment position 261, a measurement site 262, and a measurement item 263) to be displayed. Further, in a case where the heart rate is obtained, the state evaluating section 224 can cause the display section 215 to display, as heart rate information 265, (i) a calculation result of the heart rate and (ii) information on presence or absence of abnormality in terms of the heart rate. Further, the state evaluating section 224 can cause the display section 215 to display a result of evaluation of various biometric parameters which are extracted from the sound data during the state evaluating process. For example, as illustrated in FIG. 33, it is possible to cause the result of the evaluation to be displayed in a radar chart format.

Note that the measurement result information outputted from the state evaluating section 224 is transmitted to each device of the external device 203 in accordance with necessity or purpose of the measurement result information, and is displayed, stored, or used in another process by the external device 203.

Respective operations of the quality assessing section 223 and the state evaluating section 224 will be described later with reference to a specific example.

[Biometric Process Flow]

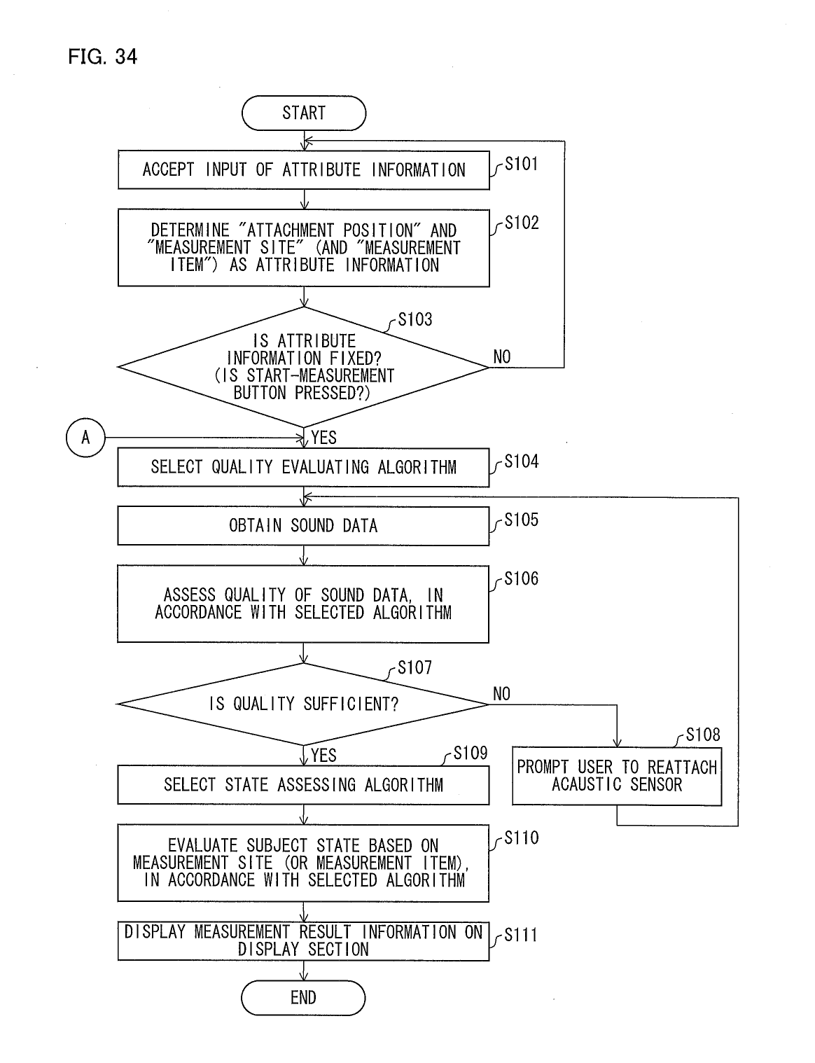

FIG. 34 is a flowchart illustrating a flow of a biometric process carried out by the analysis device 201 of the present embodiment.

Upon activation of an application for carrying out the biometric process in the analysis device 201, the attribute information determining section 221 causes the display section 215 to display an input screen such as the one illustrated in FIG. 30, and thus accepts a user's selection of attribute information (S101). The attribute information determining section 221 then determines attribute information “attachment position”, “measurement site”, and “measurement item” on the basis of options selected via the input operation section 214 (S102).

The user attaches the acoustic sensor 202 to an identical position of a subject body to the attachment position selected in S101. After having completed preparation for measurement and having made sure that there is no problem with the determined attribute information, the user instructs the analysis device 201 to start measurement by, for example, clicking the “START MEASUREMENT” button illustrated in FIG. 30. Note that the acoustic sensor 202 may alternatively be attached to a predetermined attachment position before the input of the attachment position in S101.

In response to the clicking of the “START MEASUREMENT” button via the input operation section 214 (YES in S103), the algorithm selecting section 222 selects a quality assessing algorithm corresponding to the “attachment position” and “measurement site” determined by the attribute information determining section 221 (S104). This completes preparation for start of measurement, and allows the analysis device 201 and the acoustic sensor 202 to shift to a state for carrying out the biometric process.

First, the acoustic sensor 202 gathers a biometric sound of the subject. The information obtaining section 220 obtains sound data (biometric sound signal information) of the biometric sound from the acoustic sensor 202 (S105). The quality assessing section 223 assesses, in accordance with the quality assessing algorithm selected by the algorithm selecting section 222, quality of the sound data obtained in S105 (S106). For example, the quality assessing section 223 assesses whether or not a sound at the measurement site selected in S101 has a sound volume that is equal to or higher than a predetermined sound volume in the sound data. This determines (i) whether or not the acoustic sensor 202 is attached to an appropriate position or in an appropriate state and (ii) whether or not a biometric sound based on a measurement site has been measured at high quality.

In a case where the quality assessing section 223 has determined that the quality of the sound data is insufficient (NO in S107), the quality assessing section 223 may cause the display section 215 to display an error message notifying the user that the acoustic sensor 202 is not attached to an appropriate position or in an appropriate state, thereby prompting the user to attach the acoustic sensor 202 again (S108). In addition, the quality assessing section 223 may cause the display section 215 to display a human body figure 240 of FIG. 30 so as to show the user a correct attachment position.

Meanwhile, in a case where the quality assessing section 223 has determined that the quality of the sound data (including sound data obtained again after the acoustic sensor 202 is attached again) is sufficient (YES in S107), the analysis device 201 shifts to processing for obtaining detailed health information. Specifically, the algorithm selecting section 222 selects a state evaluating algorithm on the basis of the attachment position, the measurement site, and the measurement item selected in S101 (S109). Then, the state evaluating section 224 processes, in accordance with the state evaluating algorithm selected by the algorithm selecting section 222, the sound data obtained in S105, so as to evaluate a state of the subject (S110). The state evaluating section 224 measures and evaluates the subject's state corresponding to the measurement item selected, and then supplies measurement result information thus derived to the display section 215 (S111). The measurement result information is displayed, for example, as the output screen illustrated in FIG. 33.

According to the arrangement of the analysis device 201 and the biometric method in accordance with the present embodiment, a user can carry out accurate measurement based on various measurement items with use of an acoustic sensor (a single kind of acoustic sensor) simply by an easy input operation. It is thus possible to provide a biometric system 200 that is efficient and highly convenient especially for a user who has (i) a clear desire about a sound to be measured (measurement site) and has (ii) a certain level of knowledge about a measurement method (attachment position) for measuring such a sound.

[Quality Assessing Process]

The following describes, by using a specific example, a quality assessing process carried out by the quality assessing section 223 in S106. In the following description, it is assumed that the attribute information determining section 221 determines that the attachment position is “FRONT SIDE-CHEST-UPPER LEFT” and the measurement site is “HEART SOUND”.

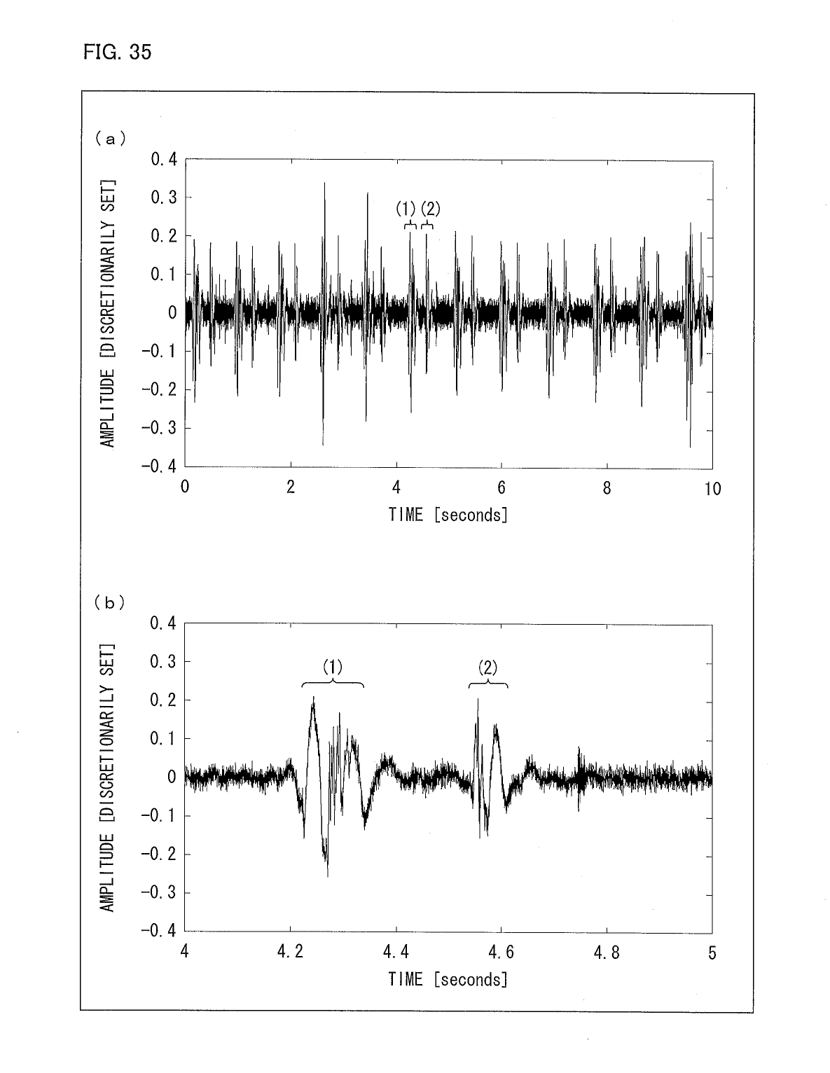

(a) and (b) of FIG. 35 are each a diagram illustrating a waveform of sound data gathered by the acoustic sensor 202. To conclude in advance, the waveform of the sound data illustrated in FIG. 35 is an example of a waveform of a normal heart sound which waveform is, however, insufficient in quality as biometric sound signal information for use in measurement due to a large background noise caused by a poor attachment state of the acoustic sensor 202. (a) of FIG. 35 illustrates a waveform obtained during a period of 10 seconds. (b) of FIG. 35 is an enlarged view of (a) of FIG. 35, and illustrates a waveform obtained during a period of 1 second between (i) a time point at which a period of 4 seconds has elapsed (relative elapsed time) and (ii) a time point at which a period of 5 seconds has elapsed (relative elapsed time). (1) in FIG. 35 indicates a waveform of a sound I of the heart sound, and (2) in FIG. 35 indicates a waveform of a sound II of the heart sound.

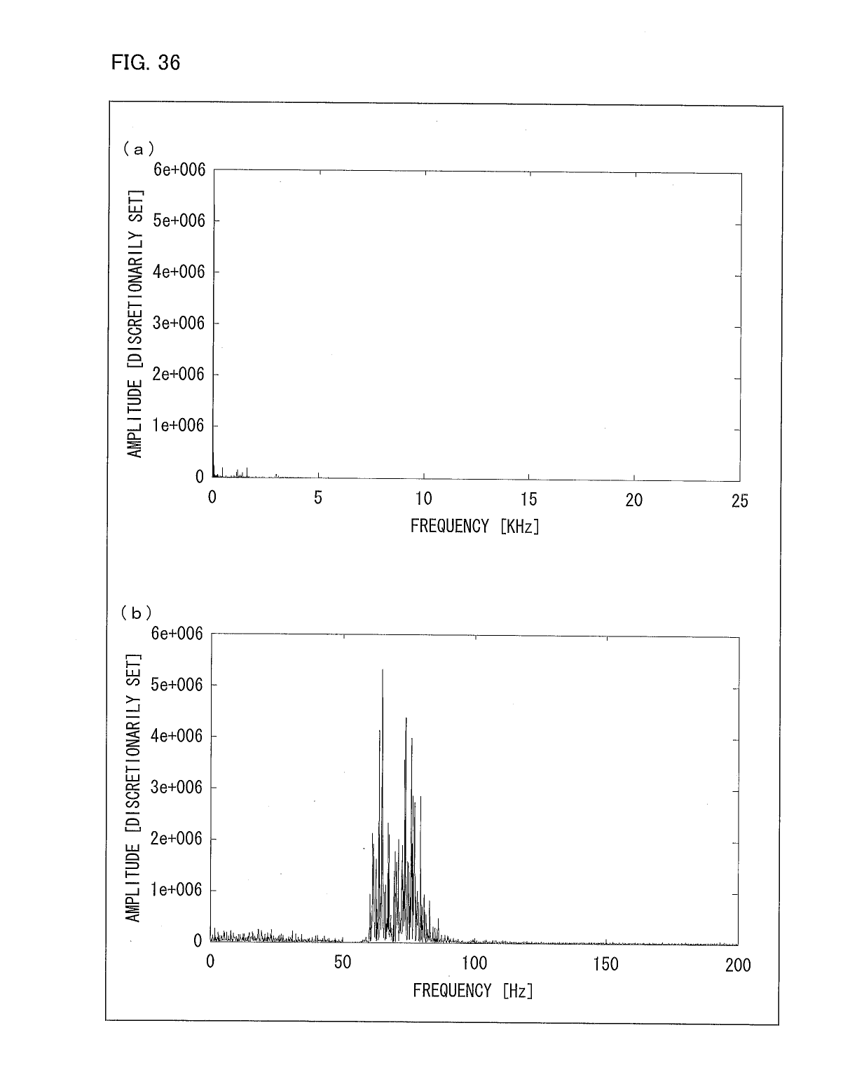

The quality assessing section 223 first carries out, in accordance with the quality assessing algorithm selected (e.g., A3), a fast Fourier transform (FFT) process with respect to the waveform of the sound data illustrated in FIG. 35. (a) and (b) of FIG. 36 are each a diagram illustrating a frequency spectrum of sound data obtained through a FFT process for the sound data illustrated in (a) and (b) of FIG. 35. (a) of FIG. 36 illustrates a frequency spectrum between the frequency 0 KHz and the frequency 25 KHz. (b) of FIG. 36 is an enlarged view of (a) of FIG. 36, and illustrates a frequency spectrum between the frequency 0 Hz and the frequency 200 Hz.

A heart sound is characterized in that its spectrum is concentrated on a band from 60 Hz to 80 Hz. This band which serves as a standard is referred to as a signal band. It is assumed that a signal band is predetermined for each measurement site. A signal band for a heart sound is 60 Hz to 80 Hz as described above.

As illustrated in (b) of FIG. 36, the spectrum is concentrated on a signal band from 60 Hz to 80 Hz. This allows the quality assessing section 223 to estimate that the sound data gathered contains a heart sound. However, as illustrated in (b) of FIG. 36, this sound data contains many components not only in the signal band from 60 Hz to 80 Hz, but also in a band of 50 Hz and lower. The quality assessing section 223 detects, as a noise, such components present in a band (e.g., band of 50 Hz and lower) other than a signal band. Note that the analysis device 201 may be arranged such that (i) the sound source storage section 232 stores in advance sample sound data that is prepared from a clear heart sound gathered in advance as a sound source and (ii) the quality assessing section 223 detects presence or absence of a noise through comparison with the sample sound data. The sound source storage section 232 may store sample sound data itself or may store features extracted from the sound data by a predetermined procedure. Such features may be obtained by carrying out predetermined processing with respect to sound data in advance or may be a statistical value obtained by carrying out statistical processing with respect to the sound data. In view of a storage capacity of the sound source storage section 232 and a processing load of the analysis device 201 which carries out the comparison, features of sample sound data are preferable to the sample sound data itself as a data to be stored in the sound source storage section 232 since a data volume of the features is far smaller than that of the sample sound data itself. It is therefore preferable to arrange the analysis device 201 to compare features with features.

Subsequently, in accordance with the quality assessing algorithm, the quality assessing section 223 finds Bsignal, which is a size of a component of a spectrum in the signal band (60 Hz to 80 Hz), and Bnoise, which is a size of a sum of (i) the component in the signal band and (ii) a component in the band other than the signal band. Then, the quality assessing section 223 calculates a ratio of Bsignal and Bnoise as SNR indicative of signal quality of the sound data. That is, the quality assessing algorithm includes the following Formula 1.

$$SNR = \vert \frac{Bsignal}{Bnoise} \vert \qquad Math . 1$$

The quality assessing section 223 assesses quality of the sound data with use of Formula 1.

In the example of the sound data illustrated in FIGS. 35 and 36, the quality assessing section 223 finds “465880448” as Bsignal and “143968” as Bnoise, and finally calculates SNR as follows:

465880448/143968=3236

The larger the value of SNR is, the higher the signal quality is. The present embodiment describes, as an example, a case where a threshold of SNR is set to 10000, sound data having SNR of 10000 or higher is determined to have good quality (determined to be measurable) and sound data having SNR of less than 10000 is determined to have insufficient quality (determined to be unmeasurable). The quality assessing algorithm thus includes an assessment condition for assessing signal quality.

As described above, the sound data illustrated in FIGS. 35 and 36 is insufficient in quality as sound data to provide measurement result information, because of much background noise due to an incomplete attachment state. The quality assessing section 223 calculates SNR of the sound data illustrated in FIGS. 35 and 36 to 3236 in accordance with the quality assessing algorithm selected, and then compares the SNR thus calculated with the threshold 10000. The result of the comparison is “SNR=3236<threshold 10000”. As such, the quality assessing section 223 determines that the SNR of the sound data does not reach the threshold and that the sound data is insufficient in quality.

In this case, the quality assessing section 223 causes the display section 215 to display a message such as “Attachment state of acoustic sensor 202 is unstable. Please attach acoustic sensor 202 again” so as to prompt the user to attach the acoustic sensor 202 again.

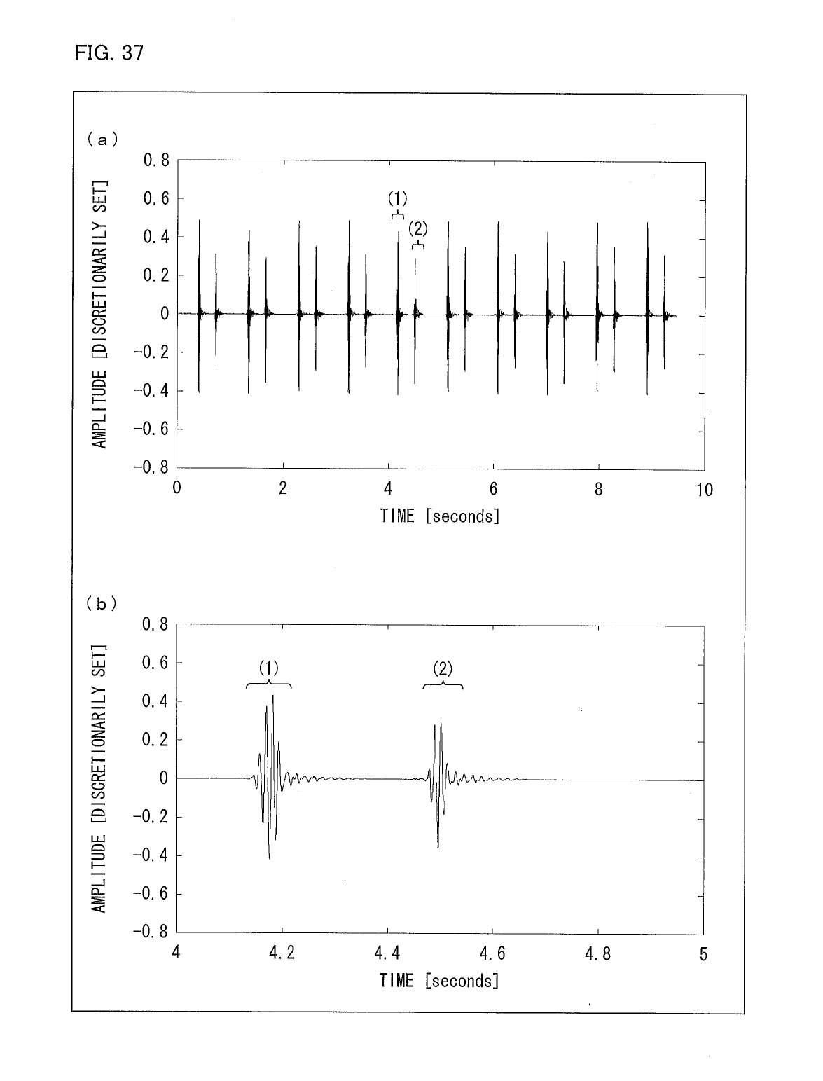

(a) and (b) of FIG. 37 are each a diagram illustrating a waveform of sound data gathered by the acoustic sensor 202 after the user attaches the acoustic sensor 202 again. To conclude in advance, the waveform of the sound data illustrated in FIG. 37 is an example of a waveform that is sufficiently good as biometric sound signal information for use in measurement as a result of a reduction in background noise achieved by improvement of the attachment state. (a) of FIG. 37 illustrates a waveform during a period of 10 seconds. (b) of FIG. 37 is an enlarged view of (a) of FIG. 37, and illustrates a waveform during a period of 1 second between (i) a time point at which a period of 4 seconds has elapsed (relative elapsed time) and (ii) a time point at which a period of 5 seconds has elapsed (relative elapsed time). (1) in FIG. 37 indicates a waveform of a sound I of the heart sound, and (2) in FIG. 37 indicates a waveform of a sound II of the heart sound.

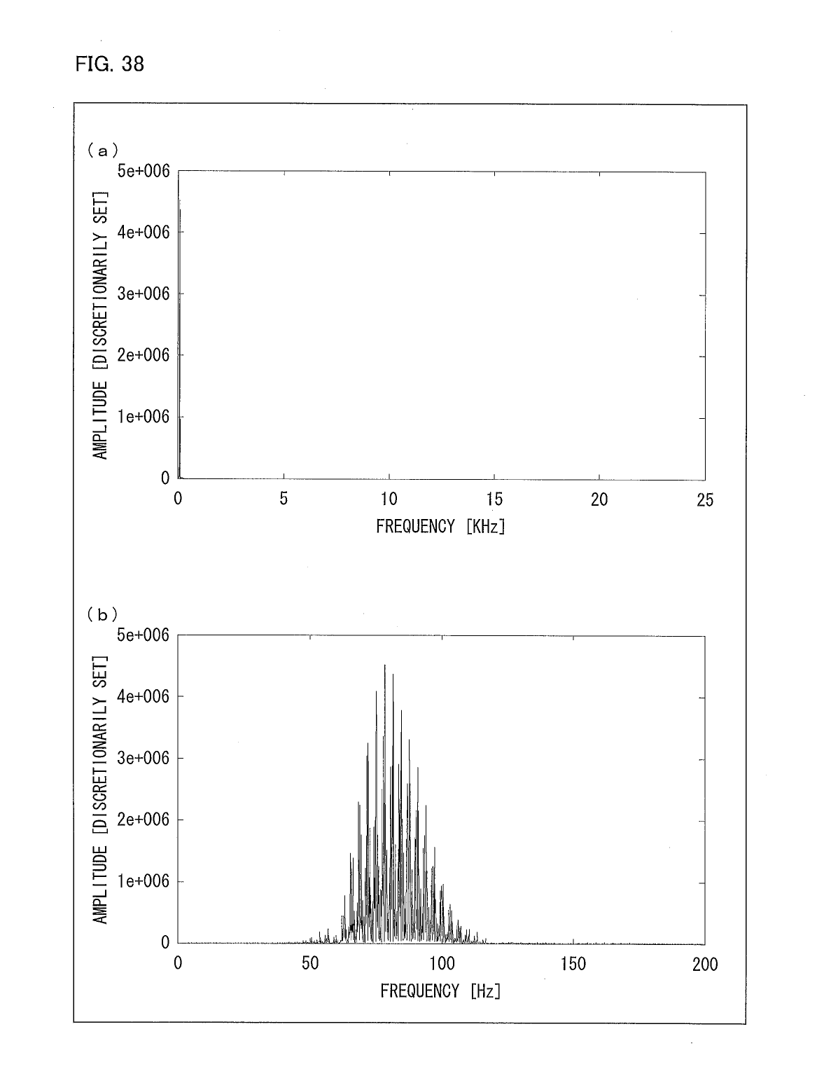

The quality assessing section 223 carries out, in accordance with the quality assessing algorithm through a procedure similar to the above, a FFT process with respect to the waveform of the sound data illustrated in FIG. 37. (a) and (b) of FIG. 38 are each a diagram illustrating a frequency spectrum of sound data obtained through the FFT process for the sound data illustrated in (a) and (b) of FIG. 37. (a) of FIG. 38 illustrates a frequency spectrum between the frequency 0 KHz and the frequency 25 KHz. (b) of FIG. 38 is an enlarged view of (a) of FIG. 38, and illustrates a frequency spectrum between the frequency 0 Hz and the frequency 200 Hz.

The quality assessing section 223 finds “589981113” as Bsignal and finds “14643” as Bnoise on the basis of the frequency spectrum obtained, and finally calculates SNR as follows:

589981113/14643=40291

The quality assessing section 223 determines that SNR of the sound data is higher than the threshold 10000 and that the sound data is sufficient in quality.

The above description deals with a case in which the threshold of SNR is included in the quality assessing algorithm in advance. Note, however, that the arrangement of the analysis device 201 is not limited to this. For example, the quality assessing algorithm may include an algorithm for matching between gathered sound data and sample sound data stored in the sound source storage section 232. In this case, the quality assessing section 223 can assess quality on the basis of a degree of matching between (i) a frequency spectrum of the gathered sound data and (ii) a frequency spectrum of the sample sound data stored in the sound source storage section 232 as a result of comparison according to the quality assessing algorithm.

[State Evaluating Processing]

The following describes state, by using a specific example, evaluating processing carried out by the state evaluating section 224 in S110. In the following description, it is assumed that the attribute information determining section 221 determines that the attachment position is “FRONT SIDE-CHEST-UPPER LEFT”, the measurement site is “HEART SOUND”, and the measurement item is “MITRAL OPENING SNAP (MITRAL INCOMPETENCE)”.

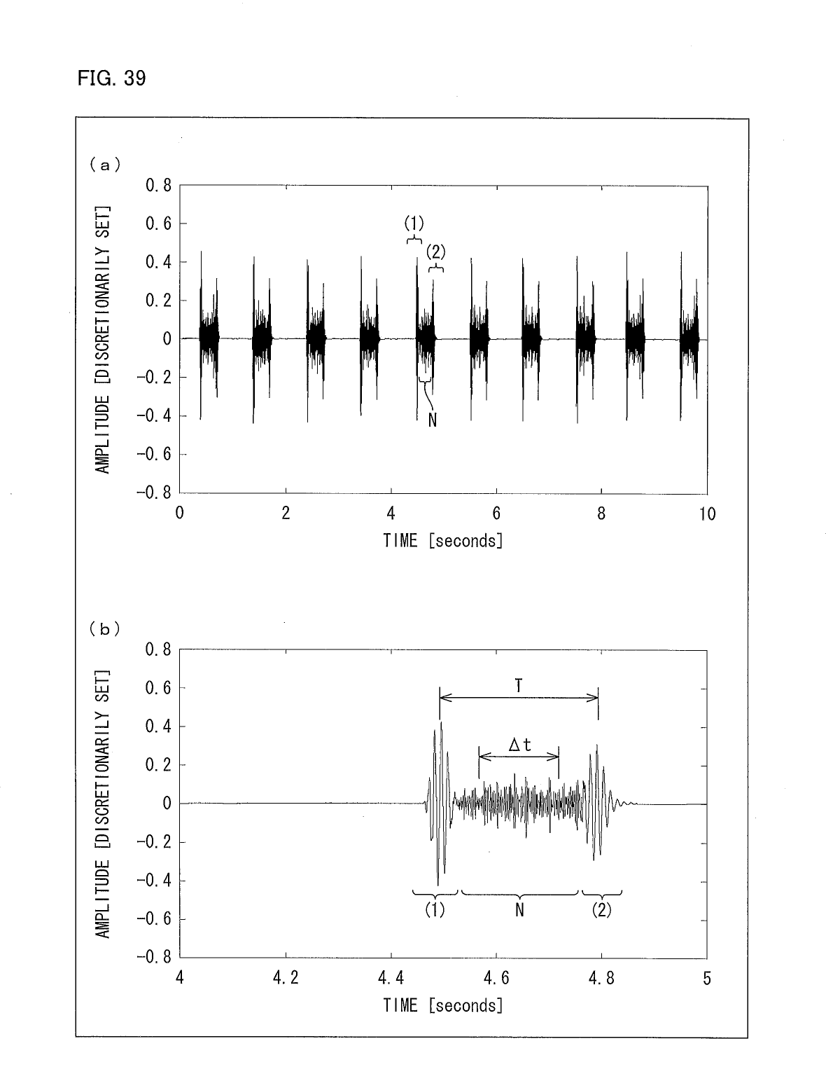

(a) and (b) of FIG. 39 are each a diagram illustrating a waveform of sound data gathered by the acoustic sensor 202. (a) of FIG. 39 illustrates a waveform during a period of 10 seconds. (b) of FIG. 39 is an enlarged view of (a) of FIG. 39, and illustrates a waveform during a period of 1 second between (i) a time point at which a period of 4 seconds has elapsed (relative elapsed time) and (ii) a time point at which a period of 5 seconds has elapsed (relative elapsed time). (1) in FIG. 39 indicates a waveform of a sound I of heart sound, and (2) in FIG. 39 indicates a waveform of a sound II of heart sound. The waveform of the sound data illustrated in FIG. 39 has a relatively large signal sound N similar to noise between the sound I and the sound II, as compared with the waveform of the normal heart sound illustrated in FIG. 37. To conclude in advance, the waveform illustrated in FIG. 39 is a typical example of abnormal heart sound. Specifically, the waveform illustrated in FIG. 39 is an example of a heart sound waveform of a subject suffering from mitral incompetence (incompetence of closing of a mitral valve between left and right ventricles of the heart).

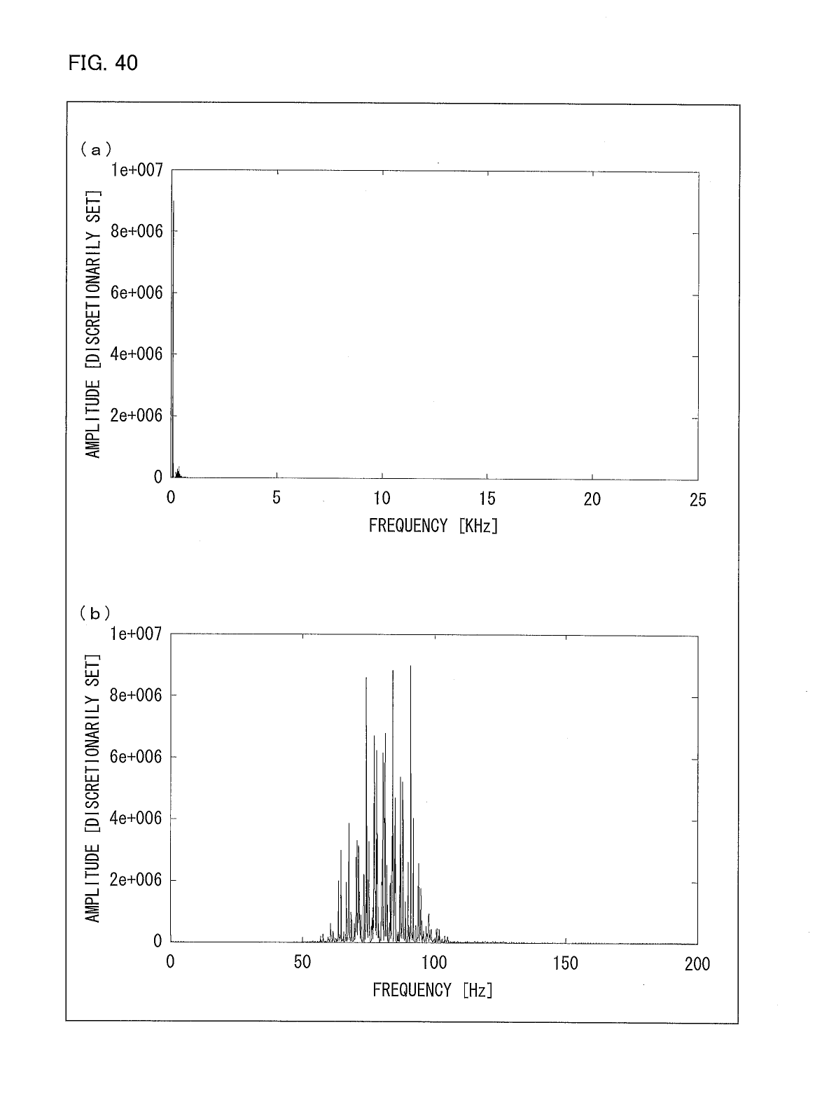

Note that the quality assessing section 223 assesses quality of the sound data before the state evaluating section 224 carries out state evaluating processing about the measurement item “MITRAL OPENING SNAP (MITRAL INCOMPETENCE)”. (a) and (b) of FIG. 40 are each a diagram illustrating a frequency spectrum of sound data obtained through a FFT process for the sound data illustrated in (a) and (b) of FIG. 39. (a) of FIG. 40 illustrates a frequency spectrum between the frequency 0 KHz and the frequency 25 KHz. (b) of FIG. 40 is an enlarged view of (a) of FIG. 40, and illustrates a frequency spectrum between the frequency 0 Hz and the frequency 200 Hz. In the example illustrated in FIG. 40, the quality assessing section 223 calculates SNR of the sound data as follows:

805504207/25943=31049

As such, the quality assessing section 223 determines that the sound data has sufficient signal quality. However, it is difficult at a glance to determine that the sound data indicates an abnormal heart sound, even through comparison between (i) the frequency spectrum of FIG. 40 obtained through the series of processing of the quality assessing section 223 and (ii) a frequency spectrum (e.g., the frequency spectrum of FIG. 38) of sample sound data stored in the sound source storage section 232. The state evaluating section 224 carries out the state evaluating processing with respect to the measurement item “MITRAL OPENING SNAP (MITRAL INCOMPETENCE)” with use of a state evaluating algorithm different from the quality assessing algorithm used by the quality assessing section 223.

The state evaluating algorithm selected by the algorithm selecting section 222 is an algorithm A3 illustrated in FIG. 32 including the evaluation function “f1(x)” and the threshold “6”, in accordance with the example of attribute information.

The state evaluating section 224 calculates the function f1(x) included in the state evaluating algorithm selected. The following formula 2:

$$f 1 ( x ) = \frac{1}{Δ t} {\sum }_{Δ t} {( A ( x ) - \overline{A ( x )} )}^{2} \qquad Math . 2$$

expresses the function f1(x).

Specifically, as illustrated in (b) of FIG. 39, the state evaluating section 224 first finds an interval Δt in a sound data string A(x) by removing, from a time interval T between the sound I and the sound II, the initial 25% and last 25% thereof. The sound data string A(x) includes sound data corresponding to one or more cycles of heartbeat. Then, the state evaluating section 224 calculates signal power of the sound data string A(x) in the interval Δt in accordance with the formula 2. The f1(x) for the sound data illustrated in FIG. 39 is calculated in accordance with the formula 2 to be 12.6.

The state evaluating algorithm includes an assessment condition for determining that the heart sound has abnormality (mitral incompetence) in a case where a value of f1(x) is equal to or larger than the threshold 6 and determining that the heart sound is normal in a case where the value of f1(x) is smaller than the threshold 6.

As a result of comparison between f1(x)=12.6 calculated above and the threshold 6, the state evaluating section 224 determines that f1(x)≧6. Based on this determination, the state evaluating section 224 evaluates a state of a subject from which the sound data illustrated in FIG. 39 was gathered to be a state of being suspected of heart sound abnormality, especially mitral incompetence. The state evaluation result derived by the state evaluating section 224 is presented to a user as the state evaluation result 264 illustrated in FIG. 33 through, for example, display on the display section 215.

The above description deals with a case in which the threshold of f1(x) is included in the state evaluating algorithm in advance. Note, however, that the arrangement of the analysis device 201 is not limited to this. For example, the state evaluating algorithm may include an algorithm for matching between gathered sound data and sample sound data stored in the sound source storage section 232. In this case, the state evaluating section 224 can assess quality on the basis of a degree of matching between (i) a value of f1(x) of the gathered sound data (“12.6” in the case of the waveform of FIG. 39) and (ii) a value of f1(x) of the sample sound data stored in the sound source storage section 232 (e.g., “0.02” assuming that the sample sound data has the waveform of FIG. 37), as a result of comparison according to the state evaluating algorithm.

The evaluation function and the threshold are merely examples of the state evaluating algorithm. The state evaluating algorithm is not limited to these, and includes all formulas and values for detecting a target disease or symptom. These state evaluating algorithms are determined as appropriate on the basis of medical knowledge and experiments.

### Embodiment 2-2

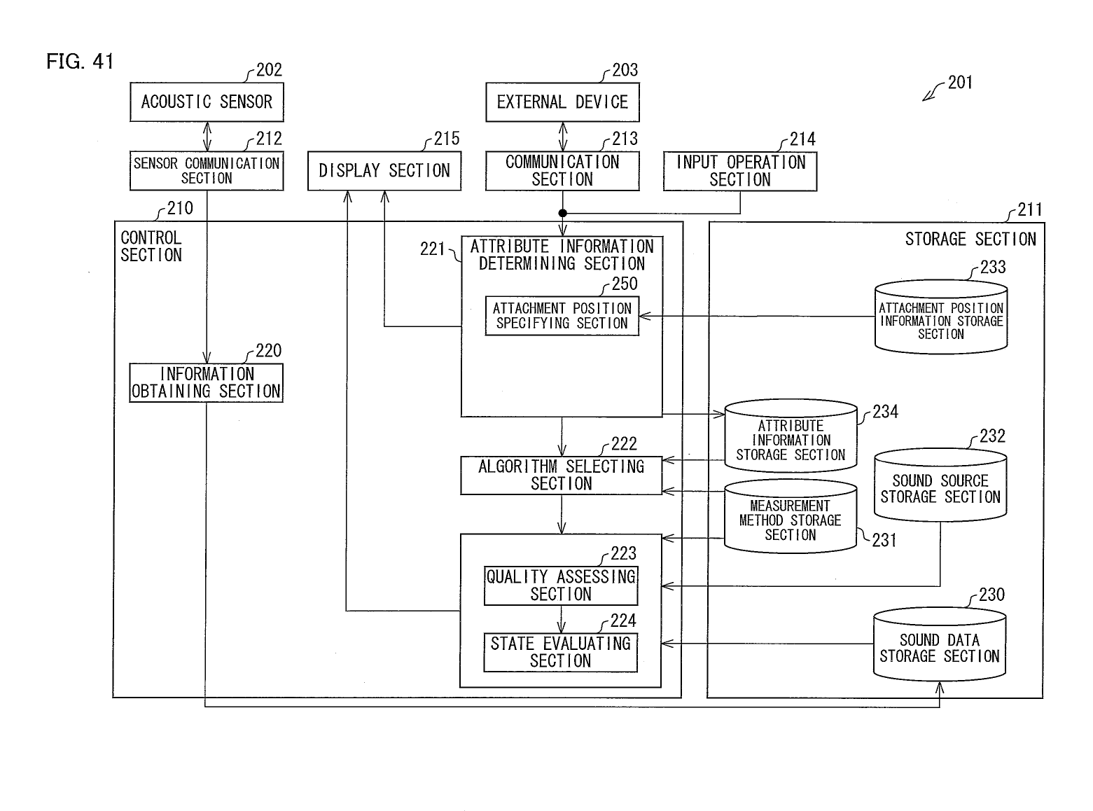

Another embodiment of the analysis device 201 of the present invention is described below with reference to FIGS. 41 through 45. For convenience of explanation, members that have functions identical to those of members illustrated in the drawings of Embodiment 2-1 are given identical reference numerals, and are not explained repeatedly.

Embodiment 2-1 deals with an arrangement in which a user manually inputs attribute information (i.e., attachment position, measurement site, and measurement item) at a stage of preparation for start of measurement. It can be said that the arrangement of Embodiment 2-1 is effective especially for a user who has a clear purpose of measurement (a measurement site or a measurement item) and has a certain level of knowledge about a measurement method (attachment position) for such a purpose.

The present Embodiment 2-2 deals with an arrangement in which (i) a user inputs a purpose of measurement, and then (ii) the analysis device 201 specifies an attachment position of the acoustic sensor 202 effective for the purpose of measurement and presents it to the user. It can be said that the arrangement of Embodiment 2-2 is effective also for a user who has a clear purpose of measurement but does not have knowledge about a measurement method (attachment position) for such a purpose.

[Arrangement of Analysis Device 201]

FIG. 41 is a block diagram illustrating an essential configuration of an analysis device 201 of an embodiment of the present invention. Differently from the analysis device 201 illustrated in FIG. 26, the analysis device 201 illustrated in FIG. 41 is configured such that (i) an attribute information determining section 221 includes an attachment position specifying section 250 for automatically specifying an attachment position of an acoustic sensor 202 and (ii) a storage section 211 includes an attachment position information storage section 233.

The attachment position specifying section 250 specifies an appropriate attachment position on the basis of a purpose of measurement (a measurement site or a measurement item) designated by a user.

The attachment position information storage section 233 stores information indicative of a correspondence relationship between (i) a measurement site and a measurement item for measurement available in the analysis device 201 and (ii) an effective attachment position of the acoustic sensor 202 for the measurement.

The attachment position specifying section 250 is capable of specifying an effective attachment position on the basis of a designated purpose of measurement by referring to the attachment position information storage section 233.

In the present embodiment, the attribute information determining section 221 first causes the display section 215 to display measurement site candidates 243 and measurement item candidates 244 included in the input screen illustrated in FIG. 30, and thus accepts selection of a measurement site (or selection of a measurement site and a measurement item). A user can vaguely select, for example, “HEART SOUND”, “BREATH SOUND”, or “BLOOD FLOW SOUND” as a sound to be measured (measurement site) from the list on the input screen as in Embodiment 2-1, or can additionally select a specific disease name (measurement item).

After the attribute information determining section 221 accepts selection of the user, and determines a measurement site (and a measurement item), the attachment position specifying section 250 specifies, as candidates, attachment positions corresponding to the measurement site (and the measurement item) by referring to the attachment position information storage section 233.

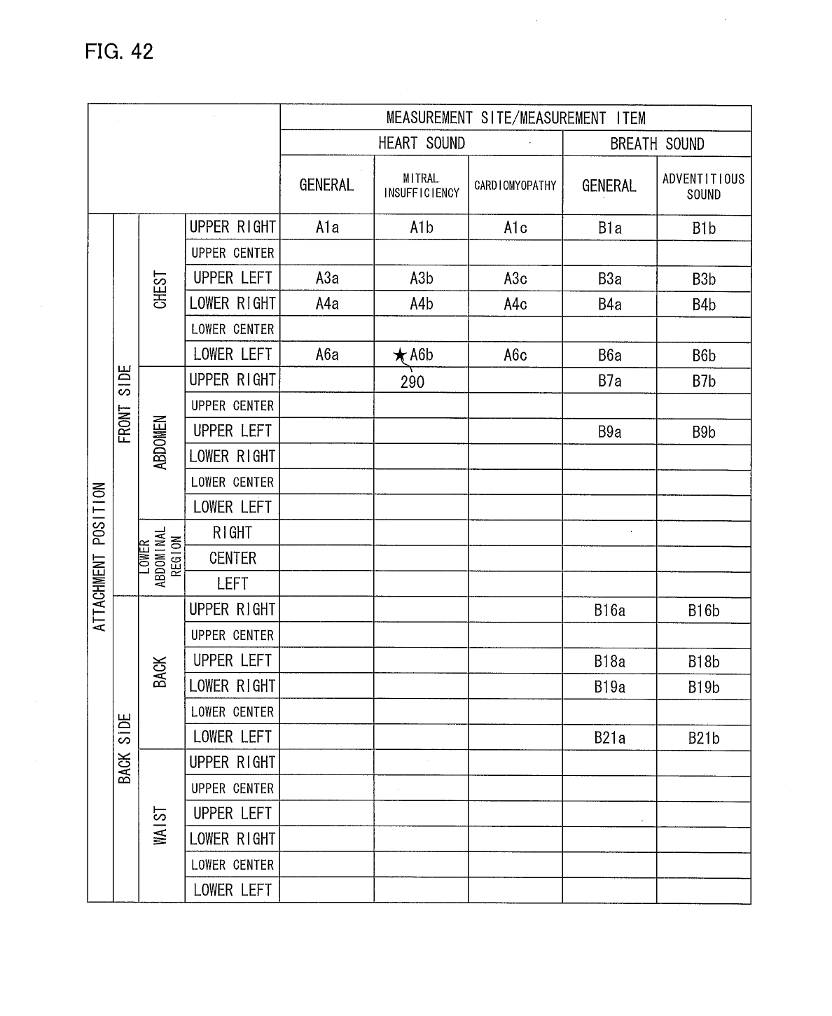

FIG. 42 is a diagram illustrating a specific example of a correspondence table that is stored in the attachment position information storage section 233 and that indicates a correspondence relationship between “MEASUREMENT SITE/MEASUREMENT ITEM” and “ATTACHMENT POSITION”.

As illustrated in FIG. 42, the correspondence table stores, for each combination of the measurement site (and the measurement item) and the attachment position, an identifier for identifying an algorithm for information processing available in the analysis device 201, if such an algorithm exists. Although only the correspondence relationships as for heart sound and breath sound are stored in the example illustrated in FIG. 42, identifiers are stored also for the other measurement sites in a similar manner so that existence of an algorithm can be shown for each attachment position.

The attachment position specifying section 250 refers to the correspondence table illustrated in FIG. 42 in order to specify an attachment position. According to the correspondence table, in a case where “HEART SOUND” has been selected as the measurement site, only four algorithms corresponding to four attachment positions of the acoustic sensor 202, i.e., “FRONT-CHEST-UPPER RIGHT”, “FRONT SIDE-CHEST-UPPER LEFT”, “FRONT SIDE-CHEST-LOWER RIGHT”, and “FRONT SIDE-CHEST-LOWER LEFT” are prepared regardless of which measurement item has been selected. This allows the attachment position specifying section 250 to specify four attachment positions “1: FRONT-CHEST-UPPER RIGHT”, “2: FRONT SIDE-CHEST-UPPER LEFT”, “3: FRONT SIDE-CHEST-LOWER RIGHT”, and “4: FRONT SIDE-CHEST-LOWER LEFT” as effective attachment positions corresponding to the measurement site “HEART SOUND”.

In the present embodiment, it is only necessary for the attachment position specifying section 250 to know existence of an algorithm. It is therefore possible that a flag indicative of existence of an algorithm be merely stored instead of an identifier for an algorithm. Since the algorithm selecting section 222 refers to information indicative of a correspondence relationship concerning an algorithm between the measurement site (and the measurement item) and the attachment position, the correspondence table illustrated in FIG. 42 is stored also in a measurement method storage section 231.

In a case where sensing at an attachment position is especially important or essential for measurement of a measurement item, a flag 290 indicative of importance of the attachment position is preferably stored in addition to the flag indicative of existence of an algorithm. In the example shown in FIG. 42, the flag 290 (indicated by the black star sign) indicates that analysis of sound data at the attachment position “FRONT SIDE-CHEST-LOWER LEFT” is especially important for measurement of the measurement item “MITRAL INCOMPETENCE”. The flag 290 thus allows the attachment position specifying section 250 to recognize importance of an attachment position for each measurement item.

After specifying candidates for the attachment position on the basis of the measurement site (and the measurement item) designated by the user, the attachment position specifying section 250 causes the display section 215 to display again the candidates for the attachment position, and thus accepts selection of the attachment position.

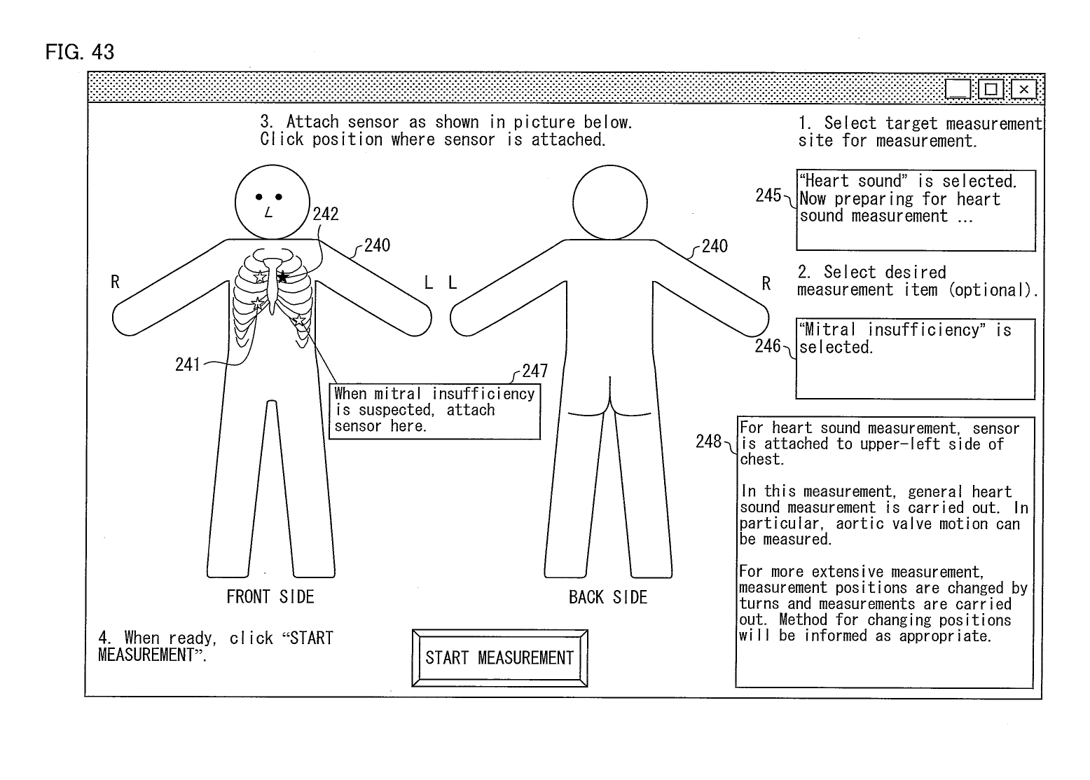

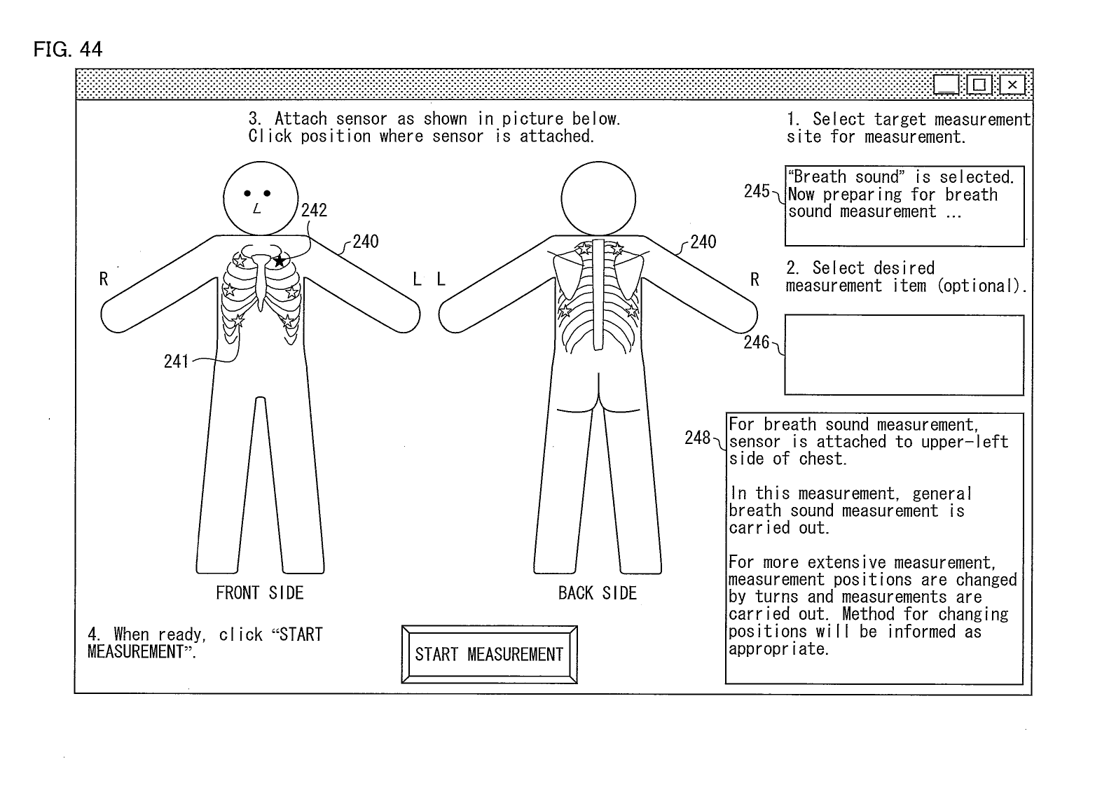

FIGS. 43 and 44 are diagrams each illustrating an example of an attachment position input screen displayed in the display section 215 after (i) the user designates the measurement site (and the measurement item) and (ii) the attachment position specifying section 250 specifies the attachment position. The example illustrated in FIG. 43 is an attachment position input screen displayed in a case where the measurement site “HEART SOUND” and the measurement item “MITRAL INCOMPETENCE” have been selected. The example illustrated in FIG. 44 is an attachment position input screen displayed in a case where the measurement site “BREATH SOUND” has been selected (in a case where no measurement item has been selected).

The attribute information determining section 221 causes the display section 215 to display, along with a human body figure 240, star signs indicative of the respective candidates for the attachment position specified by the attachment position specifying section 250, and thus accepts selection of the attachment position. The user can designate the attachment position of the acoustic sensor 202 by clicking any of the star signs displayed in the display section 215 via an input operation section (mouse) 14. In the examples illustrated in FIGS. 43 and 44, each of the white star signs 241 indicates a non-selected attachment position candidate, and the black star sign 242 indicates a selected attachment position.

As illustrated in FIGS. 43 and 44, the attribute information determining section 221 may cause the display section 215 to display determined measurement site information 245 and determined measurement item information 246.

In the example illustrated in FIG. 42, the flag 290 indicative of importance is given to a combination of the measurement item “MITRAL INCOMPETENCE” and the attachment position “FRONT SIDE-CHEST-LOWER LEFT”. Accordingly, in a case where measurement of a heart sound is carried out regarding MITRAL INCOMPETENCE, the attachment position specifying section 250 may cause the display section 215 to display, along with the star signs indicative of the respective candidates, a message 247 for prompting the user to carry out sensing at the attachment position “FRONT SIDE-CHEST-LOWER LEFT” as illustrated in FIG. 43. This makes it possible to avoid a situation in which information necessary for measurement for the designated purpose cannot be obtained, thereby preventing measurement from being carried out based on incomplete information.

In response to clicking of a star sign indicative of an attachment position candidate, the attribute information determining section 221 determines, as the attribute information “ATTACHMENT POSITION”, an attachment position (e.g., “FRONT SIDE-CHEST-UPPER LEFT”) corresponding to a position of the star sign 242 selected. Then, the attribute information determining section 221 may cause the display section 215 to additionally display guidance information 248 concerning measurement at the attachment position determined, as illustrated in FIGS. 43 and 44.

If the user has no problem with displayed contents, the user attaches the acoustic sensor 202 to a subject in accordance with the attachment position selected. The user simply clicks the measurement start button after preparation for measurement is completed.

The attribute information “ATTACHMENT POSITION”, “MEASUREMENT SITE”, and “MEASUREMENT ITEM” are thus finally determined in the attribute information determining section 221, and transmitted to the algorithm selecting section 222 (or stored in the attribute information storage section 234). The algorithm selecting section 222 selects algorithms (a quality assessing algorithm and a state evaluating algorithm) corresponding to the attribute information “ATTACHMENT POSITION”, “MEASUREMENT SITE”, and “MEASUREMENT ITEM” by referring to the correspondence table illustrated in FIG. 42 (or FIG. 31) through a procedure similar to that described in Embodiment 2-1. In the example illustrated in FIG. 43, in a case where the correspondence table illustrated in FIG. 42 is stored in the measurement method storage section 231, the algorithm selecting section 222 selects “algorithm A3b” on the basis of “ATTACHMENT POSITION: FRONT SIDE-CHEST-UPPER LEFT”, “MEASUREMENT SITE: HEART SOUND”, and “MEASUREMENT ITEM: MITRAL INCOMPETENCE”.

After completion of preparation for start of measurement, the quality assessing section 223 and the state evaluating section 224 carry out, as in Embodiment 2-1, information processing for deriving measurement result information in accordance with the algorithms selected.

[Biometric Process Flow]

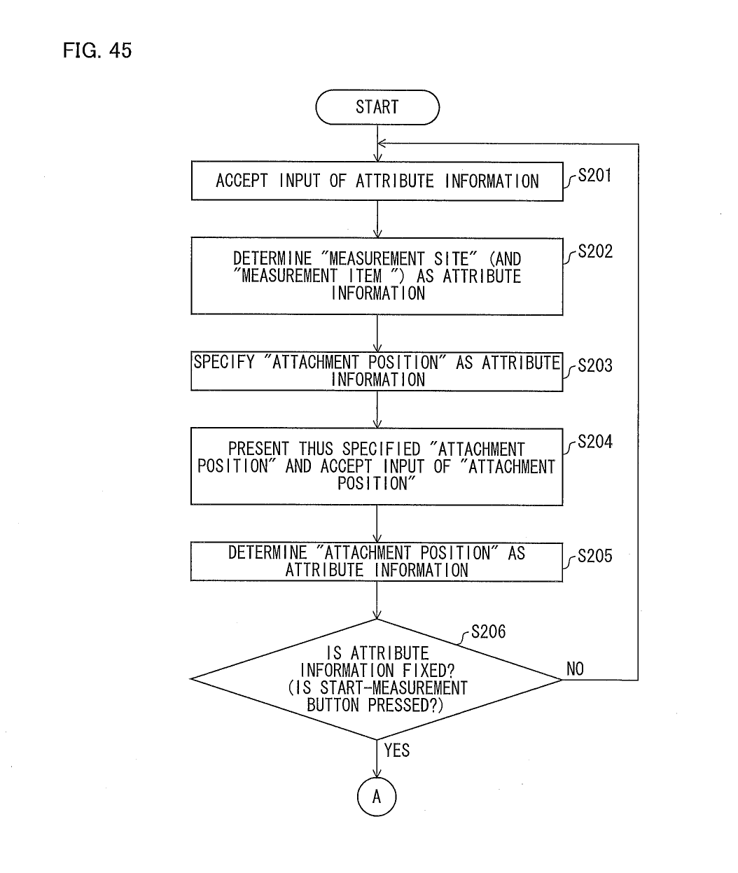

FIG. 45 is a flowchart illustrating a flow of a biometric process carried out by the analysis device 201 of the present embodiment.

Upon activation of an application for carrying out the biometric process in the analysis device 201, the attribute information determining section 221 causes the display section 215 to display an input screen for input of a measurement site and a measurement item, and thus accepts a user's selection of attribute information (S201). The attribute information determining section 221 then determines attribute information “measurement site” (or both of “measurement site” and “measurement item”) on the basis of options selected via the input operation section 214 (S202).

Next, the attachment position specifying section 250 specifies an effective “attachment position” on the basis of the “measurement site” (and the “measurement item”) by referring to the correspondence table stored in the attachment position information storage section 233 (S203).

Then, the attribute information determining section 221 causes the display section 215 to display an attachment position input screen such as the one illustrated in FIG. 43 or FIG. 44 on the basis of contents specified by the attachment position specifying section 250, and thus accepts the user's selection of an attachment position (S204). When the user has selected an attachment position, the attribute information determining section 221 determines, as the attribute information “attachment position”, the attachment position thus selected (S205).

After determination of the attribute information, when the use clicks the “START MEASUREMENT” button via the input operation section 214 (YES in S206), processing for selecting an algorithm starts, as in Embodiment 2-1. In the present embodiment, the algorithm selecting section 222 selects an algorithm corresponding to the “measurement site” (and “measurement item”) determined in S202 by the attribute information determining section 221 and the “attachment position” determined in S205 (S104). This completes preparation for start of measurement, and allows the analysis device 201 and the acoustic sensor 202 to shift to a state for carrying out the biometric process (carrying out S104 and its subsequent steps in FIG. 34).

According to the arrangement of the analysis device 201 and the biometric method in accordance with the present embodiment, even a user who has a clear desire about a sound or a disease to be measured but does not have sufficient knowledge about a measurement method (attachment position) for that purpose can carry out measurement because the user is notified of an effective attachment position by the analysis device 201. Further, by displaying an essential attachment position and a measurement guidance, it is possible to supply a user with knowledge for measurement. It is therefore possible to provide a biometric system 200 that is highly convenient even for a user with poor medical knowledge.

### Embodiment 2-3

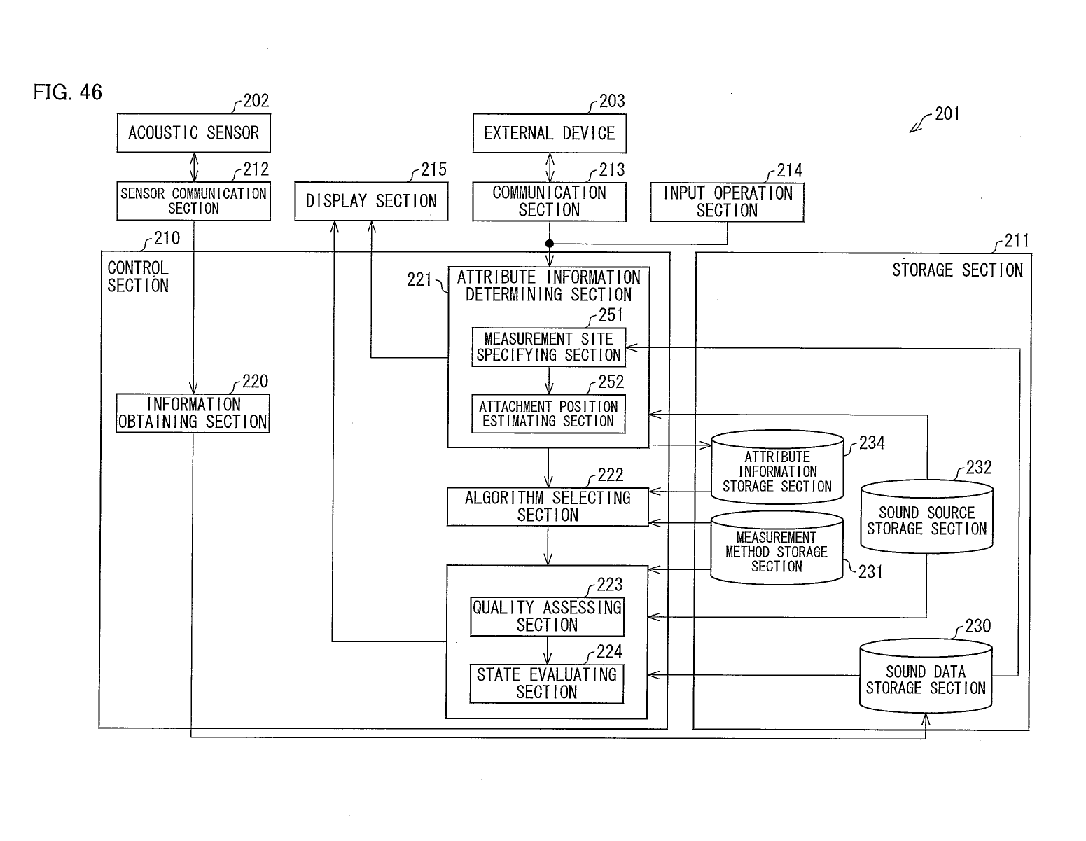

Another embodiment of the analysis device 201 of the present invention is described below with reference to FIGS. 46 through 48. For convenience of explanation, members that have functions identical to those of members illustrated in the drawings of Embodiments 2-1 and 2-2 are given identical reference numerals, and are not explained repeatedly.

Embodiment 2-2 deals with an arrangement in which attribute information is determined in such a manner that a user manually inputs information on a measurement site and a measurement item as attribute information at a stage of preparation for start of measurement so as to narrow candidates for an attachment position down to a certain degree. It can be said that the arrangement of Embodiment 2-2 is effective especially for a user who has a clear purpose of measurement but does not have knowledge about a measurement method.

In the present Embodiment 2-3, a user first attaches an acoustic sensor 202 to a subject without any input of attribute information. In the present embodiment, the user is only required to attach the acoustic sensor 202 roughly around a desired measurement site. The present embodiment describes an arrangement in which an attachment position and a measurement site are specified on the basis of sound data obtained from the acoustic sensor 202 attached. Therefore, it can be said that the arrangement of Embodiment 2-3 is effective for a user who has an approximate purpose of measurement and an approximate knowledge about a measurement method but does not have detailed knowledge. Further, since no detailed manual input operation is necessary, it is possible to further simplify a user's operation at the stage of preparation for start of measurement.

[Arrangement of Analysis Device 201]

FIG. 46 is a block diagram illustrating an essential configuration of an analysis device 201 of an embodiment of the present invention. Differently from the analysis devices 201 illustrated in FIGS. 26 and 41, the analysis device 201 illustrated in FIG. 46 is configured such that an attribute information determining section 221 further includes a measurement site specifying section 251 and an attachment position estimating section 252.

In the present embodiment, first, a user attaches an acoustic sensor 202 to a body of a subject so as to gather a biometric sound. The user can roughly determine an attachment position of the acoustic sensor 202 in the vicinity of a desired measurement site. Then, in response to an instruction via an input operation section 214 to start obtaining sound data, the acoustic sensor 202 starts gathering a sound, and sound data detected by the acoustic sensor 202 is transmitted to an information obtaining section 220 via a sensor communication section 212.

The measurement site specifying section 251 analyzes the sound data (a biometric sound of the subject) obtained as described above from the acoustic sensor 202 so as to specify from which measurement site a sound contained in the sound data has been gathered. The measurement site specifying section 251 specifies the measurement site through matching between (i) features of sample sound data stored in the sound source storage section 232 and (ii) features of the sound data obtained from the acoustic sensor 202. The following describes an example of how the measurement site specifying section 251 specifies a measurement site on the basis of sound data.

In the present embodiment, the measurement site specifying section 251 carries out, as an example, a fast Fourier transform (FFT) process with respect to the sound data obtained from the acoustic sensor 202 so as to find a frequency spectrum of a sound component contained in the sound data. A frequency distribution thus obtained exhibits a characteristic of a target sound source. Similarly, also for the other sounds to be measured such as “BREATH SOUND”, “BLOOD FLOW SOUND”, “ABDOMINAL SOUND”, and “FETAL HEART SOUND”, signal bands (frequency distributions) representative of characteristics of the sounds are determined in advance and stored, as features, in the sound source storage section 232 so in correspondence with respective measurement sites.

The measurement site specifying section 251 (i) compares the frequency spectrum of the sound data obtained from the acoustic sensor 202 with frequency distributions for respective measurement sites, and (ii) specifies, as a measurement site for the sound data obtained from the acoustic sensor 202, a measurement site associated with a frequency distribution which matches most with the frequency distribution of the frequency spectrum of the sound data obtained from the acoustic sensor 202. For example, a spectrum of sample sound data of “HEART SOUND” is concentrated on a band from 60 Hz to 80 Hz. Accordingly, in a case where the spectrum of the sound data obtained from the acoustic sensor 202 is concentrated on a band from 60 Hz to 80 Hz, the measurement site specifying section 251 can specify “HEART SOUND” as a measurement site.

In this manner, the attachment position estimating section 252 analyzes sound data (a biometric sound of a subject) obtained from the acoustic sensor 202, and estimates an attachment position. The attachment position estimating section 252 specifies an attachment position by carrying out matching between sample sound data and the sound data obtained from the acoustic sensor 202 with reference to a sound source database stored in the sound source storage section 232.

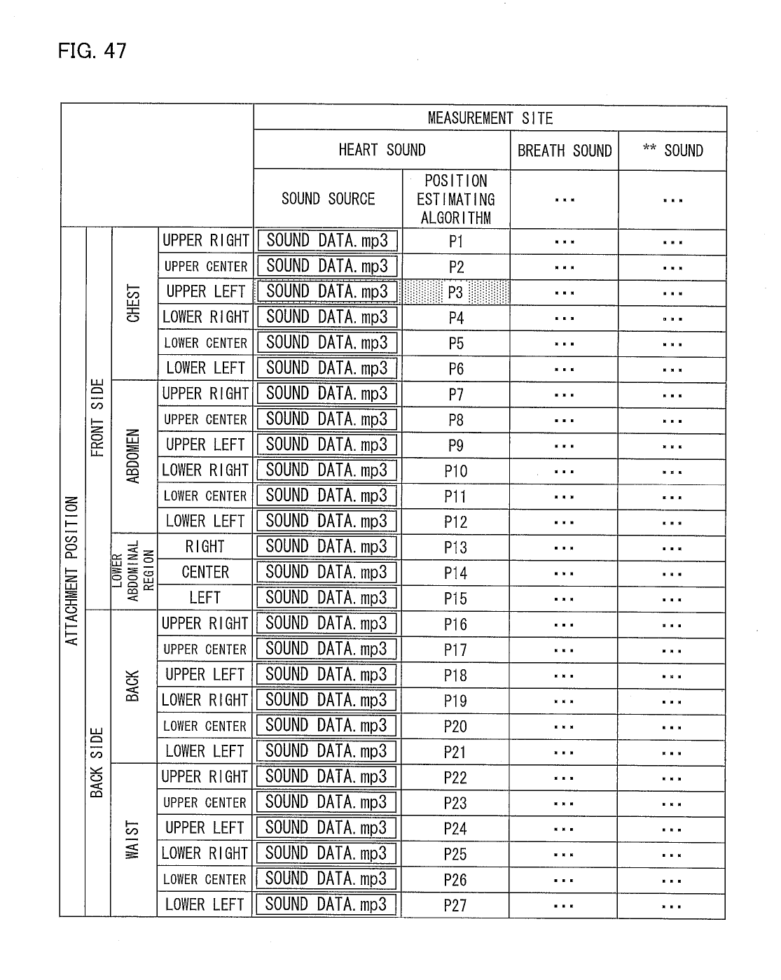

FIG. 47 is a table illustrating a data structure of the sound source database stored in the sound source storage section 232 of the analysis device 201 of the present embodiment. The sound source storage section 232 stores, for each attachment position, (i) standard sound data prepared on the basis of subject data gathered from subjects of all ages and both sexes and (ii) a position estimating algorithm describing how to analyze the sound data and how to carry out matching. Note that a single position estimating algorithm common to all the attachment positions may be prepared, but it is preferable that different position estimating algorithms associated with respective sound data are prepared for the respective attachment positions as illustrated in FIG. 47. This is because (i) a waveform of sound data varies depending on an attachment position and (ii) it is therefore possible to more accurately estimate an attachment position by changing, in accordance with the waveform, a method for evaluating a degree of matching (similarity). The position estimating algorithm mainly includes (i) a features extracting function for extracting features from sound data, (ii) a features matching function for matching between features and features, (iii) a matching degree evaluating function for evaluating matching/mismatching of sound data in accordance with a matching degree (similarity), and (iv) a correlation coefficient calculating function for calculating, on the basis of the matching degree (similarity), an index indicative of probability that gathered sound data is a sound obtained from an estimated attachment position. FIG. 47 illustrates an example of a data structure in which the sound source storage section 232 stores sample sound data itself for each attachment position. Note, however, that the data structure stored in the sound source storage section 232 of the present invention is not limited to this. The sound source storage section 232 may be configured to store, for each attachment position, features extracted from the sound data in addition to the sound data or instead of the sound data.

The attachment position estimating section 252 compares gathered sound data with each of sample sound data for the respective attachment positions illustrated in FIG. 47 so as to estimate which sound data is most similar to the gathered sound data. Specifically, the attachment position estimating section 252 carries out matching between the gathered sound data and each of the sample sound data in accordance with the position estimating algorithms P1 to P27 so as to calculate, for each attachment position, a correlation coefficient which is an index indicative of the probability. In a case where, for example, it is determined, as a result of calculation of the functions P1 to P27, that the highest correlation coefficient is obtained through matching according to the algorithm P3, the attachment position estimating section 252 can estimate that the gathered sound data is one obtained from the attachment position “FRONT SIDE-CHEST-UPPER LEFT”.

Note that the sound source database stored in the sound source storage section 232 preferably contains, also for each “measurement site”, a set of sample sound data and a position estimating algorithm. Specifically, the sound source database stored in the sound source storage section 232 preferably contains, for each measurement site, as many sets of sample sound data and a position estimating algorithm as the attachment positions 227 (e.g., position estimating algorithms P1 to P27 for the measurement site “heart sound”, position estimating algorithms Q1 to Q27 for the measurement site “breath sound”, . . . ).

According to the data structure, matching between sound data can be carried out in view of a difference in waveform which arises from a difference in measurement site. This makes it possible to more accurately estimate an attachment position. However, there is a problem that a processing load becomes enormous in a case where the attachment position estimating section 252 carries out all of the position estimating algorithms P1 to P27, Q1 to Q27 . . . stored in the sound source database. Accordingly, in such a case, the measurement site specifying section 251 first specifies a measurement site for gathered sound data, and then the attachment position estimating section 252 carries out only position estimating algorithms for the measurement site thus specified by the measurement site specifying section 251. For example, in a case where the measurement site specifying section 251 specifies “breath sound” as a measurement site, the attachment position estimating section 252 estimates an attachment position by carrying out only the position estimating algorithms Q1 to Q27 associated with “breath sound”.

According to the above arrangement, all a user has to do is to gather sound data by attaching the acoustic sensor 202 to an approximate position of a subject's body. Thereafter, on the basis of the sound data thus gathered, the measurement site specifying section 251 of the analysis device 201 specifies a measurement site, and the attachment position estimating section 252 estimates an attachment position. This allows the analysis device 201 to (i) determine attribute information without the need for a user's input operation and to (ii) carry out accurate measurement on the basis of the attribute information thus determined.

The attribute information determining section 221 preferably (i) allows the user to confirm information on the measurement site specified by the measurement site specifying section 251 by displaying it as shown by the measurement site 245 in FIG. 43, and (ii) allows the user to confirm information on the attachment position estimated by the attachment position estimating section 252 by displaying it as shown by the human body figure 240 and the star sign 242 in FIG. 30. The user clicks the measurement start button if the user has no problem with the attribute information presented on the display section 215. The attribute information determining section 221 thus finally determines the attribute information “attachment position” and “measurement site”, and then the analysis device 201 shifts to carrying out of more detailed measurement based on the attribute information.

[Biometric Process Flow]

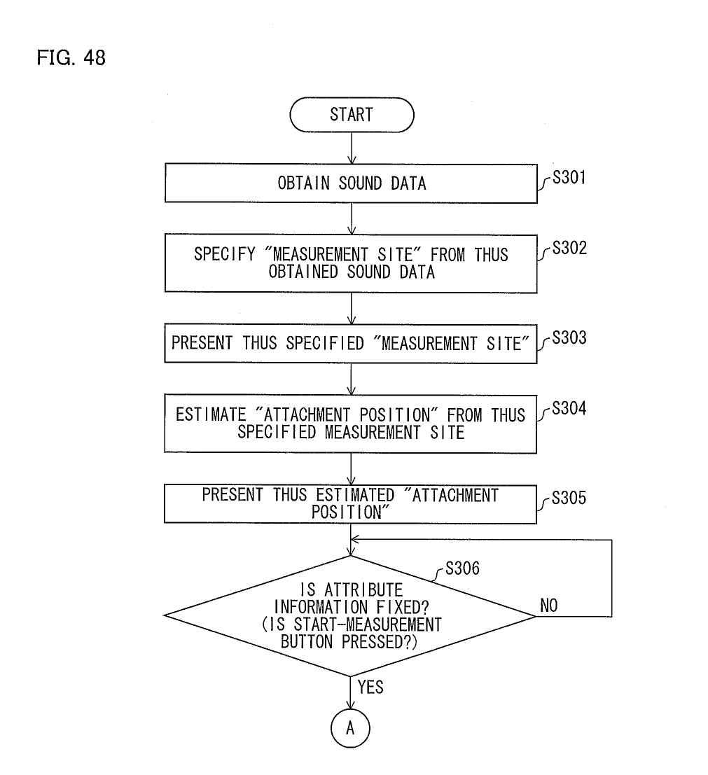

FIG. 48 is a flowchart illustrating a flow of a biometric process carried out by the analysis device 201 of the present embodiment.

Upon activation of an application for carrying out a biometric process in the analysis device 201, the attribute information determining section 221 may, for example, prompt a user to gather sound data with use of the acoustic sensor 202. The user attaches the acoustic sensor 202 somewhere on a subject's body, and carries out detection of a biometric sound. Sound data gathered by the acoustic sensor 202 is transmitted to the analysis device 201, and the information obtaining section 220 obtains the sound data thus transmitted (S301).

The measurement site specifying section 251 compares (i) features (e.g., frequency distribution) of the sound data obtained from the acoustic sensor 202 with (ii) features of sound data stored for each measurement site so as to specify a measurement site of the sound data obtained from the acoustic sensor 202 (S302). That is, the measurement site specifying section 251 specifies which site is a target for measurement of the acoustic sensor 202 which has gathered the sound data. The measurement site specifying section 251 prompts the user to confirm information on the measurement site specified by, for example, causing it to be displayed in the display section 215 (S303).

Subsequently, the attachment position estimating section 252 estimates, on the basis of the measurement site specified by the measurement site specifying section 251, an attachment position of the sound data obtained from the acoustic sensor 202 (S304). Specifically, the attachment position estimating section 252 reads out, from the sound source storage section 232, sample sound data that are stored for respective attachment positions and that correspond to the measurement site specified by the measurement site specifying section 251, and then carries out matching between the sound data obtained from the acoustic sensor 202 and the sample sound data in accordance with position estimating algorithms associated with the respective sample sound data. The attachment position estimating section 252 estimates, as an attachment position for the sound data obtained from the acoustic sensor 202, an attachment position corresponding to a position estimating algorithm by which the highest correlation coefficient has been obtained. That is, the attachment position estimating section 252 estimates a position to which the acoustic sensor 202 which has gathered the sound data is attached. The attachment position estimating section 252 prompts the user to confirm information on the attachment position estimated by, for example, causing it to be displayed in the display section 215 (S305).

This allows the user to (i) confirm the “measurement site” displayed in the display section 215 and grasp a rough purpose of measurement and (ii) grasp an accurate “attachment position” for achieving the target measurement. In a case where an actual attachment position is deviated from the “attachment position” displayed in the display section 215, the user can correct, on the basis of the “ATTACHMENT POSITION” presented to the user, the position of the acoustic sensor 202 attached to the subject. If the user has no problem with presented contents, the user instructs the analysis device 201 to start a biometric process by, for example, clicking the measurement start button illustrated in FIG. 30. Here, the attribute information determining section 221 may further accept the user's designation of a measurement item.

When the user's approval (e.g., clicking of the measurement start button) has been obtained (YES in S306), the attribute information determining section 221 finally determines the attribute information. Thereafter, processing for selecting an algorithm and processing for deriving measurement result information are carried out as in Embodiments 2-1 and 2-2.

According to the arrangement of the analysis device 201 and the biometric method in accordance with the present embodiment, a user can enjoy convenience of being able to start measurement simply by roughly attaching the acoustic sensor 202 without thinking deeply. In general, in a case where a single acoustic sensor is used for measurement of a plurality of sounds or a plurality of diseases, a user is required to have a broad knowledge about attachment positions for the respective diseases. However, according to the present invention, a sound source and a disease which a user wants to measure can be estimated and displayed on the basis of gathered sound data. It is thus possible to provide a biometric system 200 that does not require a user to have advance knowledge and that is highly convenient for the user.

### Embodiment 2-4

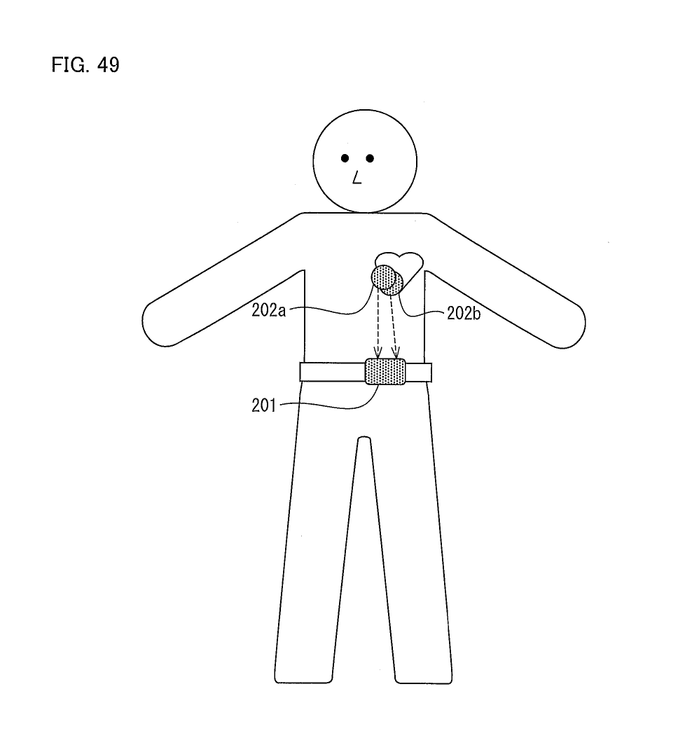

Another embodiment of the analysis device 201 of the present invention is described below with reference to FIGS. 49 through 51. For convenience of explanation, members that have functions identical to those of members illustrated in the drawings of Embodiments 2-1 to 2-3 are given identical reference numerals, and are not explained repeatedly.

In Embodiments 2-1 to 2-3, it is assumed that a single acoustic sensor 202 is used in the biometric system 200. However, the biometric system 200 of the present invention is not limited to this. It is also possible to employ an arrangement in which (i) a plurality of acoustic sensors 202 are attached to a subject and (ii) a plurality of pieces of measurement result information are derived by carrying out information processing in accordance with a plurality of pieces of attribute information of the respective acoustic sensors 202.

FIG. 49 is a diagram illustrating an example of how a plurality of acoustic sensors 202 of a biometric system 200 of an embodiment of the present invention are attached.

In the example illustrated in FIG. 49, two acoustic sensors 202 (an acoustic sensor 202a and an acoustic sensor 202b) are attached to a subject. Note that attachment positions of the acoustic sensors 202 and the number of acoustic sensors 202 can be changed depending on an intended purpose and cost.

An analysis device 201 is capable of communicating with each of the acoustic sensors 202a and 202b via a sensor communication section 212. In the present embodiment, the analysis device 201 is capable of uniquely identifying each of the acoustic sensor 202a and the acoustic sensor 202b.

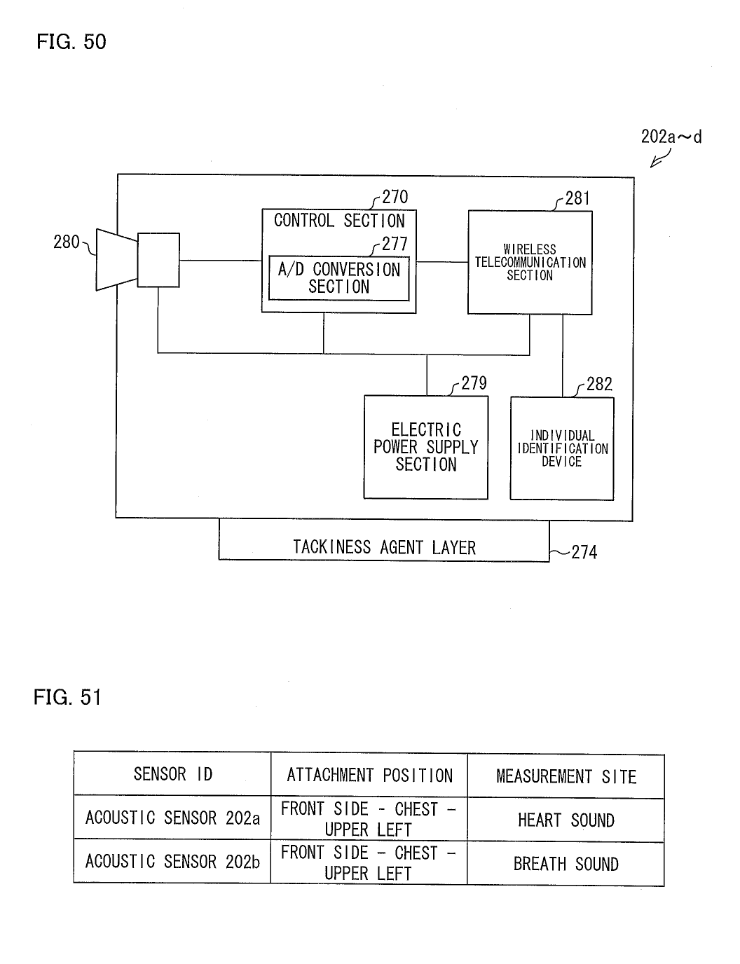

FIG. 50 is a block diagram illustrating an essential configuration of each of the acoustic sensors 202a and 202b of the present embodiment. Differently from the acoustic sensor 202 illustrated in FIG. 28, each of the acoustic sensors 202a and 202b illustrated in FIG. 50 further includes an individual identification device 282.

The individual identification device 282 possesses individual identification information, i.e., a sensor ID for allowing the analysis device 201 to uniquely identify a corresponding acoustic sensor 202. In communicating with the analysis device 201, a wireless telecommunication section 281 causes the sensor ID stored in the individual identification device 282 to be added to a header of communication data. The analysis device 201 can distinguish the acoustic sensors 202 from each other on the basis of the sensor ID contained in the header. Note that the individual identification device 282 may be in either a physical form or a logical form. For example, the individual identification device 282 may be in a physical form such as a jumper wire, or may be formed of a non-volatile memory such as EEPROM. Alternatively, the individual identification device 282 may be a part of a memory provided in a control section 270 that is formed of a microcomputer.

The sensor ID allows the analysis device 201 to individually identify each of the acoustic sensors 202. This allows the analysis device 201 to manage attribute information in the attribute information storage section 234 individually for each of the acoustic sensors 202.

FIG. 51 is a table showing a specific example of attribute information for the plurality of acoustic sensors 202 which attribute information is stored in the attribute information storage section 234. In a case where, for example, the attachment position “FRONT SIDE-CHEST-UPPER LEFT” and measurement site “HEART SOUND” are determined as attribute information for the acoustic sensor 202a on the basis of the arrangement of the analysis device 201 of any one of Embodiments 2-1 to 2-3 or a combination of Embodiments 2-1 to 2-3, the attribute information determining section 221 causes the information on the attachment position “FRONT SIDE-CHEST-UPPER LEFT” and measurement site “HEART SOUND” to be stored in correspondence with a sensor ID of the acoustic sensor 202a as illustrated in FIG. 51. Similarly, in a case where the attachment position “FRONT SIDE-CHEST-UPPER LEFT” and the measurement site “BREATH SOUND” are determined as attribute information of the acoustic sensor 202b, the attribute information determining section 221 causes the information of the attachment position “FRONT SIDE-CHEST-UPPER LEFT” and measurement site “BREATH SOUND” to be stored in correspondence with a sensor ID of the acoustic sensor 202b.

The algorithm selecting section 222 individually selects, on the basis of the attribute information stored in the attribute information storage section 234, algorithms to be applied to the acoustic sensors 202a and 202b. This is described below in detail on the basis of the examples illustrated in FIGS. 51 and 31. The acoustic sensor 202a is attached to an upper part of a left portion of the chest to measure a heart sound. Accordingly, the algorithm selecting section 222 selects the algorithm A3 for sound data gathered by the acoustic sensor 202a. Meanwhile, the acoustic sensor 202b is attached to an identical attachment position (i.e., an upper part of a left portion of the chest) to the acoustic sensor 202a, but differently from the acoustic sensor 202a, its target is to measure a measurement site “BREATH SOUND”. Accordingly, the algorithm selecting section 222 selects the algorithm B3 for sound data gathered by the acoustic sensor 202b. For example, the algorithm A3 aiming at measurement of a heart sound may include an algorithm for a “noise removing process” for removing, as a noise, a sound component other than a heart sound component from the sound data gathered. The algorithm B3 aiming at measurement of a breath sound may include an algorithm for a “noise removing process” for removing, as a noise, a sound component other than a breath sound component from the sound data gathered.

The above arrangement makes it possible to simultaneously measure different measurement sites (for example, a heart sound and a breath sound) with use of a plurality of acoustic sensors 202 of the same type. This merely requires only one measurement even for a subject having a plurality of diseases at both of the measurement sites, thereby shortening a measurement time. Further, in a case of measurement of a single disease, it is possible to simultaneously gather biometric sounds at a plurality of points by simultaneously measuring a plurality of measurement sites. This increases an amount of information, thereby carrying out measurement with higher accuracy. For example, it is possible to increase accuracy of state observation and measurement of a disease such as pneumonia or bronchitis by (i) simultaneously gathering sounds at three points, i.e., a right lung, a left lung, and a bronchial tube and by (ii) analyzing sound data at the three points.

### Embodiment 2-5

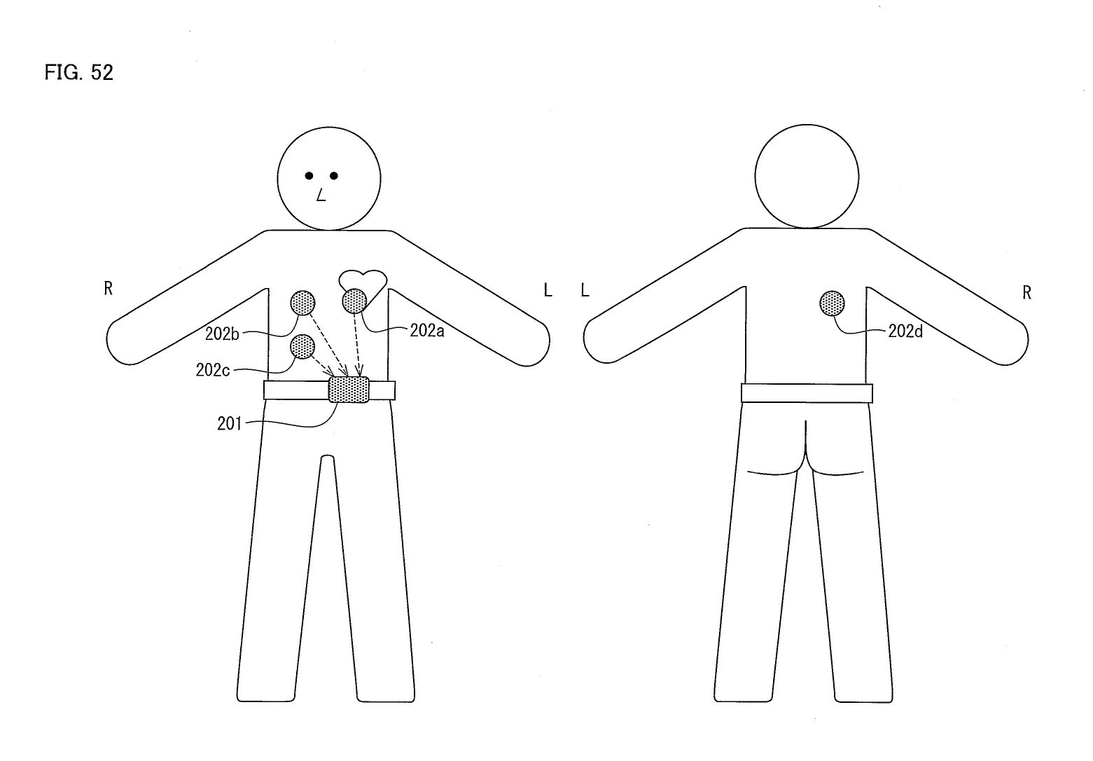

Another embodiment of the analysis device 201 of the present invention is described below with reference to FIGS. 52 through 54. For convenience of explanation, members that have functions identical to those of members illustrated in the drawings of Embodiments 2-1 to 2-4 are given identical reference numerals, and are not explained repeatedly.

Embodiment 2-3 deals with an arrangement in which the attachment position estimating section 252 of the attribute information determining section 221 estimates an attachment position of the acoustic sensor 202 with use of a position estimating algorithm. As described in Embodiment 2-4, in a case where a plurality of acoustic sensors 202 are attached, the attachment position estimating section 252 individually estimates attachment positions of the plurality of acoustic sensors 202.

The present Embodiment 2-5 deals with an arrangement in which accuracy and efficiency of attachment position estimation carried out by the attachment position estimating section 252 are improved with use of a signal for use in wireless telecommunications between a plurality of acoustic sensors 202 and an analysis device 201.

FIG. 52 is a diagram illustrating another example of how a plurality of acoustic sensors 202 of a biometric system 200 of an embodiment of the present invention are attached.

In the example illustrated in FIG. 52, four acoustic sensors 202a through 202d are attached to a subject. Specifically, the acoustic sensors 202a through 202c are attached to a front side of the subject, and the acoustic sensor 202d is attached to a back side of the subject. Since each of the acoustic sensors 202a through 202d has a configuration identical to that illustrated in FIG. 50, the analysis device 201 is capable of distinguishing between the four acoustic sensors 202a through 202d, and is capable of wirelessly telecommunicating with each of the four acoustic sensors 202a through 202d.

As illustrated in FIG. 52, data signals are exchanged between (i) the acoustic sensors 202a through 202d and (ii) the analysis device 201 via wireless telecommunications while the acoustic sensors 202a through 202d are detecting biometric sounds. A carrier intensity of a wireless signal which each of the acoustic sensors 202a through 202d receives from the analysis device 201 depends on a physical distance between the each of the acoustic sensors 202a through 202d and the analysis device 201.

In the present embodiment, each of the acoustic sensors 202a through 202d causes a wireless telecommunication section 281 provided therein to (i) find and preserve a carrier intensity of a signal received from the analysis device 201 and to (ii) notify the analysis device 201 of the carrier intensity as appropriate. Further, each of the acoustic sensors 202a through 202d can find and preserve a carrier intensity of a signal which it receives from another acoustic sensor 202 wirelessly telecommunicating with the analysis device 201. For example, in a case where the acoustic sensor 202a is wirelessly telecommunicating with the analysis device 201, each of the other acoustic sensors 202b to 202d causes the wireless telecommunication section 281 provided therein to find a carrier intensity of a wireless signal which it receives from the acoustic sensor 202a.

The attachment position estimating section 252 of the analysis device 201 collects carrier intensity information found by the acoustic sensors 202a through 202d. The attachment position estimating section 252 estimates a relative positional relationship among the acoustic sensors 202a through 202d on the basis of the collected carrier intensity information so as to help estimate an attachment position of each of the acoustic sensors 202a through 202d.

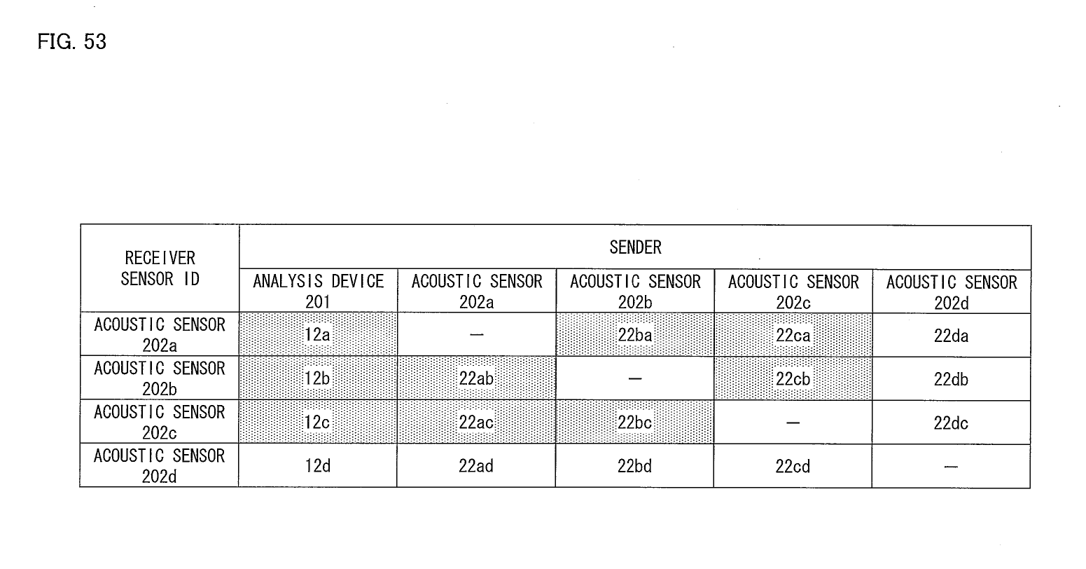

FIG. 53 is a table showing a specific example of carrier intensity information collected by the attachment position estimating section 252. The carrier intensity information is stored in a temporary storage section (not shown) until attribute information is determined. Alternatively, the carrier intensity information may be stored in any of regions of a storage section 211 in a non-volatile manner. As an example, it is assumed here that the devices (the analysis device 201 and the acoustic sensors 202) are disposed as illustrated in FIG. 52. Specifically, it is assumed that (i) the analysis device 201 is attached to a waist of a subject in the vicinity of a buckle of a belt, (ii) the acoustic sensors 202a through 202c are attached to a chest side of the subject, and (iii) only the acoustic sensor 202d is attached to a back side of the subject.

A carrier intensity is uniquely determined depending on a relation between (i) an acoustic sensor or an analysis device (transmission source) which has transmitted a signal and (ii) an acoustic sensor (recipient) which has received the signal. For example, four carrier intensities “12a”, “22ba”, “22ca”, and “22da” associated with a recipient sensor ID “ACOUSTIC SENSOR 202a” represent (i) a reception intensity of a signal received from the analysis device 201 by the acoustic sensor 202a, (ii) a reception intensity of a signal received from the acoustic sensor 202b by the acoustic sensor 202a, (iii) a reception intensity of a signal received from the acoustic sensor 202c by the acoustic sensor 202a, and (iv) a reception intensity of a signal received from the acoustic sensor 202d by the acoustic sensor 202a, respectively.

Since the acoustic sensors 202a through 202c and the analysis device 201 are attached to a front side of the subject, carrier intensities 12a to 12c, for example, are relatively large as compared with a carrier intensity 12d. The carrier intensity 12d is relatively small because the acoustic sensor 202d is attached to the back side of the subject, and is distant from the analysis device 201. That is, in the carrier intensity table illustrated in FIG. 53, carrier intensities in the shaded cells are relatively large, but carrier intensities in the other cells are small as compared with the carrier intensities in the shaded cells. Out of the carrier intensities in the shaded cells, a carrier intensity between the acoustic sensor 202c and the analysis device 201 is relatively large. Accordingly, it can be estimated that the acoustic sensor 202c is attached to a position closer to the analysis device 201 as compared with the other acoustic sensors 202a and 202b.

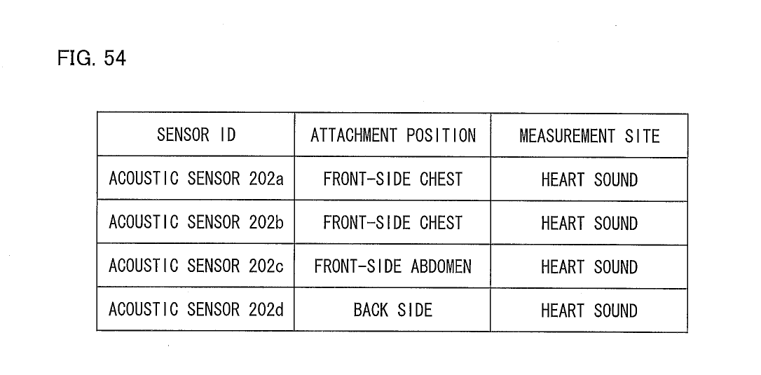

Based on the above result, the attachment position estimating section 252 can specify approximate positions of the respective acoustic sensors 202 as illustrated in FIG. 54. In the above example, (i) the acoustic sensor 202d, for example, is estimated to be attached somewhere on the back side farthest from the analysis device 201, (ii) the acoustic sensor 202c is estimated to be attached around a front abdominal region closest to the analysis device 201, and (iii) each of the acoustic sensors 202a and 202b is estimated to be attached to a front chest region farther from the analysis device 201 than the acoustic sensor 202c but closer to the analysis device 201 than the acoustic sensor 202d. A measurement site of each of the acoustic sensors 202 is determined as appropriate by a procedure described in any of Embodiments 2-1 to 2-3.

The attachment position estimating section 252 can (i) cause an intermediate result concerning attribute information (especially attachment positions) illustrated in FIG. 54 to be stored in the attribute information storage section 234 and (ii) rewrite the attachment positions into more detailed attachment positions by carrying out the position estimating algorithms shown in Embodiment 2-3.

Estimating approximate attachment positions of the respective acoustic sensors 202 as illustrated in FIG. 54 before the attachment position estimating section 252 carries out the position estimating algorithms as described above has the advantages below.

As described above, in Embodiment 2-3, the attachment position estimating section 252 is arranged to (i) sequentially apply, to obtained sound data, the position estimating algorithms P1 to P27 (in a case where the measurement site is “HEART SOUND”) for the respective attachment positions and (ii) estimate an algorithm achieving the highest correlation coefficient. In a case where the attachment position estimating section 252 estimates approximate attachment positions in advance on the basis of carrier intensities, it is possible to narrow down position estimating algorithms to be applied to the sound data. For example, in a case of estimating an attachment position of the acoustic sensor 202d, the attachment position is roughly estimated as “BACK SIDE” in advance as illustrated in FIG. 54. In this case, the attachment position estimating section 252 does not need to carry out all of the position estimating algorithms P1 to P27 and is simply required to carry out only the position estimating algorithms P16 to P27 corresponding to the attachment position “BACK SIDE”. Also in cases of estimating attachment positions of the acoustic sensors 202a through 202c, the attachment position estimating section 252 can narrow down the number of sample sound data and the number of position estimating algorithms to be applied to sound data on the basis of a roughly estimated positional relationship.

As a result, it is possible to greatly reduce a processing load of the control section 210 of the analysis device 201 and to increase efficiency of attachment position estimating processing.

<<Variation>>

Each of the above embodiments discusses a case where a biometric device of the present invention measures a state of a human (human subject) with use of a biometric sensor for sensing a state of a human (human subject) as an example of a living body. However, the biometric device of the present invention is not limited to this arrangement. The biometric device of the present invention is also capable of obtaining a biometric sound of an animal (such as a dog) other than a human as an examinee (living body) so as to measure a state of the animal. In this case, the correspondence tables illustrated in FIGS. 31, 32, 42, 47, etc. (the correspondence tables indicating a correspondence relationship between attribute information and an algorithm and the correspondence table of the sound source database) are constructed as appropriate in accordance with properties of an animal to be examined. For example, in a case where a dog is an examinee, an algorithm for detecting a disease specific to dogs and biological sound data of a sample dog are prepared.

### Embodiment 3

[Technical Problem]

The invention of Patent Literature 3 determines, solely on the basis of a cough sound that a subject emits, whether or not the subject has coughed, and as such, is low in accuracy of the determination.

Meanwhile, the invention of Patent Literature 4 determines, on the basis of both (i) a cough sound that a subject emits and (ii) a body motion that the subject makes, whether or not the subject has coughed. However, since the subject does not always have to emit a cough sound to make a body motion, the invention of Patent Literature 4 is not necessarily high in accuracy of the determination (i.e., in accuracy of cough detection).

The present invention has been accomplished in view of the above problem, and it is a further object of the present invention to provide a biometric device capable of detecting a state of a living body (e.g., a subject) with high accuracy.

### Embodiment 3-1

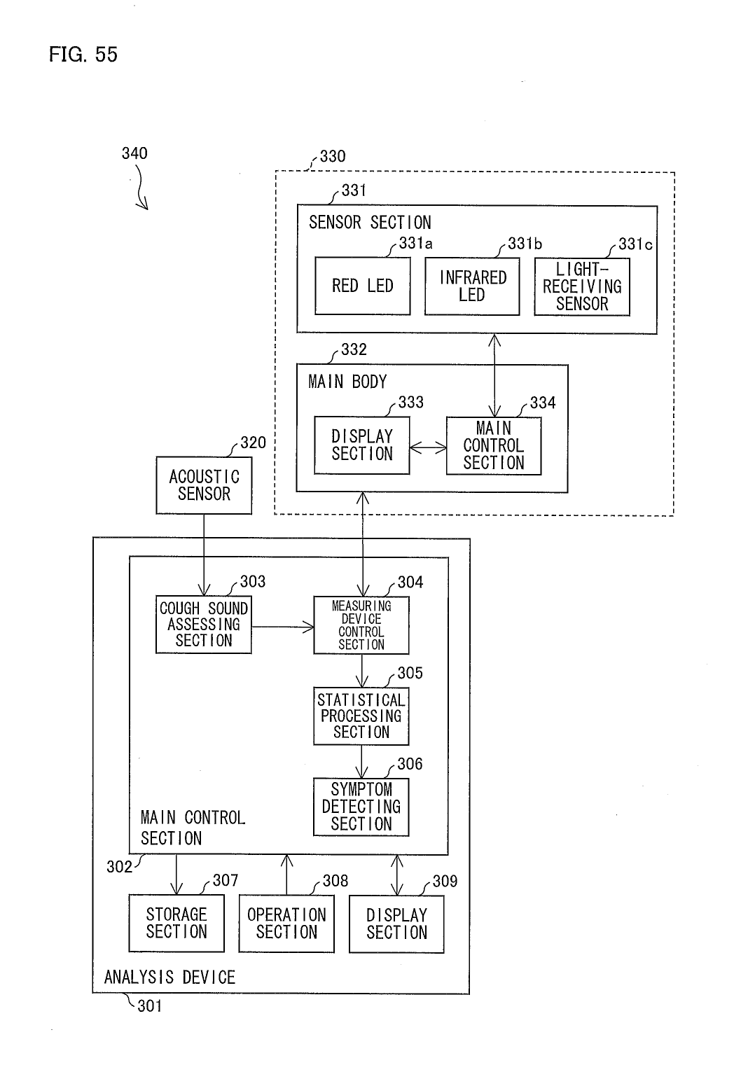

An embodiment of the present invention is described below with reference to FIGS. 55 through 59. In the present embodiment, a symptom detecting device 340 that detects a symptom of a cough is described as an example of a biometric device of the present invention. It should be noted that the present invention is not limited to such a symptom detecting device that detects a symptom of a cough, but may be achieved in the form of another detecting device that detects a state of a subject, e.g., in the form of a symptom detecting device that detects a sneeze.

Further, the following description assumes that an object to be measured by the symptom detecting device 340 is a human (subject). However, an object to be measured by the biometric device of the present invention may be a non-human animal (such as a dog). That is, it can be said that an object to be measured by the biometric device of the present invention is a living body.

(Arrangement of the Symptom Detecting Device 340)

FIG. 55 is diagram schematically illustrating a configuration of the symptom detecting device 340. As illustrated in FIG. 55, the symptom detecting device 340 includes an analysis device (biometric device) 301, an acoustic sensor (biometric sound sensor) 320, and a pulse oximeter (biometric sensor) 330.

<Acoustic Sensor 320>

The acoustic sensor 320 is a contact microphone that is attached to the chest of a subject so as to detect a cough sound that the subject emits. A usable example of the acoustic sensor 320 is a contact microphone described in Japanese Patent Application Publication, Tokukai, No. 2009-233103 A. FIG. 29 is a cross-sectional view illustrating a configuration of the acoustic sensor 320. As illustrated in FIG. 29, the acoustic sensor 320 is a sound-collecting unit based on a so-called condenser microphone, and includes a housing 271 and a diaphragm 273. The housing 271 has a cylindrical shape, and has one end face open. The diaphragm 273 is in closed contact with the housing 271 so as to close the open face of the housing 271. Further, the acoustic sensor 320 includes a first conversion section 275, an A/D conversion section 277, a substrate 278, and an electric power supply section 279. The A/D conversion section 277 serves as a second conversion section. The first conversion section 275 and the A/D conversion section 277 are mounted on the substrate 278. The electric power supply section 279 supplies electric power to the first conversion section 275 and the A/D conversion section 277.

Provided on a surface of the diaphragm 273 is a tackiness agent layer 274 that causes the acoustic sensor 320 to be attached to a body surface (H) of the subject. The acoustic sensor 320 is attached to a position such as the chest or a lower part of the throat, and only needs to be attached to any place where the acoustic sensor 320 can effectively pick up a cough sound.

When the patient emits a biometric sound e.g., by coughing, breathing, or swallowing, the diaphragm 273 minutely vibrates in accordance with the wavelength of the biometric sound. The minute vibration of the diaphragm 273 is transmitted to the first conversion section 275 via an air chamber wall 276. The air chamber wall 276 has a circular conical shape, and has upper and lower open faces.

The vibration transmitted through the air chamber wall 276 is converted into an electric signal by the first conversion section 275. The electric signal is then converted into a digital signal by the A/D conversion section 277. The digital signal is then transmitted to a cough sound determining section 303 of the analysis device 301.

The biometric sound thus detected by the acoustic sensor 320 is outputted as biometric sound data (biometric sound signal information) to the cough sound determining section 303 of the analysis device 301. The acoustic sensor 320 may output the biometric sound data to the analysis device 301 only in a case where the biometric sound detected has a sound volume that is equal to or higher than a predetermined sound volume, or may always output the biometric sound data. However, since the acoustic sensor 320 is driven by the electric power supplied from the electric power supply section 279, it is preferable, for the purpose of cutting electric power consumption and allowing the acoustic sensor 320 to be driven for a longer time period, that the acoustic sensor 320 output the biometric sound data to the analysis device 301 only in a case where the biometric sound detected has a sound volume that is equal to or higher than a predetermined sound volume.

Further, the acoustic sensor 320 may contain a timer so that the biometric sound data contains information indicative of a time point at which the biometric sound data was obtained.

The acoustic sensor 320 and the analysis device 301 only need to be communicably connected to each other, either via cable or wirelessly. The analysis device 301 may be contained in the acoustic sensor 320.

<Pulse Oximeter 330>

The pulse oximeter 330 is a measuring device that measures the percutaneous arterial blood oxygen saturation of the subject at predetermined time intervals. The arterial blood oxygen saturation is an arterial blood oxygen saturation measured percutaneously, and is a physiological index of a subject which index may vary when the subject coughs.

As illustrated in FIG. 55, the pulse oximeter 330 includes a sensor section 331 and a main body 332, and the main body 332 includes a display section 333 and a main control section 334.

The sensor section 331 includes a red LED 331a, an infrared LED 331b, and a light-receiving sensor 331c. The red LED 331a emits red light. The infrared LED 331b emits infrared light. The light-receiving sensor 331c receives transmitted light that is generated when the light emitted from these LEDs has passed through a fingertip of the subject.

The main control section 334 controls the sensor section 331 in accordance with a commend from the analysis device 301, and calculates the arterial blood oxygen saturation from the ratio of a variable component to the amount of transmitted red and infrared light as received by the light-receiving sensor 331c. The percutaneous arterial blood oxygen saturation thus calculated is displayed by the display section 333 (e.g., a liquid crystal display), and is outputted as measurement data to a measuring device control section 304 of the analysis device 301. The measurement data correlates a measured value of the arterial blood oxygen saturation to a time point at which the measured value was obtained.

The pulse oximeter 330 starts measurement of the arterial blood oxygen saturation in a case where the cough sound determining section 303 of the analysis device 301 has determined that the biometric sound data contains a cough sound. The pulse oximeter 330 may always perform measurements. However, in a case where the pulse oximeter 330 is driven by a battery contained in the pulse oximeter 330, it is preferable, for the purpose of cutting electric power consumption and allowing the pulse oximeter 330 to be driven for a longer time period, that the pulse oximeter 330 perform measurements only in a case where the pulse oximeter 330 has received, from the analysis device 301, a command to start measurement.

The pulse oximeter 330 and the analysis device 301 only need to be communicably connected to each other, either via cable or wirelessly. The analysis device 301 may be contained in the pulse oximeter 330.

<Analysis Device 301>

By using the biometric sound data (specifically, biometric sound parameter that is extracted from the biometric sound data) generated by the acoustic sensor 320 and the measurement data (biometric parameter) of percutaneous arterial blood oxygen saturation as generated by the pulse oximeter 330, the analysis device 301 detects a cough that the subject emits. Specifically, the detection, by the acoustic sensor 320, of a cough sound that the subject emits causes the analysis device 301 to detect the presence or absence of a cough on the basis of a change in arterial blood oxygen saturation of the subject as measured by the pulse oximeter 330.

The term “biometric sound parameter” is a general term for information regarding a sound that the subject emits, and can encompass information such as a sound volume, a change in sound volume over time, and the frequency of a sound. More specifically, the biometric sound parameter is information regarding a sound that the subject emits, and such information can be extracted from biometric sound data outputted from an acoustic sensor 320 attached to the subject or from an acoustic sensor 320 placed in an area around the subject.

The following description assumes that the biometric sound parameter is information that is obtained by analyzing biometric sound data (biometric sound signal information) outputted from the acoustic sensor 320.

Further, the biometric parameter is a parameter that is different from the biometric sound parameter and that reflects a physiological state of the subject. In the present embodiment, the biometric parameter is a percutaneous arterial blood oxygen saturation.

It should be noted that the biometric parameter may be based on biometric sound signal information, and may, for example, be (i) an index for heart disease as obtained by analyzing a heart sound or (ii) an index indicative of a degree of breathing as obtained by analyzing a breath sound.

In the present embodiment, as mentioned above, the pulse oximeter 330 calculates the percutaneous arterial blood oxygen saturation on the basis of the amount of light received (biometric signal information), and outputs the percutaneous arterial blood oxygen saturation thus calculated to the analysis device 301. This means that the analysis device 301 does not directly analyze the biometric signal information but obtains the biometric parameter from the pulse oximeter 330.

In the case of use of a biometric parameter other than the percutaneous arterial blood oxygen saturation, the biometric parameter may be obtained by analyzing the biometric signal information. For example, a biometric parameter regarding breathing may be obtained by analyzing the airflow through the mouth or nose (biometric signal information).

The analysis device 301 includes a main control section 302, a storage section 307, an operation section 308, and a display section 309. The main control section 302 includes the cough sound determining section (biometric sound parameter obtaining means, cough sound estimating means) 303, the measuring device control section (biometric parameter obtaining means) 304, a statistical processing section 305, and a symptom detecting section (detecting means) 306.

<Cough Sound Determining Section 303>

The cough sound determining section 303 obtains biometric sound data outputted from the acoustic sensor 320, and estimates generation of a cough sound on the basis of the biometric sound data. That is, the cough sound determining section 303 determines whether or not the biometric sound data contains a cough sound. In this case, a biometric sound parameter regarding a cough sound can be deemed as being obtained by analyzing the biometric sound data.

As the method for determining whether or not the biometric sound data contains a cough sound, a publicly known method may be used. For example, the presence or absence of a cough sound may be determined by using, as features of a cough sound, a rising slope of a sound signal and a duration of time change in the sound signal. Alternatively, it is possible to extract a plurality of bandwidth signals from sound data as described in Patent Literature 3 and determine the presence or absence of a cough sound from a correlation between the bandwidth signals thus extracted.

Further, the cough sound determining section 303 refers to a timer (not shown) that is available thereto, and records, in the storage section 307, correspondence between a time point at which biometric sound data was obtained (or a time point at which the acoustic sensor 320 detected a biometric sound) and the biometric sound data.

<Measuring Device Control Section 304>

In a case where the cough sound determining section 303 has determined that the sound data contains a cough sound, the measuring device control section 304 outputs, to the main control section 334 of the pulse oximeter 330, a command to start measurement. Upon receipt of the command to start measurement, the pulse oximeter 330 measures the percutaneous arterial blood oxygen saturation and outputs it as measurement data. Then, the measuring device control section 304 obtains the measurement data and outputs it to the statistical processing section 305. The command to start measurement may be a command that causes the pulse oximeter 330 to measure the percutaneous arterial blood oxygen saturation for a predetermined time period (e.g., 20 seconds), and a command to finish measurement may be outputted separately from the command to start measurement.

Without the cough sound determining section 303 determining whether or not the biometric sound contained in the biometric sound data contains a cough sound, the measuring device control section 304 may cause the pulse oximeter 330 to start measurement in a case where a biometric sound of any kind has been detected. That is, the measuring device control section 304 may obtain measurement data (i.e., measured values of the percutaneous arterial blood oxygen saturation) in a case where the biometric sound contained in the biometric sound data meets a predetermined condition (e.g., a predetermined sound volume or higher).

<Statistical Processing Section 305>

The statistical processing section 305 performs statistical processing of measured values of the percutaneous arterial blood oxygen saturation that have been obtained on a time-series basis. For example, the statistical processing section 305 calculates a statistical value (e.g., mean, median, or the like) of the percutaneous arterial blood oxygen saturation over a predetermined time period beginning at a time point at which a biometric sound was detected by the acoustic sensor 320 (i.e., a time point at which the biometric sound parameter changed).

More specifically, the statistical value is the mean of percutaneous arterial blood oxygen saturations over a period of approximately 20 seconds set on the basis of a time point at which a biometric sound was detected by the acoustic sensor 320. For example, the statistical value is the mean of percutaneous arterial blood oxygen saturations over a period of 20 seconds after a time point at which a biometric sound was detected by the acoustic sensor 320.

The percutaneous arterial blood oxygen saturation does not always remain constant in the same subject, but can vary from time to time. Further, it is considered that a measured percutaneous arterial blood oxygen saturation contains a measurement error.

The percutaneous arterial blood oxygen saturation in a state in which the subject is not coughing can be more accurately calculated by (i) setting a measuring period of approximately 20 seconds beginning at a time point at which the acoustic sensor 320 detected a biometric sound and (ii) performing statistical processing of measured values of the percutaneous arterial blood oxygen saturation that have been obtained during the measuring period.

Since there is a time lag of approximately 20 seconds between (i) a time point at which the subject coughed and (ii) a time point at which there is an actual change in percutaneous arterial blood oxygen saturation, the percutaneous arterial blood oxygen saturation before the subject coughs can be calculated even in a case where the mean of percutaneous arterial blood oxygen saturations over a period of 20 seconds after detection of a biometric sound is calculated.

However, in a case where the time period during which the percutaneous arterial blood oxygen saturation is measured is too long, a value of percutaneous arterial blood oxygen saturation that is low due to the influence of a cough may be included in the calculation of the mean. This problem is likely to occur especially in a case where the subject emits coughs at short intervals. Therefore, it is preferable that the time period during which the percutaneous arterial blood oxygen saturation is measured be approximately 10 to 30 seconds.

In a configuration in which the percutaneous arterial blood oxygen saturation is always measured, a value of percutaneous arterial blood oxygen saturation that had been measured before a time point at which a biometric sound was detected may be used in the calculation of the statistical value. For example, the mean of percutaneous arterial blood oxygen saturations over a period of 10 seconds before a time point at which a biometric sound is detected and a period of 10 seconds after the time point may be calculated.

<Symptom Detecting Section 306>

The symptom detecting section 306 makes a comparison between (i) the statistical value calculated by the statistical processing section 305 and (ii) the percutaneous arterial blood oxygen saturation at a predetermined time point, thereby detecting a state of emission of a cough by the subject and the severity of coughing.

Specifically, the symptom detecting section 306 detects a cough that the subject emits on the basis of a change in percutaneous arterial blood oxygen saturation over a predetermined time period beginning at a time point at which the acoustic sensor 320 detected a biometric sound. More specifically, the symptom detecting section 306 detects a state of emission of a cough on the basis of a rate of decrease (rate of change) of (i) the percutaneous arterial blood oxygen saturation measured 20 seconds after a time point at which the acoustic sensor 320 detected a biometric sound with (ii) the mean of percutaneous arterial blood oxygen saturations over a period of 20 seconds after the time point.

Insufficient breathing due to coughing causes a decrease in saturation of oxygen that is taken into the body, with the result that there is a decrease in saturation of oxygen in the arterial blood. It takes approximately 20 seconds for the percutaneous arterial blood oxygen saturation to start decreasing after the subject has emitted a cough. Therefore, a change (decrease) in percutaneous arterial blood oxygen saturation can be detected with high accuracy by (i) obtaining the statistical value (mean) of percutaneous arterial blood oxygen saturations in a state in which the subject is not coughing and the percutaneous arterial blood oxygen saturation measured 20 seconds after a time point at which a biometric sound was detected and (ii) calculating a rate of decrease of the latter with the former.

The symptom detecting section 306 only needs to detect a state of emission of a cough on the basis of a result of comparison between the statistical value and the percutaneous arterial blood oxygen saturation measured at a time point at which the percutaneous arterial blood oxygen saturation is estimated to start decreasing due to coughing. The timing “20 seconds after” is merely an example.

Further, the measured value of percutaneous arterial blood oxygen saturation to be compared with the statistical value may be a value obtained by processing a plurality of measured values of percutaneous arterial blood oxygen saturation measured over a predetermined time period beginning at a time point at which a biometric sound was detected. For example, the symptom detecting section 306 may detect a change in percutaneous arterial blood oxygen saturation by (i) calculating a statistical value (e.g., mean) of a plurality of percutaneous arterial blood oxygen saturations obtained during a period of 5 seconds between a time point at which a period of 20 seconds has elapsed since detection of a biometric sound and a time point at which a period of 25 seconds has elapsed since the detection of the biometric sound and (ii) making a comparison between a statistical value over the period of 20 seconds (value obtained before coughing exerts an influence) and a statistical value over the period of 5 seconds (value obtained after coughing has exerted an influence).

Further, detecting means of the present invention only needs to detect a state of a subject on the basis of a biometric sound parameter (of a change in biometric sound parameter over time) and a biometric parameter (or a change in biometric parameter), and is not limited to detecting a cough.

<Storage Section 307>

The storage section 307 serves to record therein (i) a control program for each component, (ii) an OS program, (iii) an application program, and (vi) various types of data that are read out when the main control section 302 executes these programs. The storage section 307 is constituted by a nonvolatile memory device such as a hard disk or a flash memory.

It should be noted that the analysis device 301 may be provided with a detachable memory device in which to store biometric sound data and measurement data.

<Operation Section 308>

The operation section 308 is an input device, such as an input button or a switch, via which to input various set values and commands to the analysis device 301.

<Display Section 309>

The display section 309 serves to display configuration information of the analysis device 301 or results of an analysis conducted by the analysis device 301. The display section 309 is, for example, a liquid crystal display.

(Flow of Process in Symptom Detecting Section 340)

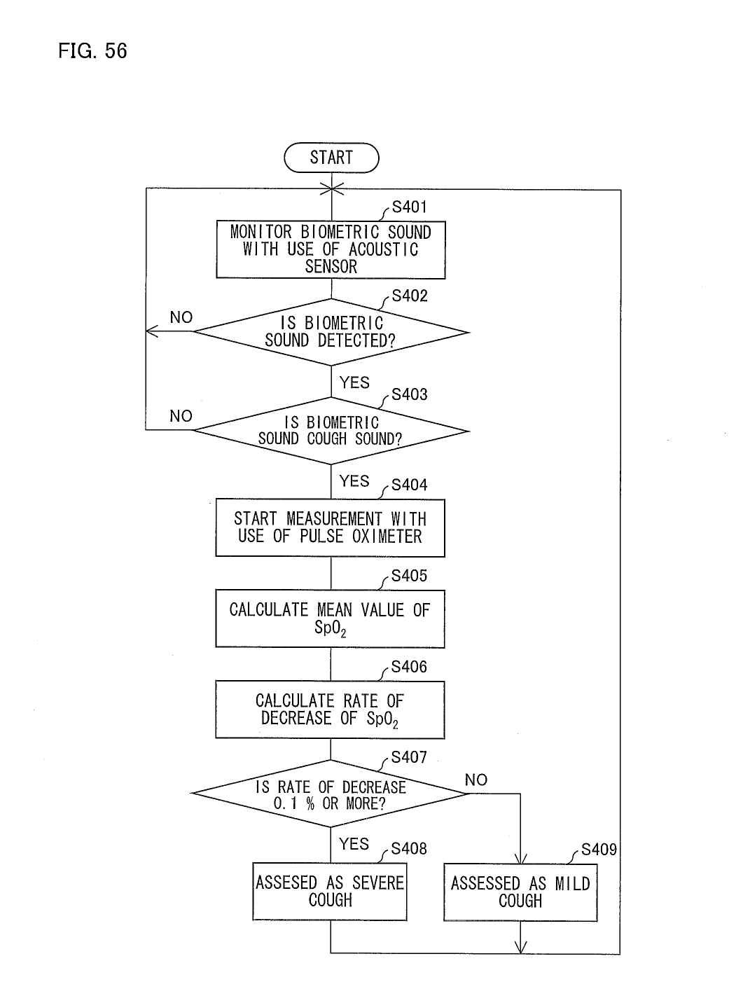

In the following, an example of the flow of a process (biometric method) in the symptom detecting device 340 is described. FIG. 56 is a flow chart illustrating an example of the flow of a process in the symptom detecting device 340.

First, the acoustic sensor 320, attached to the chest of the subject, continuously monitors biometric sounds (S401) and, upon detecting a biometric sound whose sound volume is equal to or higher than a predetermined sound volume (YES in S402), outputs biometric sound data containing the biometric sound to the cough sound determining section 303 of the analysis device 301.

Upon receipt of the biometric sound data (biometric sound parameter obtaining step), the cough sound determining section 303 records, in the storage section 307, a biometric sound detection time, which is a time point at which the biometric sound data was received, and determines whether or not the biometric sound data contains a cough sound (S403).

In a case where the cough sound determining section 303 has determined that the biometric sound data contains a cough sound (YES in S403), the measuring device control section 304 outputs a command to start measurement to the main control section 334 of the pulse oximeter 330.

Upon receipt of the command to start measurement, the main control section 334 causes the sensor section 331 to measure a percutaneous arterial blood oxygen saturation (SpO₂) for a predetermined time period (e.g., 20 seconds), and sequentially outputs, to the measuring device control section 304 of the analysis device 301, measurement data containing correspondence between (i) a measured value of percutaneous arterial blood oxygen saturation thus obtained and (ii) a time point at which the measured value was obtained (S404). It should be noted that the pulse oximeter 330 may transmit, to the analysis device 301, a set of measured values obtained during a predetermined measuring period.

On the other hand, in a case where the cough sound determining section 303 has determined that the biometric sound data does not contain a cough sound (NO in S403), the acoustic sensor 320 continues to monitor biometric sounds (that is, the process returns to S401).

Upon receipt of measured values of percutaneous arterial blood oxygen saturation (biometric parameter obtaining step) after the pulse oximeter 330 has started measuring the percutaneous arterial blood oxygen saturation, the measuring device control section 304 sequentially stores the measured values in the storage section 307.

The statistical processing section 305 calculates the mean of percutaneous arterial blood oxygen saturations measured during a period of 20 seconds having elapsed since the time of biometric sound detection as recorded in the storage section 307, and outputs the mean to the symptom detection section 306 (S405).

The symptom detecting section 306 obtains, from the storage section 307, a measured value of percutaneous arterial blood oxygen saturation measured 20 seconds after the time of biometric sound detection, and calculates a rate of decrease of the measured value with the mean calculated by the statistical processing section 305 (S406).

If the symptom detecting section 306 has determined that the rate of decrease is equal to or higher than 0.1% (YES in S407), the symptom detecting section 306 determines that a severe cough was emitted, and displays a result of the determination on the display section 309 and stores the result of determination in the storage section 307 (S408) (detecting step).

On the other hand, if the symptom detecting section 306 has determined that the rate of decrease is lower than 0.1% (NO in S407), the symptom detecting section 306 determines that a slight cough was emitted, and displays a result of the determination on the display section 309 and stores the result of determination in the storage section 307 (S409).

The result of determination stored in the storage section 307 can be later reconfirmed by the subject, and can be transmitted to another device. Alternatively, the result of determination may be stored in a detachable memory device (memory). In this case, attaching the memory device to another apparatus enables the apparatus to use the result of determination.

(Variation)

The analysis device 301 does not need to be constantly connected to the pulse oximeter 330 and the sound sensor 320. Measurement data generated by the pulse oximeter 330 and biometric sound data generated by the sound sensor 320 may be stored in an information storage device different from the pulse oximeter 330 and the sound sensor 320 so that the measurement data and the biometric sound data can be outputted from the information storage device to the analysis device 301. This configuration may be used in a case where the analysis device 301 is in the form of a personal computer. Further, the information storage device may be a storage device (e.g., a hard disk) provided in another personal computer, or may be a storage device (memory) that can be attached to and detached from the pulse oximeter 330 and/or the acoustic sensor 320. Further, the analysis device 301 may include a communication section for receiving biometric sound data and measurement data from another information storage device. The communication section, for example, serves to perform communication via a communication network such as the Internet or a LAN (local area network).

In such a case of obtaining biometric sound data and measurement data from another information memory device, it is preferable that the measurement data contain correspondence between (i) a plurality of measured values of percutaneous arterial blood oxygen saturation and (ii) time points at which the measured values were obtained respectively. Further, it is preferable that the biometric sound data contain information indicative of a time point at which the biometric sound data was obtained.

By the data containing information indicative of the time points at which the measurement data and the biometric sound data were obtained respectively, a comparison between (i) a time point at which a cough was emitted and (ii) a change in percutaneous arterial blood oxygen saturation over time can be made later than the time point at which the measurement was performed. This makes it unnecessary to determine in real time whether a cough was emitted.

Alternatively, in a case where the analysis device 301 does not determine whether or not the biometric sound contains a cough sound, the analysis device 301 does not always need to obtain biometric sound data (i.e., sound data per se) from the acoustic sensor 320, but only needs to obtain, from the acoustic sensor 320, biometric sound detection information indicating that a biometric sound has been detected. The biometric sound detection information may contain information indicative of the time point at which the biometric sound was detected. Alternatively, at a time point at which the analysis device 301 has received the biometric sound detection information, the analysis device 301 may store the time point in the storage section 307 in correspondence with the biometric sound detection information. In this case, the biometric sound detection information can be deemed as a biometric sound parameter.

A biometric sound that the acoustic sensor 320 detects is not limited to a cough sound, but may be a sound that accompanies a sneeze. Since there is a possibility of decrease in arterial blood oxygen saturation in the case of a sneeze, too, a sneeze can be detected in the same manner as a cough is detected.

In addition to a cough and a sneeze, the acoustic sensor 320 may also detect another symptom, such as asthma, which is accompanied by the generation of a sound.

### Example 1

The following describes Examples where coughs emitted by a subject were actually detected.

Sensing of biometric sounds was continuously carried out by attaching an acoustic sensor 320 to the chest of a subject. For measurement of percutaneous arterial blood oxygen saturation, a PULSOX-300i (manufactured by Konica Minolta Sensing, Inc.) serving as a pulse oximeter 330 was attached to an arm of the subject, with its sensor section attached to a fingertip of the subject.

A particular algorithm was used to detect a cough sound from among sounds detected by the acoustic sensor 320. At the same time, the percutaneous arterial blood oxygen saturation was continuously measured. Then, the mean of percutaneous arterial blood oxygen saturations over a period of 15 seconds (15-second mean) elapsed after a time point t (in seconds) at which the acoustic sensor 320 detected a biometric sound was calculated, and the rate of change of (i) percutaneous arterial blood oxygen saturation (real-time value) at t+20 (seconds) with (ii) the mean was calculated. The rate of change is represented by Equation (1):

(Rate of change)=(Real-time value)/(15-second mean)−1.0 (1)

In a case where the rate of change takes on a positive value, it means a rate of increase. In a case where the rate of change takes on a negative value, it means a rate of decrease.

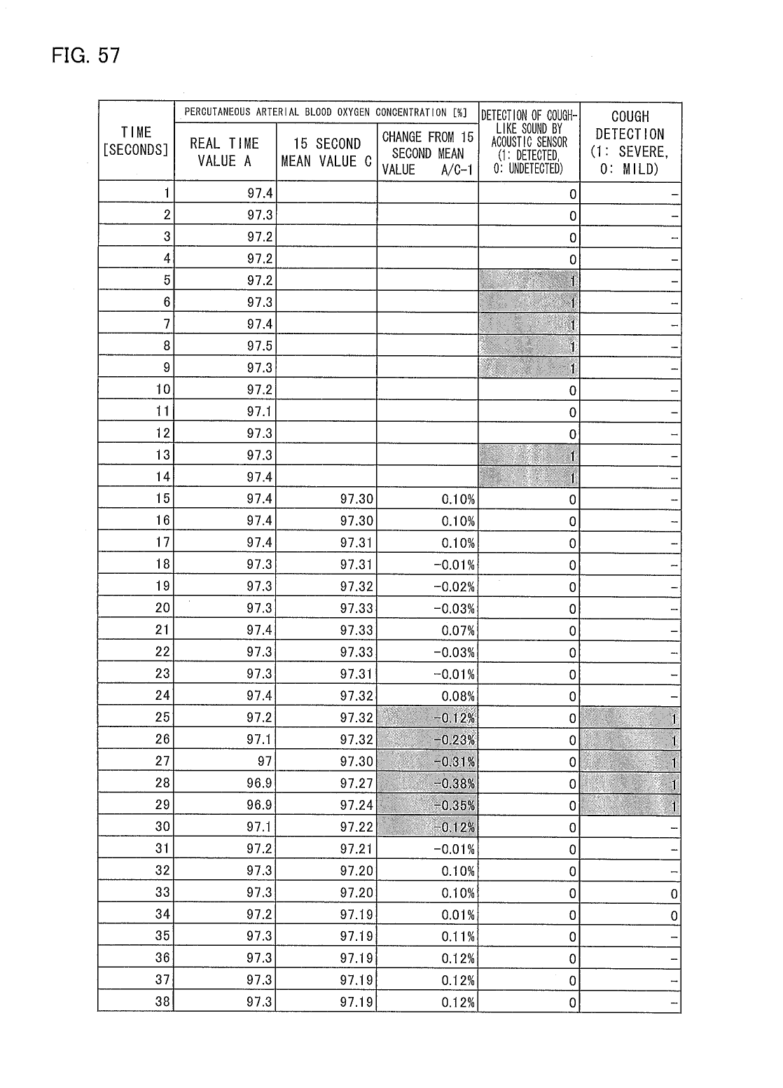

FIG. 57 shows experimental results of Example 1. As shown in FIG. 57, cough sounds were detected at time points t=5 to 9, and 20 seconds after each of the time points (t=25 to 29), decreases in percutaneous arterial blood oxygen saturation from the 15-second mean were observed. Since each of the rates of change was 0.1% or higher, it was determined that the severity of coughing was high.

Coughs were actually emitted at the time point's t=5 to 9, so it was confirmed that the coughs emitted have been surely detected.

Further, it was determined that cough sounds had been generated at t=13, 14. However, since there was no decrease in percutaneous arterial blood oxygen saturation at t=33, it was determined that the severity of coughing was low.

In actuality, however, no coughs were detected at the time points t=13, 14. This is considered to be attributable to an error made by the cough sound detection algorithm. That is, this is considered to be attributable to the fact that the noises picked up by the acoustic sensor 320 were interpreted as cough sounds.

In this case, too, there were no decreases in percutaneous arterial blood oxygen saturation at t=33, i.e., 20 seconds after t=13, and t=34, i.e., 20 seconds after t=14. Therefore, it was determined that the severity of coughing was not high but low. From this result, it is obvious that the accuracy of cough detection can be made higher by taking changes in percutaneous arterial blood oxygen saturation into account than by relying solely on the cough sound detection algorithm.

Since there is a possibility, as mentioned above, that a noise detected may be interpreted as a slight cough, the emission of a cough may be determined only in a case where there is a decrease of 0.1% or more in percutaneous arterial blood oxygen saturation. With such an algorithm, it is determined that the sounds at t=13, 14 are not due to coughing.

### Example 2

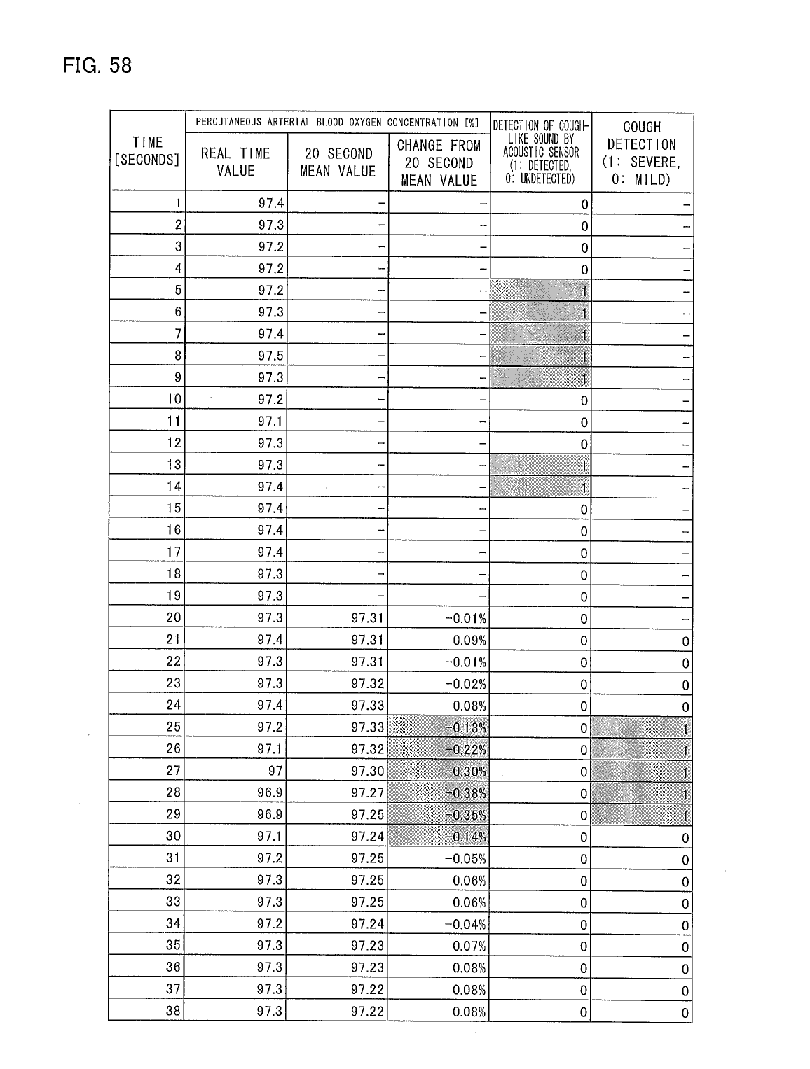

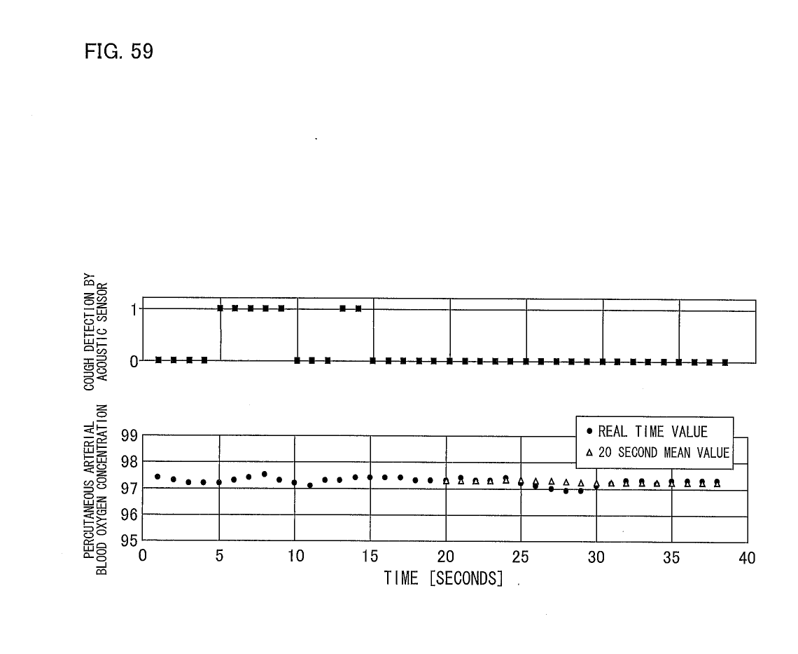

With reference to the same measurement data as those of Example 1, the following explains experimental results obtained by adopting a 20-second mean of percutaneous arterial blood oxygen saturations instead of the 15-second mean. FIG. 58 shows experimental results of Example 2. FIG. 59 shows the results of FIG. 58 in graph form.

As shown in FIGS. 58 and 59, the final results of determination are the same as those of Example 1 even in the case where the mean of percutaneous arterial blood oxygen saturations over a period of 20 seconds is calculated. However, by taking a 20-second mean, the percutaneous arterial blood oxygen saturation of the subject not coughing can be calculated with less variation. In particular, in a case where (i) a cough emitted by a subject whose percutaneous arterial blood oxygen saturation violently changes is detected or in a case where (ii) the measurement accuracy of the pulse oximeter 330 is low, it is preferable to take the mean of percutaneous arterial blood oxygen saturations over a period of 20 seconds or longer.

(Effects of Symptom Detecting Device 340)

As described above, the symptom detecting device 340 determines, on the basis of (i) biometric sound data outputted from the acoustic sensor 320 and (ii) measurement data of percutaneous arterial blood oxygen saturation outputted from the pulse oximeter 330, whether or not the subject has coughed (and whether or not coughing is severe) The percutaneous arterial blood oxygen saturation is a physiological index for a subject that may vary according to the subject's symptom of emitting a sound (i.e., coughing).

That is, in detecting a symptom, the symptom detecting device 340 does not use only information (biometric sound parameter) regarding a sound (e.g., a cough sound) that is generated due to the symptom, but also detects a change in another physiological parameter (e.g., percutaneous arterial blood oxygen saturation) that may vary according to the symptom.

The accuracy with which a symptom is detected can be made higher by this configuration than by utilizing only a biometric sound parameter directly reflecting the symptom.

Further, the symptom detecting device 340 uses, as a second parameter, the percutaneous arterial blood oxygen saturation, which can be quantitatively analyzed, and as such, can determine the severity of coughing in stages according to the rate of change in percutaneous arterial blood oxygen saturation. This makes it possible to provide medically important information, i.e., the severity of coughing, which cannot be obtained simply by determining whether or not a subject has coughed, and such information is believed to strongly support doctors in diagnosis, treatment, and the like.

Further, since the percutaneous arterial blood oxygen saturation is measured only in a case where the acoustic sensor 320 has detected a sound that may be a cough sound, the system consumes less power and is therefore suitable to mobile applications.

The invention of Patent Literature 4 determines, on the basis of (i) a cough sound that a subject emits and (ii) a body motion that the subject makes, whether or not a subject has coughed. However, information indicative of a body motion that the subject makes is not such a biometric parameter as that described above. Since the subject can often make a body motion without coughing, the accuracy of detection of coughing may not be made very high even by detecting coughing on the basis of a body motion that the subject makes.

### Embodiment 4

Alternatively, the present invention relates to an assessing device and assessing method (a measurement position assessing device, a measurement position assessment method, a control program, and a recording medium) for assessing suitability of an attachment position of a sound sensor which is attached to a living body.

[Technical Problem]

The above-described conventional configurations (in particular, Patent Literatures 5 to 7, etc.) use a pulse oximeter to measure blood oxygen saturation. In this case, a sensor is attached to a fingertip. In addition, a sensor for measuring a breath sound is attached under a nose. Therefore, subject's movement during sleep can cause, for example, detachment of the sensor(s), with the result of a failure of a precise measurement.

The above problem can be solved by attaching a sensor for detecting a biometric sound such as a breath sound onto a chest. However, it may be difficult for a user who has little knowledge of medicine to find a desirable place to attach the sensor on a chest.

The present invention has been accomplished in view of the above problem, and an object of the present invention is to provide a measurement position assessing device that determines a suitable attachment position of a biometric sound sensor which detects a biometric sound.

### Embodiment 4-1

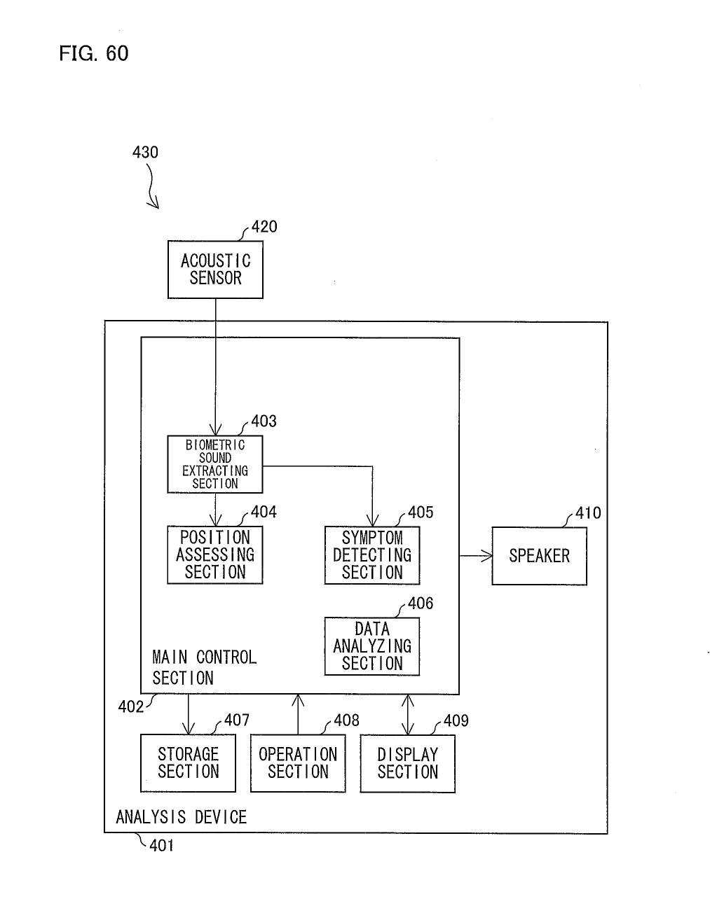

The following will describe one embodiment of the present invention with reference to FIGS. 60 through 62. The present embodiment describes a measuring device (measurement position assessing device) 430 that detects an apnea state. However, the present invention is not limited to a measuring device that detects an apnea state, and the present invention can be applied to a measuring device that detects a symptom other than apnea, as long as the measuring device includes a sound sensor that is attached to a subject (living body) and detects a biometric sound.

It should be noted that the following description assumes that the measuring device 430 is operated by a subject. However, the measuring device 430 may be operated by a user other than the subject, such as medical personnel.

The measuring device 430 notifies a subject of desirability of an attachment position of a sound sensor (biometric sound sensor) 420 by an assessment sound or by other means, thereby leading the subject to attach the sound sensor 420 to a suitable position. FIG. 60 is a diagram schematically illustrating a configuration of the measuring device 430. As illustrated in FIG. 60, the measuring device 430 includes an analysis device 401 and the sound sensor 420.

<Sound Sensor 420>

The sound sensor 420 is a contact microphone that is attached to the chest of a subject so as to detect a breath sound that the subject emits. A usable example of the sound sensor 420 is a contact microphone described in Japanese Patent Application Publication, Tokukai, No. 2009-233103 A. FIG. 29 is a cross-sectional view illustrating a configuration of the sound sensor 420. As illustrated in FIG. 29, the sound sensor 420 is a sound-collecting unit based on a so-called condenser microphone, and includes a housing 271 and a diaphragm 273. The housing 271 has a cylindrical shape, and has one end face open. The diaphragm 273 is in closed contact with the housing 271 so as to close the open face of the housing 271. Further, the sound sensor 420 includes a first conversion section 275, an A/D conversion section 277, a substrate 278, and an electric power supply section 279. The A/D conversion section 277 serves as a second conversion section. The first conversion section 275 and the A/D conversion section 277 are mounted on the substrate 278. The electric power supply section 279 supplies electric power to the first conversion section 275 and the A/D conversion section 277.

Provided on a surface of the diaphragm 273 is a tackiness agent layer 274 that causes the sound sensor 420 to be attached to a body surface (H) of the subject. The sound sensor 420 is attached to a position such as the chest, and only needs to be attached to any place where the sound sensor 420 can effectively pick up a breath sound.

When the patient emits a biometric sound e.g., by coughing, breathing, or swallowing, the diaphragm 273 minutely vibrates in accordance with the wavelength of the biometric sound. The minute vibration of the diaphragm 273 is transmitted to the first conversion section 275 via an air chamber wall 276. The air chamber wall 276 has a circular conical shape, and has upper and lower open faces.

The vibration transmitted through the air chamber wall 276 is converted into an electric signal by the first conversion section 275. The electric signal is then converted into a digital signal by the A/D conversion section 277. The digital signal is then transmitted in a form of biometric sound data to a biometric sound extracting section 403 of the analysis device 401.

The sound sensor 420 and the analysis device 401 only need to be communicably connected to each other, either via cable or wirelessly. However, wireless connection is more preferable due to its elimination of wires getting in the way. The analysis device 401 may be contained in the sound sensor 420.

Further, the sound sensor 420 only needs to be attached to a place where a measurement target sound can be picked up, and in order to pick up an abdominal sound, the sound sensor 420 should be attached to an abdominal region.

<Analysis Device 401>

The analysis device 401 detects an apnea state of a subject by using biometric sound data transmitted from the sound sensor 420. As illustrated in FIG. 60, the analysis device 401 includes a main control section 402, a storage section 407, an operation section 408, a display section 409, and a speaker (notifying section) 410. The main control section 402 includes a biometric sound extracting section (sound data obtaining means) 403, a position assessing section (assessing means) 404, a symptom detecting section 405, and a data analyzing section 406.

<Biometric Sound Extracting Section 403>

The biometric sound extracting section 403 receives biometric sound data transmitted from the sound sensor 420, and then extracts biometric sound (measurement target sound), which is a target for measurement, from the biometric sound data. In the present embodiment, the biometric sound extracting section 403 extracts, from the biometric sound data, a signal (herein referred to as “breath sound signal”) reflecting a respiration and having a low frequency (a frequency equal to or lower than 7 Hz).

<Position Assessing Section 404>

The position assessing section 404 assesses suitability of the attachment position of the sound sensor 420 on the basis of biometric sound data obtained by the biometric sound extracting section 403. More specifically, the position assessing section 404 makes a comparison between measurement target sounds extracted by the biometric sound extracting section 403, so as to relatively assess the suitability of the sound sensor 420 (first assessment method). Alternatively, the position assessing section 404 assesses suitability of the attachment position of the sound sensor 420 on the basis of a result of comparison between (i) an amplitude of a measurement target sound extracted by the biometric sound extracting section 403 and (ii) a predetermined reference value (second assessment method).

(First Assessment Method)

In the first assessment method, in a case where a search for a most suitable attachment position is made by changing an attachment position of a single sound sensor 420 to another position, an amplitude of a measurement target sound at a current attachment position is compared with an amplitude of a measurement target sound at a previous attachment position. If the current amplitude is greater than the previous amplitude, the assessment sound is emitted at shorter time intervals. Conversely, if the current amplitude is smaller than the previous amplitude, the assessment sound is emitted at longer time intervals.

Further, the biometric sound extracting section 403 may receive pieces of biometric sound data respectively from a plurality of sound sensors 420 attached at different positions. In this case, the position assessing section 404 makes comparison between measurement target sounds extracted from the respective pieces of biometric sound data, and then displays, on a display section 409, information (such as a number given to the sound sensor 420) identifying the sound sensor 420 from which a measurement target sound having the greatest amplitude has been obtained.

(Second Assessment Method)

In the second assessment method, the position assessing section 404 compares (i) amplitude ranges (amplitude levels) preset on a given scale with (ii) an amplitude of a measurement target sound extracted by the biometric sound extracting section 403, so as to assess which amplitude level the amplitude of the measurement target sound thus extracted corresponds to. Then, the position assessing section 404 controls the speaker 410 so that the speaker 410 outputs an assessment sound corresponding to the amplitude level thus assessed.

For example, the amplitude levels are staged in three levels, and settings of the amplitude levels are made in such a manner that the time interval of the assessment sound decreases with increase in amplitude.

The amplitude of the measurement target sound may be compared with one (1) reference value, which is, for example, a value equivalent to a minimal amplitude required to detect a symptom as a target for detection.

Further, since a sound volume (amplitude) of a biometric sound varies depending on subjects, the amplitude range or a maximum amplitude value may be set for each subject. Accordingly, (i) a reference value setting mode may be provided to determine a reference value for setting a desirable amplitude range, or (ii) a maximum value setting mode may be provided to set the maximum amplitude value.

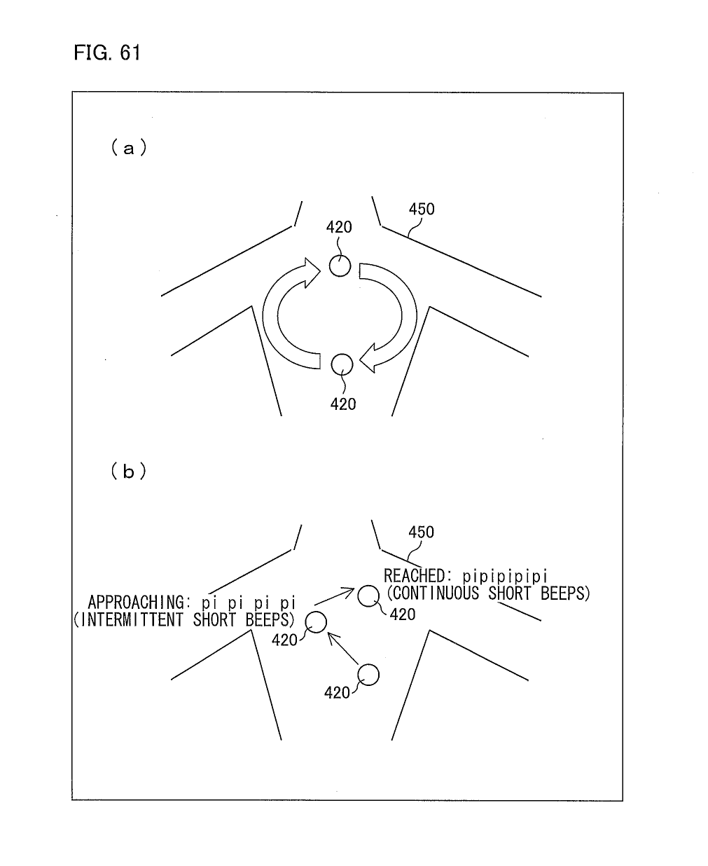

(a) of FIG. 61 is a diagram illustrating a maximum value setting method. In the maximum value setting mode, a subject causes the sound sensor 420 to pick up a biometric sound, while changing an attachment position of the sound sensor 420 on the human body 450. The biometric sound extracting section 403 sequentially extracts a biometric sound from biometric sound data transmitted by the sound sensor 420, and then outputs the biometric sound to the position assessing section 404. The position assessing section 404 measures an amplitude of an incoming biometric sound, and then causes its amplitude value to be stored in the storage section 407.

At the completion of the maximum value setting mode, the position assessing section 404 causes a greatest amplitude value among a plurality of amplitude values stored in the storage section 407 to be stored as a maximum amplitude value of the subject in the storage section 407.

In assessing suitability of the attachment position of the sound sensor 420, the position assessing section 404 makes an interval of the assessment sound shorter as an amplitude value of the biometric sound outputted from the sound sensor 420 approaches the maximum amplitude value, as illustrated in (b) of FIG. 61. (b) of FIG. 61 is a diagram illustrating an example of how the assessment sound changes as an amplitude value approaches its maximum.

On the other hand, in the reference value setting mode, for example, a reference value is assumed to be a value obtained by subtracting a predetermined value from a maximum amplitude value among amplitude values obtained from a subject, and the subject is notified of whether or not the reference value is exceeded.

Such a reference value (or maximum value) setting function may be provided to the position assessing section 404. Alternatively, a reference value setting section (or maximum value setting section), which is different from the position assessing section 404, may be provided additionally. Further, the reference value (or maximum value) setting mode may be provided only for a predetermined time period, after which a shift is made to a normal mode in which an attachment position is assessed automatically.

<Symptom Detecting Section 405>

The symptom detecting section 405 detects a particular symptom by analyzing, for example, (i) an amplitude of a measurement target sound extracted by the biometric sound extracting section 403 and (ii) an occurrence pattern of the measurement target sound. In the present embodiment, the symptom detecting section 405 detects an apnea state. For example, the symptom detecting section 405 assesses an apnea state in a case where a breath sound having an amplitude that is equal to or greater than a predetermined amplitude is not detected for 10 seconds or longer. A result of the symptom detection is stored in the form of detection record data in the storage section 407, together with information on a date and time of the detection of the symptom.

It should be noted that in the symptom detecting section 405, detection thresholds of a breath sound may be staged in two levels so that distinguishing between an apnea state and a hypopnea state can be made during detection. Apnea means a pause of aural and nasal airflows for 10 seconds or longer, and hypopnea means more than 50% decrease in ventilation for 10 seconds or longer.

In a case where the measuring device 430 is in the form of a device for detecting a symptom other than apnea syndrome, the symptom detecting section 405 may detect a symptom of a detection target from a measurement target sound. For example, symptoms such as cardiac valvular disease, congenital cardiac disease, and cardiac failure may be detected from a heart sound, and symptoms such as pneumothorax, bronchial asthma, and obstructive lung disease may be detected from an abnormal breath sound. Further, symptoms such as absent bowel sound (bowel obstruction), low-pitched bowel sound (hypofunction), and high-pitched bowel sound (hyperactive bowel sound) may be detected from an abdominal sound (bowel sound). Absence of an abdominal sound after the appearance of a symptom of high-pitched bowel sound is a sign of a very severe disease, and may result in necrosis of bowel tissue. A high-pitched bowel sound appears as a response of a bowel to a disease.

A method used by the symptom detecting section 405 for detecting the foregoing symptoms may be a publicly known method. Such a method is not directly relevant to the essence of the present invention, and an explanation of the method is therefore omitted.

<Data Analyzing Section 406>

The data analyzing section 406 analyzes, over a medium term and/or a long term, the detection record data stored in the storage section 407, and generates a graph showing change of a subject's symptom. The processing of the data analyzing section 406 may be performed as needed in accordance with instructions from the subject, or may be performed regularly.

For example, the data analyzing section 406 may display, in graph form or in other form, (i) long-term changes in frequency of occurrence of an apnea state and (ii) changes in a physiological index (a weight, a blood pressure, a duration of an excessive daytime sleep, etc.) associated with apnea and/or in subject's lifestyle habit (amount of exercise, etc.), thereby indicating the extent to which a symptom of the apnea syndrome has been relieved as a result of the changes of subject's lifestyle habit. The information on the physiological index and the lifestyle habit may be entered by the subject through the operation section 408 and stored in the storage section 407.

Further, in accordance with instructions from the subject, the data analyzing section 406 may analyze the detection record data to generate information on the number of times an apnea state has occurred during sleep on a designated date. For example, a symptom of sleep apnea syndrome may be represented in stages as (i) a mild stage when a pause for 10 seconds or longer has occurred 5 to 14 times within a one-hour period, (ii) a moderate stage when the pause has occurred 15 to 29 times within a one-hour period, and (iii) a severe stage when the pause has occurred 30 times or more within a one-hour period. The number of times an apnea state has occurred may be displayed in numerical form, in graphical form, in tabular form, or in other form on the display section 409.

It should be noted that the sleep apnea syndrome is defined as a syndrome having such a symptom that (i) an apnea state for 10 seconds or longer occurs 30 times or more during sleep of one night (for 7 hours) or that (ii) apnea and hypopnea occur 5 times or more during each hour of sleep.

Further, the sleep apnea syndrome is also defined as a syndrome showing apnea hypopnea index (AHI), which is a total number of occurrences of apnea and hypopnea during each hour of sleep, of 5 or more, and accompanied by an excessive daytime sleep or the like symptom.

Some patients complain of insomnia with repetitive hypopnea, but such insomnia does not fall under the above definitions. The patients who develop such insomnia often have severe snoring and teeth grinding, and the insomnia is therefore referred to as “insomnia with snoring and teeth grinding”.

<Storage Section 407>

The storage section 407 serves to record therein (i) a control program for each component, (ii) an OS program, (iii) an application program, and (vi) various types of data that are read out when the main control section 402 executes these programs. The storage section 407 is constituted by a nonvolatile memory device such as a hard disk or a flash memory.

It should be noted that the analysis device 401 may be provided with a detachable memory device in which to store biometric sound data.

<Operation Section 408>

The operation section 408 is an input device, such as an input button or a switch, via which to input various set values and commands to the analysis device 401.

<Display Section 409>

The display section 409 serves to display configuration information on the analysis device 401 or results of an analysis conducted by the analysis device 401. The display section 409 is, for example, a liquid crystal display.

<Speaker 410>

The speaker 410 is a notifying section that notifies a user of suitability of an attachment position of the sound sensor 420. The speaker 410 emits a sound (referred to as “assessment sound”) corresponding to a result of the assessment made by the position assessing section 404, so as to notify the user of the degree of desirability of the attachment position of the sound sensor 420.

The assessment sound indicates suitability of the attachment position with, for example, (i) varying time intervals at which a sound is emitted, (ii) varying sound volumes, or (iii) varying pitches. For example, in a case where an attachment position is not desirable, the assessment sound may be emitted at longer time intervals (sounding like “beep, . . . , beep, . . . , beep, . . . ”), whereas in a case where an attachment position is desirable, the assessment sound may be emitted at shorter time intervals (sounding like “beep, beep, beep”). Alternatively, in a case where an attachment position is not desirable, the assessment sound may be emitted at a lower pitch, whereas in a case where an attachment position is desirable, the assessment sound may be emitted at a higher pitch. Further alternatively, (i) a sound volume or a melody of the assessment sound may be varied depending on desirability of the attachment position, or (ii) desirability of the attachment position may be notified by voice.

Further, a time interval of the assessment sound may be made shorter as an amplitude of a biometric sound obtained from the sound sensor 420 approaches a predetermined maximum amplitude value.

Still further, desirability of the attachment position may be indicated by varying illumination patterns or varying light-emission colors of a light-emitting device (for example, a light-emitting diode). Yet further, desirability of the attachment position may be indicated by characters and figures in the display section 409. Further, the sound sensor 420 may be configured to vibrate in correspondence with desirability of the attachment position. In these cases, the light-emitting device, the display section 409, or the sound sensor 420 is the notifying section.

In addition, the speaker 410 may be built into the sound sensor 420.

(Flow of Process Carried Out by Measuring Device 430)

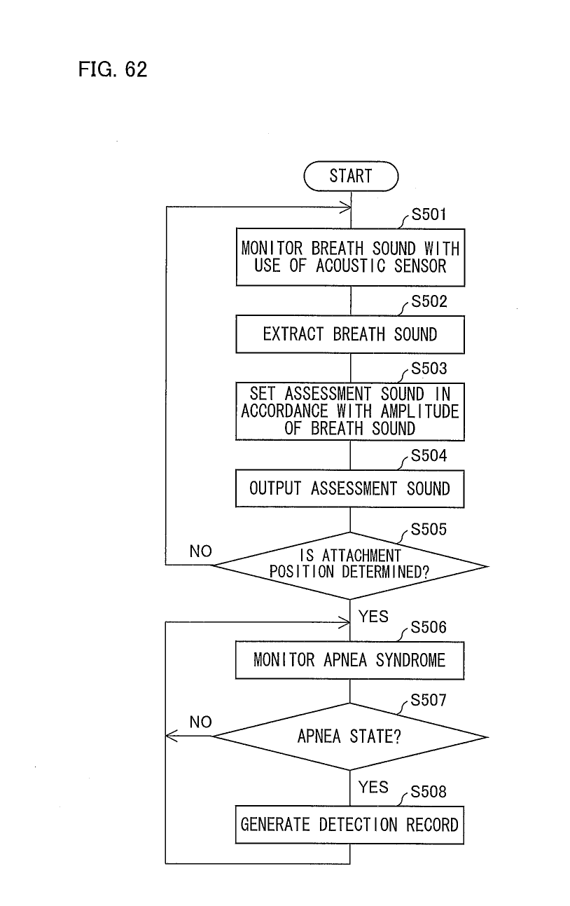

Next, an example flow of a process (measurement position assessment method) carried out by the measuring device 430 will be described. FIG. 62 is a flowchart illustrating an example flow of a process carried out by the measuring device 430. The following will describe an arrangement of a setting on a time interval of the assessment sound by the above-described second assessment method, assuming that a search for a most suitable attachment position is made by changing the attachment position of a single sound sensor 420 to another position.

As illustrated in FIG. 62, firstly, the sound sensor 420 attached to a chest of a subject continuously carries out monitoring of a biometric sound (S501), and then outputs biometric sound data containing the biometric sound thus detected to the biometric sound extracting section 403 of the analysis device 401.

Upon receipt of the biometric sound data (sound data obtaining step), the biometric sound extracting section 403 extracts a 7-Hz signal or a signal having a frequency below 7 Hz (breath sound signal) from the biometric sound data, and then outputs the breath sound signal thus extracted to the position assessing section 404 (S502).

The position assessing section 404 assesses which of the predetermined amplitude ranges an amplitude of the breath sound signal extracted by the biometric sound extracting section 403 falls within (assessment step), and controls the speaker 410 so that the speaker 410 outputs an assessment sound corresponding to the amplitude range thus assessed (S503).

Then, the assessment sound set by the position assessing section 404 is outputted from the speaker 410 (S504).

At this time, if the subject has changed the attachment position of the sound sensor 420 (NO in S505), the steps S501 through S504 are repeated.

When the subject has determined the attachment position of the sound sensor 420 (YES in S505), and entered a command for starting apnea monitoring, the biometric sound extracting section 403 extracts a breath sound signal from the biometric sound data, and then outputs the breath sound signal thus extracted to the symptom detecting section 405. The symptom detecting section 405 starts apnea monitoring with reference to the incoming breath sound signal (S506).

If a breath sound signal having an amplitude equal to or greater than a predetermined amplitude has not been detected for 10 seconds or longer, the symptom detecting section 405 determines that the subject is in apnea state (YES in S507). The symptom detecting section 405 thus generates detection record data containing (i) information on a date and time of the detection of the apnea state and (ii) a duration of the occurrence of the apnea state, and then stores the detection record data in the storage section 407 (S508).

Thereafter, the detection record data thus stored in the storage section 407 is analyzed by the data analyzing section 406.

(Advantageous Effect of Measuring Device 430)

As described above, the measuring device 430 determines a suitable attachment position of the sound sensor 420 on the basis of a breath sound detected by the sound sensor 420, so that the measuring device 430 can notify desirability of the attachment position to a subject who is confused about where to attach the sound sensor 420. This makes it possible to help the subject to make a more precise measurement.

### Embodiment 4-2

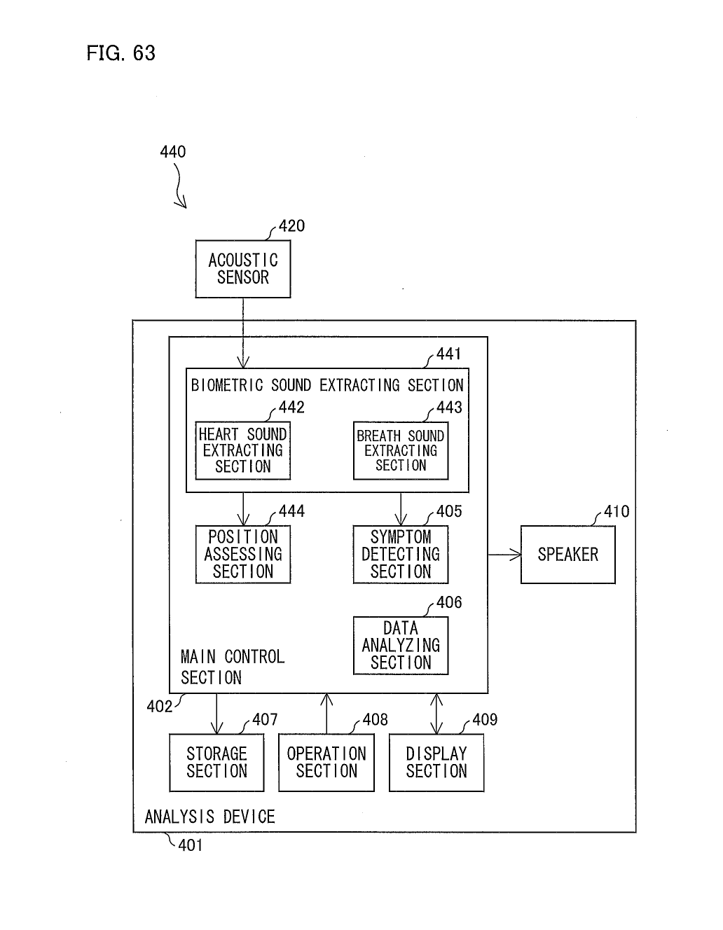

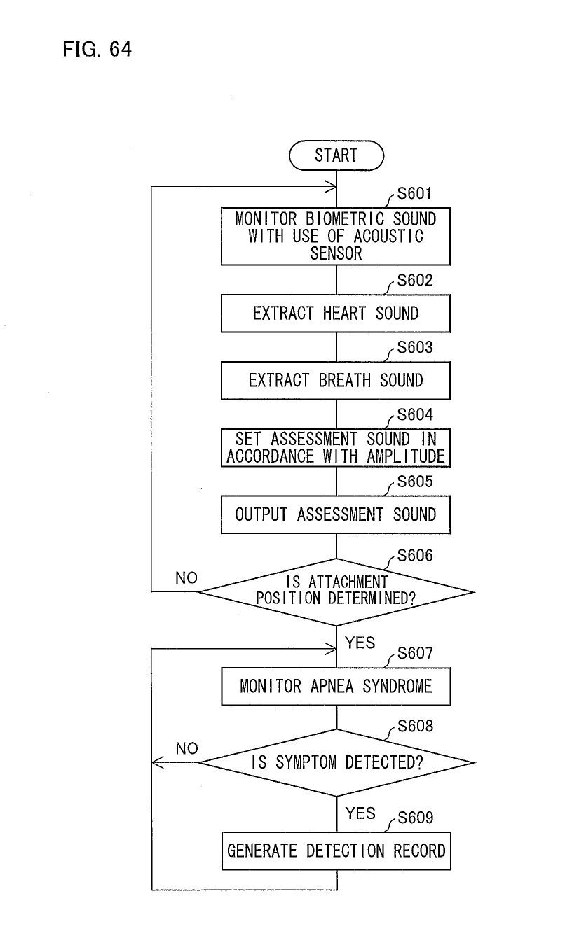

The following will describe another embodiment of the present invention with reference to FIGS. 63 and 64. It should be noted that members which are similar to those described in the Embodiment 4-1 are given the same reference numerals, and explanations thereof are omitted. The present embodiment assumes that a measuring device 440 detects an apnea state from a heart sound and a breath sound, and that the sound sensor 420 detects a heart sound and a breath sound (different types of measurement target sounds) emitted by a subject.

The sound sensor 420 is attached at a position between a chest and a throat so as to detect a heart sound and a breath sound, and the sound sensor 420 may be configured in a manner similar to that illustrated in FIG. 29.

FIG. 63 is a diagram schematically illustrating a configuration of the measuring device 440 of the present embodiment. As illustrated in FIG. 63, the measuring device 440 includes a biometric sound extracting section (sound data obtaining means) 441, by which the biometric sound extracting section 403 is replaced, and also includes a position assessing section (assessing means) 444, by which the position assessing section 404 is replaced.

<Biometric Sound Extracting Section 441>

The biometric sound extracting section 441 includes a heart sound extracting section 442 and a breath sound extracting section 443.

The heart sound extracting section 442 receives biometric sound data transmitted from the sound sensor 420, and then extracts a heart sound (cardiac sound) from the biometric sound data. A normal heart sound has two frequencies, namely 30 Hz and 70 Hz, as specific frequencies of the normal heart sound. Therefore, the heart sound extracting section 442 extracts these 30-Hz and 70-Hz signals. The breath sound extracting section 443 extracts a breath sound from the biometric sound data as in the biometric sound extracting section 403.

<Position Assessing Section 444>

The position assessing section 444 assesses suitability of an attachment position of the sound sensor 420 on the basis of whether or not different types of measurement target sounds contained in the biometric sound data each meet a predetermined requirement. Specifically, the position assessing section 444 assesses suitability of the attachment position of the sound sensor 420 on the basis of (i) whether or not an amplitude of a heart sound extracted by the heart sound extracting section 442 reaches a preset reference value of a heart sound and (ii) whether or not an amplitude of a breath sound extracted by the breath sound extracting section 443 reaches a preset reference value of a breath sound. Further, the position assessing section 444 makes a comparison between assessment scores of a plurality of attachment positions (or a plurality of sound sensors 420 which are attached at different positions) so as to determine a more desirable attachment position.

For example, the assessment scores are staged in three levels as follows: Score “3” (most suitable) is given in a case where both of amplitudes of a heart sound and a breath sound reach their respective reference values; Score “2” is given in a case where either of them reaches the reference value; and Score “1” is given in a case where neither of them reaches the reference values. In this case, an assessment sound corresponding to each score may be outputted from the speaker 410. Further, (i) the assessment scores may be staged in four or more levels, and (ii) for each amplitude of a heart sound and a breath sound, two or more reference values may be provided depending on a magnitude of the amplitude.

Alternatively, the position assessing section 444 may cause light emission modes of the light-emitting device (for example, an LED (light-emitting diode)) (not shown) to vary depending on an assessment score. Specifically, for example, assessment scores are staged in two levels each for a heart sound and a breath sound, and an LED indicating heart sound assessment scores and an LED indicating breath sound assessment scores are provided. Then, the position assessing section 444 causes the LED to illuminate green if a heart sound or a breath sound exceeds a reference value, and the position assessing section 444 causes the LED to illuminate red if a heart sound or a breath sound does not exceed the reference value.

Therefore, in a case where both a heart sound and a breath sound exceed their respective reference values, both of the two LEDs illuminate green. However, in a case where either of the sounds does not reach the reference value, the LEDs illuminate red and green and vice versa.

As in the Embodiment 4-1, (i) a reference value setting mode may be provided to determine a reference value for setting a preferred amplitude range for each subject, or (ii) a maximum value setting mode may be provided to set a maximum amplitude value for each subject.

<Symptom Detecting Section 405>

The symptom detecting section 405 detects an apnea state (and an extent of the apnea state) by analyzing an amplitudes, occurrence patterns, etc. of (i) a heart sound extracted by the heart sound extracting section 442 and (ii) a breath sound extracted by the breath sound extracting section 443. In the apnea state, oxygen saturation in arterial blood decreases, and a heart rate increases accordingly. On this account, it is possible to determine that the subject is in an apnea state in a case where (i) a breath sound is of a value smaller than a predetermined reference value and where (ii) a heart rate is greater than a predetermined reference value.

(Flow of Process Carried Out by Measuring Device 440)

Next, the following will describe an example flow of a process carried out by the measuring device 440. FIG. 64 is a flowchart illustrating an example flow of the process carried out by the measuring device 440.

As illustrated in FIG. 64, firstly, the sound sensor 420 attached to a chest of a subject continuously carries out monitoring of a biometric sound (S601), and then outputs biometric sound data containing the biometric sound to the biometric sound extracting section 441 of the analysis device 401.

Upon receipt of the biometric sound data, the heart sound extracting section 442 of the biometric sound extracting section 441 extracts 30-Hz and 70-Hz signals (heart sound signals) from the biometric sound data, and then outputs the heart sound signals thus extracted to the position assessing section 444 (S602).

Meanwhile, upon receipt of the biometric sound data, the breath sound extracting section 443 extracts a 7-Hz signal or a signal having a frequency below 7 Hz (breath sound signal) from the biometric sound data, and then outputs the breath sound signal thus extracted to the position assessing section 444 (S603).

The position assessing section 444 sets an assessment sound on the basis of (i) whether or not an amplitude of the heart sound signal extracted by the heart sound extracting section 442 reaches a reference value preset for a heart sound and (ii) whether or not an amplitude of the breath sound signal extracted by the breath sound extracting section 443 reaches a reference value preset for a breath sound, and controls the speaker 410 so that the speaker 410 outputs the assessment sound (S604).

In this manner, the assessment sound set by the position assessing section 444 is outputted from the speaker 410 (S605).

At this time, if the subject has changed the attachment position of the sound sensor 420 (NO in S606), the steps S601 through S605 are repeated. In this case, the position assessing section 444 may chronologically store, in the storage section 407, the assessment scores calculated at the respective attachment positions, so that in a case where an assessment score at a certain attachment position is higher than an assessment score at a previous attachment position, the subject can be notified as such by an assessment sound emitted at shorter time intervals or by other means. Conversely, in a case where an assessment score at a certain attachment position is lower than an assessment score at a previous attachment position, the subject can be notified as such by an assessment sound emitted at longer time intervals or by other means.

On the other hand, when the subject has determined the attachment position of the sound sensor 420 (YES in S606), and entered a command for starting apnea monitoring, the biometric sound extracting section 403 extracts a heart sound signal and a breath sound signal from the biometric sound data, and then outputs the signals thus extracted to the symptom detecting section 405. The symptom detecting section 405 assesses the presence or absence of an apnea state from the incoming heart sound signal and breath sound signal (S607).

If the symptom detecting section 405 has detected an apnea state (YES in S608), the symptom detecting section 405 generates detection record data containing (i) information on a date and time of the detection of the apnea state and (ii) an extent of the symptom of apnea, and then causes the detection record data to be stored in the storage section 407 (S609).

A method for using the detection record data stored in the storage section 407 is similar to that in the Embodiment 4-1, and an explanation thereof is therefore omitted.

(Variation)

The measuring device 440 may be provided with two sound sensors 420, one of which detects a breath sound and the other of which detects a heart sound. In this case, desirability of the attachment position of the sound sensor 420 for breath sound detection and desirability of the attachment position of the sound sensor 420 for heart sound detection are individually assessed, and assessment results are notified to the subject. A breath sound and a heart sound are different in frequency from each other, and a sound being picked up out of these two sounds can be identified by its frequency. On this account, the two sound sensors 420 does not necessarily need to be distinguished between a sound sensor for breath sound detection and a sound sensor for heart sound detection.

Further, the measuring device 440 may use a single sound sensor 420 to (i) measure the presence or absence of a cardiac disease or an extent of a cardiac disease from a heart sound and to (ii) measure the presence or absence of a respiratory disease or an extent of a respiratory disease from a breath sound. That is, one type of symptom may be detected from two types of biometric sounds, and alternatively, two types of symptoms may be detected from two types of biometric sounds.

(Advantageous Effect of Measuring Device 440)

As described above, the measuring device 440, even in such an arrangement that a single sound sensor 420 detects two types of biometric sounds, can notify a subject of a desirable attachment position of the sound sensor 420. Therefore, an appropriate measurement can be made even by a subject who does not know a desirable attachment position.

### Other Modification Example

The present invention is not limited to the aforementioned embodiments, and is susceptible of various changes within the scope of the accompanying claims. Also, any embodiment obtained by suitable combinations of technical means disclosed in the different embodiments is also included within the technical scope of the present invention.

For example, the present invention may be applied to an animal other than a human, and may be used to detect a disease condition of a pet or a livestock animal. That is, a target to which a biometric sound sensor is attached in the present invention is not limited to a human (subject), but is a living body including a human.

<<Arrangements of Present Invention>>

The following arrangements are also included in present invention.

### As to Embodiment 1

The biometric device of the present invention may preferably be arranged such that the measurement result deriving means calculates, from the one or more parameters specified by the parameter specifying information, an index indicative of the state of the living body, the state relating to the measurement item.

The above arrangement (i) causes a measurement result corresponding to a measurement item to be outputted as an index, and thus (ii) allows a user to easily understand a state of a living body on the basis of the index. Further, the expression of a measurement result as an index allows the user to, for example, analyze, compare, and manage measurement results easily, and thus improves convenience.

The biometric device of the present invention may further include: an index calculation rule storage section in which an index calculation rule for calculating, with use of the one or more parameters, the index corresponding to the measurement item is stored for each index, wherein: the index calculation rule includes information on a weight to be assigned to a parameter, the weight being assessed on a basis of a magnitude of an influence caused by the parameter on the index calculation; and the measurement result deriving means, for the index calculation, assigns the weight to each of the one or more parameters in accordance with the index calculation rule, the weight being set for the each of the one or more parameters.

The biometric device of the present invention may preferably further include: a parameter attribute storage section in which a parameter attribute indicative of the magnitude of the influence caused by said parameter on the index calculation is stored for each index and for each parameter, wherein: the weight, the information of which is included in the index calculation rule, correlates to all or part of information indicated by the parameter attribute.

With the above arrangement, a weight to be assigned to a parameter has a value that accurately reflects a difference in magnitude of an influence caused by the parameter on the index calculation. The above arrangement thus allows the measurement result deriving means to calculate an index more accurately in accordance with the index calculation rule (weighting).

The biometric device of the present invention may preferably further include: parameter attribute managing means for, in accordance with an instruction that has been entered by a user into the biometric device and that intends to change the parameter attribute, changing the parameter attribute stored in the parameter attribute storage section, wherein: the parameter attribute managing means, in addition to the change to the parameter attribute stored in the parameter attribute storage section, changes the weight, the information of which is included in the index calculation rule.

With the above arrangement, in a case where the user has changed a parameter attribute, such a change can be reflected in the value of a weight assigned to a parameter. The above arrangement thus allows the measurement result deriving means to calculate an index more accurately in accordance with (i) the index calculation rule (weighting) and (ii) the user's intention.

The biometric device of the present invention may preferably be arranged such that the measurement method storage section further stores repeated measurement instruction information specifying timing for repeating the index calculation for each measurement item; and the measurement result deriving means repeatedly calculates, at the timing specified by the repeated measurement instruction information, the index with use of the biometric parameter obtained on the basis of the biometric signal information obtained repeatedly.

With the above arrangement, the biometric device stores, in the measurement method storage section, not only parameter specifying information but also repeated measurement instruction information in correspondence with a measurement item. Repeated measurement instruction information refers to information that specifies calculation timing (for example, how often the calculation is carried out, how many times the calculation is carried out, how long each calculation operation lasts, and when the calculation is carried out) for a case where the index calculation is carried out regularly.

Simply carrying out an index calculation once may, depending on a kind of measurement, not measure and assess a state of a living body accurately. In view of this, the above arrangement specifies, for each measurement item, timing of index calculation with use of repeated measurement instruction information. The above arrangement can thus control an operation of the measurement result deriving means so that the living body is measured by a measurement method suited for the measurement purpose.

The biometric device of the present invention may preferably further include: state evaluating means for, on a basis of the index repeatedly calculated by the measurement result deriving means, evaluating a health state of the living body, the health state relating to the measurement item.

The above arrangement allows the state evaluating means to evaluate a health state of a living body accurately with use of a plurality of indexes calculated repeatedly.

The biometric device of the present invention may preferably be arranged such that the state evaluating means, by comparing (i) an index calculated by the measurement result deriving means at a predetermined time point with (ii) a plurality of indexes repeatedly calculated by the measurement result deriving means, evaluates the health state of the living body, the health state being observed at the predetermined time point.

With the above arrangement, the state evaluating means compares (i) an index obtained through a single measurement with (ii) a plurality of indexes obtained through measurements carried out repeatedly. The state evaluating means thus evaluates a health state of a living body which health state is observed at the time of the single measurement.

The above arrangement consequently makes it possible to (i) evaluate, on the basis of a history, a health state of the living body which health state is observed at the time of the single measurement and thus (ii) assess a state more accurately. The biometric device of the present invention may preferably be arranged such that the measurement method storage section stores the parameter specifying information in such a manner that (i) a parameter essential to measurement and (ii) an auxiliary parameter that is preferably used in measurement are discriminated from each other.

The above arrangement allows the measurement result deriving means to, for parameters to be used, discriminate between essential parameters and auxiliary parameters. Even if the biometric device does not have all the parameters, the measurement result deriving means, if only the biometric device has the essential parameters, derives measurement result information that is suited for a measurement item and that maintains a certain level of accuracy. The measurement result deriving means can, if the biometric device further has the auxiliary parameters, derive measurement result information that is suited for a measurement item and that has high accuracy.

The biometric device of the present invention may, as described above, be arranged such that the one or more parameters include (i) the biometric parameter reflecting a physiological state of the living body and, in addition to the biometric parameter, (ii) an external parameter reflecting an environmental condition arising from outside the living body; and the measurement method storage section stores the parameter specifying information in such a manner that the biometric parameter and the external parameter are discriminated from each other.

The above arrangement allows the measurement result deriving means to derive measurement result information with use of, as a parameter corresponding to a measurement item, not only the biometric parameter but also the external parameter. A state of a living body may be influenced by an environmental condition arising from outside the living body. Thus, in a case where such a state is to be measured, the use of the external parameter makes it possible to measure a state of a living body more accurately.

The biometric device of the present invention may be arranged such that the external parameter includes at least one of (i) information on a specification of a biometric sensor for obtaining the biometric signal information from the living body, (ii) information on a position at which the biometric sensor is disposed, (iii) examinee information on the living body, and (iv) environment information on a measurement environment in which the living body is present; the biometric parameter includes one or more biometric parameters; the external parameter includes one or more external parameters; and the measurement method storage section stores, as the parameter specifying information in correspondence with the measurement item, a combination of (i) the one or more biometric parameters and (ii) the one or more external parameters.

With the above arrangement, the measurement result deriving means derives measurement result information with use of not only the biometric parameter but also an external parameter such as (i) information on the specifications of a biometric sensor for obtaining the biometric signal information from the living body, (ii) information on the position at which the biometric sensor is disposed, (iii) examinee information on the living body, and (iv) environment information on a measurement environment in which the living body is present. This indicates that even in a case where external factors such as the above influence a state of a living body, the measurement result deriving means can derive more accurate measurement result information in view of the above external factors. The above arrangement thus makes it possible to measure a state of a living body more accurately.

The biometric device of the present invention may preferably be arranged such that the biometric parameter includes (i) a parameter indicative of a change occurring inside the living body and (ii) a parameter indicative of a change appearing outside the living body.

In a case where a state of a living body is to be measured, the biometric parameter reflecting a physiological state of the living body is mainly a parameter indicative of a change occurring inside the living body. However, further using a parameter indicative of a change appearing outside the living body makes it possible to analyze the physiological state of the living body in greater detail. This arrangement in turn makes it possible to (i) measure a state of a living body accurately and thus (ii) derive measurement result information more accurately.

It is assumed that a parameter indicative of a change occurring inside a living body is, for example, (i) a frequency of a sound (of an internal organ) caused inside the living body or (ii) a percutaneous arterial blood oxygen saturation. It is further assumed that a parameter indicative of a change appearing outside a living body is, for example, a body motion (measured with an acceleration sensor or the like) of the living body.

The biometric device of the present invention may be arranged such that the biometric parameter includes one or more biometric parameters; and the one or more biometric parameters used by the measurement result deriving means are obtained through analysis of a single item of the biometric signal information.

In other words, the biometric device may derive a measurement result with use of a plurality of kinds of biometric parameters obtained from a single biometric signal information item.

The biometric device of the present invention may be arranged such that the biometric parameter includes one or more biometric parameters; and the one or more biometric parameters used by the measurement result deriving means are obtained through analysis of a plurality of items of the biometric signal information.

In other words, the biometric device may derive a measurement result with use of a plurality of kinds of biometric parameters obtained from a plurality of kinds of biometric signal information items.

The biometric device of the present invention may further include: a communication section for communicating with a biometric sensor for obtaining the biometric signal information from the living body.

The above arrangement allows the biometric device to (i) receive biometric signal information from a biometric sensor through the communication section and thus (ii) obtain a biometric parameter from the biometric signal information obtained.

The biometric device of the present invention may be arranged such that the biometric device is contained in a biometric sensor for obtaining the biometric signal information from the living body.

With the above arrangement, the biometric device is contained in a biometric sensor and can thus obtain a biometric parameter directly from biometric signal information that the biometric device itself has obtained.

### As to Embodiment 2

In order to solve the above problem, a biometric device of the present invention includes: biometric sound processing means for deriving measurement result information indicative of a state of a living body by carrying out one or more information processes on biometric sound signal information obtained from a biometric sound sensor attached to the living body; a measurement method storage section in which attribute information of the biometric sound sensor and an algorithm are stored in correspondence with each other for each information process that the biometric sound processing means carries out; and selecting means for selecting, from among algorithms stored in the measurement method storage section for a single information process, an algorithm corresponding to the attribute information of the biometric sound sensor attached to the living body, the biometric sound processing means carrying out the information process on the biometric sound signal information in accordance with the algorithm selected by the selecting means.

According to the above arrangement, once a biometric sound that a living body emits is inputted as biometric sound signal information to the biometric device, the biometric sound processing means derives measurement result information indicative of a state of the living body by carrying out one or more information processes on the biometric sound signal information.

It should be noted here that the biometric device stores one or more algorithms in the measurement method storage section in correspondence with each piece of attribute information of the biometric sound sensor for a single information process. Accordingly, the selecting means obtains attribute information of a biometric sound sensor actually attached to the living body, and selects an algorithm corresponding to the attribute information. In a case where there are a plurality of information processes, the selecting means selects an algorithm suitable to the attribute information for each of the information processes.

The biometric sound processing means derives measurement result information by carrying out the information process in accordance with the algorithm selected by the selecting means.

With this, the content of an information process for deriving measurement result information can be varied according to attribute information of a biometric sound sensor actually attached to a living body. That is, various measurements can be performed without relying on various types of sensor. Further, since various algorithms can be applied to biometric sound signal information obtained from the biometric sound sensor, various measurements can be performed with high accuracy while avoiding such inconvenience that a measurement proceeds with the information remaining incomplete.

It is preferable that the attribute information include information on an attachment position of the biometric sensor attached to the living body, and that the selecting means select, from the measurement method storage section, an algorithm corresponding to the attachment position of the biometric sensor attached to the living body.

According to the above arrangement, once biometric sound signal information is obtained, the selecting means selects an optimum algorithm for a biometric sound sensor having obtained the biometric sound signal information, while regarding, as attribute information, an attachment position of the biometric sound sensor on the living body.

This makes it possible to perform a different process on biometric sound signal information according to a difference in attachment position of a biometric sound sensor. That is, the biometric sound processing means can derive measurement result information by applying an algorithm suitable for a position to which the biometric sound sensor has been attached. This makes it possible to improve measurement accuracy while avoiding a situation where information is rendered incomplete by a restriction of attachment position.

It is preferable that the attribute information include information on a measurement site of the living body that is to be sensed by the biometric sound sensor, and the selecting means select, from the measurement method storage section, an algorithm corresponding to the site to be measured by the biometric sound sensor attached to the living body.

Types of biometric sound that a living body emits vary from site to site within the living body. The measurement result information to be derived varies depending on what sound that is contained in the biometric sound signal information receives attention. Therefore, if the selecting means selects an algorithm in view of a site (measurement site) of the living body that is to be sensed by the biometric sound sensor, the biometric sound processing means can carry out an information process suitable for a measurement purpose and derive measurement result information with high accuracy.

It is preferable that the attribute information include information on a measurement item indicating, as a measurement purpose of the biometric sound sensor, what state of the living body is to be measured, and that the selecting means select, from the measurement method storage section, an algorithm corresponding to a measurement item to be measured by the biometric sound sensor.

Various states of the living body can be measured by analyzing the biometric sound signal information from various points of view and varying methods for analyzing the biometric sound signal information. Therefore, if the selecting means selects an algorithm in view of a detailed measurement purpose (i.e., a measurement item) indicating what state of the living body is to be measured, the biometric sound processing means can carry out an information process suitable for the measurement purpose and derive measurement result information with high accuracy.

The biometric device of the present invention may further include attachment position specifying means for specifying, as the attribute information, an attachment position of a biometric sound sensor to be attached to the living body, wherein: the attachment position specifying means specifies the attachment position of the biometric sound sensor on the basis of at least either (i) a measurement site of the living body that is to be sensed by the biometric sound sensor or (ii) a measurement item indicating, as a measurement purpose of the biometric sound sensor, what state of the living body is to be measured, with both the measurement site and the measurement item inputted to the biometric device; and the selecting means selects, from the measurement method storage section, an algorithm corresponding to the attachment position specified by the attachment position specifying means.

According to the above arrangement, first, at least either (i) information on a site (measurement site) of the living body that is to be sensed by the biometric sound sensor or (ii) information on a detailed measurement purpose (measurement item) indicating what state of the living body is to be measured is inputted to the biometric device. The attachment position specifying means specifies the attachment position of the biometric sound sensor on the basis of at least either the measurement site and the measurement item, which have been inputted to the biometric device. The measurement site and the measurement item, which have been inputted to the biometric device, indicate what the user wants to measure, i.e., a measurement purpose. Where to attach the biometric sound sensor varies from one measurement purpose to another. The attachment position specifying means determines an attachment position of the biometric sound sensor that is suitable for the measurement purpose. The selecting means can select, on the basis of the attachment position specified by the attachment position specifying means, an algorithm suitable for the attachment position.

This makes it necessary for the user to only designate a measurement purpose. Therefore, the biometric device of the present invention, which performs various measurements with high accuracy, can be made available even to a user who has a clear purpose of measurement but does not know a measurement method to accomplish the purpose.

It is preferable that the biometric device of the present invention further include a display section for displaying the attachment position specified by the attachment position specifying means.

The above arrangement makes it possible for the user to visually check the attachment position displayed by the display section, thus making it possible for the user to easily understand to which position the biometric sound sensor is supposed to be attached.

The biometric device of the present invention may further include measurement site specifying means for, on the basis of the biometric sound signal information obtained from the biometric sound sensor attached to the living body, specifying, as the attribute information, a measurement site of the living body that is to be sensed by the biometric sound sensor, wherein the selecting means selects, from the measurement method storage section, an algorithm corresponding to the measurement site specified by the measurement site specifying means.

According to the above arrangement, the measurement site specifying means specifies the measurement site on the basis of the biometric sound signal information obtained from the biometric sound sensor. Therefore, a suitable algorithm is selected in view of the measurement site without the user carrying out an operation of inputting the measurement site into the biometric device.

This makes it possible to simplify user operation, thus making it possible to improve user convenience.

The biometric device of the present invention may further include: a sound source storage section in which sample biometric sound signal information obtained in advance from the biometric sound sensor for each attachment position is stored in association with the attachment position; and attachment position estimating means for estimating, as the attribute information, the attachment position of the biometric sensor attached to the living body, wherein: the attachment position estimating means estimates the attachment position of the biometric sound sensor by making a comparison between (i) the biometric sound signal information obtained from the biometric sound sensor attached to the living body and (ii) the sample biometric sound signal information stored in the sound source storage section; and the selecting means selects, from the measurement method storage section, an algorithm corresponding to the attachment position estimated by the attachment position estimating means.

According to the above arrangement, the sound source storage section has sample biometric sound signal information stored therein for each attachment position that is imagined. The attachment position estimating means compares (i) biometric sound signal information obtained from the biometric sound sensor with (ii) each piece of sample biometric sound signal information stored in the sound source storage section, and estimates the attachment position of the biometric sound sensor on the basis of results of the comparison. For example, in a case where sample biometric sound signal information similar to the biometric sound signal information obtained is found as a result of a comparison, the attachment position associated with the biometric sound signal information can be estimated by checking if the sample biometric sound signal information is a sound associated with the attachment position. The selecting means selects a suitable algorithm in view of the attachment position thus estimated.

This eliminates the need for the user to input a measurement purpose or to know a measurement method to accomplish the purpose. This can make the biometric device available even to a user who does not know a measurement method, and also makes it possible to improve convenience by simplifying user operation.

It should be noted that biometric sound signal information to be stored in the sound source storage section may be (i) sound data itself obtained by digitalizing a biometric sound, (ii) a feature obtained by performing a predetermined process on the sound data in advance, or (iii) a feature that is a statistical value obtained by performing a statistical process on the sound data.

It is preferable that the biometric device of the present invention further include a display section for displaying the attachment position estimated by the attachment position estimating means.

The above arrangement makes it possible for the user to visually check the attachment position displayed by the display section, thus making it possible for the user to (i) easily understand to which position the biometric sound sensor is supposed to be better attached and to (ii) thereby change attachment positions.

It is preferable that the biometric sound processing means carry out, as the information process, a quality assessing process of assessing whether or not the biometric sound signal information has a sound quality sufficient to derive measurement result information indicative of a state of the living body, and that the selecting means select, from among algorithms for the quality assessing process which algorithms are stored in the measurement method storage section, an algorithm corresponding to the attribute information of the biometric sound sensor.

The above arrangement allows the biometric sound processing means to carry out the quality assessing process in accordance with the algorithm thus selected. This makes it possible for the biometric sound processing means to appropriately assess the quality in accordance with the attribute information of the biometric sound sensor.

For example, use of a result of such a quality assessing process makes it possible to avoid such inconvenience that a measurement proceeds with the biometric sound signal information remaining insufficient in quality, thus making it possible, as a result, to improve accuracy of measurement.

It is preferable that the biometric sound processing means carry out, as the information process, a state evaluating process of evaluating a state of the living body on the basis of a parameter obtained by analyzing the biometric sound signal information, and that the selecting means select, from among algorithms for the state evaluating process which algorithms are stored in the measurement method storage section, an algorithm corresponding to the attribute information of the biometric sound sensor.

The above arrangement allows the biometric sound processing means to perform the state evaluating process in accordance with the algorithm thus selected. This makes it possible for the biometric sound processing means to appropriately evaluate a state of the living body in accordance with the attribute information of the biometric sound sensor, thus making it possible, as a result, to derive measurement result information with high accuracy.

The biometric device of the present invention may further include biometric sound obtaining means for obtaining, via a communication section from a plurality of biometric sound sensors attached to the living body, the biometric sound signal information for each of the biometric sound sensors, wherein the selecting means selects an algorithm on the basis of the attribute information of each of the biometric sound sensors for each piece of biometric sound signal information obtained by the biometric sound obtaining means.

According to the above arrangement, once biometric sound signal information is obtained from each of the biometric sound sensors attached to the living body, the selecting means can select an algorithm for each piece of biometric sound signal information in view of the attribute information of each of the biometric sound sensors.

This makes it possible to carry out a process on each piece of biometric sound signal information by applying different optimum algorithms for each separate piece of biometric sound signal information even in the case of multipoint simultaneous measurement. This makes it possible to (i) improve measurement accuracy while avoiding a situation where information is rendered incomplete by a restriction of attachment position, and to (ii) simultaneously perform various measurements, thus making it possible, as a result, to carry out various measurements with high accuracy without relying on many types of sensor.

It is preferable that the biometric device of the present invention further include biometric sound obtaining means for obtaining, via a communication section from a plurality of biometric sound sensors attached to the living body, the biometric sound signal information for each of the biometric sound sensors, wherein: the attachment position estimating means estimates a positional relationship between the biometric device and each of the biometric sound sensors on the basis of a signal strength with which the communication section receives the biometric sound signal information from each of the biometric sound sensors, and on the basis of the positional relationship thus estimated, limits sample biometric sound signal information that is to serve as a target of comparison; and the selecting means selects, on the basis of an attachment position estimated for each of the biometric sound sensors, an algorithm that is applied to each separate piece of biometric sound signal information obtained by the biometric sound obtaining means.

According to the above arrangement, the aforementioned attachment position estimating means estimates the respective attachment positions of the plurality of biometric sound sensors by comparison with a sample. It should be noted here that the attachment position estimating means estimates a positional relationship between the biometric device and each of the biometric sound sensors in view of the strength of a signal that is generated by communication with a plurality of biometric sound signals. The attachment position estimating means does not need to make a comparison with every sample stored in the sound source storage section, as long as a positional relationship with a biometric sound sensor can be estimated to some extent. That is, the attachment position estimating means carries out matching limited to a sample of an attachment position corresponding to the positional relationship thus estimated.

This makes it possible to increase the processing efficiency of the biometric device by significantly reducing the processing load of the matching that is performed by the attachment position estimating means.

The biometric device may further include a communication section for communicating with a biometric sound sensor that obtains the biometric sound signal information from the living body.

The above arrangement allows the biometric device to (i) obtain biometric sound signal information from a biometric sound sensor via the communication section and (ii) process the biometric sound signal information thus obtained.

Alternatively, the biometric device may be contained in a biometric sound sensor that obtains the biometric sound signal information from the living body.

The above arrangement allows the biometric device to be contained in the biometric sound sensor to directly process biometric sound signal information obtained by the biometric device.

In order to solve the above problem, a biometric method of the present invention is a biometric method for use in a biometric device for measuring a state of a living body by processing biometric sound signal information obtained from a biometric sound sensor attached to the living body, attribute information of the biometric sound sensor and an algorithm being stored in the biometric device in correspondence with each other for each information process that is carried out on the biometric sound signal information, the biometric method including: a selecting step for selecting, from among algorithms stored for a single information process, an algorithm corresponding to the attribute information of the biometric sound sensor attached to the living body; and a step for carrying out the information process on the biometric sound signal information in accordance with the algorithm selected in the selecting step.

The biometric device may be in the form of a computer. In such a case, (i) a control program for causing a computer to function as each of the means of the biometric device and (ii) a computer-readable recording medium containing such a control program are also encompassed in the technical scope of the present invention.

This brings about an effect of making it possible to carry out various measurements with high accuracy without relying on many types of sensor.

### As to Embodiment 3

In order to solve the above problem, a biometric device of the present invention includes: biometric sound parameter obtaining means for obtaining a biometric sound parameter based on biometric sound signal information obtained from a living body; biometric parameter obtaining means for obtaining a biometric parameter based on either the biometric sound signal information or biometric signal information obtained from the living body, the biometric parameter being different from the biometric sound parameter; and detecting means for detecting a state of the living body on the basis of the biometric sound parameter and the biometric parameter.

In order to solve the above problem, a biometric method of the present invention is a biometric method for use in a biometric device for measuring a state of a living body, the biometric method including: a biometric sound parameter obtaining step for obtaining a biometric sound parameter based on biometric sound signal information obtained from the living body; a biometric parameter obtaining step for obtaining a biometric parameter based on either the biometric sound signal information or biometric signal information obtained from the living body, the biometric parameter being different from the biometric sound parameter; and a detecting step for detecting a state of the living body on the basis of the biometric sound parameter and the biometric parameter.

According to the above arrangement, the detecting means detects the state of the living body on the basis of (i) the biometric sound parameter obtained by the biometric sound parameter obtaining means and (ii) the biometric parameter obtained by the biometric parameter obtaining means.

The biometric sound parameter is one parameter that is obtained from the biometric sound signal information (e.g., a cough sound) obtained from the living body. The biometric parameter is another parameter that is different from the biometric sound parameter and that is obtained from either the biometric sound signal information on the living body or the biometric signal information on the living body.

The biometric device of the present invention detects a state of a living body by using not only a biometric sound parameter but also another biometric parameter of the living body, thus making it possible to increase the accuracy with which the state of the living body is detected.

Further, it is preferable that the biometric parameter reflect a physiological state of the living body.

The above arrangement detects a state of a living body by using not only a biometric sound parameter but also a biometric parameter reflecting a physiological state of the living body, thus making it possible to increase the accuracy with which the state of the living body is detected.

Further, it is preferable that the detecting means detect a state of a living body on the basis of changes in the biometric sound parameter and in the biometric parameter over time.

The above arrangement makes it possible to detect a change in state of a living body over time.

Further, it is preferable that the detecting means detect a state of a living body on the basis of a change in the biometric parameter over a predetermined time period beginning at a time point at which the biometric sound parameter changed.

The above arrangement detects a state of a living body on the basis of whether or not the biometric parameter has changed within a predetermined time period since a time point at which the biometric sound parameter changed.

This makes it possible to detect a state of a living body with high accuracy even in a case where there is a time lag between (i) a time point at which the biometric sound parameter changed and (ii) a time point at which the biometric parameter changes.

Further, it is preferable that in a case where the biometric sound signal information meets a predetermined condition, the biometric parameter obtaining means obtain the biometric parameter and the detecting means detect a state of the living body.

According to the above arrangement, the biometric parameter is obtained in a case where the biometric sound signal information meets a predetermined condition. This makes it possible to cut electric power consumption than in the case of a configuration in which the biometric parameter is continuously obtained.

Further, it is preferable that the biometric parameter obtaining means obtain at least a percutaneous arterial blood oxygen saturation as the biometric parameter.

Further, the detecting means may detect a state of emission of a cough by the living body.

According to the above arrangement, the at least percutaneous arterial blood oxygen saturation is obtained as the biometric parameter, and the state of emission of the cough by the living body is detected on the basis of the biometric sound parameter and the at least percutaneous arterial blood oxygen saturation.

Sounds that a living body emits (or sounds in an area surrounding the living body) may include sounds other than cough sounds. Therefore, every sound that is produced is not necessarily a cough sound.

Since coughing impairs breathing for the duration thereof, there is a high possibility of a decrease in oxygen saturation of the arterial blood. Therefore, a cough that the living body emits can be detected with high accuracy by detecting both (i) a sound that the living body emits and (ii) a change in arterial blood oxygen saturation.

It is preferable that the detecting means also detect a severity of the cough as the state of emission of the cough.

The above arrangement not only detects a cough but also detects the severity of the cough, thus making it possible to more accurately indicate the state of the living body.

Further, it is preferable that the detecting means detect a state of emission of a cough on the basis of a result of comparison between (i) a statistical value of the percutaneous arterial blood oxygen saturation over a predetermined time period beginning at a time point at which the biometric sound parameter changed and (ii) the percutaneous arterial blood oxygen saturation at a time point at which a predetermined time period has elapsed since the time point.

The percutaneous arterial blood oxygen saturation varies from time to time even within the same living body. Therefore, in a case where the percutaneous arterial blood oxygen saturation is used for detection of a cough, it is preferable to obtain the percutaneous arterial blood oxygen saturation (i) at a time point which is close to a time point at which the living body coughed and (ii) in a state in which the living body is not coughing.

According to the above arrangement, a change in biometric parameter is detected by making a comparison between (i) a statistical value of the percutaneous arterial blood oxygen saturation over a predetermined time period beginning at a time point at which the biometric sound parameter changed (e.g., the mean of percutaneous arterial blood oxygen saturations measured over a time period having elapsed since the detection of a cough) and (ii) the percutaneous arterial blood oxygen saturation at a time point at which a predetermined time period has elapsed since the time point at which the biometric sound parameter changed.

Therefore, the percutaneous arterial blood oxygen saturation in a state in which the living body is not coughing can be calculated as the statistical value, and the percutaneous arterial blood oxygen saturation changed by coughing can be obtained as the percutaneous arterial blood oxygen saturation after a predetermined time period. By making a comparison between the statistical value and the percutaneous arterial blood oxygen saturation after a predetermined time period, a change in percutaneous arterial blood oxygen saturation due to coughing can be more accurately detected.

Further, it is preferable that the statistical value of the percutaneous arterial blood oxygen saturation over a predetermined time period beginning at a time point at which the biometric sound parameter changed be the mean of percutaneous arterial blood oxygen saturations measured over a period at least 20 seconds after the time point.

By taking the mean of percutaneous arterial blood oxygen saturations over a period of 20 seconds, the influence of a change in percutaneous arterial blood oxygen saturation in a state in which the living body is not coughing and a measurement error can be reduced.

Further, it is preferable that the detecting means detect a state of emission of a cough on the basis of a rate of change of (i) the percutaneous arterial blood oxygen saturation measured 20 seconds after the time point at which the biometric sound parameter changed with (ii) the mean of percutaneous arterial blood oxygen saturations.

It takes approximately 20 seconds for the percutaneous arterial blood oxygen saturation to change (decrease) after the living body emits a cough. Therefore, a change in percutaneous arterial blood oxygen saturation as a biometric parameter can be detected with high accuracy by (i) obtaining the mean of percutaneous arterial blood oxygen saturations in a state in which the living body is not coughing and the percutaneous arterial blood oxygen saturation measured 20 seconds after a time point at which the biometric sound parameter changed and (ii) calculating a rate of change of the latter with the former.

It is preferable that the biometric device of the present invention further include cough sound estimating means for estimating generation of a cough sound on the basis of the biometric sound signal information, wherein the biometric parameter obtaining means obtains the percutaneous arterial blood oxygen saturation only in a case where the cough sound estimating means has estimated the generation of the cough sound.

According to the above arrangement, the percutaneous arterial blood oxygen saturation is obtained only in a case where the cough sound estimating means has estimated generation of a cough sound. This makes it possible to cut more electric power consumption than in the case of a configuration in which the percutaneous arterial blood oxygen saturation is continuously obtained.

Further, it is preferable that the biometric device of the present invention further include a communication section for communicating with, out of (i) a biometric sound sensor for obtaining the biometric sound signal information from the living body and (ii) a biometric sensor for obtaining the biometric signal information from the living body, at least the biometric sound sensor.

According to the above arrangement, the communication section communicates with at least the biometric sound sensor out of the biometric sound sensor and the biometric sensor. This makes it possible to obtain biometric (sound) signal from the biometric sound sensor or the biometric sensor.

Further, a biometric device contained in a biometric sound sensor for obtaining the biometric sound signal information from the living body is also encompassed in the technical scope of the present invention.

A control program for causing a computer to function as each of the means of the biometric device and a computer-readable recording medium containing such a control program are also encompassed in the technical scope of the present invention.

This brings about an effect of making it possible to increase the accuracy with which a state of a living body is detected.

### As to Embodiment 4

In order to solve the above problem, a measurement position assessing device of the present invention includes: sound data obtaining means for obtaining sound data containing a measurement target sound detected by a biometric sound sensor as attached to a living body, the biometric sound sensor detecting at least one type of measurement target sound emitted by the living body; and assessing means for assessing suitability of an attachment position of the biometric sound sensor on a basis of sound data obtained by the sound data obtaining means, the sound data obtaining means obtaining a plurality of pieces of sound data from the biometric sound sensor at different attachment positions, the assessing means relatively assessing suitability of the attachment position by making a comparison between measurement target sounds contained in the pieces of sound data obtained by the sound data obtaining means.

In order to solve the above problem, a measurement position assessing method of the present invention includes: a sound data obtaining step for obtaining sound data containing a measurement target sound detected by a biometric sound sensor as attached to a living body, the biometric sound sensor detecting at least one type of measurement target sound emitted by the living body; and an assessing step for assessing suitability of an attachment position of the biometric sound sensor on a basis of sound data obtained in the sound data obtaining step, the sound data obtaining step obtaining a plurality of pieces of sound data from the biometric sound sensor at different attachment positions, the assessing step relatively assessing suitability of the attachment position by making a comparison between measurement target sounds contained in the pieces of sound data obtained in the sound data obtaining step.

According to the above arrangement, the biometric sound sensor that detects at least one type of measurement target sound emitted by a living body is attached to a living body, and the sound data obtaining means obtains sound data of a measurement target sound detected by the biometric sound sensor. The sound data obtaining means obtains a plurality of pieces of sound data of measurement target sounds detected by the biometric sound sensor at different attachment positions. The assessing means assesses whether or not the attachment position of the biometric sound sensor is suitable by making a comparison between measurement target sounds contained in the respective pieces of sound data obtained by the sound data obtaining means.

Therefore, it is possible to notify whether or not the attachment position is suitable to a user who is confused about where to attach the biometric sound sensor.

Further, it is preferable that the assessing means assess suitability of the attachment position on the basis of a result of comparison between (i) an amplitude of a measurement target sound indicated by the sound data and (ii) a predetermined reference value.

According to the above arrangement, by making a comparison between (i) an amplitude of a measurement target sound at a certain attachment position and (ii) a reference value, suitability of the attachment position is assessed.

Therefore, even in a case where the biometric sound sensor is attached to a sole position, it is possible to notify a user of whether or not the attachment position is desirable.

Further, it is preferable that the biometric sound sensor detect a plurality of types of measurement target sounds emitted by the living body, and that the assessing means assess suitability of the attachment position on the basis of a plurality of types of measurement target sounds contained in the sound data.

According to the above arrangement, a plurality of types of biometric sounds are simultaneously detected by a single biometric sound sensor. The assessing means assesses suitability of the attachment position on the basis of a plurality of types of measurement target sounds detected by the biometric sound sensor. For example, the assessing means assesses suitability of the attachment position on the basis of whether or not the plurality of types of measurement target sounds meet a predetermined requirement.

Therefore, even in a case where there are a plurality of measurement target sounds, it is possible to notify a user of a desirable attachment position.

Further, it is preferable that the sound data obtaining means obtain a plurality of pieces of sound data obtained respectively from a plurality of the biometric sound sensor at different attachment positions.

According to the above arrangement, a plurality of biometric sound sensors are attached to a living body, and sound data is outputted from each of the biometric sound sensors. The sound data obtaining means obtains a plurality of pieces of sound data outputted in this manner. Then, the assessing means relatively assesses which of attachment positions is more desirable by making a comparison between measurement target sounds contained in the plurality of pieces of sound data thus obtained.

Therefore, by attaching the biometric sound sensor to a plurality of positions for a try, the user can learn about which position is more desirable (or most desirable) and easily learn a suitable attachment position.

Further, it is preferable that the assessing means assess suitability of the attachment position on the basis of whether or not amplitudes of the plurality of types of measurement target sounds reach predetermined reference values respectively corresponding to the types of measurement target sounds.

According to the above arrangement, predetermined reference values for amplitudes of measurement target sounds are set in correspondence with the types of measurement target sounds, so that suitability of the attachment position is assessed on the basis of whether or not the amplitudes of the measurement target sounds detected by the biometric sound sensor reach the predetermined reference values.

Therefore, even in a case where there are a plurality of measurement target sounds, it is possible to notify a user of a desirable attachment position determined with respect to amplitudes of the measurement target sounds.

In addition, it is preferable that a notifying section that notifies a result of the assessment made by the assessing means be further included.

With the above arrangement, a result of the assessment made by the assessing means can be notified to a user.

Further, (i) a control program for causing a computer to function as the foregoing means of the measurement position assessing device and (ii) a computer-readable recording medium storing the control program therein are also encompassed in the technical scope of the present invention.

This achieves the effect of notifying whether or not the attachment position is suitable to a user who is confused about where to attach the biometric sound sensor.

### <<Supplemental Remarks>>

The present invention is not limited to the aforementioned embodiments and is susceptible of various changes within the scope of the accompanying claims. Also, any embodiment obtained by suitable combinations of technical means disclosed in the different embodiments is also included within the technical scope of the present invention.

### As to Embodiment 1

The present invention may also be described as below.

The present invention provides a body information measuring device including: body information measuring means for measuring body information on a user; and deriving means for deriving an index of a measurement target (measurement item) on the basis of attribute information (for example, a measurement target, measurement information, information on the measuring means, and information on the position of the measuring means) corresponding to the measuring means (biometric sensors 2 to 6 and 8).

The body information measuring device may preferably be arranged such that the attribute information (parameter) includes measurement information (body information), information on the measuring means, and attachment position information.

The attribute information is selected on the basis of the measurement target.

The attribute information may preferably include auxiliary attribute information (auxiliary parameter) for improving accuracy of the index.

The body information measuring device may preferably be arranged to select the attribute information (essential parameter) and the auxiliary attribute information on the basis of the measurement target.

### As to Embodiment 2

For example, the analysis device 201 may include all of the following: the attachment position specifying section 250 of Embodiment 2-2 (FIG. 41); the measurement site specifying section 251 of Embodiment 2-3 (FIG. 46); and the attachment position estimating section 252 of Embodiment 2-3 (FIG. 46). According to the above arrangement, in a case where all pieces of attribute information, i.e., an attachment position, a measurement site, and a measurement item, have been designated by the user via the input operation section 214, the attribute information determining section 221 determines the attribute information in accordance with the user's input. In a case where only the measurement site (and the measurement item) has been designated, the attachment position specifying section 250 specifies the attachment position. In a case where none of the pieces of attribute information has been inputted, the measurement site specifying section 251 specifies the measurement site, and the attachment position estimating section 252 estimates the attachment position. This makes it possible to provide a biometric system 200 that does not need to be user-specific (does not need the user's expertise) and that is high in convenience and operability according to the amount of knowledge the user has.

In each of the embodiments described above, biometric sound signal information to be stored in the sound source storage section 232 has been described as sound data itself obtained by digitalizing a biometric sound. However, the present invention is not limited to this. Biometric sound signal information may be constituted by sound data and/or features that are obtained from sound data. That is, the sound source storage section 232 of the analysis device 201 may be configured such that a feature that is extracted from the sound data is stored as biometric sound signal information in the sound source storage section 232 either in addition to the sound data or instead of the sound data. The feature may be (i) information obtained by carrying out a predetermined process on the sound data, or may be (ii) a feature that is a statistical value obtained by carrying out a statistical process on the sound data. That is, a comparison that is made by the analysis device 201 between biometric sound signal information gathered and sample biometric sound signal information stored in the sound source storage section 232 may include making a comparison between sounds, or may include making a comparison between features obtained by analyzing sound data.

(Problems Raised by Conventional Technologies and Effects of Present Invention)

In a case where a subject is sensed by using a sensor and a state of the subject is measured on the basis of signal information obtained from the sensor, it is not always necessary to configure many types of sensor in a single measuring device as described in Patent Literature 1. In some cases, necessary biometric information can be obtained by performing measurements at different places with a single sensor of one type. In other cases, necessary biometric information can be obtained by performing measurements simultaneously at multiple points with a plurality of sensors of one type, as described in Embodiment 2-4 or Embodiment 2-5 of the present invention.

For example, in a case where attention is focused on sounds that are emitted from a living body, it is very meaningful to measure biometric sounds from the respiratory organs or the heart simultaneously at multiple points. In a conventional case where a doctor diagnoses a patient, it is necessary to apply a stethoscope to monitor breath sounds from a wide area including the chest and the upper back. Actually, the doctor performs a stethoscopic examination by applying the stethoscope to ten or more spots on the body in sequence.

Even in the case of a health monitoring device for measuring the state of a user's physical health individually without a doctor, it is desirable to perform measurements at multiple points as a doctor would do in order to monitor the respiratory state. However, a method that requires a user to apply a stethoscope to measuring spots in sequence by him/herself as a doctor would do makes it very difficult for a user who is poor in medical knowledge to perform measurements with sufficient measurement accuracy. Even if the user carefully perform measurements, it is not hard to imagine that it takes a long time to do so.

Further, according to the technique described in Patent Literature 1, a biometric information measuring device contains a plurality of measuring means for measuring pulse waves, a pulse, GSR, skin temperature, a blood sugar level, acceleration, etc., but, unlike the present invention, does not include a sound sensor for obtaining a biometric sound.

Further, even in a case where not biometric sounds but pulse waves at multiple points on the body of a user are measured by a device described in Patent Literature 1, a plurality of such devices must be attached to the entire body. However, since each of the devices is equipped with a GSR sensor, a temperature sensor, a blood sugar level sensor, an acceleration sensor, etc. which are not necessary for pulse wave measurement, the device is bulky and therefore has a problem with attachability and there is concern that cost may need to be paid for such unnecessary sensors.

According to the technique described in Patent Literature 1, a body attachment belt enables a biometric information measuring device to be attached by hanging from a wrist, a head, or a neck. For example, an attempt to newly provide the biometric information measuring device with an acoustic sensor for measuring biometric sounds such as heart sounds and breath sounds requires the body attachment belt to be worn around the chest, in which case the use may have difficulty in attaching the biometric information measuring device all by him/herself. Further, an operation of, in a case where the sensor was attached off the point and therefore was not able to correctly measure biometric information, making position corrections several times makes it very difficult for the user to use the sensor. Further, in a case where biometric sounds are measured from an area around the lungs, it is necessary to wind the body attachment belt several times over. In reality, this causes the user a lot of difficulties.

In order to solve the above problem, the biometric system 200 of the present invention uses (i) an acoustic sensor including a biometric sound microphone which acoustic sensor digitalizes a sound and outputs it to an external device, (ii) a unit for gathering, analyzing, and evaluating biometric sound data from a single or plurality of the acoustic sensor, and (iii) an external device for either receiving health information obtained by analyzing the biometric sound data outputted from the unit or supplying the unit with setting information for measuring a biometric sound.

According to the present invention, an acoustic sensor can be configured to be limited to a function of merely (i) digitalizing biometric sound information obtained from a microphone and (ii) outputting the biometric sound information thus digitalized, as illustrated in FIGS. 28 and 29. This makes it possible to provide an inexpensive, small-sized acoustic sensor, thus providing the user with easy attachability. Further, since acoustic sensors are inexpensive, preparing a plurality of acoustic sensors is not a burden on the user. In this case, biometric sounds can be measured simultaneously at multiple points, so that an improvement in measurement accuracy and a reduction in measuring time can be achieved. Further, as mentioned above, the analysis device 201 guides a correct attachment position of each acoustic sensor. This makes it possible to provide a large population of users with a biometric system 200 for monitoring biometric sounds that is easy to use even for a user with poor medical knowledge.

Further, according to the present invention, by simply attaching an acoustic sensor to a place to which a user vaguely intends to attach the acoustic sensor, the analysis device 201 is caused to determine, from sound data obtained, which biometric sound to analyze and evaluate, and outputs measurement result information. Therefore, the user is not required to have deep medical knowledge.

Further, by specifying, from the sound data obtained, an attachment position and a sound to be measured (measurement site), the analysis device 201 gives the user a suggestion for a more correct attachment position of the acoustic sensor which attachment position is necessary for a more detailed analysis. This brings about an improvement in measurement accuracy.

It should be noted that the present invention can also be expressed as below.

That is, the present invention is directed to a sound monitoring device (analysis device 201 or external device 203) including selecting means for selecting sound data processing from (i) sound data (biometric sound signal information) and (ii) attribute information based on the sound data.

Further, the attribute information may be information on a measurement site on which the sound data was measured.

Further, the attribute information may be a measurement parameter of the sound data.

Further, the sound data processing may include a process of assessing the quality of the sound data.

Further, the sound data processing may include a process of specifying a sound source (measurement site) of the sound data.

Further, the sound data processing may include a process of, in a case where the attribute information does not contain positional information, specifying a measurement site on which the sound data was measured.

It should be noted that examples of the measurement parameter encompass a heart sound, a breath sound, a blood flow sound, an abdominal sound, etc.

Further, the sound data is obtained by a sound sensor.

Further, the sound data may be obtained by a plurality of sound sensors (acoustic sensors 202).

Further, the sound sensor(s) may include means for communicating with an external device (analysis device 201 or external device 203).

Further, it is preferable that the external device include the selecting means and display means for displaying a result of the sound data processing.

A health state monitoring device (biometric system 200) that presents the state (normal or abnormal) of health of a subject on the basis of information from the aforementioned sound monitoring device of the present invention is also encompassed in the scope of the present invention.

### As to Embodiment 3

It should be noted that the present invention can also be expressed as below.

That is, the present invention is directed to a cough detecting sensor for detecting a cough from both (i) sound data detected by an acoustic sensor and (ii) data on a change in percutaneous arterial blood oxygen concentration.

Further, it is preferable that the cough detecting sensor detect a change from the mean of percutaneous arterial blood oxygen concentrations over a period of 20 seconds.

Further, it is preferable that the cough detecting sensor detect a cough from a correlation between (i) a value read by the acoustic sensor and (ii) the mean of percutaneous arterial blood oxygen concentrations measured over a period of 20 seconds or longer from a time point that is 20 seconds after detection of a cough.

Further, it is preferable that the cough detecting sensor measure the percutaneous arterial blood oxygen concentration only when the acoustic sensor has detected a sound estimated to be a cough sound.

Further, the present invention can also be expressed as a detecting device for detecting a state of a subject from a plurality of parameters including sound data.

Further, it is preferable that the detecting device detect the state of the subject from changes in the parameters over a given time period.

Further, it is preferable that the detecting device detect the state of the subject from a correlation between the parameters.

Further, it is preferable that the detecting device detect the state of the subject by measuring the parameters in a case where the sound data meets a given condition.

Further, it is preferable that the parameters include the percutaneous arterial blood oxygen concentration.

Further, the state of the subject is coughing.

Further, it is preferable that the detecting device detect a cough from a change from the mean of percutaneous arterial blood oxygen concentrations over a period of 20 seconds.

Further, it is preferable that the detecting device detect a cough from a correlation between (i) the sound data and (ii) the mean of percutaneous arterial blood oxygen concentrations measured over a period of 20 seconds or longer after 20 seconds from a time point that is 20 seconds after detection of a cough.

Further, it is preferable that the detecting device measure the percutaneous arterial blood oxygen concentration only when a sound estimated to be a cough sound has been detected in the sound data.

Further, it is preferable that the parameters be data detected by a single or multiple sensor(s) including a sound sensor.

It is preferable that the sound sensor be attached to a given position on a human body according to a state of a subject which state needs to be detected.

### As to Embodiment 4

It should be noted that the present invention can also be expressed as below.

That is, a body information measuring device of the present invention is characterized by including means for obtaining a measurement position best-suited to observe a particular state of health.

Further, it is preferable that the body information measuring device accumulate detection values of the means provided in the body information measuring device so as to determine, as a most suitable measurement position, a position at which a maximum detection value is obtained.

Still further, it is preferable that with a plurality of obtaining means provided, an observation accuracy of a health state be increased.

Yet further, it is preferable that the body information measuring device accumulate data obtained by the body information measuring device, so that a change of state can be displayed.

Further, it is preferable that the body information measuring device can display the degree of improvement in state of health with use of (i) data obtained by the body information measuring device and (ii) behavior information as entered.

Still further, it is preferable that the body information measuring device present how many times an apnea state has occurred during sleep.

Yet further, it is preferable that the body information measuring device accept an entry such as a weight or an excessive daytime sleep.

### Software Implementation Example

Finally, the blocks of the analysis device 1, in particular, the information obtaining section 20, the parameter extracting section 21, the parameter selecting section 22, the index calculating section 23, the state assessing section 24, the measurement item determining section 25, and the parameter attribute managing section 26 may be constituted by hardware logic or may be realized by software as executed by a CPU as below.

Further, the blocks of the analysis device 201, in particular, the attribute information determining section 221, the algorithm selecting section 222, the quality assessing section 223, and the state evaluating section 224 may be constituted by hardware logic or may be realized by software as executed by a CPU as below.

Still further, the foregoing blocks of the symptom detecting device 340, in particular, the main control section 302 of the analysis device 301 may be constituted by hardware logic or may be realized by software as executed by a CPU as below.

Yet further, the foregoing blocks of the measuring device 430 and the measuring device 440, in particular, the main control section 402 of the analysis device 401 may be constituted by hardware logic or may be realized by software as executed by a CPU as below.

Specifically, the analysis device 1, the analysis device 201, the symptom detecting device 340, the measuring device 430, and the measuring device 440 each include a CPU (central processing unit) and memory devices (memory media). The CPU executes instructions in a control program for realizing the functions. The memory devices (recording media) include a ROM (read only memory) which contains the program, a RAM (random access memory) to which the program is loaded, and a memory containing the program and various data. The objective of the present invention can also be achieved by mounting to the analysis device 1, the analysis device 201, the symptom detecting device 340, the measuring device 430, and the measuring device 440 a computer-readable recording medium containing control program code (executable program, intermediate code program, or source program) for the analysis device 1, the analysis device 201, the symptom detecting device 340, the measuring device 430, and the measuring device 440, which is software realizing the aforementioned functions, in order for the computer (or CPU, MPU) to read and execute the program code contained in the recording medium.

The recording medium may be, for example, a tape, such as a magnetic tape or a cassette tape; a magnetic disk, such as a Floppy® disk or a hard disk, or an optical disk, such as CD-ROM/MO/MD/DVD/CD-R; a card, such as an IC card (including memory card) or an optical card; or a semiconductor memory, such as a mask ROM/EPROM/EEPROM/flash ROM.

Further, the analysis device 1, the analysis device 201, the symptom detecting device 340, the measuring device 430, and the measuring device 440 may be arranged to be connectable to a communications network so that the program code may be delivered over the communications network. The communications network is not limited in any particular manner, and may be, for example, the Internet, intranet, extranet, LAN, ISDN, VAN, CATV communications network, virtual dedicated network (virtual private network), telephone line network, mobile communications network, or satellite communications network. The transfer medium which makes up the communications network is not limited in any particular manner, and may be, for example, wired line, such as IEEE 1394, USB, power line carrier, cable TV line, telephone line, or ADSL line; or wireless, such as infrared radiation (IrDA, remote control), Bluetooth®, 802.11 wireless, HDR, mobile telephone network, satellite line, or terrestrial digital network. It should be noted that the present invention can also be implemented in the form of a computer data signal embedded in a carrier wave which is embodied by electronic transmission.

### INDUSTRIAL APPLICABILITY

The biometric device (analysis device) of the present invention can measure a state of a subject with high accuracy, and is thus usable as, for example, (i) a patient monitoring device at a medical institution or (ii) household health-care equipment for self-diagnosis.

The biometric device (analysis device) of the present invention is used as a measuring device for recognizing a health state of a person. More specifically, the biometric device is used widely in society as a piece of health-care equipment particularly for the purchase of measuring a biometric sound. Further, the biometric device of the present invention not only finds its application in observation of symptoms in a patient with a chronic cardiac disease, a chronic respiratory disease, or a chronic circulatory disease, but also is widely used for a healthy person as a means of understanding a health state for disease prevention.

In addition, the measurement position assessing device (analysis device) of the present invention can let a user know of a preferable attachment position for a biometric sound sensor, and is thus usable as, for example, a diagnosis device or health-care device for use by a general user with no expert knowledge.

### REFERENCE SIGNS LIST

1 analysis device (biometric device)
2a acoustic sensor (biometric sensor)
2b acoustic sensor (biometric sensor)
3 pulse oximeter (biometric sensor)
4 pulse wave sensor (biometric sensor)
5 clinical thermometer (biometric sensor)
6 acceleration sensor (biometric sensor)
7 information providing device
8 electrocardiograph (biometric sensor)
10 control section
11 storage section
12 wireless telecommunication section (communication section)
13 communication section (communication section)
14 input operation section
15 display section
20 information obtaining section
21 parameter extracting section
22 parameter selecting section
23 index calculating section (measurement result deriving means)
24 state assessing section (state evaluating means)
25 measurement item determining section
26 parameter attribute managing section (parameter attribute managing means)
30 parameter storage section
31 measurement method storage section
32 index calculation rule storage section
33 index storage section
34 parameter attribute storage section
100 biometric system
d1 measurement item
d2 presence or absence of waveform
d3 sound volume
d4 waveform length
d5 number of waveforms
d7 heart rate
d8 apnea degree calculation rule
d9 apnea degree
d10 assessment criterion information
d11 state assessment result
201 analysis device (biometric device)
202 acoustic sensor (biometric sound sensor)
202a acoustic sensor (biometric sound sensor)
202b acoustic sensor (biometric sound sensor)
202c acoustic sensor (biometric sound sensor)
202d acoustic sensor (biometric sound sensor)
203 external device
203a portable terminal device
203b laptop personal computer
203c data accumulation device
210 control section
211 storage section
212 wireless telecommunication section (communication section)
213 communication section
214 input operation section
215 display section
220 information obtaining section (biometric sound obtaining means)
221 attribute information determining section
222 algorithm selecting section (selecting means)
223 quality assessing section (biometric sound processing means)
224 state evaluating section (biometric sound processing means)
230 sound data storage section
231 measurement method storage section
232 sound source storage section
233 attachment position information storage section
234 attribute information storage section
200 biometric system
270 control section
271 housing section
273 diaphragm
274 tackiness agent layer
275 first conversion section
276 air chamber wall
277 A/D conversion section
278 substrate
279 electric power supply section
280 microphone section
281 wireless telecommunication section
282 individual identification device
250 attachment position specifying section (attachment position specifying means)
251 measurement site specifying section
252 attachment position estimating section (biometric sound processing means)
301 analysis device (biometric device)
302 main control section
303 cough sound assessing section (cough sound estimating means, biometric sound parameter obtaining means)
304 measuring device control section (biometric parameter obtaining means)
305 statistical processing section
306 symptom detecting section (detecting means)
307 storage section
308 operation section
309 display section
320 acoustic sensor (biometric sound sensor)
330 pulse oximeter (biometric sensor) 331 sensor section (biometric sensor)
332 main body
333 display section
334 main control section
340 symptom detecting device (biometric device)
401 analysis device (measurement position assessing device)
402 main control section
403 biometric sound extracting section
404 position assessing section (assessing means)
405 symptom detecting section
406 data analyzing section
407 storage section
408 operation section
409 display section (notifying section)
410 speaker (notifying section)
420 sound sensor (biometric sound sensor)
430 measuring device (measurement position assessing device)
440 measuring device (measurement position assessing device)
440 measuring device
441 biometric sound extracting section
442 heart sound extracting section
443 breath sound extracting section
444 position assessing section (assessing means)
450 human body (subject)

1. A biometric device for measuring a state of a living body with use of biometric signal information obtained from the living body,
the biometric device comprising:
measurement result deriving means for deriving, with use of one or more parameters including at least a biometric parameter obtained on a basis of the biometric signal information, measurement result information indicative of the state of the living body; and
a measurement method storage section in which (i) a measurement item measurable by the biometric device and (ii) parameter specifying information specifying a parameter for use in measurement of the measurement item are stored in correspondence with each other,
the measurement result deriving means deriving the measurement result information for the measurement item with use of the parameter specified by the parameter specifying information corresponding to the measurement item.

2. The biometric device according to claim 1,
wherein:
the measurement result deriving means calculates, from the one or more parameters specified by the parameter specifying information, an index indicative of the state of the living body, the state relating to the measurement item.

3. The biometric device according to claim 2, further comprising:
an index calculation rule storage section in which an index calculation rule for calculating, with use of the one or more parameters, the index corresponding to the measurement item is stored for each index,
wherein:
the index calculation rule includes information on a weight to be assigned to a parameter, the weight being assessed on a basis of a magnitude of an influence caused by said parameter on the index calculation; and
the measurement result deriving means, for the index calculation, assigns the weight to each of the one or more parameters in accordance with the index calculation rule, the weight being set for said each of the one or more parameters.

4. The biometric device according to claim 3, further comprising:
a parameter attribute storage section in which a parameter attribute indicative of the magnitude of the influence caused by said parameter on the index calculation is stored for each index and for each parameter,
wherein:
the weight, the information of which is included in the index calculation rule, correlates to all or part of information indicated by the parameter attribute.

5. The biometric device according to claim 4, further comprising:
parameter attribute managing means for, in accordance with an instruction that has been entered by a user into the biometric device and that intends to change the parameter attribute, changing the parameter attribute stored in the parameter attribute storage section,
wherein:
the parameter attribute managing means, in addition to the change to the parameter attribute stored in the parameter attribute storage section, changes the weight, the information of which is included in the index calculation rule.

6. The biometric device according to claim 2,
wherein:
the measurement method storage section further stores repeated measurement instruction information specifying timing for repeating the index calculation for each measurement item; and
the measurement result deriving means repeatedly calculates, at the timing specified by the repeated measurement instruction information, the index with use of the biometric parameter obtained on the basis of the biometric signal information obtained repeatedly.

7. The biometric device according to claim 6, further comprising:
state evaluating means for, on a basis of the index repeatedly calculated by the measurement result deriving means, evaluating a health state of the living body, the health state relating to the measurement item.

8. The biometric device according to claim 7,
wherein:
the state evaluating means, by comparing (i) an index calculated by the measurement result deriving means at a predetermined time point with (ii) a plurality of indexes repeatedly calculated by the measurement result deriving means, evaluates the health state of the living body, the health state being observed at the predetermined time point.

9. The biometric device according to claim 1,
wherein:
the measurement method storage section stores the parameter specifying information in such a manner that (i) a parameter essential to measurement and (ii) an auxiliary parameter that is preferably used in measurement are discriminated from each other.

10. The biometric device according to claim 1,
wherein:
the one or more parameters include (i) the biometric parameter reflecting a physiological state of the living body and (ii) an external parameter reflecting an environmental condition arising from outside the living body; and
the measurement method storage section stores the parameter specifying information in such a manner that the biometric parameter and the external parameter are discriminated from each other.

11. The biometric device according to claim 10,
wherein:
the external parameter includes at least one of (i) information on a specification of a biometric sensor for obtaining the biometric signal information from the living body, (ii) information on a position at which the biometric sensor is disposed, (iii) examinee information on the living body, and (iv) environment information on a measurement environment in which the living body is present;
the biometric parameter includes one or more biometric parameters;
the external parameter includes one or more external parameters; and
the measurement method storage section stores, as the parameter specifying information in correspondence with the measurement item, a combination of (i) the one or more biometric parameters and (ii) the one or more external parameters.

12. The biometric device according to claim 1,
wherein:
the biometric parameter includes (i) a parameter indicative of a change occurring inside the living body and (ii) a parameter indicative of a change appearing outside the living body.

13. The biometric device according to claim 1,
wherein:
the biometric parameter includes one or more biometric parameters; and
the one or more biometric parameters used by the measurement result deriving means are obtained through analysis of a single item of the biometric signal information.

14. The biometric device according to claim 1,
wherein:
the biometric parameter includes one or more biometric parameters; and
the one or more biometric parameters used by the measurement result deriving means are obtained through analysis of a plurality of items of the biometric signal information.

15. The biometric device according to claim 1, further comprising:
a communication section for communicating with a biometric sensor for obtaining the biometric signal information from the living body.

16. The biometric device according to claim 1,
wherein:
the biometric device is contained in a biometric sensor for obtaining the biometric signal information from the living body.

17. A biometric method for use by a biometric device for measuring a state of a living body with use of biometric signal information obtained from the living body,
(i) a measurement item measurable by the biometric device and (ii) parameter specifying information specifying one or more parameters for use in measurement of the measurement item being stored in the biometric device in correspondence with each other,
the parameter specifying information specifying at least one biometric parameter obtained on a basis of the biometric signal information,
the biometric method comprising the steps of:
(a) identifying the one or more parameters specified by the parameter specifying information corresponding to the measurement item; and
(b) deriving, with use of the one or more parameters identified in the step (a), measurement result information indicative of the state of the living body, the state relating to the measurement item.

18. (canceled)

19. A non-transitory computer-readable recording medium on which a control program for causing a computer to function as the means of the biometric device according to claim 1 is stored.
